SEQUENCE LISTING (126 sequences)

SEQ ID NO: 1 - DNA, 14351 bp, Mus musculus <s300> Genbank/AY004877.1 2000-07-28 (1)..(14351)
gccgcgtcgacagccggctccgctgtccgcagtcggccgcgggttagcgggcagagcgtctcctgtcgccggttcccctcgcttactgctctctcgtgccggaggccgctcctctcggctctcgctctctctctttctatctctctcccttccgcggatccgccatgtcggagccgggcggcggcgaggacggctcggcaggcctggaggtgtcggcggtgcagaatgtggcggacgtggcggtgctgcagaagcacctgcgtaagctggtgccgctgctgctggaggacggcggcgacgcgccggctgcgctggaggcggcgctggaggagaagagcgccctggagcagatgcgcaagttcctgtcagacccgcaggtccacacggtcctggtggagcgctccaccctcaaagaggacgttggtgatgaaggagaagaggagaaagaattcatttcctataacatcaacatagacattcattacggggtaaagtccaacagcttggcgttcatcaagcgagctcctgtgattgatgcagacaagccagtgtcgtcccagctccgagtcctcactctgagtgaagactcgccgtatgagacactgcactctttcatcagcaatgcagtggctcctttctttaagtcctacatcagagagtctggcaaagcagacagggatggtgataagatggctccttcagttgaaaaaaagattgcagagcttgaaatgggactcctccacctgcagcagaacattgagatcccagagatcagcttgccaattcatcccatcatcacgaacgttgcgaagcagtgctatgagcgcggagaaaagcccaaagttacagattttggtgataaggttgaagacccaactttccttaaccaattacagtctggagttaatcggtggatccgagaaattcaaaaggtgaccaagcttgaccgggatccagcatcaggaactgccttgcaggaaatcagcttttggctaaacttggaacgtgcgttgtaccgcatccaagagaagcgggagagccccgaggtgctcctgactctggacatcctgaagcatggcaaacgtttccatgccactgtcagttttgacactgacacaggtctgaagcaggcgttggaaacggtgaatgactacaaccctctgatgaaagacttccctctgaatgacttgctgtctgccacggagctggacaagataaggcaggcccttgttgcaattttcacacatctgagaaagatccgcaacacgaagtacccgattcagcgggccctgcgtctcgtggaggccatctccagagacctgagttctcagctgctcaaggtcttgggcactcggaagttgatgcatgtagcctatgaagagtttgaaaaggtcatggtggcttgcttcgaagtcttccagacgtgggacgacgagtacgagaagctccaggtgctgctgcgagacatcgtcaagaggaagcgggaggagaacctgaagatggtgtggcgcatcaaccccgctcacaggaagcttcaggcccgcctcgaccagatgaggaagttccgccgccagcacgagcagctgagggctgtcattgtcagggtcctgaggccacaggtcacagcagtcgcacaacagaaccaaggagaagcacctgaaccccaagacatgaaagtggccgaggtgctctttgatgctgccgacgccaacaccattgaggaggtgaacctggcctacgagaatgtcaaggaagtcgatggtctggatgtttccaaagaagggacggaggcctgggaggccgcgatgaagagatacgatgagaggatcgaccgtgtggagacccgcatcaccgcccgcctccgagatcagctcggcacggccaagaatgccaatgagatgttcaggattttctccaggttcaatgcactgttcgtccgcccacacatccgaggggccattcgtgaataccagacccagctgatccaacgtgtgaaagatgacatcgaatctctgcacgacaagttcaaggtccagtacccgcaaagccaagcttgtaaaatgagtcatgtccgtgacctgccccccgtgtcagggtctatcatctgggctaaacagatcgacagacagctcactgcctacatgaagcgagtggaggatgtcctgggcaaaggctgggagaaccacgtggaagggcagaagctgaagcaggatggagacagcttccgaatgaagctcaacacccaggagatcttcgatgactgggcgaggaaggttcagcagcgcaaccttggtgtctccgggcgcatcttcaccattgagagcgctcgcgtgcggggccgcactgggaatgtcctcaagctaaaggtgaacttccttccagagatcataacactctccaaagaagtccggaacctcaagtggcttggcttccgagtgccacttgcaattgtgaacaaggctcaccaagccaaccagctctacccatttgccatctcgctgattgaaagcgtccggacttacgagcgcacctgtgagaaggtggaggagcgcaataccatttccctgctagtggccggcctaaagaaggaggtgcaggccttaatcgcagaaggcatcgcgttggtgtgggagtcctacaagctcgacccctacgtgcagcgcctggcagagacggttttcaacttccaggagaaggtggacgacctactgattattgaggaaaagatagacctggaggtccgctccttggaaacctgcatgtatgaccacaagacattctcagagatcttgaacagagtgcagaaagcagtggacgacctaaacctgcactcctattccaatctgcccatctgggtcaacaagctggacatggagattgaaaggatactgggcgtccgcctgcaagctggccttcgagcttggacccaggttcttcttggacaagctgaggataaagcagaagtcgacatggatacagatgctccacaagtcagtcacaagcctggtggagagcccaagatcaaaaacgtggttcacgagctgaggataacaaatcaggtcatctatctgaacccacccattgaggagtgcaggtacaagctgtaccaggagatgttcgcgtggaagatggttgtgctgtccctgcccaggatccagagccagaggtaccaggtgggagtgcattacgagttgacggaggaagagaagttctatcggaatgcactgacaaggatgcccgacggccctgtcgccctggaagagtcctactctgcggtcatgggcatagtgactgaagttgagcagtatgttaaggtttggcttcagtaccagtgtttgtgggatatgcaggcagaaaacatttacaacaggctaggggaagatctcaacaagtggcaagctctcctggtccagataaggaaagccagaggaacctttgacaatgcggaaaccaagaaagagtttggtccggtggtgatagattacggcaaggtccagtccaaggtgaacttgaagtatgactcctggcataaggaggttctcagcaagtttgggcagatgctgggctcaaacatgacagaattccactcccagatctcaaagtctcgccaagagctggagcagcactccgttgacacagccagtacctctgacgcggtgaccttcatcacctatgtgcagtctctgaaacggaagatcaaacagtttgagaagcaagttgaactctaccgcaatggtcagcgtctgctggaaaaacaaaggttccagttcccgccttcttggctttacattgacaacatcgagggtgagtggggggccttcaatgacatcatgcggcggaaggactctgccattcagcaacaggtggcaaacctgcaaatgaagatcgtacaggaggacagagctgtggaaagccggaccacggatctgttgacagactgggagaagaccaaacctgtcacaggcaatctgcgtcctgaggaggctcttcaggccctcaccatatatgaagggaagttcggccggctgaaggacgacagagagaaatgtgcaaaggctaaggaagcgttggaactcacagacacaggcctcctcagtggcagtgaagagcgtgtacaggtggccttggaggagttacaagacctcaaaggtgtgtggtcagagctttctaaggtctgggaacaaattgatcaaatgaaggagcagccgtgggtctccgttcagcctcggaagcttcgacaaaacctagacggcctcttgaaccagctgaagaacttccctgcgaggcttcggcagtatgcttcctatgagtttgttcagaggctgctgaaaggctacatgaagataaacatgttggtaattgaactgaaatcagaagcactcaaagaccggcactggaaacagctgatgaagaggcttcatgttaactgggtggtttctgagctgacgctgggccagatctgggacgtggaccttcagaagaacgaagctgtcgtcaaggatgtgctgctggtggcacaaggcgagatggccttggaagaatttttgaagcagatacgagaagtgtggaacacatacgaactagacttggtcaactatcagaacaaatgccgcctcatccgaggctgggatgacctcttcaacaaggtcaaggagcatatcaacagcgtctctgccatgaaactctctccctattacaaggtttttgaagaagatgccctgagctgggaggacaagctgaaccgaatcatggctctctttgacgtgtggattgacgtgcagaggcgctgggtctacttggaaggcatcttcacaggcagtgcggacatcaaacatctgctgcctgtggaaacccagcgcttccagagtatcagcacggagtttttggctctgatgaaaaaagtctccaagtctccacttgtgatggatgttctgaacatacagggagtccagaggtctctggaaaggttggcagacctgctggggaagatccagaaagcattgggggagtacctggaacgggagcgctcgtctttccccaggttctactttgtgggtgatgaggatttgcttgagatcattgggaatagcaagaacgtggctaagttgcagaagcacttcaagaaaatgttcgccggcgtttccagcatcatcttaaatgaggataactctgtggtcctcggcatctcatcccgagaaggggaggaggttatgtttaaaactccagtgtccatcactgaacaccccaaaatcaatgagtggctcactctggtggaaaaggagatgagagtcaccttggctaaacttctggcagagtctgttacagaagtggagatttttggaaaagcaacttctattgacccaaatacctacattacttggattgataaataccaggcccagctagtggtattgtcagcccagatagcctggtccgagaacgtggagaatgcgctgagcaacgttgggggaggtggtgatgtggggcccttgcagtctgtgctgagcaatgtggaggtcacactcaacgtcttagcggactctgtcctcatggagcagcctcctctccggagacggaagctggagcacttgatcacagagctggttcaccaaagagacgttaccaggtccttgatcaaaagcaagatcgacaacgccaaatctttcgaatggctcagccagatgcgattttactttgaccctaagcaaacggacgtgttacagcagctgtccattcagatggccaatgccaagtttaactacggctttgagtacctgggtgttcaagacaagctggttcagacgcccctcactgaccgttgctatttgacaatgacacaagctttggaggccaggcttgggggctccccatttggacctgctggaaccggaaagacggagtccgtcaaagccctcggccatcagctggggcggttcgtcttagttttcaactgtgacgaaacctttgatttccaggcgatgggacgtatctttgtggggctttgccaagtgggcgcgtggggctgctttgatgagtttaaccgcctggaggagcggatgctctcagctgtatcccagcaggtgcagtgcatacaagaggcactgcgggagcattccaaccccaactacgacaagacctcagcccccattacttgtgagctgctgaacaaacaagtcaaggttagcccagacatggctatcttcatcaccatgaacccaggctatgcaggtcgctcaaacctccccgacaacctcaagaagctcttccggagcttggcgatgaccaagcctgaccggcagctgatcgcccaggtcatgctgtactcgcagggcttccggactgctgaggtccttgccaacaagatcgtcccgttcttcaaactgtgtgatgagcaactgtcttctcaaagccattatgactttgggcttcgggctttgaagagtgtgcttgtaagtgcaggcaatgtgaagagggagaggatccagaagataaagagggagaaagaagaacggggggaagcagttgatgaaggagagatcgctgagaacctacctgagcaggagattctgatccagagtgtctgcgagacaatggtgcccaagctggtggcagaagatatcccgctgctcttcagcctcctgtctgacgtgttccctggagtccagtaccaccgtggggagatgacagcccttcgagaagagctgaagaaagtgtgtcaggagatgtacttgacatatggcgacggagaagaagttggcgggatgtgggttgaaaaggttctccagctctatcagatcacgcagattaaccacggcctgatgatggttgggccttcaggaagtgggaagagcatggcctggcgagtgctgctgaaggctctagagaggcttgagggtgtggagggcgtggcccacataattgaccccaaggccatcagcaaagaccacctctacggaaccttggatcccaacaccagggagtggacagacggactcttcacacacgtgctgagaaagatcatagacaatgtgagaggtgagctgcagaaacgccagtggattgtctttgatggcgatgtggaccccgagtgggtggagaacctgaactccgtgctggatgacaacaagctcctgacgctgcctaatggagagcgccttagcctgcctcccaatgtgcggataatgtttgaagtgcaggacttgaaatacgcgacgctggccacggtgtctcgctgtggcatggtgtggttcagtgaggacgtcctgagcacagacatgatcttaaataacttcctggccaggctgcgcagcattcccctggatgaaggggaggacgaggcacagcgtaggcgaaaaggcaaagaagacgagggcgaagaggcggcttctccaatgctacagattcaaagagatgcagcaacgatcatgcagccgtacttcacgtccaacggcctggtcaccaaggcgctggagcacgccttcaagctggagcacatcatggacctgacgcgcctgcgctgcctgggctcgctcttctccatgctgcaccaggcctgccgcaacgtggcgcagtacaatgccaaccatcctgatttccccatgcagattgagcagctggagcgctacattcagcgctatctggtttacgccatcctctggtccttatctggagacagccggctgaagatgagagcagagctgggcgagtacatccggaggatcaccactgtgccgctgcccactgcgcccaacgtccccatcattgactatgaggtgtccatcagtggagagtggtcgccgtggcaggccaaggtgcctcagattgaagtggagacacacaaggtggcagccccagatgtcgtcgtgccaaccttagatacagtccgccatgaagctctcctgtacacttggctggctgaacacaaacccctggtcttatgtggccctcctgggtctggcaagaccatgacactcttcagtgctctccgggccttgcctgacatggaggttgtgggcctaaacttctcaagtgccactacaccagagctgttgctgaagacgtttgaccactactgcgaatacaggcgcacacctaacggtgttgtcttggctcctgttcagcttggaaagtggctggtcttgttctgtgatgaaatcaacttgcccgatatggataaatacggcacacagagagtcatctccttcatcagacagatggtggagcatggagccttctatcgtacctcagaccaaacgtgggtaaagctagagagaatccagtttgttggggcttgcaatccacccactgaccctgggagaaagcccctctcacacaggttcttgcgccacgtgcctgttgtatatgtggattacccaggaccagcctccctgacgcaaatctatggcaccttcaaccgcgccatgctgaggctcattccctctctccgtacctatgcagaacccctcactgccgccatggtggagttctataccatgtcacaggagcggtttactcaggacacacagccacactatatctactcgccccgtgagatgactcggtgggtaagaggaatctttgaagcactgagacccctggagacattgcctgttgaaggtctcattcgtatttgggcacatgaagccctccgtctcttccaagacaggcttgtggaggatgaggagcggcgctggactgacgaaaacattgacatggttgccttgaagcacttccctaacatagacaaagagaaagccatgagccggcctatcctgtacagcaactggctgtcaaaggattatatcccagttgaccaagaagagctaagggattatgtcaaggctaggctgaaggtcttctatgaagaagaacttgatgttcccttggtcttgttcaatgaagttctggaccatgtgctgaggatagacagaatattccgacaacctcaaggccatctgcttctgattggtgttagtggagctggaaaaaccaccctgtctcgctttgtcgcctggatgaatggcttgagtgtataccagattaaggtccacaggaagtacactggggaagacttcgatgaagatcttaggacagtgctgagacggtctggctgtaaaaatgaaaagatagcattcataatggatgagtccaacgtcttagattccggattcctggaacgaatgaatacccttctggctaatggcgaggttcctgggctctttgaaggggatgagtacgccacgctgatgacacagtgcaaagagggggcccagaaggaggggctcatgctggactcccacgaggagctctacaagtggttcaccagccaggtgatccgcaacctgcacgtggtcttcaccatgaacccgtcctcagaggggctcaaggacagggcggccacctcaccagcccttttcaacaggtgtgtgctgaactggtttggtgactggtccactgaagcactgtatcaagtcggcaaagaattcaccagtaagatggacctggagaagccgaactatatagtgcctgactacatgccagttgtgtatgataaactgccgcagcccccaacacaccgggaagccattgtgaacagctgtgtgtttgtccatcagactcttcaccaggctaatgcgcgcctggcgaagcgaggtggcagaacgatggccatcacacctcggcactacctggacttcatcaaccactacgccaacctcttccatgagaagcgcagcgagttggaggagcagcagatgcacctgaatgttgggctccgaaaaatcaaagagaccgtggaccaggtggaagaactgcgccgtgacctgaggatcaagagccaagagctggaggtcaagaacgcagcggccaatgacaagctgaagaaaatggtgaaagaccagcaagaggctgagaagaagaaggtcatgagccaagaaatccaggagcagctacacaaacagcaggaagtgatcgcagacaagcagatgagtgtcaaggaggacctggataaagtggagcctgctgtcatcgaggcccagaacgctgtgaagtccatcaagaagcagcacctggtggaggtgaggtccatggccaaccctcctgcagctgtgaagctggctctggagtccatctgcctgctgcttggtgagagcaccacggactggaagcagatccgctccatcatcatgagagagaacttcatccccaccatcgtcaacttctcggctgaggagatcagtgatgccatacgagagaagatgaagaaaaactacatgtccaaccccagttacaactatgagatcgtcaaccgggcttccctggcttgtggtcctatggtgaagtgggcgattgcacagctcaattatgcagacatgttaaagcgagtggagcccctgaggaatgagctgcagaagctggaagatgacgccaaggacaaccagcagaaagccaatgaggtggagcagatgatcagggacctggaagccagcattgcccgctacaaggaggagtacgctgtcctcatctctgaggcccaggccatcaaggcagacctggcagctgtggaagcaaaggtaaaccggagcactgcacttctaaagagtttatctgccgaaagggaacgttgggaaaagacgagtgaaactttcaaaaaccagatgtctaccattgctggggactgcctcctgtcggcggccttcattgcttacgctggctactttgaccagcaaatgcgccagaacttgttcaccacctggtctcatcacttacagcaagccaacatccagttccgcacagacattgcaaggacggagtacctctccaatgctgacgaacgcctccgctggcaggccagctctctgcctgccgacgacctgtgcacagagaatgccatcatgctaaaacgattcaataggtacccgctgatcatcgacccctccggacaggccacagaattcatcatgaatgaatataaggaccggaagatcacaaggaccagcttcttagatgatgccttccggaagaatttggagagtgctctgagatttggcaatccccttcttgtccaggatgtggaaagctacgatccagttttgaatccagtactgaaccgtgaagttcggcgaacaggagggagggtgctgattaccctcggtgaccaagacatagacctgtcaccatcctttgtcatctttctgtctacccgagacccaactgtggagttcccaccagacctctgctcccgggtgacctttgtgaacttcaccgtcacccgcagcagtttacagagccagtgtctcaatgaagtgcttaaagcagaaaggccagacgtggacgagaagcgctccgacctcctgaaactgcaaggggagttccaactccgtttacggcagcttgaaaagtctctgctccaagctctgaatgaggtaaaaggccgcattctggttgacgacaccatcattacaaccctggagaatctgaagcgggaagctgccgaggttaccagaaaggtggaggagacagatattgtcatgcaggaggtggagaccgtgtcccagcagtacctgccgctgtccactgcctgcagcagcatctacttcactatggagtccttgaagcaggttcacttcctgtaccagtactctctgcagttcttcctggacatctaccacaacgtgctgtatgagaaccccaacctgaagggtgccaccgatcacacacagcgcctctccgtcataaccaaggacctcttccaggtggcctttaaccgagtggctcgaggcatgctgcaccaggaccacatcaccttcgccatgctgctagcgaggatcaagctgaaaggcaccgtgggggagcccacctatgatgcagagttccagcacttcctcagagggaaggagattgtgctgagtgctggctccacccccaaaatccagggcctgaccgtagagcaggcagaagctgtcgtgaggttgagctgtctgcctgcatttaaagatctgattgccaaggtgcaggcagatgagcagtttggcatttggctggatagcagttcaccagagcagaccgtgccatacctctggagcgaggaaacgcctacaactcccattgggcaggccatccaccgcctgctcctgattcaagccttccgacccgaccgcctgttggccatggcccacatgtttgtttccacaaacctcggagagtccttcatgtccatcatggagcagcctctcgacctcacccacattgtgggcacagaggtgaagccaaacacacctgtcctgatgtgctctgtgcctgggtatgatgccagcggacatgttgaggacctcgcagcagagcagaacacgcagatcacgtctattgccatcggctcagcagaaggctttaaccaagcagataaggccatcaacaccgccgtgaagtcaggcaggtgggtgatgttgaagaacgtgcacctggccccgggctggctgatgcagctggagaagaagctgcactccctgcagccccacgcctgcttccgacttttcctcaccatggagatcaaccccaaggtgcccgtgaacctgctccgtgctggtcggatctttgtgtttgagccaccacccggagtgaaagccaacatgctgaggacatttagcagcatccctgtctctaggatatgcaagtctccaaatgagcgcgcccgcttgtatttcctgctggcttggttccatgccatcatccaagagcgcctgcgctatgctccactggggtggtcaaagaagtacgaatttggagaatctgaccttcgatcagcttgtgacacggtggacacgtggctggacgacacagccaagggccggcagaacatctcaccggataagatcccatggtctgccctgaagaccctgatggcgcagtccatctatggtgggcgggtggacaatgagttcgaccagcgtctgctcaacaccttcctggagcggctgttcaccacccgcagcttcgacagtgaattcaagctggcgtgcaaggttgacgggcacaaggacatccagatgcctgatggtatcaggcgagaggagtttgtgcagtgggtggagctgctgcctgacgcccagacaccctcatggctgggcctccccaacaatgctgaaagggtcctgcttaccacccaaggcgtggacatgatcagcaagatgctgaagatgcagatgctggaggacgaggatgacctggcctacgcagagacggagaagaaggcaaggacggactccacctccgatggccgtccagcctggatgaggacactgcacaccacggcgtccaactggctgcacctcatcccacagacactgagccccctgaagcgcaccgtggagaacatcaaggatcctttgtttaggttctttgagagagaagtaaagatgggggccaagttactccaagatgtgcgccaagaccttgctgacgtcgttcaagtgtgcgaggggaaaaagaagcagacaaactacctgcgcacgctcatcaatgagctggtgaaagggatcttgcctcggagctggtcccattacacagtgcctgcaggcatgacggtgatccagtgggtgtcagatttcagcgagaggatcaaacagctgcagaacatctcacaagcagctgcatctggtggtgccaaggaactgaagaacatccacgtgtgcctgggtggcctgtttgtacctgaggcctacatcactgccaccagacagtatgtggcacaagctaacagctggtccctggaggaactctgcctggaagtcaatgtcaccgcctcacagagtgccacccttgatgcctgcagctttggagtcacaggtctgaagctccaaggggccacgtgcagcaacaacaagctatcactctccaatgccatctccacagtcctccccctcacacagctgcgctgggtgaaacaaacaagcgccgagaagaaggccagcgtggtgaccttacctgtctacctgaacttcacccgggcagacctcatcttcaccgtggactttgaaattgctacaaaggaagatcctcgcagcttctatgagcgtggagttgccgttctgtgcactgagtaaagctagacttttctccagtcctctttctggaataggaaactcccaatatttaaccttcattcatccttaacggtgtggaagggttgatggactggaaagcaagtggttggttcgcgttggcgggtgacaggtgctgcggttggagacggtgggatgggagaagagaagagtgcgctgtccgagggctgaccgaatggctgggtttagtaaataaaacactaagcatgacgggcaaaaaaaaaaaaaaaaaaa

SEQ ID NO: 2 - protein, 4644 aa, Mus musculus <s300> Genbank/AAF91078 2000-07-28 (1)..(4644)
MSEPGGGEDGSAGLEVSAVQNVADVAVLQKHLRKLVPLLLEDGGDAPAALEAALEEKSALEQMRKFLSDPQVHTVLVERSTLKEDVGDEGEEEKEFISYNINIDIHYGVKSNSLAFIKRAPVIDADKPVSSQLRVLTLSEDSPYETLHSFISNAVAPFFKSYIRESGKADRDGDKMAPSVEKKIAELEMGLLHLQQNIEIPEISLPIHPIITNVAKQCYERGEKPKVTDFGDKVEDPTFLNQLQSGVNRWIREIQKVTKLDRDPASGTALQEISFWLNLERALYRIQEKRESPEVLLTLDILKHGKRFHATVSFDTDTGLKQALETVNDYNPLMKDFPLNDLLSATELDKIRQALVAIFTHLRKIRNTKYPIQRALRLVEAISRDLSSQLLKVLGTRKLMHVAYEEFEKVMVACFEVFQTWDDEYEKLQVLLRDIVKRKREENLKMVWRINPAHRKLQARLDQMRKFRRQHEQLRAVIVRVLRPQVTAVAQQNQGEAPEPQDMKVAEVLFDAADANTIEEVNLAYENVKEVDGLDVSKEGTEAWEAAMKRYDERIDRVETRITARLRDQLGTAKNANEMFRIFSRFNALFVRPHIRGAIREYQTQLIQRVKDDIESLHDKFKVQYPQSQACKMSHVRDLPPVSGSIIWAKQIDRQLTAYMKRVEDVLGKGWENHVEGQKLKQDGDSFRMKLNTQEIFDDWARKVQQRNLGVSGRIFTIESARVRGRTGNVLKLKVNFLPEIITLSKEVRNLKWLGFRVPLAIVNKAHQANQLYPFAISLIESVRTYERTCEKVEERNTISLLVAGLKKEVQALIAEGIALVWESYKLDPYVQRLAETVFNFQEKVDDLLIIEEKIDLEVRSLETCMYDHKTFSEILNRVQKAVDDLNLHSYSNLPIWVNKLDMEIERILGVRLQAGLRAWTQVLLGQAEDKAEVDMDTDAPQVSHKPGGEPKIKNVVHELRITNQVIYLNPPIEECRYKLYQEMFAWKMVVLSLPRIQSQRYQVGVHYELTEEEKFYRNALTRMPDGPVALEESYSAVMGIVTEVEQYVKVWLQYQCLWDMQAENIYNRLGEDLNKWQALLVQIRKARGTFDNAETKKEFGPVVIDYGKVQSKVNLKYDSWHKEVLSKFGQMLGSNMTEFHSQISKSRQELEQHSVDTASTSDAVTFITYVQSLKRKIKQFEKQVELYRNGQRLLEKQRFQFPPSWLYIDNIEGEWGAFNDIMRRKDSAIQQQVANLQMKIVQEDRAVESRTTDLLTDWEKTKPVTGNLRPEEALQALTIYEGKFGRLKDDREKCAKAKEALELTDTGLLSGSEERVQVALEELQDLKGVWSELSKVWEQIDQMKEQPWVSVQPRKLRQNLDGLLNQLKNFPARLRQYASYEFVQRLLKGYMKINMLVIELKSEALKDRHWKQLMKRLHVNWVVSELTLGQIWDVDLQKNEAVVKDVLLVAQGEMALEEFLKQIREVWNTYELDLVNYQNKCRLIRGWDDLFNKVKEHINSVSAMKLSPYYKVFEEDALSWEDKLNRIMALFDVWIDVQRRWVYLEGIFTGSADIKHLLPVETQRFQSISTEFLALMKKVSKSPLVMDVLNIQGVQRSLERLADLLGKIQKALGEYLERERSSFPRFYFVGDEDLLEIIGNSKNVAKLQKHFKKMFAGVSSIILNEDNSVVLGISSREGEEVMFKTPVSITEHPKINEWLTLVEKEMRVTLAKLLAESVTEVEIFGKATSIDPNTYITWIDKYQAQLVVLSAQIAWSENVENALSNVGGGGDVGPLQSVLSNVEVTLNVLADSVLMEQPPLRRRKLEHLITELVHQRDVTRSLIKSKIDNAKSFEWLSQMRFYFDPKQTDVLQQLSIQMANAKFNYGFEYLGVQDKLVQTPLTDRCYLTMTQALEARLGGSPFGPAGTGKTESVKALGHQLGRFVLVFNCDETFDFQAMGRIFVGLCQVGAWGCFDEFNRLEERMLSAVSQQVQCIQEALREHSNPNYDKTSAPITCELLNKQVKVSPDMAIFITMNPGYAGRSNLPDNLKKLFRSLAMTKPDRQLIAQVMLYSQGFRTAEVLANKIVPFFKLCDEQLSSQSHYDFGLRALKSVLVSAGNVKRERIQKIKREKEERGEAVDEGEIAENLPEQEILIQSVCETMVPKLVAEDIPLLFSLLSDVFPGVQYHRGEMTALREELKKVCQEMYLTYGDGEEVGGMWVEKVLQLYQITQINHGLMMVGPSGSGKSMAWRVLLKALERLEGVEGVAHIIDPKAISKDHLYGTLDPNTREWTDGLFTHVLRKIIDNVRGELQKRQWIVFDGDVDPEWVENLNSVLDDNKLLTLPNGERLSLPPNVRIMFEVQDLKYATLATVSRCGMVWFSEDVLSTDMILNNFLARLRSIPLDEGEDEAQRRRKGKEDEGEEAASPMLQIQRDAATIMQPYFTSNGLVTKALEHAFKLEHIMDLTRLRCLGSLFSMLHQACRNVAQYNANHPDFPMQIEQLERYIQRYLVYAILWSLSGDSRLKMRAELGEYIRRITTVPLPTAPNVPIIDYEVSISGEWSPWQAKVPQIEVETHKVAAPDVVVPTLDTVRHEALLYTWLAEHKPLVLCGPPGSGKTMTLFSALRALPDMEVVGLNFSSATTPELLLKTFDHYCEYRRTPNGVVLAPVQLGKWLVLFCDEINLPDMDKYGTQRVISFIRQMVEHGAFYRTSDQTWVKLERIQFVGACNPPTDPGRKPLSHRFLRHVPVVYVDYPGPASLTQIYGTFNRAMLRLIPSLRTYAEPLTAAMVEFYTMSQERFTQDTQPHYIYSPREMTRWVRGIFEALRPLETLPVEGLIRIWAHEALRLFQDRLVEDEERRWTDENIDMVALKHFPNIDKEKAMSRPILYSNWLSKDYIPVDQEELRDYVKARLKVFYEEELDVPLVLFNEVLDHVLRIDRIFRQPQGHLLLIGVSGAGKTTLSRFVAWMNGLSVYQIKVHRKYTGEDFDEDLRTVLRRSGCKNEKIAFIMDESNVLDSGFLERMNTLLANGEVPGLFEGDEYATLMTQCKEGAQKEGLMLDSHEELYKWFTSQVIRNLHVVFTMNPSSEGLKDRAATSPALFNRCVLNWFGDWSTEALYQVGKEFTSKMDLEKPNYIVPDYMPVVYDKLPQPPTHREAIVNSCVFVHQTLHQANARLAKRGGRTMAITPRHYLDFINHYANLFHEKRSELEEQQMHLNVGLRKIKETVDQVEELRRDLRIKSQELEVKNAAANDKLKKMVKDQQEAEKKKVMSQEIQEQLHKQQEVIADKQMSVKEDLDKVEPAVIEAQNAVKSIKKQHLVEVRSMANPPAAVKLALESICLLLGESTTDWKQIRSIIMRENFIPTIVNFSAEEISDAIREKMKKNYMSNPSYNYEIVNRASLACGPMVKWAIAQLNYADMLKRVEPLRNELQKLEDDAKDNQQKANEVEQMIRDLEASIARYKEEYAVLISEAQAIKADLAAVEAKVNRSTALLKSLSAERERWEKTSETFKNQMSTIAGDCLLSAAFIAYAGYFDQQMRQNLFTTWSHHLQQANIQFRTDIARTEYLSNADERLRWQASSLPADDLCTENAIMLKRFNRYPLIIDPSGQATEFIMNEYKDRKITRTSFLDDAFRKNLESALRFGNPLLVQDVESYDPVLNPVLNREVRRTGGRVLITLGDQDIDLSPSFVIFLSTRDPTVEFPPDLCSRVTFVNFTVTRSSLQSQCLNEVLKAERPDVDEKRSDLLKLQGEFQLRLRQLEKSLLQALNEVKGRILVDDTIITTLENLKREAAEVTRKVEETDIVMQEVETVSQQYLPLSTACSSIYFTMESLKQVHFLYQYSLQFFLDIYHNVLYENPNLKGATDHTQRLSVITKDLFQVAFNRVARGMLHQDHITFAMLLARIKLKGTVGEPTYDAEFQHFLRGKEIVLSAGSTPKIQGLTVEQAEAVVRLSCLPAFKDLIAKVQADEQFGIWLDSSSPEQTVPYLWSEETPTTPIGQAIHRLLLIQAFRPDRLLAMAHMFVSTNLGESFMSIMEQPLDLTHIVGTEVKPNTPVLMCSVPGYDASGHVEDLAAEQNTQITSIAIGSAEGFNQADKAINTAVKSGRWVMLKNVHLAPGWLMQLEKKLHSLQPHACFRLFLTMEINPKVPVNLLRAGRIFVFEPPPGVKANMLRTFSSIPVSRICKSPNERARLYFLLAWFHAIIQERLRYAPLGWSKKYEFGESDLRSACDTVDTWLDDTAKGRQNISPDKIPWSALKTLMAQSIYGGRVDNEFDQRLLNTFLERLFTTRSFDSEFKLACKVDGHKDIQMPDGIRREEFVQWVELLPDAQTPSWLGLPNNAERVLLTTQGVDMISKMLKMQMLEDEDDLAYAETEKKARTDSTSDGRPAWMRTLHTTASNWLHLIPQTLSPLKRTVENIKDPLFRFFEREVKMGAKLLQDVRQDLADVVQVCEGKKKQTNYLRTLINELVKGILPRSWSHYTVPAGMTVIQWVSDFSERIKQLQNISQAAASGGAKELKNIHVCLGGLFVPEAYITATRQYVAQANSWSLEELCLEVNVTASQSATLDACSFGVTGLKLQGATCSNNKLSLSNAISTVLPLTQLRWVKQTSAEKKASVVTLPVYLNFTRADLIFTVDFEIATKEDPRSFYERGVAVLCTE

SEQ ID NO: 3 - DNA, 14351 bp, Mus musculus
gccgcgtcgacagccggctccgctgtccgcagtcggccgcgggttagcgggcagagcgtctcctgtcgccggttcccctcgcttactgctctctcgtgccggaggccgctcctctcggctctcgctctctctctttctatctctctcccttccgcggatccgccatgtcggagccgggcggcggcgaggacggctcggcaggcctggaggtgtcggcggtgcagaatgtggcggacgtggcggtgctgcagaagcacctgcgtaagctggtgccgctgctgctggaggacggcggcgacgcgccggctgcgctggaggcggcgctggaggagaagagcgccctggagcagatgcgcaagttcctgtcagacccgcaggtccacacggtcctggtggagcgctccaccctcaaagaggacgttggtgatgaaggagaagaggagaaagaattcatttcctataacatcaacatagacattcattacggggtaaagtccaacagcttggcgttcatcaagcgagctcctgtgattgatgcagacaagccagtgtcgtcccagctccgagtcctcactctgagtgaagactcgccgtatgagacactgcactctttcatcagcaatgcagtggctcctttctttaagtcctacatcagagagtctggcaaagcagacagggatggtgataagatggctccttcagttgaaaaaaagattgcagagcttgaaatgggactcctccacctgcagcagaacattgagatcccagagatcagcttgccaattcatcccatcatcacgaacgttgcgaagcagtgctatgagcgcggagaaaagcccaaagttacagattttggtgataaggttgaagacccaactttccttaaccaattacagtctggagttaatcggtggatccgagaaattcaaaaggtgaccaagcttgaccgggatccagcatcaggaactgccttgcaggaaatcagcttttggctaaacttggaacgtgcgttgtaccgcatccaagagaagcgggagagccccgaggtgctcctgactctggacatcctgaagcatggcaaacgtttccatgccactgtcagttttgacactgacacaggtctgaagcaggcgttggaaacggtgaatgactacaaccctctgatgaaagacttccctctgaatgacttgctgtctgccacggagctggacaagataaggcaggcccttgttgcaattttcacacatctgagaaagatccgcaacacgaagtacccgattcagcgggccctgcgtctcgtggaggccatctccagagacctgagttctcagctgctcaaggtcttgggcactcggaagttgatgcatgtagcctatgaagagtttgaaaaggtcatggtggcttgcttcgaagtcttccagacgtgggacgacgagtacgagaagctccaggtgctgctgcgagacatcgtcaagaggaagcgggaggagaacctgaagatggtgtggcgcatcaaccccgctcacaggaagcttcaggcccgcctcgaccagatgaggaagttccgccgccagcacgagcagctgagggctgtcattgtcagggtcctgaggccacaggtcacagcagtcgcacaacagaaccaaggagaagcacctgaaccccaagacatgaaagtggccgaggtgctctttgatgctgccgacgccaacaccattgaggaggtgaacctggcctacgagaatgtcaaggaagtcgatggtctggatgtttccaaagaagggacggaggcctgggaggccgcgatgaagagatacgatgagaggatcgaccgtgtggagacccgcatcaccgcccgcctccgagatcagctcggcacggccaagaatgccaatgagatgttcaggattttctccaggttcaatgcactgttcgtccgcccacacatccgaggggccattcgtgaataccagacccagctgatccaacgtgtgaaagatgacatcgaatctctgcacgacaagttcaaggtccagtacccgcaaagccaagcttgtaaaatgagtcatgtccgtgacctgccccccgtgtcagggtctatcatctgggctaaacagatcgacagacagctcactgcctacatgaagcgagtggaggatgtcctgggcaaaggctgggagaaccacgtggaagggcagaagctgaagcaggatggagacagcttccgaatgaagctcaacacccaggagatcttcgatgactgggcgaggaaggttcagcagcgcaaccttggtgtctccgggcgcatcttcaccattgagagcgctcgcgtgcggggccgcactgggaatgtcctcaagctaaaggtgaacttccttccagagatcataacactctccaaagaagtccggaacctcaagtggcttggcttccgagtgccacttgcaattgtgaacaaggctcaccaagccaaccagctctacccatttgccatctcgctgattgaaagcgtccggacttacgagcgcacctgtgagaaggtggaggagcgcaataccatttccctgctagtggccggcctaaagaaggaggtgcaggccttaatcgcagaaggcatcgcgttggtgtgggagtcctacaagctcgacccctacgtgcagcgcctggcagagacggttttcaacttccaggagaaggtggacgacctactgattattgaggaaaagatagacctggaggtccgctccttggaaacctgcatgtatgaccacaagacattctcagagatcttgaacagagtgcagaaagcagtggacgacctaaacctgcactcctattccaatctgcccatctgggtcaacaagctggacatggagattgaaaggatactgggcgtccgcctgcaagctggccttcgagcttggacccaggttcttcttggacaagctgaggataaagcagaagtcgacatggatacagatgctccacaagtcagtcacaagcctggtggagagcccaagatcaaaaacgtggttcacgagctgaggataacaaatcaggtcatctatctgaacccacccattgaggagtgcaggtacaagctgtaccaggagatgttcgcgtggaagatggttgtgctgtccctgcccaggatccagagccagaggtaccaggtgggagtgcattacgagttgacggaggaagagaagttctatcggaatgcactgacaaggatgcccgacggccctgtcgccctggaagagtcctactctgcggtcatgggcatagtgactgaagttgagcagtatgttaaggtttggcttcagtgccagtgtttgtgggatatgcaggcagaaaacatttacaacaggctaggggaagatctcaacaagtggcaagctctcctggtccagataaggaaagccagaggaacctttgacaatgcggaaaccaagaaagagtttggtccggtggtgatagattacggcaaggtccagtccaaggtgaacttgaagtatgactcctggcataaggaggttctcagcaagtttgggcagatgctgggctcaaacatgacagaattccactcccagatctcaaagtctcgccaagagctggagcagcactccgttgacacagccagtacctctgacgcggtgaccttcatcacctatgtgcagtctctgaaacggaagatcaaacagtttgagaagcaagttgaactctaccgcaatggtcagcgtctgctggaaaaacaaaggttccagttcccgccttcttggctttacattgacaacatcgagggtgagtggggggccttcaatgacatcatgcggcggaaggactctgccattcagcaacaggtggcaaacctgcaaatgaagatcgtacaggaggacagagctgtggaaagccggaccacggatctgttgacagactgggagaagaccaaacctgtcacaggcaatctgcgtcctgaggaggctcttcaggccctcaccatatatgaagggaagttcggccggctgaaggacgacagagagaaatgtgcaaaggctaaggaagcgttggaactcacagacacaggcctcctcagtggcagtgaagagcgtgtacaggtggccttggaggagttacaagacctcaaaggtgtgtggtcagagctttctaaggtctgggaacaaattgatcaaatgaaggagcagccgtgggtctccgttcagcctcggaagcttcgacaaaacctagacggcctcttgaaccagctgaagaacttccctgcgaggcttcggcagtatgcttcctatgagtttgttcagaggctgctgaaaggctacatgaagataaacatgttggtaattgaactgaaatcagaagcactcaaagaccggcactggaaacagctgatgaagaggcttcatgttaactgggtggtttctgagctgacgctgggccagatctgggacgtggaccttcagaagaacgaagctgtcgtcaaggatgtgctgctggtggcacaaggcgagatggccttggaagaatttttgaagcagatacgagaagtgtggaacacatacgaactagacttggtcaactatcagaacaaatgccgcctcatccgaggctgggatgacctcttcaacaaggtcaaggagcatatcaacagcgtctctgccatgaaactctctccctattacaaggtttttgaagaagatgccctgagctgggaggacaagctgaaccgaatcatggctctctttgacgtgtggattgacgtgcagaggcgctgggtctacttggaaggcatcttcacaggcagtgcggacatcaaacatctgctgcctgtggaaacccagcgcttccagagtatcagcacggagtttttggctctgatgaaaaaagtctccaagtctccacttgtgatggatgttctgaacatacagggagtccagaggtctctggaaaggttggcagacctgctggggaagatccagaaagcattgggggagtacctggaacgggagcgctcgtctttccccaggttctactttgtgggtgatgaggatttgcttgagatcattgggaatagcaagaacgtggctaagttgcagaagcacttcaagaaaatgttcgccggcgtttccagcatcatcttaaatgaggataactctgtggtcctcggcatctcatcccgagaaggggaggaggttatgtttaaaactccagtgtccatcactgaacaccccaaaatcaatgagtggctcactctggtggaaaaggagatgagagtcaccttggctaaacttctggcagagtctgttacagaagtggagatttttggaaaagcaacttctattgacccaaatacctacattacttggattgataaataccaggcccagctagtggtattgtcagcccagatagcctggtccgagaacgtggagaatgcgctgagcaacgttgggggaggtggtgatgtggggcccttgcagtctgtgctgagcaatgtggaggtcacactcaacgtcttagcggactctgtcctcatggagcagcctcctctccggagacggaagctggagcacttgatcacagagctggttcaccaaagagacgttaccaggtccttgatcaaaagcaagatcgacaacgccaaatctttcgaatggctcagccagatgcgattttactttgaccctaagcaaacggacgtgttacagcagctgtccattcagatggccaatgccaagtttaactacggctttgagtacctgggtgttcaagacaagctggttcagacgcccctcactgaccgttgctatttgacaatgacacaagctttggaggccaggcttgggggctccccatttggacctgctggaaccggaaagacggagtccgtcaaagccctcggccatcagctggggcggttcgtcttagttttcaactgtgacgaaacctttgatttccaggcgatgggacgtatctttgtggggctttgccaagtgggcgcgtggggctgctttgatgagtttaaccgcctggaggagcggatgctctcagctgtatcccagcaggtgcagtgcatacaagaggcactgcgggagcattccaaccccaactacgacaagacctcagcccccattacttgtgagctgctgaacaaacaagtcaaggttagcccagacatggctatcttcatcaccatgaacccaggctatgcaggtcgctcaaacctccccgacaacctcaagaagctcttccggagcttggcgatgaccaagcctgaccggcagctgatcgcccaggtcatgctgtactcgcagggcttccggactgctgaggtccttgccaacaagatcgtcccgttcttcaaactgtgtgatgagcaactgtcttctcaaagccattatgactttgggcttcgggctttgaagagtgtgcttgtaagtgcaggcaatgtgaagagggagaggatccagaagataaagagggagaaagaagaacggggggaagcagttgatgaaggagagatcgctgagaacctacctgagcaggagattctgatccagagtgtctgcgagacaatggtgcccaagctggtggcagaagatatcccgctgctcttcagcctcctgtctgacgtgttccctggagtccagtaccaccgtggggagatgacagcccttcgagaagagctgaagaaagtgtgtcaggagatgtacttgacatatggcgacggagaagaagttggcgggatgtgggttgaaaaggttctccagctctatcagatcacgcagattaaccacggcctgatgatggttgggccttcaggaagtgggaagagcatggcctggcgagtgctgctgaaggctctagagaggcttgagggtgtggagggcgtggcccacataattgaccccaaggccatcagcaaagaccacctctacggaaccttggatcccaacaccagggagtggacagacggactcttcacacacgtgctgagaaagatcatagacaatgtgagaggtgagctgcagaaacgccagtggattgtctttgatggcgatgtggaccccgagtgggtggagaacctgaactccgtgctggatgacaacaagctcctgacgctgcctaatggagagcgccttagcctgcctcccaatgtgcggataatgtttgaagtgcaggacttgaaatacgcgacgctggccacggtgtctcgctgtggcatggtgtggttcagtgaggacgtcctgagcacagacatgatcttaaataacttcctggccaggctgcgcagcattcccctggatgaaggggaggacgaggcacagcgtaggcgaaaaggcaaagaagacgagggcgaagaggcggcttctccaatgctacagattcaaagagatgcagcaacgatcatgcagccgtacttcacgtccaacggcctggtcaccaaggcgctggagcacgccttcaagctggagcacatcatggacctgacgcgcctgcgctgcctgggctcgctcttctccatgctgcaccaggcctgccgcaacgtggcgcagtacaatgccaaccatcctgatttccccatgcagattgagcagctggagcgctacattcagcgctatctggtttacgccatcctctggtccttatctggagacagccggctgaagatgagagcagagctgggcgagtacatccggaggatcaccactgtgccgctgcccactgcgcccaacgtccccatcattgactatgaggtgtccatcagtggagagtggtcgccgtggcaggccaaggtgcctcagattgaagtggagacacacaaggtggcagccccagatgtcgtcgtgccaaccttagatacagtccgccatgaagctctcctgtacacttggctggctgaacacaaacccctggtcttatgtggccctcctgggtctggcaagaccatgacactcttcagtgctctccgggccttgcctgacatggaggttgtgggcctaaacttctcaagtgccactacaccagagctgttgctgaagacgtttgaccactactgcgaatacaggcgcacacctaacggtgttgtcttggctcctgttcagcttggaaagtggctggtcttgttctgtgatgaaatcaacttgcccgatatggataaatacggcacacagagagtcatctccttcatcagacagatggtggagcatggagccttctatcgtacctcagaccaaacgtgggtaaagctagagagaatccagtttgttggggcttgcaatccacccactgaccctgggagaaagcccctctcacacaggttcttgcgccacgtgcctgttgtatatgtggattacccaggaccagcctccctgacgcaaatctatggcaccttcaaccgcgccatgctgaggctcattccctctctccgtacctatgcagaacccctcactgccgccatggtggagttctataccatgtcacaggagcggtttactcaggacacacagccacactatatctactcgccccgtgagatgactcggtgggtaagaggaatctttgaagcactgagacccctggagacattgcctgttgaaggtctcattcgtatttgggcacatgaagccctccgtctcttccaagacaggcttgtggaggatgaggagcggcgctggactgacgaaaacattgacatggttgccttgaagcacttccctaacatagacaaagagaaagccatgagccggcctatcctgtacagcaactggctgtcaaaggattatatcccagttgaccaagaagagctaagggattatgtcaaggctaggctgaaggtcttctatgaagaagaacttgatgttcccttggtcttgttcaatgaagttctggaccatgtgctgaggatagacagaatattccgacaacctcaaggccatctgcttctgattggtgttagtggagctggaaaaaccaccctgtctcgctttgtcgcctggatgaatggcttgagtgtataccagattaaggtccacaggaagtacactggggaagacttcgatgaagatcttaggacagtgctgagacggtctggctgtaaaaatgaaaagatagcattcataatggatgagtccaacgtcttagattccggattcctggaacgaatgaatacccttctggctaatggcgaggttcctgggctctttgaaggggatgagtacgccacgctgatgacacagtgcaaagagggggcccagaaggaggggctcatgctggactcccacgaggagctctacaagtggttcaccagccaggtgatccgcaacctgcacgtggtcttcaccatgaacccgtcctcagaggggctcaaggacagggcggccacctcaccagcccttttcaacaggtgtgtgctgaactggtttggtgactggtccactgaagcactgtatcaagtcggcaaagaattcaccagtaagatggacctggagaagccgaactatatagtgcctgactacatgccagttgtgtatgataaactgccgcagcccccaacacaccgggaagccattgtgaacagctgtgtgtttgtccatcagactcttcaccaggctaatgcgcgcctggcgaagcgaggtggcagaacgatggccatcacacctcggcactacctggacttcatcaaccactacgccaacctcttccatgagaagcgcagcgagttggaggagcagcagatgcacctgaatgttgggctccgaaaaatcaaagagaccgtggaccaggtggaagaactgcgccgtgacctgaggatcaagagccaagagctggaggtcaagaacgcagcggccaatgacaagctgaagaaaatggtgaaagaccagcaagaggctgagaagaagaaggtcatgagccaagaaatccaggagcagctacacaaacagcaggaagtgatcgcagacaagcagatgagtgtcaaggaggacctggataaagtggagcctgctgtcatcgaggcccagaacgctgtgaagtccatcaagaagcagcacctggtggaggtgaggtccatggccaaccctcctgcagctgtgaagctggctctggagtccatctgcctgctgcttggtgagagcaccacggactggaagcagatccgctccatcatcatgagagagaacttcatccccaccatcgtcaacttctcggctgaggagatcagtgatgccatacgagagaagatgaagaaaaactacatgtccaaccccagttacaactatgagatcgtcaaccgggcttccctggcttgtggtcctatggtgaagtgggcgattgcacagctcaattatgcagacatgttaaagcgagtggagcccctgaggaatgagctgcagaagctggaagatgacgccaaggacaaccagcagaaagccaatgaggtggagcagatgatcagggacctggaagccagcattgcccgctacaaggaggagtacgctgtcctcatctctgaggcccaggccatcaaggcagacctggcagctgtggaagcaaaggtaaaccggagcactgcacttctaaagagtttatctgccgaaagggaacgttgggaaaagacgagtgaaactttcaaaaaccagatgtctaccattgctggggactgcctcctgtcggcggccttcattgcttacgctggctactttgaccagcaaatgcgccagaacttgttcaccacctggtctcatcacttacagcaagccaacatccagttccgcacagacattgcaaggacggagtacctctccaatgctgacgaacgcctccgctggcaggccagctctctgcctgccgacgacctgtgcacagagaatgccatcatgctaaaacgattcaataggtacccgctgatcatcgacccctccggacaggccacagaattcatcatgaatgaatataaggaccggaagatcacaaggaccagcttcttagatgatgccttccggaagaatttggagagtgctctgagatttggcaatccccttcttgtccaggatgtggaaagctacgatccagttttgaatccagtactgaaccgtgaagttcggcgaacaggagggagggtgctgattaccctcggtgaccaagacatagacctgtcaccatcctttgtcatctttctgtctacccgagacccaactgtggagttcccaccagacctctgctcccgggtgacctttgtgaacttcaccgtcacccgcagcagtttacagagccagtgtctcaatgaagtgcttaaagcagaaaggccagacgtggacgagaagcgctccgacctcctgaaactgcaaggggagttccaactccgtttacggcagcttgaaaagtctctgctccaagctctgaatgaggtaaaaggccgcattctggttgacgacaccatcattacaaccctggagaatctgaagcgggaagctgccgaggttaccagaaaggtggaggagacagatattgtcatgcaggaggtggagaccgtgtcccagcagtacctgccgctgtccactgcctgcagcagcatctacttcactatggagtccttgaagcaggttcacttcctgtaccagtactctctgcagttcttcctggacatctaccacaacgtgctgtatgagaaccccaacctgaagggtgccaccgatcacacacagcgcctctccgtcataaccaaggacctcttccaggtggcctttaaccgagtggctcgaggcatgctgcaccaggaccacatcaccttcgccatgctgctagcgaggatcaagctgaaaggcaccgtgggggagcccacctatgatgcagagttccagcacttcctcagagggaaggagattgtgctgagtgctggctccacccccaaaatccagggcctgaccgtagagcaggcagaagctgtcgtgaggttgagctgtctgcctgcatttaaagatctgattgccaaggtgcaggcagatgagcagtttggcatttggctggatagcagttcaccagagcagaccgtgccatacctctggagcgaggaaacgcctacaactcccattgggcaggccatccaccgcctgctcctgattcaagccttccgacccgaccgcctgttggccatggcccacatgtttgtttccacaaacctcggagagtccttcatgtccatcatggagcagcctctcgacctcacccacattgtgggcacagaggtgaagccaaacacacctgtcctgatgtgctctgtgcctgggtatgatgccagcggacatgttgaggacctcgcagcagagcagaacacgcagatcacgtctattgccatcggctcagcagaaggctttaaccaagcagataaggccatcaacaccgccgtgaagtcaggcaggtgggtgatgttgaagaacgtgcacctggccccgggctggctgatgcagctggagaagaagctgcactccctgcagccccacgcctgcttccgacttttcctcaccatggagatcaaccccaaggtgcccgtgaacctgctccgtgctggtcggatctttgtgtttgagccaccacccggagtgaaagccaacatgctgaggacatttagcagcatccctgtctctaggatatgcaagtctccaaatgagcgcgcccgcttgtatttcctgctggcttggttccatgccatcatccaagagcgcctgcgctatgctccactggggtggtcaaagaagtacgaatttggagaatctgaccttcgatcagcttgtgacacggtggacacgtggctggacgacacagccaagggccggcagaacatctcaccggataagatcccatggtctgccctgaagaccctgatggcgcagtccatctatggtgggcgggtggacaatgagttcgaccagcgtctgctcaacaccttcctggagcggctgttcaccacccgcagcttcgacagtgaattcaagctggcgtgcaaggttgacgggcacaaggacatccagatgcctgatggtatcaggcgagaggagtttgtgcagtgggtggagctgctgcctgacgcccagacaccctcatggctgggcctccccaacaatgctgaaagggtcctgcttaccacccaaggcgtggacatgatcagcaagatgctgaagatgcagatgctggaggacgaggatgacctggcctacgcagagacggagaagaaggcaaggacggactccacctccgatggccgtccagcctggatgaggacactgcacaccacggcgtccaactggctgcacctcatcccacagacactgagccccctgaagcgcaccgtggagaacatcaaggatcctttgtttaggttctttgagagagaagtaaagatgggggccaagttactccaagatgtgcgccaagaccttgctgacgtcgttcaagtgtgcgaggggaaaaagaagcagacaaactacctgcgcacgctcatcaatgagctggtgaaagggatcttgcctcggagctggtcccattacacagtgcctgcaggcatgacggtgatccagtgggtgtcagatttcagcgagaggatcaaacagctgcagaacatctcacaagcagctgcatctggtggtgccaaggaactgaagaacatccacgtgtgcctgggtggcctgtttgtacctgaggcctacatcactgccaccagacagtatgtggcacaagctaacagctggtccctggaggaactctgcctggaagtcaatgtcaccgcctcacagagtgccacccttgatgcctgcagctttggagtcacaggtctgaagctccaaggggccacgtgcagcaacaacaagctatcactctccaatgccatctccacagtcctccccctcacacagctgcgctgggtgaaacaaacaagcgccgagaagaaggccagcgtggtgaccttacctgtctacctgaacttcacccgggcagacctcatcttcaccgtggactttgaaattgctacaaaggaagatcctcgcagcttctatgagcgtggagttgccgttctgtgcactgagtaaagctagacttttctccagtcctctttctggaataggaaactcccaatatttaaccttcattcatccttaacggtgtggaagggttgatggactggaaagcaagtggttggttcgcgttggcgggtgacaggtgctgcggttggagacggtgggatgggagaagagaagagtgcgctgtccgagggctgaccgaatggctgggtttagtaaataaaacactaagcatgacgggcaaaaaaaaaaaaaaaaaaa

SEQ ID NO: 4 - protein, 4644 aa, Mus musculus
MSEPGGGEDGSAGLEVSAVQNVADVAVLQKHLRKLVPLLLEDGGDAPAALEAALEEKSALEQMRKFLSDPQVHTVLVERSTLKEDVGDEGEEEKEFISYNINIDIHYGVKSNSLAFIKRAPVIDADKPVSSQLRVLTLSEDSPYETLHSFISNAVAPFFKSYIRESGKADRDGDKMAPSVEKKIAELEMGLLHLQQNIEIPEISLPIHPIITNVAKQCYERGEKPKVTDFGDKVEDPTFLNQLQSGVNRWIREIQKVTKLDRDPASGTALQEISFWLNLERALYRIQEKRESPEVLLTLDILKHGKRFHATVSFDTDTGLKQALETVNDYNPLMKDFPLNDLLSATELDKIRQALVAIFTHLRKIRNTKYPIQRALRLVEAISRDLSSQLLKVLGTRKLMHVAYEEFEKVMVACFEVFQTWDDEYEKLQVLLRDIVKRKREENLKMVWRINPAHRKLQARLDQMRKFRRQHEQLRAVIVRVLRPQVTAVAQQNQGEAPEPQDMKVAEVLFDAADANTIEEVNLAYENVKEVDGLDVSKEGTEAWEAAMKRYDERIDRVETRITARLRDQLGTAKNANEMFRIFSRFNALFVRPHIRGAIREYQTQLIQRVKDDIESLHDKFKVQYPQSQACKMSHVRDLPPVSGSIIWAKQIDRQLTAYMKRVEDVLGKGWENHVEGQKLKQDGDSFRMKLNTQEIFDDWARKVQQRNLGVSGRIFTIESARVRGRTGNVLKLKVNFLPEIITLSKEVRNLKWLGFRVPLAIVNKAHQANQLYPFAISLIESVRTYERTCEKVEERNTISLLVAGLKKEVQALIAEGIALVWESYKLDPYVQRLAETVFNFQEKVDDLLIIEEKIDLEVRSLETCMYDHKTFSEILNRVQKAVDDLNLHSYSNLPIWVNKLDMEIERILGVRLQAGLRAWTQVLLGQAEDKAEVDMDTDAPQVSHKPGGEPKIKNVVHELRITNQVIYLNPPIEECRYKLYQEMFAWKMVVLSLPRIQSQRYQVGVHYELTEEEKFYRNALTRMPDGPVALEESYSAVMGIVTEVEQYVKVWLQCQCLWDMQAENIYNRLGEDLNKWQALLVQIRKARGTFDNAETKKEFGPVVIDYGKVQSKVNLKYDSWHKEVLSKFGQMLGSNMTEFHSQISKSRQELEQHSVDTASTSDAVTFITYVQSLKRKIKQFEKQVELYRNGQRLLEKQRFQFPPSWLYIDNIEGEWGAFNDIMRRKDSAIQQQVANLQMKIVQEDRAVESRTTDLLTDWEKTKPVTGNLRPEEALQALTIYEGKFGRLKDDREKCAKAKEALELTDTGLLSGSEERVQVALEELQDLKGVWSELSKVWEQIDQMKEQPWVSVQPRKLRQNLDGLLNQLKNFPARLRQYASYEFVQRLLKGYMKINMLVIELKSEALKDRHWKQLMKRLHVNWVVSELTLGQIWDVDLQKNEAVVKDVLLVAQGEMALEEFLKQIREVWNTYELDLVNYQNKCRLIRGWDDLFNKVKEHINSVSAMKLSPYYKVFEEDALSWEDKLNRIMALFDVWIDVQRRWVYLEGIFTGSADIKHLLPVETQRFQSISTEFLALMKKVSKSPLVMDVLNIQGVQRSLERLADLLGKIQKALGEYLERERSSFPRFYFVGDEDLLEIIGNSKNVAKLQKHFKKMFAGVSSIILNEDNSVVLGISSREGEEVMFKTPVSITEHPKINEWLTLVEKEMRVTLAKLLAESVTEVEIFGKATSIDPNTYITWIDKYQAQLVVLSAQIAWSENVENALSNVGGGGDVGPLQSVLSNVEVTLNVLADSVLMEQPPLRRRKLEHLITELVHQRDVTRSLIKSKIDNAKSFEWLSQMRFYFDPKQTDVLQQLSIQMANAKFNYGFEYLGVQDKLVQTPLTDRCYLTMTQALEARLGGSPFGPAGTGKTESVKALGHQLGRFVLVFNCDETFDFQAMGRIFVGLCQVGAWGCFDEFNRLEERMLSAVSQQVQCIQEALREHSNPNYDKTSAPITCELLNKQVKVSPDMAIFITMNPGYAGRSNLPDNLKKLFRSLAMTKPDRQLIAQVMLYSQGFRTAEVLANKIVPFFKLCDEQLSSQSHYDFGLRALKSVLVSAGNVKRERIQKIKREKEERGEAVDEGEIAENLPEQEILIQSVCETMVPKLVAEDIPLLFSLLSDVFPGVQYHRGEMTALREELKKVCQEMYLTYGDGEEVGGMWVEKVLQLYQITQINHGLMMVGPSGSGKSMAWRVLLKALERLEGVEGVAHIIDPKAISKDHLYGTLDPNTREWTDGLFTHVLRKIIDNVRGELQKRQWIVFDGDVDPEWVENLNSVLDDNKLLTLPNGERLSLPPNVRIMFEVQDLKYATLATVSRCGMVWFSEDVLSTDMILNNFLARLRSIPLDEGEDEAQRRRKGKEDEGEEAASPMLQIQRDAATIMQPYFTSNGLVTKALEHAFKLEHIMDLTRLRCLGSLFSMLHQACRNVAQYNANHPDFPMQIEQLERYIQRYLVYAILWSLSGDSRLKMRAELGEYIRRITTVPLPTAPNVPIIDYEVSISGEWSPWQAKVPQIEVETHKVAAPDVVVPTLDTVRHEALLYTWLAEHKPLVLCGPPGSGKTMTLFSALRALPDMEVVGLNFSSATTPELLLKTFDHYCEYRRTPNGVVLAPVQLGKWLVLFCDEINLPDMDKYGTQRVISFIRQMVEHGAFYRTSDQTWVKLERIQFVGACNPPTDPGRKPLSHRFLRHVPVVYVDYPGPASLTQIYGTFNRAMLRLIPSLRTYAEPLTAAMVEFYTMSQERFTQDTQPHYIYSPREMTRWVRGIFEALRPLETLPVEGLIRIWAHEALRLFQDRLVEDEERRWTDENIDMVALKHFPNIDKEKAMSRPILYSNWLSKDYIPVDQEELRDYVKARLKVFYEEELDVPLVLFNEVLDHVLRIDRIFRQPQGHLLLIGVSGAGKTTLSRFVAWMNGLSVYQIKVHRKYTGEDFDEDLRTVLRRSGCKNEKIAFIMDESNVLDSGFLERMNTLLANGEVPGLFEGDEYATLMTQCKEGAQKEGLMLDSHEELYKWFTSQVIRNLHVVFTMNPSSEGLKDRAATSPALFNRCVLNWFGDWSTEALYQVGKEFTSKMDLEKPNYIVPDYMPVVYDKLPQPPTHREAIVNSCVFVHQTLHQANARLAKRGGRTMAITPRHYLDFINHYANLFHEKRSELEEQQMHLNVGLRKIKETVDQVEELRRDLRIKSQELEVKNAAANDKLKKMVKDQQEAEKKKVMSQEIQEQLHKQQEVIADKQMSVKEDLDKVEPAVIEAQNAVKSIKKQHLVEVRSMANPPAAVKLALESICLLLGESTTDWKQIRSIIMRENFIPTIVNFSAEEISDAIREKMKKNYMSNPSYNYEIVNRASLACGPMVKWAIAQLNYADMLKRVEPLRNELQKLEDDAKDNQQKANEVEQMIRDLEASIARYKEEYAVLISEAQAIKADLAAVEAKVNRSTALLKSLSAERERWEKTSETFKNQMSTIAGDCLLSAAFIAYAGYFDQQMRQNLFTTWSHHLQQANIQFRTDIARTEYLSNADERLRWQASSLPADDLCTENAIMLKRFNRYPLIIDPSGQATEFIMNEYKDRKITRTSFLDDAFRKNLESALRFGNPLLVQDVESYDPVLNPVLNREVRRTGGRVLITLGDQDIDLSPSFVIFLSTRDPTVEFPPDLCSRVTFVNFTVTRSSLQSQCLNEVLKAERPDVDEKRSDLLKLQGEFQLRLRQLEKSLLQALNEVKGRILVDDTIITTLENLKREAAEVTRKVEETDIVMQEVETVSQQYLPLSTACSSIYFTMESLKQVHFLYQYSLQFFLDIYHNVLYENPNLKGATDHTQRLSVITKDLFQVAFNRVARGMLHQDHITFAMLLARIKLKGTVGEPTYDAEFQHFLRGKEIVLSAGSTPKIQGLTVEQAEAVVRLSCLPAFKDLIAKVQADEQFGIWLDSSSPEQTVPYLWSEETPTTPIGQAIHRLLLIQAFRPDRLLAMAHMFVSTNLGESFMSIMEQPLDLTHIVGTEVKPNTPVLMCSVPGYDASGHVEDLAAEQNTQITSIAIGSAEGFNQADKAINTAVKSGRWVMLKNVHLAPGWLMQLEKKLHSLQPHACFRLFLTMEINPKVPVNLLRAGRIFVFEPPPGVKANMLRTFSSIPVSRICKSPNERARLYFLLAWFHAIIQERLRYAPLGWSKKYEFGESDLRSACDTVDTWLDDTAKGRQNISPDKIPWSALKTLMAQSIYGGRVDNEFDQRLLNTFLERLFTTRSFDSEFKLACKVDGHKDIQMPDGIRREEFVQWVELLPDAQTPSWLGLPNNAERVLLTTQGVDMISKMLKMQMLEDEDDLAYAETEKKARTDSTSDGRPAWMRTLHTTASNWLHLIPQTLSPLKRTVENIKDPLFRFFEREVKMGAKLLQDVRQDLADVVQVCEGKKKQTNYLRTLINELVKGILPRSWSHYTVPAGMTVIQWVSDFSERIKQLQNISQAAASGGAKELKNIHVCLGGLFVPEAYITATRQYVAQANSWSLEELCLEVNVTASQSATLDACSFGVTGLKLQGATCSNNKLSLSNAISTVLPLTQLRWVKQTSAEKKASVVTLPVYLNFTRADLIFTVDFEIATKEDPRSFYERGVAVLCTE

SEQ ID NO: 5 - DNA, 13941 bp, Homo sapiens
atgtcggagcccgggggcggcggcggcgaggacggctcggccggattggaagtgtcggccgtgcagaatgtggcggacgtgtcggtgctgcagaagcacctgcgcaagctggtgccgctgctgctggaggacggcggcgaggcgccggccgcgctggaggcggcgctggaggagaagagcgccctggagcagatgcgcaagttcctttcggacccgcaggtccacacggtgctggtggagcgctccacgctcaaagaggacgtcggtgatgaaggagaagaagaaaaagaattcatttcctataacatcaacatagacattcattatggggttaaatccaatagcttggcattcattaaacgtactcccgtgattgatgcagataaacccgtgtcttctcagctccgggtccttacactcagtgaagactcgccctacgaaactttgcattctttcattagcaatgcagtggctcctttttttaagtcctacattagagagtctggcaaggcagacagggatggtgataaaatggctccttcagttgaaaagaagattgcagaactcgaaatgggactccttcacttgcagcaaaatattgaaattccggagatcagcctgccgattcatccaatgatcacaaatgttgcaaaacagtgttatgagcgtggagaaaagccaaaagttacagactttggtgataaggttgaagacccaacatttcttaatcagttacaatctggagttaaccgctggatccgagaaattcaaaaagtgaccaaactggatcgagatcctgcatcaggaactgccttacaggaaattagtttttggctaaacttggaacgtgcgttataccgcatccaggagaaacgggagagcccggaagttctcctgactctggatatcttgaaacatggcaagcgcttccatgccaccgtcagttttgacactgacacaggtctaaaacaggctttggaaactgtgaatgactacaatcctctgatgaaagatttccctctgaatgatttgctgtctgccacggagctggacaaaataagacaggcgcttgttgccattttcacacatttgagaaagatccgaaacacaaaatatcctattcagagggcactgcgtttggtggaggcaatttcaagagacttgagttctcaattactcaaagtattgggcactaggaaattgatgcatgttgcttatgaagaatttgaaaaagttatggtagcatgctttgaagtttttcagacttgggatgatgagtatgagaaacttcaggtattgttgagagacatcgtcaaaagaaaaagggaagaaaatctgaagatggtgtggcgtatcaaccctgcccacaggaagctgcaggcccgccttgaccagatgagaaaatttagacgccagcatgaacagctaagagctgttatcgtcagggtcctgaggccacaggtcacggcagttgcacaacagaatcaaggagaggtccctgaaccccaagatatgaaagtggctgaggttctctttgatgctgcagatgcaaatgccattgaggaagtaaaccttgcttatgagaacgtcaaggaagtggatggactggatgtttccaaagagggcacggaagcctgggaggctgctatgaagaggtacgatgagaggatcgacagagtggagacccggatcaccgctcgccttcgggatcagcttggcacagccaagaatgccaacgagatgtttaggattttctccaggtttaatgcactgtttgtcaggcctcacatccgtggggccattcgcgaataccagacccagctgatccagcgcgtgaaagatgacattgagtctcttcacgacaagttcaaggtccagtacccacagagtcaggcttgtaagatgagtcacgttcgtgacttgccccctgtgtcagggtctatcatctgggctaaacagatcgacaggcagctgacggcctacatgaagcgggtggaagatgtccttggcaagggctgggagaatcacgtggaggggcagaagctgaagcaggatggagacagcttccgcatgaagctcaacacgcaggagatctttgatgactgggcaaggaaggtgcagcagcgcaacctcggtgtctcggggcgcattttcaccatcgaaagtactcgggttcggggccgaactggaaatgtgcttaagctgaaagttaactttcttcctgagattatcacactatccaaagaagtccggaacctcaaatggcttggtttccgcgtcccactggcgattgtgaacaaagcccatcaagcaaaccagctttacccgtttgccatctcactgatcgagagcgttcgtacctatgaacggacctgcgagaaggtggaggagcggaacaccatttcccttttggtggctggcttgaaaaaggaagtgcaggccctgatcgcagaaggcattgcgttggtgtgggagtcctacaaacttgacccatatgtacagcgcttagcagagactgtcttcaacttccaagaaaaggtggatgatctgctgatcattgaagaaaaaatagacctagaagtccgttccttggaaacttgtatgtatgaccataagacattctcggaaatcttgaacagagtccagaaagcagtggatgacttaaatctgcactcctattccaatttgcccatctgggtcaacaagcttgacatggagattgaaagaatattgggcgtccgtctgcaagctggcctgagagcttggacgcaggttcttcttggacaagctgaagataaagcagaagttgacatggacacagatgctccacaagttagtcacaagcctggtggagagccaaagatcaaaaatgtcgttcatgagctaagaataaccaatcaggtaatctacttgaatccaccaattgaagagtgcagatacaagctgtatcaggaaatgtttgcctggaagatggttgtactgtctctccccaggatccagagtcagaggtaccaggtgggtgtacattacgaattgactgaggaagagaaattctatcggaatgctttaacacggatgcctgatggccctgttgccctggaagagtcgtattctgctgtcatgggcattgtatctgaagttgaacagtatgtcaaggtttggcttcagtgtcagtgtttatgggatatgcaagctgaaaacatctataacagacttggagaagatctcaacaaatggcaggctctcctggtccaaataaggaaggccagaggaacctttgacaatgcagaaaccaagaaagagtttggaccagtagttatagattatggcaaggtacaatctaaggtgaacttgaaatatgactcttggcataaggaggttcttagcaaatttgggcagatgctaggatcaaacatgacggaattccattcccagatctcaaagtcccgccaagagttggagcagcactcagtagacacggccagcacctccgatgcagtgaccttcatcacctatgtgcagtctttgaaacggaagatcaagcagtttgagaagcaagttgagctctaccgcaatggccagcgcttactggaaaagcaaaggttccagttcccaccttcctggctttatattgacaacatcgagggagagtggggagccttcaatgacatcatgcggcgaaaggactctgccattcagcagcaggtggcaaacctgcaaatgaagattgtccaggaggatcgggccgtggaaagccgcaccaccgacctgctgactgactgggagaagaccaagcctgtcacgggcaaccttcgcccagaagaggcacttcaggctctcaccatatatgaggggaagtttggtaggctgaaggacgacagagagaagtgtgcaaaggccaaggaggcgctggaattgacagatactgggcttctcagtggcagtgaagagcgcgtgcaggtggccttagaagaattacaggacctcaaaggcgtttggtcagaactttctaaggtttgggagcaaatcgatcagatgaaggagcaaccctgggtttcagtacagcctcgaaagcttcgacaaaatttggatgccctcctgaaccagctgaaaagcttccctgcccggttgcgacagtatgcgtcctatgagtttgttcagaggcttctgaaaggttacatgaagataaatatgctggtgattgaactgaaatccgaagcacttaaagaccgccattggaaacagctcatgaaaaggcttcacgttaattgggttgtttctgagctaacccttggccaaatctgggatgttgacttgcagaaaaatgaagcgattgtcaaggatgtactgcttgtggcacaaggggagatggctttggaagaatttttgaagcagataagagaagtgtggaatacttatgaactagacttggttaattatcagaacaagtgccgcttgatccgtggctgggatgacctcttcaacaaggtcaaagaacacatcaacagcgtctcggccatgaagctctctccgtattacaaggtttttgaagaggatgctctcagctgggaagataagctgaacaggatcatggctctctttgatgtgtggattgatgtgcagaggcggtgggtctacctggaaggtatcttcacaggcagtgcagatatcaagcacctgctgccagtggaaacccagcggtttcagagcatcagcactgagtttttggctctaatgaaaaaagtgtccaagtctccccttgttatggatgttctgaacatccagggagtacagaggtctctggaaagattggcagacctgctaggaaagatccagaaagcattgggagaatatctggaaagagagcggtcatctttccccaggttctattttgtgggtgatgaagatttgcttgaaatcattggaaacagcaagaatgtcgctaaattacagaaacacttcaagaagatgtttgctggagtttcgagcatcatcctgaacgaggataactctgttgttttgggtatttcatctcgggaaggagaggaggttatgtttaaaactcctgtgtcaattactgaacatcccaaaatcaatgagtggctcacattggtagaaaaggagatgagagtcaccctggccaaactgcttgctgagtctgttacggaagttgagatttttggtaaagcaacttcaattgacccaaatacctacatcacttggattgataaataccaggcccagcttgtggttttgtcagcccagatagcctggtctgagaacgtggagaccgcactgagcagcatgggcggaggtggagatgccgcgcccttgcactctgtgctgagcaatgtggaggtcaccctcaatgtgttagcagactctgtcctcatggagcagcccccactccgaaggcggaagctagaacacttgattacagagttggttcaccagagagatgttacaaggtccttgatcaaaagcaagattgacaacgccaaatcttttgaatggctcagccagatgcgattttactttgaccctaagcaaactgatgtgttacagcagttgtcaattcaaatggcaaatgccaaatttaactatggctttgagtacctgggtgttcaggacaaactggtccagacccccctcactgaccgctgctatttgacaatgacacaagccttggaggccaggctggggggttccccatttggacctgctggaactgggaaaacagagtctgtcaaagctcttggccatcagcttggacggtttgttttagttttcaactgtgatgaaacctttgatttccaggcaatgggccggatctttgtgggcctttgccaggtgggtgcctggggctgctttgacgagttcaaccgcctggaggagcggatgctctcggctgtgtcccagcaggtgcagtgcatacaggaagcactgcgtgaacattccaaccccaactacgacaagacctctgcccccattacttgtgagctgctgaacaaacaagtcaaggtgagcccggacatggccatcttcatcaccatgaaccctggctacgcgggccggtctaaccttcctgacaacttgaagaagctgttccggagcttggccatgaccaagcccgaccggcagttaatcgcccaggtcatgctgtactcacagggtttccgcactgctgaagtgcttgccaacaaaatcgtcccgttttttaaactatgcgatgagcagctctcttcccaaagccattatgacttcggtcttcgggctttgaagagtgtgctggtgagtgcaggcaatgtgaagagagagagaatccagaagataaagagggagaaagaggaacgaggggaagcagttgatgaaggagaaattgctgaaaatctccctgaacaagagattctgatacagagcgtctgtgagacgatggtgccaaagctggtggcagaggacatcccgctgctcttcagcctcctgtcggacgtgttccctggagtccagtatcacaggggtgagatgactgcccttcgagaggagctgaagaaagtgtgtcaggagatgtatttgacatatggagatggagaagaagttggtggaatgtgggttgaaaaggttctccagctctatcagatcacccagatcaatcatggcctgatgatggtggggccctcgggaagtgggaagagcatggcctggcgtgtcctgctgaaggcattggagagactcgagggtgtggaaggtgtggcccatatcatcgaccccaaggccatcagcaaagaccacctctacggaaccctggaccccaacaccagggaatggacagatgggctcttcacacacgtgctgagaaagatcatcgacagcgtgagaggcgagctgcagaagcgccagtggatcgtcttcgatggcgatgtggatccagagtgggttgagaacttgaactcagtgctggatgacaataagctcctaactttgcccaatggagagcgcctcagtcttccacccaatgtgagaataatgtttgaggtacaggacttgaaatacgcgaccttggccacagtgtcgcgctgcggcatggtctggttcagtgaggatgtgctgagcaccgacatgatcttcaacaacttcctggccaggctgcgcagcatcccgctggatgaaggggaggatgaggcacagcggcggcgtaagggcaaagaggatgagggggaggaggccgcttcccccatgctgcagatccaaagagatgcagctacgatcatgcaaccgtacttcacgtccaacggcctggtcaccaaggcgctagagcacgccttccagctggagcacatcatggacctaacacgcctgcgctgcctgggctcgctcttctccatgctgcaccaggcctgccgcaacgtggcgcagtataacgccaaccatcccgacttccccatgcagatcgagcagctggagcgctacattcagcgatatctggtttatgccatactctggtccctgtctggagacagccggctaaaaatgagagcagagctgggtgaatacatcagaagaatcacgaccgtgcctctgcccactgcgcccaacatacccattatcgattatgaggtgtccatcagcggagaatggtctccgtggcaggccaaggtgcctcagattgaagtggagacgcacaaggtggcagcccctgatgtcgtcgtgccaacgctggacacagtccgccacgaagccctcttgtacacttggctggccgaacacaagcccctggtcttgtgtggccctcctgggtctggcaagaccatgacactcttcagcgccctccgggccttgcctgacatggaggtggtgggtctcaacttctccagtgctactactccagagctgcttctgaagacttttgatcactactgcgagtacaggcgcacacctaatggggtggttttggctcctgttcaacttggaaagtggctggtgttgttctgtgatgaaatcaacttgccagatatggataaatatgggacccagagggtcatatccttcatcagacagatggtggagcacggaggcttttaccgtacctcagatcaaacatgggtgaagctggagagaatccagtttgttggggcttgtaatccccccacagaccctggaagaaagcccctctcacacaggttcctgcgccacgtgcctgtcgtgtatgtggattacccgggccccgcctccctcacacagatctacggcaccttcaaccgcgccatgctgaggctcattccatccctgcggacgtatgcagagccgctcactgctgccatggtggagttctacaccatgtctcaggagagattcacccaggatacacaacctcactatatctattcaccccgtgaaatgactaggtgggtgagaggcatctttgaagcgctgagacctctggagaccctgcctgttgaaggcctcattcggatttgggcacatgaagctctgcgtctcttccaagatagactcgtagaggatgaggagaggcgttggactgatgagaacatcgacacggttgctctgaagcacttccctaacatcgacagagagaaggcaatgagccgacccatcttgtacagcaactggctgtcaaaggattacatcccagtagaccaagaagagttaagagattatgtcaaagctaggctgaaggtcttttatgaagaagaacttgatgttccgctggtgctgtttaatgaagtcctagaccacgtgctgaggattgacagaatattccgtcaacctcaaggccacttgcttctgattggtgttagtggagcaggaaaaactaccctgtctcgtttcgtcgcctggatgaacggtttgagtgtgtaccagattaaggtccataggaagtacacaggggaagactttgatgaagatctacggacagtgttgagacgttctggctgtaaaaatgaaaagatagcatttataatggatgaatctaatgtgttagattctggattcctggagcgaatgaatacccttctggccaatggagaggtgcctggtctctttgaaggagacgagtatgccaccttgatgacgcagtgcaaagagggggcacagaaggaaggcctgatgctggactcgcacgaggagctctacaagtggttcactagccaggttatccgcaacctccacgtcgtgttcaccatgaacccgtcctcggagggactcaaggaccgggcagctacatcaccagcacttttcaacaggtgtgtgttgaattggtttggagactggtccaccgaagcactgtatcaggttggcaaagaattcacaagtaagatggatctggagaagccaaattacatcgtgcctgattacatgccagttgtgtatgataagctgccgcagccaccatcccatcgggaagccattgtgaacagctgtgtgtttgttcatcagactcttcaccaggcgaatgctcggctagcaaagcgaggcggcagaacgatggccatcacccctcgccactacctggacttcatcaatcactatgccaacctgttccacgagaagcggagcgagctggaggagcagcagatgcacttgaacgtggggctcaggaagatcaaagagacagtcgaccaggtagaagaactgcgtcgtgacttgaggataaagagccaagagctggaggtgaagaatgcagcagccaatgacaagctgaaaaagatggtgaaagaccagcaggaggctgaaaagaagaaggttatgagccaagaaatccaggaacagctgcataagcagcaggaggtaattgcagacaaacagatgagtgtcaaagaagatcttgataaggtggaacctgccgtcattgaggcccagaatgctgtgaagtcgatcaagaagcagcacctggtggaggtgaggtccatggccaaccctcctgctgctgtgaagctggcgctggagtccatctgcctgctgctgggggaaagcaccacagactggaagcagatccgctccatcatcatgcgggagaacttcatccccaccatcgtcaacttctctgcagaggagatcagtgacgccataagggagaagatgaagaaaaattacatgtccaatccaagttacaattatgaaattgtgaatcgggcttccctggcttgcggccctatggtgaaatgggcaattgcacagcttaactatgcagacatgttaaagagagtggagcccctacgcaatgagctgcagaagctggaagatgacgccaaggacaaccagcagaaggccaacgaggtggagcagatgatccgagacctggaagccagcatcgcccgctacaaggaggaatacgccgtcctgatctcagaggcccaggccatcaaggcagacctggcagctgtcgaggcaaaagtaaaccggagcactgctcttctgaagagcttgtctgctgaacgtgaacgatgggaaaaaacaagtgaaactttcaaaaaccagatgtccaccattgctggggactgtctcttgtcagctgcgttcattgcctacgcgggttactttgaccagcagatgcgtcagaacttgttcactacctggtcccatcacctacagcaagccaacatccagttccgtacagatattgccaggacggaatacctttccaatgctgatgagcgtcttcgctggcaggccagctccttgcctgctgatgacctttgcacagaaaatgccatcatgctgaaacgattcaataggtatccgctgatcattgacccctctggacaggccacagaattcattatgaatgaatataaggatcgtaagatcacacggaccagcttcctggatgacgccttcagaaagaacttagagagtgcactgagattcggtaacccccttctggtccaggatgtggaaagctacgatccagttttgaacccggtgctgaaccgtgaagtgcggcgaacaggggggagagtgctgatcactctcggggaccaggacatagacctgtcgccatcgtttgtcatcttcctgtccacccgggatccaactgtcgagttcccaccagatctctgttcccgggttacttttgtaaacttcacagttacccgtagcagtttacaaagccagtgtctaaatgaagtacttaaagcagaaagacctgatgtggacgagaaacgatctgatcttcttaaacttcaaggggaatttcagctccgtttgcgtcagctggaaaaatctctactacaagctctgaacgaggtgaaagggcgcattttggatgacgacacgatcataaccactctggagaacctgaagagagaggctgcagaggtcaccaggaaagttgaggagacggacattgtcatgcaggaggtggagaccgtgtcccagcagtacctcccgctctccaccgcctgcagcagcatctacttcaccatggagtccctcaagcagatacacttcttgtaccagtactccctccagtttttcctggacatttatcacaacgtcctatacgagaacccgaacctgaagggtgtcaccgaccacacacagcgcctgtccattataacaaaggacctcttccaggtggcgtttaaccgagtggctcgaggcatgctgcatcaggaccacattacctttgccatgctgctggcaagaatcaaactgaagggcaccgtgggggagcccacctacgatgcagaattccagcacttcttgagaggaaatgagattgtcctgagtgctggctccacccccaggatccagggcctgactgtggagcaggcggaggcggtggtgaggctgagctgccttcccgcgtttaaggacttgattgcaaaggttcaggcagacgagcaatttggcatctggctggacagcagctccccggagcagactgtgccctacctctggagtgaagaaacacctgcaacacccattggccaggccatccaccgcctgctcctgatccaggctttccggcccgatcgcctgttggccatggcccacatgtttgtttcaacaaaccttggggagtctttcatgtccatcatggagcagccgctcgacctgacccacattgtgggcacagaggtgaagcccaacactcctgtcttaatgtgctctgtgcctggttatgatgccagtggacatgtcgaggaccttgcagccgagcagaacacgcagatcacttcaattgcaatcggctctgcagaaggctttaaccaagcagataaggcaataaacaccgctgtaaagtcgggcaggtgggtgatgctgaagaatgtgcatctggccccagggtggctgatgcagctggagaagaagttgcattccctgcagccgcatgcctgcttccgactcttcctcaccatggagatcaaccccaaggtgcctgtgaatctgctccgtgcgggccgcatctttgtgttcgagccaccgccaggggtgaaggccaacatgctgaggacgttcagcagcattcccgtctcacggatatgcaagtctcccaacgagcgtgcccgcttgtacttcctgctggcctggtttcatgcgatcatccaagaacgcttacgatacgcaccactggggtggtcaaagaagtatgaatttggagagtctgacctgcggtcagcttgcgatacggtggacacgtggctggatgacacggccaagggcaggcagaacatctcaccggataagatcccgtggtctgcactaaagaccttaatggcccagtccatttatggcgggcgcgtggacaacgagtttgaccagcgtctgctcaacaccttcctggagcgcctgttcacaaccaggagtttcgacagtgagtttaagctggcatgcaaggtcgacggacataaagacattcaaatgccagatggcatcaggcgagaggagtttgtgcagtgggtggagttgctccccgacacccagacgccctcctggctgggcctgcccaacaacgccgagagagtcctccttaccacacagggtgtggacatgatcagtaaaatgctgaagatgcagatgttggaggatgaggacgacctggcctacgcagagactgagaagaagacgaggacagactccacgtccgacgggcgccctgcctggatgcggacactgcacaccaccgcgtccaactggctgcacctcatcccccagacgctgagccacctcaagcgcaccgtggagaatatcaaggatcctttgttcaggttctttgagagagaagtgaagatgggcgcaaagctgcttcaggacgttcgccaggaccttgcagatgtcgtccaggtgtgcgaaggaaagaagaagcagaccaactacttgcgcacgctgatcaacgagctagtgaaagggatcttgcctcggagctggtcccactacacggtgcctgccggcatgaccgtcatccagtgggtgtccgacttcagcgagaggatcaaacagctgcagaacatctcactggcagctgcatctggtggcgccaaggagctaaagaacatccacgtgtgcctgggtggcctgttcgtgcctgaggcgtacatcactgccaccaggcagtatgtggcccaggccaacagctggtccctggaggagctctgcctggaagtcaacgtcaccacctcacagggcgccacccttgacgcttgcagcttcggagtcacgggtttgaaacttcaaggggccacgtgcaacaacaacaagctgtcactgtccaatgccatctcaaccgcccttcccctgacgcagctgcgctgggtcaagcagacaaacaccgagaagaaggccagtgtggtaaccttacctgtctacctgaacttcacccgtgcagacctcatcttcaccgtggacttcgaaattgctacaaaggaggatcctcgcagcttctacgagcggggtgtcgcagtcttgtgcacagagtaa

SEQ ID NO: 6 - protein, 4646 aa, Homo sapiens
MSEPGGGGGEDGSAGLEVSAVQNVADVSVLQKHLRKLVPLLLEDGGEAPAALEAALEEKSALEQMRKFLSDPQVHTVLVERSTLKEDVGDEGEEEKEFISYNINIDIHYGVKSNSLAFIKRTPVIDADKPVSSQLRVLTLSEDSPYETLHSFISNAVAPFFKSYIRESGKADRDGDKMAPSVEKKIAELEMGLLHLQQNIEIPEISLPIHPMITNVAKQCYERGEKPKVTDFGDKVEDPTFLNQLQSGVNRWIREIQKVTKLDRDPASGTALQEISFWLNLERALYRIQEKRESPEVLLTLDILKHGKRFHATVSFDTDTGLKQALETVNDYNPLMKDFPLNDLLSATELDKIRQALVAIFTHLRKIRNTKYPIQRALRLVEAISRDLSSQLLKVLGTRKLMHVAYEEFEKVMVACFEVFQTWDDEYEKLQVLLRDIVKRKREENLKMVWRINPAHRKLQARLDQMRKFRRQHEQLRAVIVRVLRPQVTAVAQQNQGEVPEPQDMKVAEVLFDAADANAIEEVNLAYENVKEVDGLDVSKEGTEAWEAAMKRYDERIDRVETRITARLRDQLGTAKNANEMFRIFSRFNALFVRPHIRGAIREYQTQLIQRVKDDIESLHDKFKVQYPQSQACKMSHVRDLPPVSGSIIWAKQIDRQLTAYMKRVEDVLGKGWENHVEGQKLKQDGDSFRMKLNTQEIFDDWARKVQQRNLGVSGRIFTIESTRVRGRTGNVLKLKVNFLPEIITLSKEVRNLKWLGFRVPLAIVNKAHQANQLYPFAISLIESVRTYERTCEKVEERNTISLLVAGLKKEVQALIAEGIALVWESYKLDPYVQRLAETVFNFQEKVDDLLIIEEKIDLEVRSLETCMYDHKTFSEILNRVQKAVDDLNLHSYSNLPIWVNKLDMEIERILGVRLQAGLRAWTQVLLGQAEDKAEVDMDTDAPQVSHKPGGEPKIKNVVHELRITNQVIYLNPPIEECRYKLYQEMFAWKMVVLSLPRIQSQRYQVGVHYELTEEEKFYRNALTRMPDGPVALEESYSAVMGIVSEVEQYVKVWLQCQCLWDMQAENIYNRLGEDLNKWQALLVQIRKARGTFDNAETKKEFGPVVIDYGKVQSKVNLKYDSWHKEVLSKFGQMLGSNMTEFHSQISKSRQELEQHSVDTASTSDAVTFITYVQSLKRKIKQFEKQVELYRNGQRLLEKQRFQFPPSWLYIDNIEGEWGAFNDIMRRKDSAIQQQVANLQMKIVQEDRAVESRTTDLLTDWEKTKPVTGNLRPEEALQALTIYEGKFGRLKDDREKCAKAKEALELTDTGLLSGSEERVQVALEELQDLKGVWSELSKVWEQIDQMKEQPWVSVQPRKLRQNLDALLNQLKSFPARLRQYASYEFVQRLLKGYMKINMLVIELKSEALKDRHWKQLMKRLHVNWVVSELTLGQIWDVDLQKNEAIVKDVLLVAQGEMALEEFLKQIREVWNTYELDLVNYQNKCRLIRGWDDLFNKVKEHINSVSAMKLSPYYKVFEEDALSWEDKLNRIMALFDVWIDVQRRWVYLEGIFTGSADIKHLLPVETQRFQSISTEFLALMKKVSKSPLVMDVLNIQGVQRSLERLADLLGKIQKALGEYLERERSSFPRFYFVGDEDLLEIIGNSKNVAKLQKHFKKMFAGVSSIILNEDNSVVLGISSREGEEVMFKTPVSITEHPKINEWLTLVEKEMRVTLAKLLAESVTEVEIFGKATSIDPNTYITWIDKYQAQLVVLSAQIAWSENVETALSSMGGGGDAAPLHSVLSNVEVTLNVLADSVLMEQPPLRRRKLEHLITELVHQRDVTRSLIKSKIDNAKSFEWLSQMRFYFDPKQTDVLQQLSIQMANAKFNYGFEYLGVQDKLVQTPLTDRCYLTMTQALEARLGGSPFGPAGTGKTESVKALGHQLGRFVLVFNCDETFDFQAMGRIFVGLCQVGAWGCFDEFNRLEERMLSAVSQQVQCIQEALREHSNPNYDKTSAPITCELLNKQVKVSPDMAIFITMNPGYAGRSNLPDNLKKLFRSLAMTKPDRQLIAQVMLYSQGFRTAEVLANKIVPFFKLCDEQLSSQSHYDFGLRALKSVLVSAGNVKRERIQKIKREKEERGEAVDEGEIAENLPEQEILIQSVCETMVPKLVAEDIPLLFSLLSDVFPGVQYHRGEMTALREELKKVCQEMYLTYGDGEEVGGMWVEKVLQLYQITQINHGLMMVGPSGSGKSMAWRVLLKALERLEGVEGVAHIIDPKAISKDHLYGTLDPNTREWTDGLFTHVLRKIIDSVRGELQKRQWIVFDGDVDPEWVENLNSVLDDNKLLTLPNGERLSLPPNVRIMFEVQDLKYATLATVSRCGMVWFSEDVLSTDMIFNNFLARLRSIPLDEGEDEAQRRRKGKEDEGEEAASPMLQIQRDAATIMQPYFTSNGLVTKALEHAFQLEHIMDLTRLRCLGSLFSMLHQACRNVAQYNANHPDFPMQIEQLERYIQRYLVYAILWSLSGDSRLKMRAELGEYIRRITTVPLPTAPNIPIIDYEVSISGEWSPWQAKVPQIEVETHKVAAPDVVVPTLDTVRHEALLYTWLAEHKPLVLCGPPGSGKTMTLFSALRALPDMEVVGLNFSSATTPELLLKTFDHYCEYRRTPNGVVLAPVQLGKWLVLFCDEINLPDMDKYGTQRVISFIRQMVEHGGFYRTSDQTWVKLERIQFVGACNPPTDPGRKPLSHRFLRHVPVVYVDYPGPASLTQIYGTFNRAMLRLIPSLRTYAEPLTAAMVEFYTMSQERFTQDTQPHYIYSPREMTRWVRGIFEALRPLETLPVEGLIRIWAHEALRLFQDRLVEDEERRWTDENIDTVALKHFPNIDREKAMSRPILYSNWLSKDYIPVDQEELRDYVKARLKVFYEEELDVPLVLFNEVLDHVLRIDRIFRQPQGHLLLIGVSGAGKTTLSRFVAWMNGLSVYQIKVHRKYTGEDFDEDLRTVLRRSGCKNEKIAFIMDESNVLDSGFLERMNTLLANGEVPGLFEGDEYATLMTQCKEGAQKEGLMLDSHEELYKWFTSQVIRNLHVVFTMNPSSEGLKDRAATSPALFNRCVLNWFGDWSTEALYQVGKEFTSKMDLEKPNYIVPDYMPVVYDKLPQPPSHREAIVNSCVFVHQTLHQANARLAKRGGRTMAITPRHYLDFINHYANLFHEKRSELEEQQMHLNVGLRKIKETVDQVEELRRDLRIKSQELEVKNAAANDKLKKMVKDQQEAEKKKVMSQEIQEQLHKQQEVIADKQMSVKEDLDKVEPAVIEAQNAVKSIKKQHLVEVRSMANPPAAVKLALESICLLLGESTTDWKQIRSIIMRENFIPTIVNFSAEEISDAIREKMKKNYMSNPSYNYEIVNRASLACGPMVKWAIAQLNYADMLKRVEPLRNELQKLEDDAKDNQQKANEVEQMIRDLEASIARYKEEYAVLISEAQAIKADLAAVEAKVNRSTALLKSLSAERERWEKTSETFKNQMSTIAGDCLLSAAFIAYAGYFDQQMRQNLFTTWSHHLQQANIQFRTDIARTEYLSNADERLRWQASSLPADDLCTENAIMLKRFNRYPLIIDPSGQATEFIMNEYKDRKITRTSFLDDAFRKNLESALRFGNPLLVQDVESYDPVLNPVLNREVRRTGGRVLITLGDQDIDLSPSFVIFLSTRDPTVEFPPDLCSRVTFVNFTVTRSSLQSQCLNEVLKAERPDVDEKRSDLLKLQGEFQLRLRQLEKSLLQALNEVKGRILDDDTIITTLENLKREAAEVTRKVEETDIVMQEVETVSQQYLPLSTACSSIYFTMESLKQIHFLYQYSLQFFLDIYHNVLYENPNLKGVTDHTQRLSIITKDLFQVAFNRVARGMLHQDHITFAMLLARIKLKGTVGEPTYDAEFQHFLRGNEIVLSAGSTPRIQGLTVEQAEAVVRLSCLPAFKDLIAKVQADEQFGIWLDSSSPEQTVPYLWSEETPATPIGQAIHRLLLIQAFRPDRLLAMAHMFVSTNLGESFMSIMEQPLDLTHIVGTEVKPNTPVLMCSVPGYDASGHVEDLAAEQNTQITSIAIGSAEGFNQADKAINTAVKSGRWVMLKNVHLAPGWLMQLEKKLHSLQPHACFRLFLTMEINPKVPVNLLRAGRIFVFEPPPGVKANMLRTFSSIPVSRICKSPNERARLYFLLAWFHAIIQERLRYAPLGWSKKYEFGESDLRSACDTVDTWLDDTAKGRQNISPDKIPWSALKTLMAQSIYGGRVDNEFDQRLLNTFLERLFTTRSFDSEFKLACKVDGHKDIQMPDGIRREEFVQWVELLPDTQTPSWLGLPNNAERVLLTTQGVDMISKMLKMQMLEDEDDLAYAETEKKTRTDSTSDGRPAWMRTLHTTASNWLHLIPQTLSHLKRTVENIKDPLFRFFEREVKMGAKLLQDVRQDLADVVQVCEGKKKQTNYLRTLINELVKGILPRSWSHYTVPAGMTVIQWVSDFSERIKQLQNISLAAASGGAKELKNIHVCLGGLFVPEAYITATRQYVAQANSWSLEELCLEVNVTTSQGATLDACSFGVTGLKLQGATCNNNKLSLSNAISTALPLTQLRWVKQTNTEKKASVVTLPVYLNFTRADLIFTVDFEIATKEDPRSFYERGVAVLCTE

SEQ ID NO: 7 - protein, 60 aa, Artificial Sequence
  partial amino acid sequence of Mus musculus cytoplasmic dynein heavy chain 1
IVTEVEQYVKVWLQYQCLWDMQAENIYNRLGEDLNKWQALLVQIRKARGTFDNAETKKEF

SEQ ID NO: 8 - protein, 60 aa, Artificial Sequence
  partial amino acid sequence of Rattus norvegicus  cytoplasmic dynein heavy chain 1
IVTEVEQYVKVWLQYQCLWDMQAENIYNRLGEDLSKWQALLVQIRRARGTFDNAETKKEF

SEQ ID NO: 9 - protein, 60 aa, Artificial Sequence
  partial amino acid sequence of Homo sapiens cytoplasmic dyneineavy chain 1
IVSEVEQYVKVWLQYQCLWDMQAENIYNRLGEDLNKWQALLVQIRKARGTFDNAETKKEF

SEQ ID NO: 10 - protein, 60 aa, Artificial Sequence
  partial amino acid sequence of Drosophila melanogaster cytoplasmic dynein heavy chain 1
KVSEVRNYVDEWLRYQSLWDLQADMLYGRLGEDVNLWIKCLNDIKQSRTTFDTSDTRRAY

SEQ ID NO: 11 - protein, 60 aa, Artificial Sequence
  partial amino acid sequence of Paramecium cytoplasmic dynein heavy chain 1
LLEDMEQYVQTWLNYQSLWELDIKQVEQILQDDIEKWQQMLTDIKQGRATFDNSTTEEHF

SEQ ID NO: 12 - protein, 60 aa, Artificial Sequence
  partial amino acid sequence of C. elegans cytoplasmic dynein heavy chain 1
IMSDLEEYLSEWLSYQSLWVLQAEQLFEMLGTSLSKWMKTLMEIRKGRLVFDTQDTRKVI

SEQ ID NO: 13 - DNA, 364 bp, Artificial Sequence
  nucleotide position +3029 to +3392 of Mus musculus wild type cytoplasmic dynein heavy chain 1 cDNA
tccaagactttgaagtgaggttctctaatggtgtgaagacatggactgttgacatggtgggttctagcatattaatagtctacagcactgcagactagaattgagctgtgtttcctttgcaggtttggcttcagtaccagtgtttgtgggatatgcaggcagaaaacatttacaacaggctaggggaagatctcaacaagtggcaagctctcctggtccagataaggaaagccagaggaacctttgacaatgcggaaaccaagaaagagtttggtccggtggtgatagattacggcaaggtgagcttggcttgttgtcaaaaggcaccaggcagccgtaagatcagtattggtgtttggccctg

SEQ ID NO: 14 - DNA, 364 bp, Artificial Sequence
  nucleotide position +3029 to +3392 of Mus musculus mutant cytoplasmic dynein heavy chain 1 cDNA
tccaagactttgaagtgaggttctctaatggtgtgaagacatggactgttgacatggtgggttctagcatattaatagtctacagcactgcagactagaattgagctgtgtttcctttgcaggtttggcttcagtgccagtgtttgtgggatatgcaggcagaaaacatttacaacaggctaggggaagatctcaacaagtggcaagctctcctggtccagataaggaaagccagaggaacctttgacaatgcggaaaccaagaaagagtttggtccggtggtgatagattacggcaaggtgagcttggcttgttgtcaaaaggcaccaggcagccgtaagatcagtattggtgtttggccctg

SEQ ID NO: 15 - DNA, 21 bp, Artificial Sequence
  oligonucleotide primer, Mus musculus
tccaagactttgaagtgaggt

SEQ ID NO: 16 - DNA, 20 bp, Artificial Sequence
  oligonucleotide primer, Mus musculus
cagggccaaacaccaatact

SEQ ID NO: 17 - DNA, 13941 bp, Homo sapiens
atgtcggagcccgggggcggcggcggcgaggacggctcggccggattggaagtgtcggccgtgcagaatgtggcggacgtgtcggtgctgcagaagcacctgcgcaagctggtgccgctgctgctggaggacggcggcgaggcgccggccgcgctggaggcggcgctggaggagaagagcgccctggagcagatgcgcaagttcctttcggacccgcaggtccacacggtgctggtggagcgctccacgctcaaagaggacgtcggtgatgaaggagaagaagaaaaagaattcatttcctataacatcaacatagacattcattatggggttaaatccaatagcttggcattcattaaacgtactcccgtgattgatgcagataaacccgtgtcttctcagctccgggtccttacactcagtgaagactcgccctacgaaactttgcattctttcattagcaatgcagtggctcctttttttaagtcctacattagagagtctggcaaggcagacagggatggtgataaaatggctccttcagttgaaaagaagattgcagaactcgaaatgggactccttcacttgcagcaaaatattgaaattccggagatcagcctgccgattcatccaatgatcacaaatgttgcaaaacagtgttatgagcgtggagaaaagccaaaagttacagactttggtgataaggttgaagacccaacatttcttaatcagttacaatctggagttaaccgctggatccgagaaattcaaaaagtgaccaaactggatcgagatcctgcatcaggaactgccttacaggaaattagtttttggctaaacttggaacgtgcgttataccgcatccaggagaaacgggagagcccggaagttctcctgactctggatatcttgaaacatggcaagcgcttccatgccaccgtcagttttgacactgacacaggtctaaaacaggctttggaaactgtgaatgactacaatcctctgatgaaagatttccctctgaatgatttgctgtctgccacggagctggacaaaataagacaggcgcttgttgccattttcacacatttgagaaagatccgaaacacaaaatatcctattcagagggcactgcgtttggtggaggcaatttcaagagacttgagttctcaattactcaaagtattgggcactaggaaattgatgcatgttgcttatgaagaatttgaaaaagttatggtagcatgctttgaagtttttcagacttgggatgatgagtatgagaaacttcaggtattgttgagagacatcgtcaaaagaaaaagggaagaaaatctgaagatggtgtggcgtatcaaccctgcccacaggaagctgcaggcccgccttgaccagatgagaaaatttagacgccagcatgaacagctaagagctgttatcgtcagggtcctgaggccacaggtcacggcagttgcacaacagaatcaaggagaggtccctgaaccccaagatatgaaagtggctgaggttctctttgatgctgcagatgcaaatgccattgaggaagtaaaccttgcttatgagaacgtcaaggaagtggatggactggatgtttccaaagagggcacggaagcctgggaggctgctatgaagaggtacgatgagaggatcgacagagtggagacccggatcaccgctcgccttcgggatcagcttggcacagccaagaatgccaacgagatgtttaggattttctccaggtttaatgcactgtttgtcaggcctcacatccgtggggccattcgcgaataccagacccagctgatccagcgcgtgaaagatgacattgagtctcttcacgacaagttcaaggtccagtacccacagagtcaggcttgtaagatgagtcacgttcgtgacttgccccctgtgtcagggtctatcatctgggctaaacagatcgacaggcagctgacggcctacatgaagcgggtggaagatgtccttggcaagggctgggagaatcacgtggaggggcagaagctgaagcaggatggagacagcttccgcatgaagctcaacacgcaggagatctttgatgactgggcaaggaaggtgcagcagcgcaacctcggtgtctcggggcgcattttcaccatcgaaagtactcgggttcggggccgaactggaaatgtgcttaagctgaaagttaactttcttcctgagattatcacactatccaaagaagtccggaacctcaaatggcttggtttccgcgtcccactggcgattgtgaacaaagcccatcaagcaaaccagctttacccgtttgccatctcactgatcgagagcgttcgtacctatgaacggacctgcgagaaggtggaggagcggaacaccatttcccttttggtggctggcttgaaaaaggaagtgcaggccctgatcgcagaaggcattgcgttggtgtgggagtcctacaaacttgacccatatgtacagcgcttagcagagactgtcttcaacttccaagaaaaggtggatgatctgctgatcattgaagaaaaaatagacctagaagtccgttccttggaaacttgtatgtatgaccataagacattctcggaaatcttgaacagagtccagaaagcagtggatgacttaaatctgcactcctattccaatttgcccatctgggtcaacaagcttgacatggagattgaaagaatattgggcgtccgtctgcaagctggcctgagagcttggacgcaggttcttcttggacaagctgaagataaagcagaagttgacatggacacagatgctccacaagttagtcacaagcctggtggagagccaaagatcaaaaatgtcgttcatgagctaagaataaccaatcaggtaatctacttgaatccaccaattgaagagtgcagatacaagctgtatcaggaaatgtttgcctggaagatggttgtactgtctctccccaggatccagagtcagaggtaccaggtgggtgtacattacgaattgactgaggaagagaaattctatcggaatgctttaacacggatgcctgatggccctgttgccctggaagagtcgtattctgctgtcatgggcattgtatctgaagttgaacagtatgtcaaggtttggcttcagtatcagtgtttatgggatatgcaagctgaaaacatctataacagacttggagaagatctcaacaaatggcaggctctcctggtccaaataaggaaggccagaggaacctttgacaatgcagaaaccaagaaagagtttggaccagtagttatagattatggcaaggtacaatctaaggtgaacttgaaatatgactcttggcataaggaggttcttagcaaatttgggcagatgctaggatcaaacatgacggaattccattcccagatctcaaagtcccgccaagagttggagcagcactcagtagacacggccagcacctccgatgcagtgaccttcatcacctatgtgcagtctttgaaacggaagatcaagcagtttgagaagcaagttgagctctaccgcaatggccagcgcttactggaaaagcaaaggttccagttcccaccttcctggctttatattgacaacatcgagggagagtggggagccttcaatgacatcatgcggcgaaaggactctgccattcagcagcaggtggcaaacctgcaaatgaagattgtccaggaggatcgggccgtggaaagccgcaccaccgacctgctgactgactgggagaagaccaagcctgtcacgggcaaccttcgcccagaagaggcacttcaggctctcaccatatatgaggggaagtttggtaggctgaaggacgacagagagaagtgtgcaaaggccaaggaggcgctggaattgacagatactgggcttctcagtggcagtgaagagcgcgtgcaggtggccttagaagaattacaggacctcaaaggcgtttggtcagaactttctaaggtttgggagcaaatcgatcagatgaaggagcaaccctgggtttcagtacagcctcgaaagcttcgacaaaatttggatgccctcctgaaccagctgaaaagcttccctgcccggttgcgacagtatgcgtcctatgagtttgttcagaggcttctgaaaggttacatgaagataaatatgctggtgattgaactgaaatccgaagcacttaaagaccgccattggaaacagctcatgaaaaggcttcacgttaattgggttgtttctgagctaacccttggccaaatctgggatgttgacttgcagaaaaatgaagcgattgtcaaggatgtactgcttgtggcacaaggggagatggctttggaagaatttttgaagcagataagagaagtgtggaatacttatgaactagacttggttaattatcagaacaagtgccgcttgatccgtggctgggatgacctcttcaacaaggtcaaagaacacatcaacagcgtctcggccatgaagctctctccgtattacaaggtttttgaagaggatgctctcagctgggaagataagctgaacaggatcatggctctctttgatgtgtggattgatgtgcagaggcggtgggtctacctggaaggtatcttcacaggcagtgcagatatcaagcacctgctgccagtggaaacccagcggtttcagagcatcagcactgagtttttggctctaatgaaaaaagtgtccaagtctccccttgttatggatgttctgaacatccagggagtacagaggtctctggaaagattggcagacctgctaggaaagatccagaaagcattgggagaatatctggaaagagagcggtcatctttccccaggttctattttgtgggtgatgaagatttgcttgaaatcattggaaacagcaagaatgtcgctaaattacagaaacacttcaagaagatgtttgctggagtttcgagcatcatcctgaacgaggataactctgttgttttgggtatttcatctcgggaaggagaggaggttatgtttaaaactcctgtgtcaattactgaacatcccaaaatcaatgagtggctcacattggtagaaaaggagatgagagtcaccctggccaaactgcttgctgagtctgttacggaagttgagatttttggtaaagcaacttcaattgacccaaatacctacatcacttggattgataaataccaggcccagcttgtggttttgtcagcccagatagcctggtctgagaacgtggagaccgcactgagcagcatgggcggaggtggagatgccgcgcccttgcactctgtgctgagcaatgtggaggtcaccctcaatgtgttagcagactctgtcctcatggagcagcccccactccgaaggcggaagctagaacacttgattacagagttggttcaccagagagatgttacaaggtccttgatcaaaagcaagattgacaacgccaaatcttttgaatggctcagccagatgcgattttactttgaccctaagcaaactgatgtgttacagcagttgtcaattcaaatggcaaatgccaaatttaactatggctttgagtacctgggtgttcaggacaaactggtccagacccccctcactgaccgctgctatttgacaatgacacaagccttggaggccaggctggggggttccccatttggacctgctggaactgggaaaacagagtctgtcaaagctcttggccatcagcttggacggtttgttttagttttcaactgtgatgaaacctttgatttccaggcaatgggccggatctttgtgggcctttgccaggtgggtgcctggggctgctttgacgagttcaaccgcctggaggagcggatgctctcggctgtgtcccagcaggtgcagtgcatacaggaagcactgcgtgaacattccaaccccaactacgacaagacctctgcccccattacttgtgagctgctgaacaaacaagtcaaggtgagcccggacatggccatcttcatcaccatgaaccctggctacgcgggccggtctaaccttcctgacaacttgaagaagctgttccggagcttggccatgaccaagcccgaccggcagttaatcgcccaggtcatgctgtactcacagggtttccgcactgctgaagtgcttgccaacaaaatcgtcccgttttttaaactatgcgatgagcagctctcttcccaaagccattatgacttcggtcttcgggctttgaagagtgtgctggtgagtgcaggcaatgtgaagagagagagaatccagaagataaagagggagaaagaggaacgaggggaagcagttgatgaaggagaaattgctgaaaatctccctgaacaagagattctgatacagagcgtctgtgagacgatggtgccaaagctggtggcagaggacatcccgctgctcttcagcctcctgtcggacgtgttccctggagtccagtatcacaggggtgagatgactgcccttcgagaggagctgaagaaagtgtgtcaggagatgtatttgacatatggagatggagaagaagttggtggaatgtgggttgaaaaggttctccagctctatcagatcacccagatcaatcatggcctgatgatggtggggccctcgggaagtgggaagagcatggcctggcgtgtcctgctgaaggcattggagagactcgagggtgtggaaggtgtggcccatatcatcgaccccaaggccatcagcaaagaccacctctacggaaccctggaccccaacaccagggaatggacagatgggctcttcacacacgtgctgagaaagatcatcgacagcgtgagaggcgagctgcagaagcgccagtggatcgtcttcgatggcgatgtggatccagagtgggttgagaacttgaactcagtgctggatgacaataagctcctaactttgcccaatggagagcgcctcagtcttccacccaatgtgagaataatgtttgaggtacaggacttgaaatacgcgaccttggccacagtgtcgcgctgcggcatggtctggttcagtgaggatgtgctgagcaccgacatgatcttcaacaacttcctggccaggctgcgcagcatcccgctggatgaaggggaggatgaggcacagcggcggcgtaagggcaaagaggatgagggggaggaggccgcttcccccatgctgcagatccaaagagatgcagctacgatcatgcaaccgtacttcacgtccaacggcctggtcaccaaggcgctagagcacgccttccagctggagcacatcatggacctaacacgcctgcgctgcctgggctcgctcttctccatgctgcaccaggcctgccgcaacgtggcgcagtataacgccaaccatcccgacttccccatgcagatcgagcagctggagcgctacattcagcgatatctggtttatgccatactctggtccctgtctggagacagccggctaaaaatgagagcagagctgggtgaatacatcagaagaatcacgaccgtgcctctgcccactgcgcccaacatacccattatcgattatgaggtgtccatcagcggagaatggtctccgtggcaggccaaggtgcctcagattgaagtggagacgcacaaggtggcagcccctgatgtcgtcgtgccaacgctggacacagtccgccacgaagccctcttgtacacttggctggccgaacacaagcccctggtcttgtgtggccctcctgggtctggcaagaccatgacactcttcagcgccctccgggccttgcctgacatggaggtggtgggtctcaacttctccagtgctactactccagagctgcttctgaagacttttgatcactactgcgagtacaggcgcacacctaatggggtggttttggctcctgttcaacttggaaagtggctggtgttgttctgtgatgaaatcaacttgccagatatggataaatatgggacccagagggtcatatccttcatcagacagatggtggagcacggaggcttttaccgtacctcagatcaaacatgggtgaagctggagagaatccagtttgttggggcttgtaatccccccacagaccctggaagaaagcccctctcacacaggttcctgcgccacgtgcctgtcgtgtatgtggattacccgggccccgcctccctcacacagatctacggcaccttcaaccgcgccatgctgaggctcattccatccctgcggacgtatgcagagccgctcactgctgccatggtggagttctacaccatgtctcaggagagattcacccaggatacacaacctcactatatctattcaccccgtgaaatgactaggtgggtgagaggcatctttgaagcgctgagacctctggagaccctgcctgttgaaggcctcattcggatttgggcacatgaagctctgcgtctcttccaagatagactcgtagaggatgaggagaggcgttggactgatgagaacatcgacacggttgctctgaagcacttccctaacatcgacagagagaaggcaatgagccgacccatcttgtacagcaactggctgtcaaaggattacatcccagtagaccaagaagagttaagagattatgtcaaagctaggctgaaggtcttttatgaagaagaacttgatgttccgctggtgctgtttaatgaagtcctagaccacgtgctgaggattgacagaatattccgtcaacctcaaggccacttgcttctgattggtgttagtggagcaggaaaaactaccctgtctcgtttcgtcgcctggatgaacggtttgagtgtgtaccagattaaggtccataggaagtacacaggggaagactttgatgaagatctacggacagtgttgagacgttctggctgtaaaaatgaaaagatagcatttataatggatgaatctaatgtgttagattctggattcctggagcgaatgaatacccttctggccaatggagaggtgcctggtctctttgaaggagacgagtatgccaccttgatgacgcagtgcaaagagggggcacagaaggaaggcctgatgctggactcgcacgaggagctctacaagtggttcactagccaggttatccgcaacctccacgtcgtgttcaccatgaacccgtcctcggagggactcaaggaccgggcagctacatcaccagcacttttcaacaggtgtgtgttgaattggtttggagactggtccaccgaagcactgtatcaggttggcaaagaattcacaagtaagatggatctggagaagccaaattacatcgtgcctgattacatgccagttgtgtatgataagctgccgcagccaccatcccatcgggaagccattgtgaacagctgtgtgtttgttcatcagactcttcaccaggcgaatgctcggctagcaaagcgaggcggcagaacgatggccatcacccctcgccactacctggacttcatcaatcactatgccaacctgttccacgagaagcggagcgagctggaggagcagcagatgcacttgaacgtggggctcaggaagatcaaagagacagtcgaccaggtagaagaactgcgtcgtgacttgaggataaagagccaagagctggaggtgaagaatgcagcagccaatgacaagctgaaaaagatggtgaaagaccagcaggaggctgaaaagaagaaggttatgagccaagaaatccaggaacagctgcataagcagcaggaggtaattgcagacaaacagatgagtgtcaaagaagatcttgataaggtggaacctgccgtcattgaggcccagaatgctgtgaagtcgatcaagaagcagcacctggtggaggtgaggtccatggccaaccctcctgctgctgtgaagctggcgctggagtccatctgcctgctgctgggggaaagcaccacagactggaagcagatccgctccatcatcatgcgggagaacttcatccccaccatcgtcaacttctctgcagaggagatcagtgacgccataagggagaagatgaagaaaaattacatgtccaatccaagttacaattatgaaattgtgaatcgggcttccctggcttgcggccctatggtgaaatgggcaattgcacagcttaactatgcagacatgttaaagagagtggagcccctacgcaatgagctgcagaagctggaagatgacgccaaggacaaccagcagaaggccaacgaggtggagcagatgatccgagacctggaagccagcatcgcccgctacaaggaggaatacgccgtcctgatctcagaggcccaggccatcaaggcagacctggcagctgtcgaggcaaaagtaaaccggagcactgctcttctgaagagcttgtctgctgaacgtgaacgatgggaaaaaacaagtgaaactttcaaaaaccagatgtccaccattgctggggactgtctcttgtcagctgcgttcattgcctacgcgggttactttgaccagcagatgcgtcagaacttgttcactacctggtcccatcacctacagcaagccaacatccagttccgtacagatattgccaggacggaatacctttccaatgctgatgagcgtcttcgctggcaggccagctccttgcctgctgatgacctttgcacagaaaatgccatcatgctgaaacgattcaataggtatccgctgatcattgacccctctggacaggccacagaattcattatgaatgaatataaggatcgtaagatcacacggaccagcttcctggatgacgccttcagaaagaacttagagagtgcactgagattcggtaacccccttctggtccaggatgtggaaagctacgatccagttttgaacccggtgctgaaccgtgaagtgcggcgaacaggggggagagtgctgatcactctcggggaccaggacatagacctgtcgccatcgtttgtcatcttcctgtccacccgggatccaactgtcgagttcccaccagatctctgttcccgggttacttttgtaaacttcacagttacccgtagcagtttacaaagccagtgtctaaatgaagtacttaaagcagaaagacctgatgtggacgagaaacgatctgatcttcttaaacttcaaggggaatttcagctccgtttgcgtcagctggaaaaatctctactacaagctctgaacgaggtgaaagggcgcattttggatgacgacacgatcataaccactctggagaacctgaagagagaggctgcagaggtcaccaggaaagttgaggagacggacattgtcatgcaggaggtggagaccgtgtcccagcagtacctcccgctctccaccgcctgcagcagcatctacttcaccatggagtccctcaagcagatacacttcttgtaccagtactccctccagtttttcctggacatttatcacaacgtcctatacgagaacccgaacctgaagggtgtcaccgaccacacacagcgcctgtccattataacaaaggacctcttccaggtggcgtttaaccgagtggctcgaggcatgctgcatcaggaccacattacctttgccatgctgctggcaagaatcaaactgaagggcaccgtgggggagcccacctacgatgcagaattccagcacttcttgagaggaaatgagattgtcctgagtgctggctccacccccaggatccagggcctgactgtggagcaggcggaggcggtggtgaggctgagctgccttcccgcgtttaaggacttgattgcaaaggttcaggcagacgagcaatttggcatctggctggacagcagctccccggagcagactgtgccctacctctggagtgaagaaacacctgcaacacccattggccaggccatccaccgcctgctcctgatccaggctttccggcccgatcgcctgttggccatggcccacatgtttgtttcaacaaaccttggggagtctttcatgtccatcatggagcagccgctcgacctgacccacattgtgggcacagaggtgaagcccaacactcctgtcttaatgtgctctgtgcctggttatgatgccagtggacatgtcgaggaccttgcagccgagcagaacacgcagatcacttcaattgcaatcggctctgcagaaggctttaaccaagcagataaggcaataaacaccgctgtaaagtcgggcaggtgggtgatgctgaagaatgtgcatctggccccagggtggctgatgcagctggagaagaagttgcattccctgcagccgcatgcctgcttccgactcttcctcaccatggagatcaaccccaaggtgcctgtgaatctgctccgtgcgggccgcatctttgtgttcgagccaccgccaggggtgaaggccaacatgctgaggacgttcagcagcattcccgtctcacggatatgcaagtctcccaacgagcgtgcccgcttgtacttcctgctggcctggtttcatgcgatcatccaagaacgcttacgatacgcaccactggggtggtcaaagaagtatgaatttggagagtctgacctgcggtcagcttgcgatacggtggacacgtggctggatgacacggccaagggcaggcagaacatctcaccggataagatcccgtggtctgcactaaagaccttaatggcccagtccatttatggcgggcgcgtggacaacgagtttgaccagcgtctgctcaacaccttcctggagcgcctgttcacaaccaggagtttcgacagtgagtttaagctggcatgcaaggtcgacggacataaagacattcaaatgccagatggcatcaggcgagaggagtttgtgcagtgggtggagttgctccccgacacccagacgccctcctggctgggcctgcccaacaacgccgagagagtcctccttaccacacagggtgtggacatgatcagtaaaatgctgaagatgcagatgttggaggatgaggacgacctggcctacgcagagactgagaagaagacgaggacagactccacgtccgacgggcgccctgcctggatgcggacactgcacaccaccgcgtccaactggctgcacctcatcccccagacgctgagccacctcaagcgcaccgtggagaatatcaaggatcctttgttcaggttctttgagagagaagtgaagatgggcgcaaagctgcttcaggacgttcgccaggaccttgcagatgtcgtccaggtgtgcgaaggaaagaagaagcagaccaactacttgcgcacgctgatcaacgagctagtgaaagggatcttgcctcggagctggtcccactacacggtgcctgccggcatgaccgtcatccagtgggtgtccgacttcagcgagaggatcaaacagctgcagaacatctcactggcagctgcatctggtggcgccaaggagctaaagaacatccacgtgtgcctgggtggcctgttcgtgcctgaggcgtacatcactgccaccaggcagtatgtggcccaggccaacagctggtccctggaggagctctgcctggaagtcaacgtcaccacctcacagggcgccacccttgacgcttgcagcttcggagtcacgggtttgaaacttcaaggggccacgtgcaacaacaacaagctgtcactgtccaatgccatctcaaccgcccttcccctgacgcagctgcgctgggtcaagcagacaaacaccgagaagaaggccagtgtggtaaccttacctgtctacctgaacttcacccgtgcagacctcatcttcaccgtggacttcgaaattgctacaaaggaggatcctcgcagcttctacgagcggggtgtcgcagtcttgtgcacagagtaa

SEQ ID NO: 18 - protein, 4646 aa, Homo sapiens [sequence omitted]

SEQ ID NO: 19 - DNA, 177 bp, Artificial Sequence
  Mus musculus exon13, cytoplasmic dynein heavy chain 1
gtttggcttcagtaccagtgtttgtgggatatgcaggcagaaaacatttacaacaggctaggggaagatctcaacaagtggcaagctctcctggtccagataaggaaagccagaggaacctttgacaatgcggaaaccaagaaagagtttggtccggtggtgatagattacggcaag

SEQ ID NO: 20 - DNA, 318 bp, Artificial Sequence
  Mus musculus exon 12 and 13, cytoplasmic dynein heavy chain 1
gtgggagtgcattacgagttgacagaggaagagaagttctatcggaatgcactgacaaggatgcccgacggccctgtcgccctggaagagtcctactctgcggtcatgggcatagtgactgaagttgagcagtatgttaaggtttggcttcagtaccagtgtttgtgggatatgcaggcagaaaacatttacaacaggctaggggaagatctcaacaagtggcaagctctcctggtccagataaggaaagccagaggaacctttgacaatgcggaaaccaagaaagagtttggtccggtggtgatagattacggcaag

SEQ ID NO: 21 - protein, 59 aa, Artificial Sequence
  cytoplasmic dynein heavy chain 1, exon 12 derived amino acid sequence, Mus musculus
VWLQYQCLWDMQAENIYNRLGEDLNKWQALLVQIRKARGTFDNAETKKEFGPVVIDYGK

SEQ ID NO: 22 - protein, 106 aa, Artificial Sequence
  cytoplasmic dynein heavy chain 1, exon 12 and exon 13 derived amino acid sequence, Mus musculus
VGVHYELTEEEKFYRNALTRMPDGPVALEESYSAVMGIVTEVEQYVKVWLQYQCLWDMQAENIYNRLGEDLNKWQALLVQIRKARGTFDNAETKKEFGPVVIDYGK

SEQ ID NO: 23 - protein, 59 aa, Artificial Sequence; identical to SEQ ID NO: 21
  cytoplasmic dynein heavy chain 1, exon 13 derived amino acid sequence, Homo sapiens

SEQ ID NO: 24 - protein, 106 aa, Artificial Sequence
  cytoplasmic dynein heavy chain 1, exon 12 and exon 13 derived amino acid sequence, Homo sapiens
VGVHYELTEEEKFYRNALTRMPDGPVALEESYSAVMGIVSEVEQYVKVWLQYQCLWDMQAENIYNRLGEDLNKWQALLVQIRKARGTFDNAETKKEFGPVVIDYGK

SEQ ID NO: 25 - protein, 21 aa, Artificial Sequence
  cytoplasmic dynein heavy chain 1 DNA derived 21 amino acid stretch, Mus musculus
VEQYVKVWLQYQCLWDMQAEN

SEQ ID NO: 26 - DNA, 22 bp, Artificial Sequence
  oligonucleotide primer
tgtgaacataagaggtgccagt

SEQ ID NO: 27 - DNA, 20 bp, Artificial Sequence
  oligonucleotide primer
ctacccgtgacacctttcaa

SEQ ID NO: 28 - DNA, 150 bp, Mus musculus
ggtgggcaggaggcaacagatattgcccttcttctccgagacctggaaacatcatgtagtgacacccgtcagttctgcaagaagatccgaaggcggatgccggggacggatgctcctgggatcccagcagcgctggcctttggctcacag

SEQ ID NO: 29 - DNA, 162 bp, Mus musculus
gtatccgacacactcctggactgcaggaagcacttgacgtgggtggtagctgttctgcaggaggtggcagctgcagccgcccagcttattgcccccttggcagagaacgaggggctgcctgtggctgcactggaggagctggccttcaaagcaagcgagcag

SEQ ID NO: 30 - DNA, 132 bp, Mus musculus
atctacgggagcccctccagcagcccctatgagtgtctacgccagtcatgcaccatcctcatcagcacgatgaacaagctggccacagccatgcaagaaggcgagtatgacgcagagcgacccccgagcaag

SEQ ID NO: 31 - DNA, 2402 bp, Mus musculus
agggagccgatgcagctgcaaagaccagcaaactgcggggactgaagcctaagaaggcaccgacagcccgaaagaccacaactcgacggcccaagcctactcgcccagccagcactggggtggctgggcccagtagctcccttggcccctctggctcagcgtcagccggggaactaagcagcagtgagcccagcaccccagctcagactccgctggcagcacccatcatccccacaccggccctcacctctcctggagcagcacccccacttccatctccctctaaggaagaggaagggctgagggctcaggtacgggacctggaggagaagctggagaccctgcgcctaaaacgctcagaagacaaagcaaagctgaaagagctggagaagcacaagatccagctggagcaggtgcaggaatggaagagcaaaatgcaggagcagcaggcagacctgcagcggcgcctcaaggaggctcggaaggaagccaaggaggcgctagaggcaaaggaacgctacatggaggagatggccgacacagccgacgctatcgagatggccactctggacaaggagatggctgaagagcgcgctgagtctctgcagcaagaggtggaggcactgaaggaacgggtagacgagctcaccacagacctggagattctcaaggctgaaatcgaagagaaaggctctgatggggccgcatcaagctaccagctcaagcagctggaggagcagaatgcccgcctgaaggatgccctggtgaggatgcgagacctctcttcctcagagaagcaggagcacgtgaagctgcagaaactcatggaaaagaaaaaccaggagctggaggtcgtgcggcagcagcgcgagcgtcttcaggaggagctgagccaggctgagagcaccatcgatgagctcaaagagcaggtggacgccgctctgggagccgaggagatggtggagatgctgaccgaccggaacctgaatctagaggagaaagtgcgggagttacgggagactgtgggggacttggaagccatgaacgagatgaacgatgagctgcaggagaacgcacgggagacggagctggaactccgagagcagctggacatggcgggcgcccgagtgagggaagcgcagaagcgagtggaagccgcccaggagacagtcgccgactaccagcagaccatcaagaagtaccgccagttgactgcccacctacaggatgtcaatcgggagctgacaaaccagcaggaagcgtctgtagagaggcagcagcagccgccgccagagacttttgatttcaaaatcaagtttgctgagaccaaggctcatgccaaggccattgagatggagttgagacagatggaagttgcccaggccaaccggcacatgtccctgctgacagcctttatgcctgacagcttccttcggccaggtggagaccacgactgtgtcctggtgctgctgctcatgccccgactcatttgcaaggcagagctcatccggaagcaggcccaggagaagtttgacctgagcgagaactgttcggagcggcccgggctgcggggagctgccggggagcagctgagctttgctgctggactggtgtactcgctgagtctgctgcaggccacgctgcaccgctatgagcatgccctctctcagtgcagtgtggacgtgtataagaaggtcggcagcctgtaccccgagatgagcgcccacgagcgctccttagatttcctcattgagctgctgcacaaggatcagctggatgagactgtcaacgtggagcccctcamcaaggscatcaagtattaccagcatctgtacagsatccacctcgctgaacaacccgaggactccaccatgcagctggctgaccacatcaagttcacccagagtgccctggactgcatgggcgtggaggtggggcggctgcgtgccttcttgcagggtgggcaggaggcaacagatattgcccttcttctccgagacctggaaacatcatgtagtgacacccgtcagttctgcaagaagatccgaaggcggatgccggggacggatgctcctgggatcccagcagcgctggcctttggctcacaggtatccgacacactcctggactgcaggaagcacttgacgtgggtggtagctgttctgcaggaggtggcagctgcagccgcccagcttattgcccccttggcagagaacgaggggctgcctgtggctgcactggaggagctggccttcaaagcaagcgagcagatctacgggagcccctccagcagcccctatgagtgtctacgccagtcatgcaccatcctcatcagcacgatgaacaagctggccacagccatgcaagaaggcgagtatgacgcagagcgacccccgagcaag

SEQ ID NO: 32 - DNA, 143 bp, Mus musculus
ggagaggagctgagtgaggccaacgtgcggctcagcctcctggagaagaagttggacagcgctgccaaggatgcagacgagcgaatcgagaaagttcagacacggctggacgagactcagaccctgctgcggaagaaggagaa

SEQ ID NO: 33 - DNA, 167 bp, Mus musculus
agactttgaggagacaatggacgcactccaggctgacatcgaccagctggaggcagagaaggcagagctcaagcagcgcctgaacagccagtccaagcgcacaatcgaggggctccggggcccccctccgtcaggcatcgctaccctggtctctggcatcgctggtg

SEQ ID NO: 34 - DNA, 310 bp, Mus musculus
ggagaggagctgagtgaggccaacgtgcggctcagcctcctggagaagaagttggacagcgctgccaaggatgcagacgagcgaatcgagaaagttcagacacggctggacgagactcagaccctgctgcggaagaaggagaaagactttgaggagacaatggacgcactccaggctgacatcgaccagctggaggcagagaaggcagagctcaagcagcgcctgaacagccagtccaagcgcacaatcgaggggctccggggcccccctccgtcaggcatcgctaccctggtctctggcatcgctggtg

SEQ ID NO: 35 - DNA, 246 bp, Homo sapiens
acgcccagcggcagcaggatgagtgcggaggcaagcgcccggcctctgcgggtgggctcccgtgtagaggtgattggaaaaggccaccgaggcactgtggcctatgttggagccacactgtttgccactggcaaatgggtaggcgtgattctggatgaagcaaagggcaaaaatgatggaactgttcaaggcaggaagtacttcacttgtgatgaagggcatggcatctttgtgcgccagtcccag

SEQ ID NO: 36 - DNA, 79 bp, Homo sapiens
atccaggtatttgaagatggagcagatactacttccccagagacacctgattcttctgcttcaaaagtcctcaaaagag

SEQ ID NO: 37 - DNA, 35 bp, Homo sapiens
agggaactgatacaactgcaaagactagcaaactg

SEQ ID NO: 38 - DNA, 21 bp, Homo sapiens
accacaactcggcgacccaag

SEQ ID NO: 39 - DNA, 420 bp, Homo sapiens
acgcccagcggcagcaggatgagtgcggaggcaagcgcccggcctctgcgggtgggctcccgtgtagaggtgattggaaaaggccaccgaggcactgtggcctatgttggagccacactgtttgccactggcaaatgggtaggcgtgattctggatgaagcaaagggcaaaaatgatggaactgttcaaggcaggaagtacttcacttgtgatgaagggcatggcatctttgtgcgccagtcccagatccaggtatttgaagatggagcagatactacttccccagagacacctgattcttctgcttcaaaagtcctcaaaagagagggaactgatacaactgcaaagactagcaaactgcggggactgaagcctaagaaggcaccgacagcccgaaagaccacaactcggcgacccaag

SEQ ID NO: 40 - DNA, 192 bp, Homo sapiens
cccacgcgcccagccagtactggggtggctggggccagtagctccctgggcccctctggctcagcgtcagcaggtgagctgagcagcagtgagcccagcaccccggctcagactccgctggcagcacccatcatccccacgccggtcctcacctctcctggagcagtccccccgcttccttccccatccaag

SEQ ID NO: 41 - DNA, 198 bp, Homo sapiens
gaggaggagggactaagggctcaggtgcgggacctggaggagaaactagagaccctgagactgaaacgggcagaagacaaagcaaagctaaaagagctggagaaacacaaaatccagctggagcaggtgcaggaatggaagagcaaaatgcaggagcagcaggccgacctgcagcggcgcctcaaggaggcgagaaag

SEQ ID NO: 42 - DNA, 205 bp, Homo sapiens
gaagccaaggaggcgctggaggcaaaggaacgctatatggaggagatggctgatactgctgatgccattgagatggccactttggacaaggagatggctgaagagcgggctgagtccctgcagcaggaggtggaggcactgaaggagcgggtggacgagctcactactgacttagagatcctcaaggctgagattgaagagaagg

SEQ ID NO: 43 - DNA, 79 bp, Homo sapiens
gctcagatggcgctgcatccagttatcagctcaagcagcttgaggagcagaatgcccgcctgaaggatgccctggtgag

SEQ ID NO: 44 - DNA, 160 bp, Homo sapiens
gatgcgggatctttcttcctcagagaagcaggagcatgtgaagctccagaagctcatggaaaagaagaaccaagagctggaagttgtgaggcaacagcgggagcgtctgcaggaggagctaagccaggcagagagcaccattgatgagctcaaggagcag

SEQ ID NO: 45 - DNA, 105 bp, Homo sapiens
gtggatgctgctctgggtgctgaggagatggtggagatgctgacagatcggaacctgaatctggaagagaaagtgcgcgagttgagggagactgtgggagacttg

SEQ ID NO: 46 - DNA, 192 bp, Homo sapiens
gaagcgatgaatgagatgaacgatgagctgcaggagaatgcacgtgagacagaactggagctgcgggagcagctggacatggcaggcgcgcgggttcgtgaggcccagaagcgtgtggaggcagcccaggagacggttgcagactaccagcagaccatcaagaagtaccgccagctgaccgcccatctacag

SEQ ID NO: 47 - DNA, 117 bp, Homo sapiens
gatgtgaatcgggaactgacaaaccagcaggaagcatctgtggagaggcaacagcagccacctccagagacctttgacttcaaaatcaagtttgctgagactaaggcccatgccaag

SEQ ID NO: 48 - DNA, 153 bp, Homo sapiens
gcaattgagatggaattgaggcagatggaggtggcccaggccaatcgacacatgtccctgctgacagccttcatgcctgacagcttccttcggccaggtggggaccatgactgcgttctggtgctgttgctcatgcctcgtctcatttgcaag

SEQ ID NO: 49 - DNA, 161 bp, Homo sapiens
gcagagctgatccggaagcaggcccaggagaagtttgaactaagtgagaactgttcagagcggcctgggctgcgaggagctgctggggagcaactcagctttgctgctggactggtgtactcgctgagcctgctgcaggccacgctacaccgctatgagca

SEQ ID NO: 50 - DNA, 169 bp, Homo sapiens
tgccctctctcagtgcagtgtggatgtgtataagaaagtgggcagcctgtaccctgagatgagtgcccatgagcgctccttggatttcctcattgaactgctgcacaaggatcagctggatgagactgtcaatgtggagcctctcaccaaggccatcaagtactatcag

SEQ ID NO: 51 - DNA, 69 bp, Homo sapiens
catctgtacagcatccaccttgccgaacagcctgaggactgtactatgcagctggctgaccacattaag

SEQ ID NO: 52 - DNA, 63 bp, Homo sapiens
ttcacgcagagtgctctggactgcatgagtgtggaggtaggacggctgcgtgccttcttgcag

SEQ ID NO: 53 - DNA, 150 bp, Homo sapiens
ggtgggcaggaggctacagatattgccctcctgctccgggatctggaaacttcatgcagtgacatccgccagttctgcaagaagatccgaaggcgaatgccagggacagatgctcctgggatcccagctgcactggcctttggaccacag

SEQ ID NO: 54 - DNA, 162 bp, Homo sapiens
gtatctgacacgctcctagactgcaggaaacacttgacgtgggtcgtggctgtgctgcaggaggtggcagctgctgctgcccagctcattgccccactggcagagaatgaggggctacttgtggctgctctggaggaactggctttcaaagcaagcgagcag

SEQ ID NO: 55 - DNA, 132 bp, Homo sapiens
atctatgggaccccctccagcagcccctatgagtgtctgcgccagtcatgcaacatcctcatcagtaccatgaacaagctggccacagccatgcaggagggggagtatgatgcagagcggccccccagcaag

SEQ ID NO: 56 - DNA, 2402 bp, Homo sapiens
agggaactgatacaactgcaaagactagcaaactgcggggactgaagcctaagaaggcaccgacagcccgaaagaccacaactcggcgacccaagcccacgcgcccagccagtactggggtggctggggccagtagctccctgggcccctctggctcagcgtcagcaggtgagctgagcagcagtgagcccagcaccccggctcagactccgctggcagcacccatcatccccacgccggtcctcacctctcctggagcagtccccccgcttccttccccatccaaggaggaggagggactaagggctcaggtgcgggacctggaggagaaactagagaccctgagactgaaacgggcagaagacaaagcaaagctaaaagagctggagaaacacaaaatccagctggagcaggtgcaggaatggaagagcaaaatgcaggagcagcaggccgacctgcagcggcgcctcaaggaggcgagaaaggaagccaaggaggcgctggaggcaaaggaacgctatatggaggagatggctgatactgctgatgccattgagatggccactttggacaaggagatggctgaagagcgggctgagtccctgcagcaggaggtggaggcactgaaggagcgggtggacgagctcactactgacttagagatcctcaaggctgagattgaagagaagggctcagatggcgctgcatccagttatcagctcaagcagcttgaggagcagaatgcccgcctgaaggatgccctggtgaggatgcgggatctttcttcctcagagaagcaggagcatgtgaagctccagaagctcatggaaaagaagaaccaagagctggaagttgtgaggcaacagcgggagcgtctgcaggaggagctaagccaggcagagagcaccattgatgagctcaaggagcaggtggatgctgctctgggtgctgaggagatggtggagatgctgacagatcggaacctgaatctggaagagaaagtgcgcgagttgagggagactgtgggagacttggaagcgatgaatgagatgaacgatgagctgcaggagaatgcacgtgagacagaactggagctgcgggagcagctggacatggcaggcgcgcgggttcgtgaggcccagaagcgtgtggaggcagcccaggagacggttgcagactaccagcagaccatcaagaagtaccgccagctgaccgcccatctacaggatgtgaatcgggaactgacaaaccagcaggaagcatctgtggagaggcaacagcagccacctccagagacctttgacttcaaaatcaagtttgctgagactaaggcccatgccaaggcaattgagatggaattgaggcagatggaggtggcccaggccaatcgacacatgtccctgctgacagccttcatgcctgacagcttccttcggccaggtggggaccatgactgcgttctggtgctgttgctcatgcctcgtctcatttgcaaggcagagctgatccggaagcaggcccaggagaagtttgaactaagtgagaactgttcagagcggcctgggctgcgaggagctgctggggagcaactcagctttgctgctggactggtgtactcgctgagcctgctgcaggccacgctacaccgctatgagcatgccctctctcagtgcagtgtggatgtgtataagaaagtgggcagcctgtaccctgagatgagtgcccatgagcgctccttggatttcctcattgaactgctgcacaaggatcagctggatgagactgtcaatgtggagcctctcaccaaggccatcaagtactatcagcatctgtacagcatccaccttgccgaacagcctgaggactgtactatgcagctggctgaccacattaagttcacgcagagtgctctggactgcatgagtgtggaggtaggacggctgcgtgccttcttgcagggtgggcaggaggctacagatattgccctcctgctccgggatctggaaacttcatgcagtgacatccgccagttctgcaagaagatccgaaggcgaatgccagggacagatgctcctgggatcccagctgcactggcctttggaccacaggtatctgacacgctcctagactgcaggaaacacttgacgtgggtcgtggctgtgctgcaggaggtggcagctgctgctgcccagctcattgccccactggcagagaatgaggggctacttgtggctgctctggaggaactggctttcaaagcaagcgagcagatctatgggaccccctccagcagcccctatgagtgtctgcgccagtcatgcaacatcctcatcagtaccatgaacaagctggccacagccatgcaggagggggagtatgatgcagagcggccccccagcaag

SEQ ID NO: 57 - DNA, 143 bp, Homo sapiens
ggagaggagctaagtgaggccaatgtgcggctgagcctcctggagaagaagttggacagtgctgccaaggatgcagatgagcgcatcgagaaagtccagactcggctggaggagacccaggcactgctgcgaaagaaggagaa

SEQ ID NO: 58 - DNA, 167 bp, Homo sapiens
agagtttgaggagacaatggatgcactccaggctgacatcgaccagctggaggcagagaaggcagaactaaagcagcgtctgaacagccagtccaaacgcacgattgagggactccggggccctcctccttcaggcattgctactctggtctctggcattgctggtg

SEQ ID NO: 59 - DNA, 310 bp, Homo sapiens
ggagaggagctaagtgaggccaatgtgcggctgagcctcctggagaagaagttggacagtgctgccaaggatgcagatgagcgcatcgagaaagtccagactcggctggaggagacccaggcactgctgcgaaagaaggagaaagagtttgaggagacaatggatgcactccaggctgacatcgaccagctggaggcagagaaggcagaactaaagcagcgtctgaacagccagtccaaacgcacgattgagggactccggggccctcctccttcaggcattgctactctggtctctggcattgctggtg

SEQ ID NO: 60 - DNA, 1653 bp, Mus musculus
gtcgcctttgctggggacccggacccagcctctcccccactcggtcaccagccccggctcctccgaggcctcggtcccccagccccgcctcgccgccgccatggcggaccctaaatacgccgatctccccggcattgccaggaatgagccagacgtttatgaaaccagcgacctacctgaggatgatcaagcagagtttgatgcggaggaactgagcagcacaagcgtggaacacatcattgtcaaccccaacgctgcctacgacaagttcaaggacaagagagtgggaacaaaggggcttgatttctcagatcgcattggaaagaccaagaggacaggatatgagtctggagactatgaaatgcttggagagggtctgggagtgaaggagacaccgcagcagaagtaccaacgactactgcatgaagtccaagagctgacaactgaggtggagaagatcaagacgacagtgaaggagtcagccaccgaggagaagctcacccctgtggtgctggctaagcagctggctgccctgaagcagcagctggtggcttcccacctggagaagcttctgggaccagatgctgcaatcaaccttgccgacccagacggagccctagctaagcgcttactgctgcagctggaagcaactaagagcagcaaaggcagttcagggggtaaggctacagctggggcccccccagacagcagcctggtcacctatgaactccactcacggcctgagcaggacaagttctctcaagctgccaaagttgcagaactcgagaagcgtctgacagagctggaggccactgtccgatgtgaccaggatgctcagaatcccctttctgcaggtctacagggagcctgccttatggaaacggtagaactgttgcaagccaaagtgagtgccctggaccttgcagttctggaccaagtagaggctcggcttcagagtgtcctgggaaaggtgaatgagattgctaagcacaaggcatccgtggaggacgcagacactcagaacaaggtgcaccagctctatgagaccatccagcgctggagcccagtcgcctccaccctccctgaactggtacagcgacttgtcaccatcaagcagctgcatgagcaagctatgcagtttggtcagctcctgacacacttggataccacccagcagatgatggccagttcccttaaggataatactgctctcctgacccaggtgcagacaacgatgcgtgaaaacttggccacagttgaggggaactttgccagcattgatgctcggatgaagaggctgggaaagtgagcatcactgggagctggggagcggggatgacccttgcccctgaactttgtcagacccttccacagggtcactcacagccccagcccacctgacaagggcaagggttcttgcatgtggggtacacaccctcccgacaaacagagtgggagctgggggctcttattggatgaggttgtgggcccagccacatgcagttcccgtccagtaagtgctttgcctgctgtgggagagggactggatcccgtcctctaacgtctgtctctggccgagtctaacactgtacaactgtctctgaccattaaatgctgttgtaccgtggaaaaaaaaaaaaaaaaaaaaaaaaa

SEQ ID NO: 61 - protein, 628 aa, Mus musculus
MSDKSDLKAELERKKQRLAQIREEKKRKEEERKKKEADMQQKKEPVQDDSDLDRKRRETEALLQSIGISPEPPLVPTPMSPSSKSVSTPSDAGSQDSGDLGPLTRTLQWDTDPSVLQLQSDSELGRRLHKLGVSKVTQVDFLPREVVSYSKETQTPLATHQSEEDEEDEEMVEPKIGHDSELENQEKKQETKEAPPRELTEEEKQQILHSEEFLIFFDRTIRVIERALAEDSDIFFDYSGRELEEKDGDVQAGANLSFNRQFYDEHWSKHRVVTCMDWSLQYPELMVASYSNNEDAPHEPDGVALVWNMKFKKTTPEYVFHCQSSVMSVCFARFHPNLVVGGTYSGQIVLWDNRSHRRTPVQRTPLSAAAHTHPVYCVNVVGTHNARNLITVSTDGKMCSWSLDMLSTPQESMELVYNKSKPVAVTGMAFPTGDVNNFVVGSEEGTVYTACRHGSKAGIGEVFEGHQGPVTGINCHMAVGPIDFSHLFVTSSFDWTVKLWTTKHNKPVYSSEDNADYVYDVMWSPVHPALFACVDGMGRLDLWNLNSDTEVPTASVAIEGASALNRVRWAQGGKEVAVGDSEGRIWIYDVGELAVPHNDEWTRFARTLVEIRANRADSEEEGAVELAA

SEQ ID NO: 62 - protein, 645 aa, Homo sapiens <s300> Genbank/NP_004402.1 2000-11-01 (1)..(645)
MSDKSDLKAELERKKQRLAQIREEKKRKEEERKKKEADMQQKKEPVQDDSDLDRKRRETEALLQSIGISPEPPLVQPLHFLTWDTCYFHYLVPTPMSPSSKSVSTPSEAGSQDSGDLGPLTRTLQWDTDPSVLQLQSDSELGRRLHKLGVSKVTQVDFLPREVVSYSKETQTPLATHQSEEDEEDEEMVESKVGQDSELENQDKKQEVKEAPPRELTEEEKQQILHSEEFLIFFDRTIRVIERALAEDSDIFFDYSGRELEEKDGDVQAGANLSFNRQFYDEHWSKHRVVTCMDWSLQYPELMVASYNNNEDAPHEPDGVALVWNMKFKKTTPEYVFHCQSSVMSVCFARFHPNLVVGGTYSGQIVLWDNRSHRRTPVQRTPLSAAAHTHPVYCVNVVGTQNAHNLITVSTDGKMCSWSLDMLSTPQESMELVYNKSKPVAVTGMAFPTGDVNNFVVGSEEGTVYTACRHGSKAGIGEVFEGHQGPVTGINCHMAVGPIDFSHLFVTSSFDWTVKLWTTKHNKPLYSFEDNADYVYDVMWSPVHPALFACVDGMGRLDLWNLNNDTEVPTASVAIEGASALNRVRWAQAGKEVAVGDSEGRIWVYDVGELAVPHNDEWTRFARTLVEIRANRADSEEEGTVELSA

SEQ ID NO: 63 - protein, 643 aa, Rattus norvegicus <s300> Genbank/NP_062107.1 2002-11-05 (1)..(643)
MSDKSDLKAELERKKQRLAQIREEKKRKEEERKKKEADMQQKKEPVPDDSDLDRKRRETEALLQSIGISPEPPLVQPLHFLTWDTCYFHYLVPTPMSPSSKSVSTPSEAGSQDDLGPLTRTLQWDTDPSVLQLQSDSELGRRLNKLGVSKVTQVDFLPREVVSYSKETQTPLATHQSEEDEEDEEMVEPKVGHDSELENQDKKQETKEAPPRELTEEEKQQILHSEEFLIFFDRTIRVIERALAEDSDIFFDYSGRELEEKDGDVQAGANLSFNRQFYDEHWSKHRVVTCMDWSLQYPELMVASYSNNEDAPHEPDGVALVWNMKFKKTTPEYVFHCQSSVMSVCFARFHPNLVVGGTYSGQIVLWDNRSHRRTPVQRTPLSAAAHTHPVYCVNVVGTQNAHNLITVSTDGKMCSWSLDMLSTPQESMELVYNKSKPVAVTGMAFPTGDVNNFVVGSEEGTVYTACRHGSKAGIGEVFEGHQGPVTGINCHMAVGPIDFSHLFVTSSFDWTVKLWTTKHNKPLYSFEDNADYVYDVMWSPVHPALFACVDGMGRLDLWNLNSDTEVPTASVAIEGAYALNRVRWAQGGKEVAVGDSEGRIWIYDVGELAVPHNDEWTRFARTLVEIRANRADSEEEGAVELAA

SEQ ID NO: 64 - protein, 612 aa, Mus musculus <s300> Genbank/NP_034194 2002-01-07 (1)..(612)
MSDKSDLKAELERKKQRLAQIREEKKRKEEERKKKETDQKKEAAVSVQEESDLEKKRREAEALLQSMGLTTDSPIVPPPMSPSSKSVSTPSEAGSQDSGDGAVGSRRGPIKLGMAKITQVDFPPREIVTYTKETQTPVTAQPKEDEEEEDDVATPKPPVEPEEEKTLKKDEENDSKAPPHELTEEEKQQILHSEEFLSFFDHSTRIVERALSEQINIFFDYSGRDLEDKEGEIQAGAKLSLNRQFFDERWSKHRVVSCLDWSSQYPELLVASYNNNEEAPHEPDGVALVWNMKYKKTTPEYVFHCQSAVMSATFAKFHPNLVVGGTYSGQIVLWDNRSNKRTPVQRTPLSAAAHTHPVYCVNVVGTQNAHNLISISTDGKICSWSLDMLSHPQDSMELVHKQSKAVAVTSMSFPVGDVNNFVVGSEEGSVYTACRHGSKAGISEMFEGHQGPITGIHCHAAVGAVDFSHLFVTSSFDWTVKLWTTKNNKPLYSFEDNSDYVYDVMWSPTHPALFACVDGMGRLDLWNLNNDTEVPTASISVEGNPALNRVRWTHSGREIAVGDSEGQIVIYDVGEQIAVPRNDEWARFGRTLAEINANRADAEEEAATRIPA

SEQ ID NO: 65 - protein, 638 aa, Homo sapiens <s300> Genbank/NP_001369 200-12-23 (1)..(638)
MSDKSELKAELERKKQRLAQIREEKKRKEEERKKKETDQKKEAVAPVQEESDLEKKRREAEALLQSMGLTPESPIVFSEYWVPPPMSPSSKSVSTPSEAGSQDSGDGAVGSRTLHWDTDPSVLQLHSDSDLGRGPIKLGMAKITQVDFPPREIVTYTKETQTPVMAQPKEDEEEDDDVVAPKPPIEPEEEKTLKKDEENDSKAPPHELTEEEKQQILHSEEFLSFFDHSTRIVERALSEQINIFFDYSGRDLEDKEGEIQAGAKLSLNRQFFDERWSKHRVVSCLDWSSQYPELLVASYNNNEDAPHEPDGVALVWNMKYKKTTPEYVFHCQSAVMSATFAKFHPNLVVGGTYSGQIVLWDNRSNKRTPVQRTPLSAAAHTHPVYCVNVVGTQNAHNLISISTDGKICSWSLDMLSHPQDSMELVHKQSKAVAVTSMSFPVGDVNNFVVGSEEGSVYTACRHGSKAGISEMFEGHQGPITGIHCHAAVGAVDFSHLFVTSSFDWTVKLWTTKNNKPLYSFEDNADYVYDVMWSPTHPALFACVDGMGRLDLWNLNNDTEVPTASISVEGNPALNRVRWTHSGREIAVGDSEGQIVIYDVGEQIAVPRNDEWARFGRTLAEINANRADAEEEAATRIPA

SEQ ID NO: 66 - protein, 638 aa, Rattus norvegicus <s300> Genbank/NP_446332 2001-11-07 (1)..(638)
MSDKSELKAELERKKQRLAQIREEKKRKEEERKKKETDQKKEAAVSVQEESDLEKKRREAEALLQSMGLTTDSPIVFSEHWVPPPMSPSSKSVSTPSEAGSQDSGDGAVGSRTLHWDTDPSALQLHSDSDLGRGPIKLGMAKITQVDFPPREIVTYTKETQTPVTAQPKEDEEEEDDVAAPKPPVEPEEEKILKKDEENDSKAPPHELTEEEKQQILHSEEFLSFFDHSTRIVERALSEQINIFFDYSGRDLEDKEGEIQAGAKLSLNRQFFDERWSKHRVVSCLDWSSQYPELLVASYNNNEEAPHEPDGVALVWNMKYKKTTPEYVFHCQSAVMSATFAKFHPNLVVGGTYSGQIVLWDNRSNKRTPVQRTPLSAAAHTHPVYCVNVVGTQNAHNLISISTDGKICSWSLDMLSHPQDSMELVHKQSKAVAVTSMSFPVGDVNNFVVGSEEGSVYTACRHGSKAGISEMFEGHQGPITGIHCHAAVGAVDFSHLFVTSSFDWTVKLWSTKNNKPLYSFEDNSDYVYDVIGSPTHPALFACVDGMGRLDLWNLNNDTEVPTASISVEGNPALNRVRWTHSGREIAVGDSEGQIVIYDVGEQIAVPRNDEWARFGRTLAEINASRADAEEEAATRIPA

SEQ ID NO: 67 - protein, 1281 aa, Mus musculus
  misc_feature (473)..(473): Xaa can be any naturally occurring amino acid
  misc_feature (973)..(973): Xaa can be any naturally occurring amino acid <s300> Genbank/NP_031861 2002-09-19 (1)..(1281)
MAQSRRHMSSRTPSGSRMSTEASARPLRVGSRVEVIGKGHRGTVAYVGATLFATGKWVGVILDEAKGKNDGTVQGRKYFTCDEGHGIFVRQSQIQVFEDGADTTSPETPDSSASKVLKREGADAAAKTSKLRGLKPKKAPTARKTTTRRPKPTRPASTGVAGPSSSLGPSGSASAGELSSSEPSTPAQTPLAAPIIPTPALTSPGAAPPLPSPSKEEEGLRAQVRDLEEKLETLRLKRSEDKAKLKELEKHKIQLEQVQEWKSKMQEQQADLQRRLKEARKEAKEALEAKERYMEEMADTADAIEMATLDKEMAEERAESLQQEVEALKERVDELTTDLEILKAEIEEKGSDGAASSYQLKQLEEQNARLKDALVRMRDLSSSEKQEHVKLQKLMEKKNQELEVVRQQRERLQEELSQAESTIDELKEQVDAALGAEEMVEMLTDRNLNLEEKVRELRETVGDLEAMNEMNDXLQENARETELELREQLDMAGARVREAQKRVEAAQETVADYQQTIKKYRQLTAHLQDVNRELTNQQEASVERQQQPPPETFDFKIKFAETKAHAKAIEMELRQMEVAQANRHMSLLTAFMPDSFLRPGGDHDCVLVLLLMPRLICKAELIRKQAQEKFDLSENCSERPGLRGAAGEQLSFAAGLVYSLSLLQATLHRYEHALSQCSVDVYKKVGSLYPEMSAHERSLDFLIELLHKDQLDETVNVEPLNKGIKYYQHLYRIHLAEQPEDSTMQLADHIKFTQSALDCMGVEVGRLRAFLQGGQEATDIALLLRDLETSCSDTRQFCKKIRRRMPGTDAPGIPAALAFGSQVSDTLLDCRKHLTWVVAVLQEVAAAAAQLIAPLAENEGLPVAALEELAFKASEQIYGSPSSSPYECLRQSCTILISTMNKLATAMQEGEYDAERPPSKPPPVELRAAALRAEITDAEGLGLKLEDRETVIKELKKSLKIKGEELSEANVRXSLLEKKLDSAAKDADERIEKVQTRLDETQTLLRKKEKDFEETMDALQADIDQLEAEKAELKQRLNSQSKRTIEGLRGPPPSGIATLVSGIAGEEPQRGGAPGQAPGALPGPGLVKDSPLLLQQISAMRLHISQLQHENSILRGAQMKASLAALPPLHVAKLSLPPHEGPGGNLVAGALYRKTSQLLEKLNQLSTHTHVVDITRSSPAAKSPSAQLMEQVAQLKSLSDTIEKLKDEVLKETVTQRPGATVPTDFATFPSSAFLRAKEEQQDDTVYMGKVTFSCAAGLGQRHRLVLTQEQLHQLHSRLIS

SEQ ID NO: 68 - protein, 1278 aa, Homo sapiens <s300> Genbank/AAD55811 1999-09-21 (1)..(1278)
MAQSKRHVYSRTPSGSRMSAEASARPLRVGSRVEVIGKGHRGTVAYVGATLFATGKWVGVILDEAKGKNDGTVQGRKYFTCDEGHGIFVRQSQIQVFEDGADTTSPETPDSSASKVLKREGTDTTAKTSKLRGLKPKKAPTARKTTTRRPKPTRPASTGVAGASSSLGPSGSASAGELSSSEPSTPAQTPLAAPIIPTPVLTSPGAVPPLPSPSKEEEGLRAQVRDLEEKLETLRLKRAEDKAKLKELEKHKIQLEQVQEWKSKMQEQQADLQRRLKEARKEAKEALEAKERYMEEMADTADAIEMATLDKEMAEERAESLQQEVEALKERVDELTTDLEILKAEIEEKGSDGAASSYQLKQLEEQNARLKDALVRMRDLSSSEKQEHVKLQKLMEKKNQELEVVRQQRERLQEELSQAESTIDELKEQVDAALGAEEMVEMLTDRNLNLEEKVRELRETVGDLEAMNEMNDELQENARETELELREQLDMAGARVREAQKRVEAAQETVADYQQTIKKYRQLTAHLQDVNRELTNQQEASVERQQQPPPETFDFKIKFAETKAHAKAIEMELRQMEVAQANRHMSLLTAFMPDSFLRPGGDHDCVLVLLLMPRLICKAELIRKQAQEKFELSENCSERPGLRGAAGEQLSFAAGLVYSLSLLQATLHRYEHALSQCSVDVYKKVGSLYPEMSAHERSLDFLIELLHKDQLDETVNVEPLTKAIKYYQHLYSIHLAEQPEDCTMQLADHIKFTQSALDCMSVEVGRLRAFLQGGQEATDIALLLRDLETSCSDIRQFCKKIRRRMPGTDAPGIPAALAFGPQVSDTLLDCRKHLTWVVAVLQEVAAAAAQLIAPLAENEGLLVAALEELAFKASEQIYGTPSSSPYECLRQSCNILISTMNKLATAMQEGEYDAERPPSKPPPVELRAAALRAEITDAEGLGLKLEDRETVIKELKKSLKIKGEELSEANVRLSLLEKKLDSAAKDADERIEKVQTRLEETQALLRKKEKEFEETMDALQADIDQLEAEKAELKQRLNSQSKRTIEGLRGPPPSGIATLVSGIAGEEQQRGAIPGQAPGSVPGPGLVKDSPLLLQQISAMRLHISQLQHENSILKGAQMKASLASLPPLHVAKLSHEGPGSELPAGALYRKTSQLLETLNQLSTHTHVVDITRTSPAAKSPSAQLMEQVAQLKSLSDTVEKLKDEVLKETVSQRPGATVPTDFATFPSSAFLRAKEEQQDDTVYMGKVTFSCAAGFGQRHRLVLTQEQLHQLHSRLIS

SEQ ID NO: 69 - protein, 1280 aa, Rattus norvegicus <s300> Genbank/NP_077044 2002-11-05 (1)..(1280)
MAQSKRHMYNRTPSGSRMSTEASARPLRVGSRVEVIGKGHRGTVAYVGATLFATGKWVGVILDEAKGKNDGTVQGRKYFTCDEGHGIFVRQSQIQVFEDGADTTSPETPDSSASKILKREGADAAAKTSKLRGLKPKKAPTARKTTTRRPKPTRPASTGVAGPSSSLGPSGSASAGELSSSEPSTPAQTPLAAPIIPTPALTSPGAAPPLPSPSKEEEGLRDQVRDLEEKLETLRLKRSEDKAKLKELEKHKIQLEQVQEWKSKMQEQQADLQRRLKEAKEAKEALEAKERYMEEMADTADAIEMATLDKEMAEERAESLQQEVEALKERVDELTTDLEILKAEIEEKGSDGAASSYQLKQLEEQNARLKDALVRMRDLSSSEKQEHVKLQKLMEKKNQELEVVRQQRERLQEELSQAESTIDELKEQVDAALGAEEMVEMLTDRNLNLEEKVRELRETVGDLEAMNEMNDELQENARETELELREQLDMAGARVREAQKRVEAAQETVADYQQTIKKYRQLTAHLQDVNRELTNQQEASVERQQQPPPETFDFKIKFAETKAHAKAIEMELRQMEVAQANRHMSLLTAFMPDSFLRPGGDHDCVLVLLLMPRLICKAELIRKQAQEKFDLSENCSERPGLRGAAGEQLSFAAGLVYSLSLLQATLHRYEHALSQCSVDVYKKVGSLYPEMSAHERSLDFLIELLHKDQLDETVNVEPLTKAIKYYQHLYSIHLAEQPEESTMQLADHIKFTQSALDCMSVEVGRLRAFLQGGQEATDIALLLRDLETSCSDIRQFCKKIRRRMPGTDAPGIPAALAFGSQVSDTLLDCRKHLTWVVAVLQEVAAAAAQLIAPLAENEGLPVAALEELAFKASEQIYGSPSSSPYECLRQSCSILISTMNKLATAMQEGEYDAERPPSKPPPVEPWPAALRAEITDAEGLGLKLEDRETVIKELKKSLKIKGEELSEANVRLSLLEKKLDSAAKDADERIEKVQTRLEETQTLLRKKEKEFEETMDALQADIDQLEAEKTELKQRLNSQSKRTIEGLRGPPPSGIATLVSGIAGEEQQRGGTPGQAPGALPGPGPVKDSPLLLQQISAMRLHISQLQHENSILRGAQMKASLAALPPLHVAKFSLPPHEGPGGNLLSGALYRKTSQLLEKLNQLSTYTHVVDITRSSPACKSPSAQLMEQVAQLKSLSDTIEKLKDEVLKETVTQRPGATVPTDFATFPSSAFLRAKEEQQDDTVYMGKVTFSCAAGLGQRHRLVLTQEQLHQLHGRLIS

SEQ ID NO: 70 - DNA, 108 bp, Mus musculus
atgtctgacaagagcgacctaaaggccgagctggagcgcaaaaagcagcgcttagcacagataagagaggagaagaaacggaaggaagaggagaggaagaagaaagag

SEQ ID NO: 71 - DNA, 115 bp, Mus musculus
gcagatatgcagcaaaagaaagagcccgttcaagatgactccgatctggatcgcaaacgacgggagacagaagctttgcttcagagcattggcatatcaccggagccccctctag

SEQ ID NO: 72 - DNA, 91 bp, Mus musculus
tcccaacccctatgtctccctcttcgaaatcagtgagcactcccagtgatgctggaagccaagactcgggcgatctggggccattaacaag

SEQ ID NO: 73 - DNA, 60 bp, Mus musculus
gaccctgcagtgggacacagacccctcagtgctccagctgcagtcagactcagaacttgg

SEQ ID NO: 74 - DNA, 374 bp, Mus musculus
atgtctgacaagagcgacctaaaggccgagctggagcgcaaaaagcagcgcttagcacagatwagagaggagaagaaacggaaggaagaggagaggaagaagaaagaggcagatatgcagcaraagaaagagcccgttcaagatgactccgatctggatcgcaaacgacgggagacagaagctttgcttcagagcattggcatatcaccggagccccctctagtcccaacccctatgtctccctcttcgaaatcagtgagcactcccagtgatgctggaagccaagactcgggcgatctggggccattaacaaggaccctgcagtgggacacagacccctcagtgctccagctgcagtcagactcagaacttgg

SEQ ID NO: 75 - DNA, 116 bp, Mus musculus
gagacgactgcacaagctgggcgtgtcaaaggtgacccaggtggatttcctgcctagggaagtagtatcctactccaaggagacacagactcctcttgcaacacatcagtctgaag

SEQ ID NO: 76 - DNA, 163 bp, Mus musculus
cccctccaagagagttgacggaggaagaaaaacagcagatcctgcactcagaagaattcctcatcttctttgaccggacaatccgagtaattgaaagagctcttgcagaggactcggacatcttttttgactacagcggtcgagagctggaggagaaagatgg

SEQ ID NO: 77 - DNA, 101 bp, Mus musculus
ggatgtgcaggctggagccaacctgtctttcaaccgtcagttctacgatgaacattggtctaagcatcgggtggtcacatgtatggactggtctctccagg

SEQ ID NO: 78 - DNA, 126 bp, Mus musculus
taccctgagctgatggttgcttcttatagcaacaacgaagatgctccccacgagccagatggagtggccttggtttggaacatgaagttcaagaaaaccacaccagaatatgtcttccactgtcag

SEQ ID NO: 79 - DNA, 390 bp, Mus musculus
cccctccaagagagttgacggaggaagaaaaacagcagatcctgcactcagaagaattcctcatcttctttgaccggacaatccgagtaattgaaagagctcttgcagaggactcggacatcttttttgactacagcggtcgagagctggaggagaaagatggggatgtgcaggctggagccaacctgtctttcaaccgtcagttctacgatgaacattggtctaagcatcgggtggtcacatgtatggactggtctctccaggtaccctgagctgatggttgcttcttatagcaacaacgaagatgctccccacgagccagatggagtggccttggtttggaacatgaagttcaagaaaaccacaccagaatatgtcttccactgtcag

SEQ ID NO: 80 - DNA, 123 bp, Homo sapiens
tccaaggaaaccaacatgtctgacaaaagtgacttaaaagctgagctagagcgcaaaaagcagcgcttagcacagataagagaagagaagaaacggaaggaagaggagaggaaaaagaaagag

SEQ ID NO: 81 - DNA, 115 bp, Homo sapiens
gctgatatgcagcagaagaaagaacccgttcaggacgactctgatctggatcgcaaacgacgagagacagaggctttgctgcaaagcattggtatctcaccggagccgcctctag

SEQ ID NO: 82 - DNA, 142 bp, Homo sapiens
tgcagccgctgcattttttaacatgggatacctgttattttcattatttagtcccaacccctatgtctccctcctcgaaatcagtgagcactcccagtgaagctggaagccaagactcaggcgatctggggccattaacaag

SEQ ID NO: 83 - DNA, 60 bp, Homo sapiens; identical to SEQ ID NO: 73

SEQ ID NO: 84 - DNA, 440 bp, Homo sapiens
tccaaggaaaccaacatgtctgacaaaagtgacttaaaagctgagctagagcgcaaaaagcagcgcttagcacagataagagaagagaagaaacggaaggaagaggagaggaaaaagaaagaggctgatatgcagcagaagaaagaacccgttcaggacgactctgatctggatcgcaaacgacgagagacagaggctttgctgcaaagcattggtatctcaccggagccgcctctagtgcagccgctgcattttttaacatgggatacctgttattttcattatttagtcccaacccctatgtctccctcctcgaaatcagtgagcactcccagtgaagctggaagccaagactcaggcgatctggggccattaacaaggaccctgcagtgggacacagacccctcagtgctccagctgcagtcagactcagaacttgg

SEQ ID NO: 85 - DNA, 116 bp, Homo sapiens
aagaagactgcataaactgggcgtgtcaaaggtcacccaagtggatttcctgccaagggaagtagtgtcctactcaaaggagacccagactcctcttgccacgcatcagtctgaag

SEQ ID NO: 86 - DNA, 163 bp, Homo sapiens
cccctccaagagagttgacagaggaagaaaaacagcagatccttcattcagaggaatttctcatcttttttgaccggacaatacgggtaattgaaagagccctggctgaagattccgacatcttttttgactacagcggccgagagttagaggaaaaagatgg

SEQ ID NO: 87 - DNA, 100 bp, Homo sapiens
ggatgttcaggctggagccaatctttctttcaatcgtcagttctatgatgaacattggtccaagcatcgagtggtcacttgtatggactggtccctccag

SEQ ID NO: 88 - DNA, 126 bp, Homo sapiens
taccctgagctgatggtggcttcttacaacaacaatgaagatgctccccatgaaccagatggagtggccttggtttggaacatgaagtttaagaaaaccacaccagaatacgtcttccactgtcag

SEQ ID NO: 89 - DNA, 389 bp, Homo sapiens
cccctccaagagagttgacagaggaagaaaaacagcagatccttcattcagaggaatttctcatcttttttgaccggacaatacgggtaattgaaagagccctggctgaagattccgacatcttttttgactacagcggccgagagttagaggaaaaagatggggatgttcaggctggagccaatctttctttcaatcgtcagttctatgatgaacattggtccaagcatcgagtggtcacttgtatggactggtccctccagtaccctgagctgatggtggcttcttacaacaacaatgaagatgctccccatgaaccagatggagtggccttggtttggaacatgaagtttaagaaaaccacaccagaatacgtcttccactgtcag

SEQ ID NO: 90 - DNA, 117 bp, Mus musculus
gtcacaaccatgtcggacaaaagtgatttaaaagctgagttggaacgtaagaagcaacggttggcccaaatcagagaagaaaagaagaggaaagaagaggaaaggaaaaaaaaggaa

SEQ ID NO: 91 - DNA, 118 bp, Mus musculus
actgatcagaagaaggaagctgctgtttctgtacaagaagagtctgatcttgaaaaaaaaagaagagaagctgaggcattgcttcaaagcatgggactaactacggactcccctattg

SEQ ID NO: 92 - DNA, 91 bp, Mus musculus
tccctcctcctatgtctccatcctccaagtcggtgagcacgccaagtgaagctggaagccaggattctggagatggcgccgtgggatctag

SEQ ID NO: 93 - DNA, 116 bp, Mus musculus
acgaggacctattaaacttggaatggccaaaattactcaagttgactttccccctcgagaaatcgtcacatacacaaaggaaactcagaccccggttacagctcaacccaaagaag

SEQ ID NO: 94 - DNA, 442 bp, Mus musculus
gtcacaaccatgtcggacaaaagtgatttaaaagctgagttggaacgtaagaagcaacggttggcccaaatcagagaagaaaagaagaggaaagaagaggaaaggaaaaaaaaggaaactgatcagaagaaggaagctgctgtttctgtacaagaagagtctgatcttgaaaaaaaaagaagagaagctgaggcattgcttcaaagcatgggactaactacggactcccctattgtccctcctcctatgtctccatcctccaagtcggtgagcacgccaagtgaagctggaagccaggattctggagatggcgccgtgggatctagacgaggacctattaaacttggaatggccaaaattactcaagttgactttccccctcgagaaatcgtcacatacacaaaggaaactcagaccccggttacagctcaacccaaagaag

SEQ ID NO: 95 - DNA, 163 bp, Mus musculus
ctccacctcatgaactaactgaagaagaaaagcaacagattttacactcagaagaatttttaagtttctttgaccattctacgagaattgtagaaagagccctttctgagcagattaacatcttctttgactacagtgggagagacttggaagacaaagaagg

SEQ ID NO: 96 - DNA, 100 bp, Mus musculus
ggagattcaagcaggtgctaagctgtcattaaatcgacagttctttgatgagcgttggtcaaagcatcgagttgttagttgtttggattggtcatcccag

SEQ ID NO: 97 - DNA, 126 bp, Mus musculus
tatccagaattacttgtggcttcctataataacaatgaagaggctcctcatgagcctgatggtgtggccctcgtgtggaatatgaagtacaaaaaaactaccccagagtatgtgttccactgccag

SEQ ID NO: 98 - DNA, 389 bp, Mus musculus
ctccacctcatgaactaactgaagaagaaaagcaacagattttacactcagaagaatttttaagtttctttgaccattctacgagaattgtagaaagagccctttctgagcagattaacatcttctttgactacagtgggagagacttggaagacaaagaaggggagattcaagcaggtgctaagctgtcattaaatcgacagttctttgatgagcgttggtcaaagcatcgagttgttagttgtttggattggtcatcccagtatccagaattacttgtggcttcctataataacaatgaagaggctcctcatgagcctgatggtgtggccctcgtgtggaatatgaagtacaaaaaaactaccccagagtatgtgttccactgccag

SEQ ID NO: 99 - DNA, 112 bp, Homo sapiens
atgtcagacaaaagtgaattaaaggctgagttggaacgtaagaagcagcgactggcccaaatcagagaggaaaagaagagaaaagaagaagaaaggaaaaaaaaagaaacag

SEQ ID NO: 100 - DNA, 114 bp, Homo sapiens
accagaagaaggaagctgttgctcctgtgcaagaagaatcagatcttgaaaaaaaaaggagagaagctgaagcattgcttcaaagcatggggctaactccagaatcccccattg

SEQ ID NO: 101 - DNA, 91 bp, Homo sapiens
tccctcctcctatgtctccatcctccaaatctgtgagcactccaagtgaagctggaagccaagactctggagatggcgccgtgggatctag

SEQ ID NO: 102 - DNA, 116 bp, Homo sapiens
acgaggacctattaaacttggaatggctaaaatcacgcaagtcgactttcctcctcgagaaattgtcacgtatacaaaggaaactcagactccagttatggctcaacccaaagaag

SEQ ID NO: 103 - DNA, 433 bp, Homo sapiens
atgtcagacaaaagtgaattaaaggctgagttggaacgtaagaagcagcgactggcccaaatcagagaggaaaagaagagaaaagaagaagaaaggaaaaaaaaagaaacagaccagaagaaggaagctgttgctcctgtgcaagaagaatcagatcttgaaaaaaaaaggagagaagctgaagcattgcttcaaagcatggggctaactccagaatcccccattgtccctcctcctatgtctccatcctccaaatctgtgagcactccaagtgaagctggaagccaagactctggagatggcgccgtgggatctagacgaggacctattaaacttggaatggctaaaatcacgcaagtcgactttcctcctcgagaaattgtcacgtatacaaaggaaactcagactccagttatggctcaacccaaagaag

SEQ ID NO: 104 - DNA, 163 bp, Homo sapiens
ctccccctcatgagctgactgaagaagaaaagcaacaaatcctgcactctgaggaatttttaagtttctttgaccattctacaagaattgtagaaagagctctttctgagcagattaacatcttctttgactatagtgggagagatttggaagacaaagaagg

SEQ ID NO: 105 - DNA, 100 bp, Homo sapiens
agagattcaagcaggtgctaaactgtcattaaatcgacaattttttgacgaacgttggtcaaagcatcgggtggttagttgtttggattggtcatctcag

SEQ ID NO: 106 - DNA, 126 bp, Homo sapiens
tatccggagttactcgtggcttcctataacaacaatgaagatgcccctcatgagcctgatggtgtggcccttgtatggaatatgaaatacaaaaaaactaccccagagtatgtgtttcactgccag

SEQ ID NO: 107 - DNA, 389 bp, Homo sapiens
ctccccctcatgagctgactgaagaagaaaagcaacaaatcctgcactctgaggaatttttaagtttctttgaccattctacaagaattgtagaaagagctctttctgagcagattaacatcttctttgactatagtgggagagatttggaagacaaagaaggagagattcaagcaggtgctaaactgtcattaaatcgacaattttttgacgaacgttggtcaaagcatcgggtggttagttgtttggattggtcatctcagtatccggagttactcgtggcttcctataacaacaatgaagatgcccctcatgagcctgatggtgtggcccttgtatggaatatgaaatacaaaaaaactaccccagagtatgtgtttcactgccag

SEQ ID NO: 108 - DNA, 279 bp, Mus musculus
atggcccagagcaggaggcacatgtccagtcggacgccgagtggcagcaggatgagtacggaggcaagcgcccggcccctgcgggttggctcccgcgtggaggtgattgggaagggccaccgaggcactgtggcctatgttggagccacactctttgccactggcaaatgggtgggcgtgattctggatgaagcaaaaggcaaaaatgatggcactgtccagggaaggaagtatttcacatgtgatgaaggccacggcatctttgtacgccagtcccag

SEQ ID NO: 109 - DNA, 79 bp, Mus musculus
atccaagtatttgaagatggagcagatactacttccccagagactcctgattcttctgcttcaaaggtcctcaagagag

SEQ ID NO: 110 - DNA, 35 bp, Mus musculus
agggagccgatgcagctgcaaagaccagcaaactg

SEQ ID NO: 111 - DNA, 21 bp, Mus musculus
accacaactcgacggcccaag

SEQ ID NO: 112 - DNA, 453 bp, Mus musculus
atggcccagagcaggaggcacatgtccagtcggacgccgagtggcagcaggatgagtacggaggcaagcgcccggcccctgcgggttggctcccgcgtggaggtgattgggaagggccaccgaggcactgtggcctatgttggagccacactctttgccactggcaaatgggtgggcgtgattctggatgaagcaaaaggcaaaaatgatggcactgtccagggaaggaagtatttcacatgtgatgaaggccacggcatctttgtacgccagtcccagatccaagtatttgaagatggagcagatactacttccccagagactcctgattcttctgcttcaaaggtcctcaagagagagggagccgatgcagctgcaaagaccagcaaactgcggggactgaagcctaagaaggcaccgacagcccgaaagaccacaactcgacggcccaag

SEQ ID NO: 113 - protein, 21 aa, Homo sapiens
VEQYVKVWLQYQCLWDMQAEN

SEQ ID NO: 114 - DNA, 192 bp, Mus musculus
cctactcgcccagccagcactggggtggctgggcccagtagctcccttggcccctctggctcagcgtcagccggggaactaagcagcagtgagcccagcaccccagctcagactccgctggcagcacccatcatccccacaccggccctcacctctcctggagcagcacccccacttccatctccctctaag

SEQ ID NO: 115 - DNA, 198 bp, Mus musculus
gaagaggaagggctgagggctcaggtacgggacctggaggagaagctggagaccctgcgcctaaaacgctcagaagacaaagcaaagctgaaagagctggagaagcacaagatccagctggagcaggtgcaggaatggaagagcaaaatgcaggagcagcaggcagacctgcagcggcgcctcaaggaggctcggaag

SEQ ID NO: 116 - DNA, 205 bp, Mus musculus
gaagccaaggaggcgctagaggcaaaggaacgctacatggaggagatggccgacacagccgacgctatcgagatggccactctggacaaggagatggctgaagagcgcgctgagtctctgcagcaagaggtggaggcactgaaggaacgggtagacgagctcaccacagacctggagattctcaaggctgaaatcgaagagaaag

SEQ ID NO: 117 - DNA, 79 bp, Mus musculus
gctctgatggggccgcatcaagctaccagctcaagcagctggaggagcagaatgcccgcctgaaggatgccctggtgag

SEQ ID NO: 118 - DNA, 160 bp, Mus musculus
gatgcgagacctctcttcctcagagaagcaggagcacgtgaagctgcagaaactcatggaaaagaaaaaccaggagctggaggtcgtgcggcagcagcgcgagcgtcttcaggaggagctgagccaggctgagagcaccatcgatgagctcaaagagcag

SEQ ID NO: 119 - DNA, 105 bp, Mus musculus
gtggacgccgctctgggagccgaggagatggtggagatgctgaccgaccggaacctgaatctagaggagaaagtgcgggagttacgggagactgtgggggacttg

SEQ ID NO: 120 - DNA, 192 bp, Mus musculus
gaagccatgaacgagatgaacgatgagctgcaggagaacgcacgggagacggagctggaactccgagagcagctggacatggcgggcgcccgagtgagggaagcgcagaagcgagtggaagccgcccaggagacagtcgccgactaccagcagaccatcaagaagtaccgccagttgactgcccacctacag

SEQ ID NO: 121 - DNA, 117 bp, Mus musculus
gatgtcaatcgggagctgacaaaccagcaggaagcgtctgtagagaggcagcagcagccgccgccagagacttttgatttcaaaatcaagtttgctgagaccaaggctcatgccaag

SEQ ID NO: 122 - DNA, 153 bp, Mus musculus
gccattgagatggagttgagacagatggaagttgcccaggccaaccggcacatgtccctgctgacagcctttatgcctgacagcttccttcggccaggtggagaccacgactgtgtcctggtgctgctgctcatgccccgactcatttgcaag

SEQ ID NO: 123 - DNA, 161 bp, Mus musculus
gcagagctcatccggaagcaggcccaggagaagtttgacctgagcgagaactgttcggagcggcccgggctgcggggagctgccggggagcagctgagctttgctgctggactggtgtactcgctgagtctgctgcaggccacgctgcaccgctatgagca

SEQ ID NO: 124 - DNA, 169 bp, Mus musculus
tgccctctctcagtgcagtgtggacgtgtataagaaggtcggcagcctgtaccccgagatgagcgcccacgagcgctccttagatttcctcattgagctgctgcacaaggatcagctggatgagactgtcaacgtggagcccctcaccaaggccatcaagtattaccag

SEQ ID NO: 125 - DNA, 169 bp, Mus musculus; identical to SEQ ID NO: 124

SEQ ID NO: 126 - DNA, 169 bp, Mus musculus; identical to SEQ ID NO: 124

(19) United States
(12) Patent Application Publication
(10) Pub. No.: US 2007/0010657 A1
(43) Pub. Date: Jan. 11, 2007

(54) Cytoplasmic dynein heavy chain 1 genes, expression products, non-human animal model uses in human neurological diseases

(76) Inventors: Rainer Klocke, Martinsried (DE); Andreas Marquardt, Martinsried (DE); Gisela Peraus, Martinsried (DE); Gabriele Stumm, Martinsried (DE); Philipp Wabnitz, Martinsried (DE)

Correspondence Address: FISH & RICHARDSON PC, P.O. BOX 1022, MINNEAPOLIS, MN, 55440-1022, US

(21) Appl. No.: 10/527,769

(22) PCT Filed: Sep. 11, 2003
(86) PCT No.: PCT/EP03/10118

§ 371 Date: Dec. 5, 2005

(30) Foreign Application Priority Data
Sep. 13, 2002 (US) 6041076
Nov. 15, 2002 (US) 60426609
Feb. 4, 2003 (US) 60444920

(51) Int. Cl. C07K 14/705; C12Q 1/68; C07H 21/02; C12P 21/06; A61K 48/00; A61K 38/17

(52) U.S. Cl. 530/350; 530/388.22; 435/6; 435/69.1; 435/320.1; 435/325; 514/44; 536/23.1; 514/12

(57) ABSTRACT

A non-human animal model is provided, particularly a mouse model for movement hyperactivity, excitoxicity disorders (e.g. myoclonic cramping) and neurodegeneration, in which modified cytoplasmic dynein heavy chain1 is expressed. Modified human and mouse cytoplasmic dynein heavy chain1 proteins and nucleic adds are also provided, including the corresponding recombinant proteins. The invention further provides uses for the non-human animal model and the modified cytoplasmic dynein heavy chain1 proteins and nucleic acids, in particular for the diagnosis and treatment of medical conditions associated with over-expression of cytoplasmic dynein heavy chain1.

### FIELD OF THE INVENTION

The present invention inter alia relates to a non-human animal model for movement hyperactivity, hyperexcitability disorders (e.g. myoclonic cramping, epilepsy), excitoxicity disorders, and neurodegeneration. This animal model bears a mutation in the cytoplasmic dynein heavy chain1 gene. The invention also relates to modified peptides and the corresponding nucleic acid sequences of the modified mouse and human cytoplasmic cytoplasmic dynein heavy chain1. Furthermore, the invention relates to the use of these peptides and nucleic acids for manufacturing therapeutics suitable for the treatment of diseases, such as Alzheimer's disease, Huntington's disease, Parkinson's disease and amyotropic lateral sclerosis (ALS), as well as other diseases associated with overexpression, over-activity, or undesirable activity of cytoplasmic dynein heavy chain1.

### BACKGROUND OF THE INVENTION

Eukaryotic cells are characterized by biochemical and physiological processes which under normal conditions are exquisitely balanced to achieve the preservation and propagation of the cells. When such cells are components of multicellular organisms such as vertebrates, or more particularly organisms such as mammals, the regulation of the biochemical and physiological processes involves intricate signaling pathways. Frequently, such signaling pathways consist of extracellular signaling proteins, cellular receptors that bind the signaling proteins and signal transducing components located within the cells.

Signaling processes may elicit a variety of effects on cells and tissues, including by way of nonlimiting example induction of cell or tissue proliferation, suppression of growth or proliferation and induction or suppression of differentiation or maturation of a cell or tissue.

Many pathological conditions involve dysregulation of expression of important effector proteins. In certain classes of pathologies the dysregulation is manifested as a diminished or suppressed level of synthesis and secretion of protein effectors. In a clinical setting a subject may be suspected of suffering from a condition brought on by diminished or suppressed levels of a protein effector of interest. Therefore, there is a need to assay for the level of the protein effector of interest in a biological sample from such a subject and to compare the level with that characteristic of a nonpathological condition. There is a further need to provide the protein effector as a product of manufacture. Administration of the effector to a subject in need thereof is useful in treatment of the pathological condition. Accordingly, there is a need for a method of treatment of a pathological condition brought on by a diminished, suppressed, or in some cases elevated, level of the protein effector of interest.

Motor neuron degenerative diseases are severely debilitating and largely fatal in humans as well as other mammalian species. Several hypotheses have been proposed to explain the pathogenesis of the diseases, but none addressed the underlying cause for the specificity of disease in motor neurons, the late onset of the disease, and the cumulative progressive nature of the disease.

Degenerative disorders of motor neurons include a range of progressive diseases such as amyotrophic lateral sclerosis (ALS) and spinal muscular atrophy (SMA), the most common genetic cause of death of children (Nicole, S. et al., J. Muscle Nerve 26, 4-13, 2002).

In ALS, commonly known as Lou Gehrig's Disease in the US, the motor neurons (nerve cells in the brain, brain stem, and spinal cord that control movement of the skeletal muscles) gradually degenerate, resulting in progressive weakness and functional loss of involved muscles. Diseases that cause selective progressive death of motor neurons are surprisingly common—ALS is the third most frequent neurodegenerative cause of adult death, after Alzheimer disease and Parkinson disease, and is significantly more common than multiple sclerosis (Motor Neurone Disease Association Information Sheet Number 9, Motor Neurone Disease Association, 1998). Targeted at adults in their prime years of life, the average age of onset is the mid-fifties, although adults of all ages can be affected. Men get the disease slightly more often than women. Worldwide, the incidences of ALS is 0.5 to 2.4 cases per 100,000, with a prevalence of 2.5 to 7 cases per 100,000 population (Cleveland, D. W. and Rothstein, J. D. Nat. Rev. Neurosci 2, 806-819, 2001). The prevalence of ALS in the United States will probably increase dramatically as the baby boomer generation ages. In ALS the majority of patients die within 2-5 years of clinical onset (Motor Neurone Disease Association Information Sheet Number 9, Motor Neurone Disease Association (1998). ALS and related motor neuron disorders are not contagious but in some cases known to be hereditarily transmitted. Up to 10% of ALS is familial (FALS) and causative mutations have been found in the SOD1 gene that account for up to 20% of these FALS cases (Julien, J. P., Cell 104, 581-591, 2001). SOD1 encodes the ubiquitously expressed enzyme superoxide dismutase 1 which takes on an unknown dominant gain of function in ALS that results in the selective death of motor neurons. Mutations have also been found in alsin, probably a GTPase regulatory protein, in rare juvenile recessive forms of motor neuron disease (Hadano, S. et al. Nat. Genet. 29, 166-173, 2001; Yang, Y. et al. Nat. Genet. 29, 160-165, 2001). In a few sporadic ALS patients and one FALS case mutations have been identified in the NFH (neurofilament heavy chain) gene (Robberecht, W. J. Neurol 247, 2-6, 2000). Approximately, 90% of all ALS is without history and is referred to as sporadic ALS, although the clinical course and the neuropathological alterations are often indistinguishable from the proven cases of genetic disease. If a genetic cause would be responsible also for a subgroup of so called sporadic ALS, the mutation would likely be germline or very early embryonic, as mosaic type pathological alterations expected in later acquired mutations are not reported nor expectable in ALS.

Currently, diagnosing ALS is a combination of medical history and physical and neurological examinations performed by a clinical neurologist (Brooks, Benjamin R., El Escorial J Neurol Sci 124 (Suppl.), pages 96-107, 1994; Karitzky J, Ludolph A C. J Neurol Sci, October 15; 191(1-2):35-41, 2001; Ludolph A C, and Knirsch U, J Neurol Sci, June; 165 Suppl 1:S14-20, 1999). An electromyogram is a diagnostic test which is done to determine abnormal nerve and muscle activity. The clinical examination and tests also rule out other conditions that might mimic motor neuron disease. The certainty of the diagnosis is determined by the clinical evidence of upper and lower motor neuron signs, according to the criteria established by the World Federation of Neurology. Therefore, a definitive diagnosis of ALS is performed when clearly detectable classical clinical signs such as muscular fibrillation of distal muscle groups or tongue, difficulty to swallow, and predominantly distal muscular atrophy with the typical histological picture of grouped angular shaped atrophic muscle fibres indicative of muscle denervation are present which means that the disease course is already proceeded.

Some persons are diagnosed to have other clinical variant forms of motor neuron disease which may later evolve into classical ALS over time. These clinical subtypes of motor neuron disease include: Progressive bulbar palsy (PBP) which affects the brain stem (referred to as bulbar involvement), causes weakness of the speech and swallowing muscles. Progressive muscular atrophy (PMA) which affects the lower motor neurons causes skeletal muscle wasting. Primary lateral sclerosis (PLS) which affects the upper motor neurons causes muscle spasticity and may be slowly progressive over many years (Brooks, B R.: El Escorial J Neurol Sci 124 (Suppl.), 1994, pages 96-107).

The most common form of spinal muscular atrophy (SMA) affects up to 1 in 6,000 newborns and is the leading hereditary cause of infant mortality worldwide, causing death before the age of 2 years. Defects in the widely expressed SMN1 (survival motor neuron 1) gene are responsible for most SMA (Hofmann, Y. and Wirth, B. Hum. Mol. Genet. 11, 2037-2049, 2002; Lefebvre, S. et al. Cell 80, 155-165, 1995).

Another inherited motor neuron degeneration of early adulthood is the spinobulbar muscular atrophy (SBMA), which in some families is linked to a X-chromosomal trait and CAG triplet repeat expansion within the androgen receptor (Sparfeld A D et al., Arch. Neurol. 59:1921-1926, 2002). SBMA x-linked is also termed Kennedy disease. However, there are also SBMA like syndromes following autosomal inheritance, the underlying mutations are not yet identified.

Current hypothesis to the pathogenesis of motor neuron degeneration address a variety of cellular insults that may intersect, leading individually or in concert to motor neuron degeneration, motor neuron death and finally ALS (Julien, J. P. Cell 104, 581-591, 2001). A faculty gene and excess glutamate may lead to damaging free radicals, which can harm the nerve cell's DNA (Munch, C et al., J Neurochem, Aug. 82(3):594-603, 2002; Ludolph A C, and Meyer T, Riepe M W J Neurol 2000 March; 247 Suppl 1:I7-16; Meyer T et al. J Neurol Neurosurg Psychiatry, December; 65(6):954, 1998; Meyer T. et al., J Neurol Neurosurg Psychiatry, October; 65(4):594-6, 1998). Glutamate also may lead to the production of detrimental calcium, which can chum out its own supply of DNA-harming free radicals. The free radicals also may injure neurofilaments, proteins that serve as the skeleton of the cell. In addition, the immune system (Hofmann, Y. and Wirth, B. Hum. Mol. Genet. 11, 2037-2049, 2002) may be involved in harming neurons. Abnormalities can lead to an accumulation of the toxic calcium.

Glutamate-induced excitotoxicity is a potential contributor to ALS pathogenesis. Depolarization of the neuronal membrane after activation of neuronal glutamate receptors activates voltage-dependent Ca²⁺ channels, allowing Ca²⁺ entry into the cell. Thus, excess activation of neuronal glutamate receptors can cause cell death via alterations in cytosolic free Ca²⁺ homeostasis. For spinal motor neurons, rapid recovery of synaptic glutamate is accomplished by the glutamate transporter EAAT2 present in astrocytes. Loss of EAAT2 transporter could lead to increased extracellular concentrations of glutamate and excitotoxic degeneration of motor neurons (Julien, Review; Cell. 2001 Feb. 23; 104(4):581-91).

ALS researchers at the John Hopkins University have recently enabled mouse ALS models to make an excess of glutamate transport proteins in both brains and spinal cords. Preliminary studies of these mice show huge increases in their survival time, which led to the development of an “EAAT2 replacement therapy” for ALS patients at the John Hopkins University (The Robert Packard Center for ALS Research at John Hopkins, www.alscenter.org 2002).

The excitotoxicity hypothesis is supported by the observation that the majority of sporadic ALS cases (˜65%) have a reduction in the astroglial glutamate transporter EAAT2 in motor cortex and spinal cord (Rothstein et al., Ann Neurol. 1995 July; 38(1):73-84). Germline mutations in the EAAT2 gene are very rare. Only one sporadic ALS case was identified with an EAAT2 gene variant that affects N-linked glycosylation and glutamate clearance capacity (Aoki et al., Ann Neurol. 1998 May; 43(5):645-53; Trotti et al., J Biol Chem. 2001 Jan. 5; 276(1):576-82.).

One major support for the excitotoxicity hypothesis comes from the first drug therapy for ALS which is Riluzole (Rilutek, (Rhone-Poulenc Rorer Pharmaceuticals Inc., College-ville, PA) approved by the FDA in 1995), an antiglutamate compound. Riluzole retards nerve cells' release of glutamate. Although the effects of Riluzole are clearly modest, it has been the only drug that reliably shows clinical efficacy compared to the dozens of drugs studied by clinicans around the world in thousands of ALS patiens (Nervous Breakdown, A detailed analysis of the neurology market, UBS Warburg, June 2001). Riluzole was reported to extend survival of ALS diagnosed patients for a few months. However, all therapeutic approaches tested so far are prone to fail when applied in later stages of the disease (Al-Chalabi A, and Leigh P N. Curr Opin Neurol, August; 13(4):397-405, 2000; Munch C, and Ludolph A C. Neurol Neurochir Pol; 35(1 Suppl):41-50, 2001; Ludolph A C. J Neurol, December; 247:13-18, 2000). Therefore, an early or even subclinical diagnostic marker might also have implications for therapeutic effectiveness.

It has been postulated that defects in axonal transport may be an underlying common pathway that leads to the degeneration of motor neurons in ALS patients and in relevant spontaneous and transgenic mouse models such as wobbler, nmd or SOD1 (Yang, Y. et al. Nat. Genet. 29, 160-165, 2001; Cleveland D W. Neuron, November; 24(3):515-20, 1999; Jonsson P A et al. Neurobiol Dis., August; 10(3):327-33, 2002; Winter S M et al. J Neurol., October; 247(10):783-6, 2000) For example, SOD mice with a mutation in the SOD-1 gene display a motor neuron degenerative phenotype with a decreased rate of slow axonal transport (Collard et al., Nature 375:61-64, 1995; Zhang et al., J. Cell Biol. 139:1307-1315, 1997; Williamson and Cleveland, Nat. Neurosci. 2:50-56, 1999.

Motor neurons of the brain and spinal cord are characterized by the length of their axons, which can reach a meter in length in an adult human. The significant length of these neuronal projections makes active axonal transport essential for normal cellular function. The axonal transport is microtubule dependant and includes both, an anterograde transport of organelles to the axonal synapse and the retrograde transport of multivesicular bodies and trophic factors back to the neuron cell body. The retrograde transport is mediated by the dynein-dynactin complex.

The Dynein-Dynactin Complex

Dyneins are cytoskeletal motor proteins. These can be defined as molecules that convert chemical energy, originating from nucleotide hydrolysis, into the mechanical force necessary for them to move along cytoskeletal polymers (cf Vallee and Howard (1990) Annu. Rev. Biochem 59: 909-932). Dyneins and kinesins constitute the superfamily of microtubule-dependent motor proteins, (Hirokawa (1998) Science 279, 519-526). Although the superfamily of dyneins is probably less diverse than that of kinesins, the family, which contributes to the structure and function of flagellar and cilliary axonems (axonemal dyneins), comprises more than a dozen dynein heavy chain isoforms (Milisav (1998) Cell Motil Cytoskeleton 39, 261-272). Only four dynein heavy chain isoforms, contributing to different forms of the protein complex, called cytoplasmic dynein, are known in mammals.

The most abundant form of cytoplasmic dynein, whose identity is defined by the cytoplasmic dynein heavy chain1, is involved in a wide range of cellular functions. In mouse and human, the homologous proteins are referred to as cytoplasmic dynein heavy chain1. These proteins correspond, respectively, to the translation product of the transcript of the mouse gene Dnchc1 (Genbank Accession No. AY004877) and the translation product of the full-length cDNA of the human gene DNCH1. The full-length human cytoplasmic dynein heavy chain1 cDNA is disclosed herein (SEQ ID NO:17). Several lines of evidence (e.g. immunodepletion of cytoplasmic dynein heavy chain1 in in vitro motility studies and antibody injection) revealed that it functions as a molecular motor in prometaphase chromosome movement, mitotic spindle pole organization, Golgi organization, spindle orientation, nuclear migration, microtubule organization and reorganization and intracellular trafficking, including retrograde axonal transport, of membraneous organelles, (e.g. lysosomes and endosomes) (for review see Milisav (1998) Cell Motil Cytoskeleton 39, 261-272). Another isoform has been reported to be involved in intraflagellar protein transport (Pazour et al. (1999) J Cell Biol 144, 473-481). Cytoplasmic dynein was initially identified in nervous tissue (Paschal et al. (1987) J Cell Biol 105, 1273-1282) although it is expressed in several tissues (Mikami et al. (1993) Neuron 10, 787-796).

Harada et al. ((1998) J Cell Biol 141, 51-59) generated mice lacking cytoplasmic dynein heavy chain 1 by targeted disruption of the cDHC (=Dnchc1) gene. Whereas cDHC−/− preimplantation embryos were microscopically indistinguishable from cDHC+/+ and cDHC+/+ littermates, by 8.5 days p.c., no cDHC−/− embryo were found. This observation demonstrates that cytoplasmic dynein is essential for embryonic development. In conjunction with the functional data mentioned above, Harada et al. found that cells from cDHC−/− blastocysts, in contrast to those from cDHC+/+ blastocysts, were incapable of dividing in culture. Additionally, the Golgi complex was highly vesiculated and distributed in these cells as were the endosomes and lysosomes.

The cytoplasmic dynein complex consists of at least four classes of subunits: heavy, intermediate, light intermediate and light dynein chains. The dynein intermediate chain, dynein light intermediate chain, and dynein light chain specify the intracellular location of the dynein complex and regulate its motor activity, whereas the dynein heavy chains contain the machinery which imparts the motor activity.

The architecture of the complex is dominated by two heavy chains of 530 kDA each, which form a stem by the interaction of a large portion (amino acids 300-1140) of their N-termini. This stem also binds the dynein chains of lower molecular weight, e.g. two 74 kDa intermediate chains (between amino acids 446 and 701 of the heavy chains) and four 53-59 kDa light intermediate chains (between amino acids 649 and 800 of the heavy chains) (Tynan et al. (2000) J Biol Chem. 275, 32769-32774). Several 8-22 kDa light chains bind to the complex via interaction with the intermediate or light intermediate chains (cf Makoka et al. (2002) Biochemistry 41, 4302-4311). The intermediate chains probably impart the tethering of the dynein complex to the cargos (review: Asai and Koonce (2001) Trends Cell Biol 11, 196-202). Intracellular transport processes mediated by cytoplasmic dynein additionally involve a variety of accessory proteins that are thought to target the cytoplasmic dynein complex to the intracellular cargos. Another large complex formed by several of these accessory proteins is named dynactin. Dynactin binds to dynein by the interaction of a subunit called p150Glued with the dynein intermediate chains (Vaughan and Vallee (1995) J Cell Biol 131, 1507-1516). This interaction implicates dynactin as a receptor or adaptor that mediates the association of cytoplasmic dynein with cargo. Subsequent studies have demonstrated the essential nature of the dynein-dynactin interaction for most examples of dynein-mediated transport (Holleran et al., Int. Rev. Cytol. 182:69-109, 1998)

The binding of dynactin to dynein is essential for dynein-dependent intracellular movements (Gill et al. (1991) J Cell Biol 115, 1639-1650; Schroer and Sheetz (1991) J Cell Biol 115, 1309-1318) including those required for neuronal function. It has been shown that antibodies which specifically disrupt the binding of dynactin to dynein block vesicle motility along microtubules in extruded squid axoplasm (Waterman-Storer et al. (1997) Proc Natl Acad Sci USA 94, 12180-12185). Additionally, an overexpression of the dynamitin subunit of dynactin, which results in the dissociation of the dynactin complex, (Eckley et al. (1999) J Cell Biol 147, 307-320) causes an inhibition of dynein-mediated processes in the cell (Burkhardt et al. (1997) J Cell Biol 139, 469-484).

Dynein-mediated intracellular movements are directed along microtubules towards their proximal (−) ends. This process is accomplished by ATP hydrolysis, which is predominantly catalysed by the first of four (counted from the one nearest to the N-terminus) P-loop motifs of each of two C-terminal globular heads which are formed by approximately 3000 amino acids of each of the two dynein heavy chains. Both globular heads also contain a microtubule binding domain, which is thought to be located at the tip of a stalk (Asai and Koonce (2001) Trends Cell Biol 11, 196-202).

The Dynein-Dynactin Complex and Neurodegeneration

Recently, disruption of the dynein-dynactin complex in postnatal motor neurons of transgenic mice that overexpress dynamitin has been shown to result in reduction of microtubule based axonal transport and degeneration of the motor neurons and muscle atrophy over a time-course of many months (LaMonte et al. (2002) Neuron 34, 715-727) The pathology described therein is the result of a highly artificial transgenic approach for regional and time dependent disruption of the dynein-dynactin complex selectively in adult motor neurons. This situation is not comparable with any clinical situation with an underlying genetic cause, but would only address motor neuron specific stress factors for the dynein-dynactin system. The model described by LaMonte et al. is thus quite different from the model described in the present invention.

No effective therapy exists for the neurodegenerative disorders described herein. The selective nature of the degeneration is unexplained, but may relate to the highly specialized form and function of the motor neuron. There are known gene mutations in some familial cases of ALS and in almost all SMA, but the underlying defect in the majority of fanilial and sporadic ALS and a small proportion of SMA remains unknown. As mentioned before, in ALS the pathogenetic link between causal mutations in SOD1 (superoxide dismutase) and the death of motor neurons is not clear, although a variety of mechanisms have been proposed, including pathways disrupting neurofilament secondary structure (Miller (2002) Cell Mol Life Sci 59, 323-330) that result in slowing of axonal transport. Only in 2% of ALS cases a preclinical genetic diagnosis by detection of SOD1 mutations responsible for 20% of familial ALS is possible. No preclinical or subclinical diagnostic tool for the broad majority of ALS cases is available, therefore treatment can so far only start late in the course of the disease when already a large number of motor neurons have died. Therapy might not be suitable to stop the ongoing disease process. Therefore, new diagnostic tools such as a genetic test for disease relevant genes are urgently needed.

The present invention provides an animal model for correlating mutations or defects in the cytoplasmic dynein heavy chain1 gene with human disorders, including movement hyperactivity, hyperexcitability disorders (e.g. myoclonic cramping, epilepsy), excitotoxicity disorders, and neurodegeneration, such as Alzheimer's disease, Parkinson's disease, and Huntington's disease, and specifically motor neuron degeneration, such as ALS (Amyotrophic Lateral Sclerosis), SMA (Spinal Muscular Atrophy), SBMA (Bulbo-Spinal Muscular Atrophy), PBP (progressive Bulbar Palpsy), PMA (Progressive Muscular Atrophy), and PLS (Primary Lateral Sclerosis), resulting in whole or in part from these mutations or defects in the cytoplasmic dynein heavy chain1 gene or the protein it encodes. The Cra1 (cramping 1) mutation in cytoplasmic dynein itself results in progressive motor neuron degeneration in heterozygous mice. Specifically, this invention provides an animal model that demonstrates the role of cytoplasmic dynein heavy chain1 in movement hyperactivity, hyperexcitability disorders (e.g. myoclonic cramping, epilepsy), excitotoxicity disorders and neurodegeneration, specifically α-motor neuron degeneration, resulting from a mutation or defect in cytoplasmic dynein heavy chain1. The Cra1 animal model described herein demonstrates a germline missense mutation that causes subtle changes in the cytoplasmic dynein heavy chain 1, and is sufficient to specifically induce age dependent motor neuron degeneration without grossly affecting the housekeeping function of the dynein-dynactin complex in other cell types. The Cra1 mutation in cytoplasmic dynein heavy chain 1 allows normal development and normal function of the organism in early adulthood. The animal model provided herein meets the currently unfulfilled need for a validated model to study the causes and precise physiological effects of such diseases associated with aberrant cytoplasmic dynein heavy chain1 function. Similarly, this invention provides a means for identifying antagonists and inhibitors for cytoplasmic dynein heavy chain1 in such disorders.

The present invention also provides significant data useful in neurobiology and clinical research, as it describes in an animal model a new pathway involved in neurodegeneration, and also suggests a possible genetic cause for ALS/Lou Gehrig's disease.

Furthermore, the present invention provides diagnostic tests for latent or progressive diseases, or a propensity to develope a disease or passing on a contributing or causatory recessive genetic mutation to progeny, wherein the disease is selected from, but not limited to, the group of human disorders, including movement hyperactivity, hyperexcitability disorders (e.g. myoclonic cramping, epilepsy), excitotoxicity disorders, and neurodegeneration, in particular Alzheimer s disease, Parkinson's disease, Huntington's disease, amyotropic lateral sclerosis, spinal muscular atrophy, and fiber type grouping in Musculus tibialis anterior. More specifically the present invention provides diagnostic tests for latent or progressive diseases, or a propensity to develope a disease or passing on a contributing or causatory recessive genetic mutation to progeny, wherein the disease is selected from, but not limited to, the group of human motor neuron degeneration disorders, including ALS (Amyotrophic Lateral Sclerosis), SMA (Spinal Muscular Atrophy), SBMA (Bulbo-Spinal Muscular Atrophy), PBP (progressive Bulbar Palpsy), PMA (Progressive Muscular Atrophy), and PLS (Primary Lateral Sclerosis).

### SUMMARY OF THE INVENTION

This invention provides inter alia a non-human animal useful as a model of dynein heavy chain disorders in humans, particularly cytoplasmic dynein heavy chain1 disorders.

In one embodiment, the invention provides an animal model which carries a mutated cytoplasmic dynein heavy chain1 gene encoding a cytoplasmic dynein heavy chain1 protein with a modified amino acid sequence compared to the wild type sequence. The invention also provides cell lines derived from the animal model of the present invention.

The present invention also relates to the use of the animal model of the invention for the study of disorders associated with deficiencies or malfunctions in cytoplasmic dynein heavy chain1. In one embodiment, the invention provides methods of diagnosis for deficiencies or malfunction in cytoplasmic dynein heavy chain1 or the gene encoding it. In another embodiment, the invention provides a method for screening of preventive or therapeutic agents of disorders and symptoms associated with movement hyperactivity, hyperexcitability disorders (e.g. myoclonic cramping, epilepsy), excitotoxicity disorders and neurodegenerative diseases; overactivity or undesirable activity of endogenous cytoplasmic dynein heavy chain1; overexpression, over-production or under-production of endogenous cytoplasmic dynein heavy chain1; an excessive or undesirable condition shown to be modulated by endogenous cytoplasmic dynein heavy chain1.

Furthermore, the present invention provides mutated cytoplasmic dynein heavy chain1 nucleic acids and amino acids having modified sequences compared to the wild type sequence, as well as vectors and cell lines for expressing the muteins recombinantly. These mutated nucleic acids and amino acids may also be used in the diagnostic and therapeutic methods contemplated herein. In a specific embodiment, the cytoplasmic dynein heavy chain1 mutein has an amino acid substitution at a position that corresponds to a conserved amino acid of mouse or human cytoplasmic dynein heavy chain 1 as described in an individualized manner in connection with the preferred muteins of the invention below. In a specific embodiment, the cytoplasmic dynein heavy chain1 mutein has an amino acid substitution, e.g., at position 1055 resulting from a point mutation at nucleotide +3169 of the coding sequence, corresponding to position 3328 of SEQ ID NO:1 (Genbank Acc. No. AY004877; murine Dnchc1 mRNA). Also provided are recombinantly generated cytoplasmic dynein heavy chain1 muteins of the invention (both the murine and human othologs), as well as antibodies binding to these muteins, and chimeric protein derivatives of these cytoplasmic dynein heavy chain1 muteins.

Uses of these muteins as modulators (e.g. agonists or antagonists) of endogenous cytoplasmic dynein heavy chain1 activity are also contemplated. Consequently, pharmaceutical compositions comprising the cytoplasmic dynein heavy chain1 muteins of this invention with or without a pharmaceutically acceptable carrier are also contemplated. Specifically, the invention includes the use of the cytoplasmic dynein heavy chain1 muteins of the present invention, the polynucleotides encoding them, and vectors bearing said polynucleotides, for the prevention, treatment or amelioration of a medical condition in a mammalian subject, particularly a human subject. In particular, the invention provides the use of these muteins for the manufacture of a medicament for prevention, treatment or amelioration of any medical conditions characterized by movement hyperactivity, hyperexcitability disorders (e.g. myoclonic cramping, epilepsy), excitotoxicity disorders and neurodegeneration.

The invention also includes a method of gene delivery and expression in a target cell of a mammal, comprising the step of introducing a viral vector into the target cell, wherein the viral vector is derived from a virus that has a low replicative efficiency in the target cell and has at least one insertion site containing a wild type or mutant cytoplasmic dynein heavy chain1 nucleic acid, wherein the nucleic acid is operably linked to a promoter capable of expression in the host. In a preferred embodiment, the viral vector is a non-lytic viral vector.

The invention also provides a method of gene delivery and expression in a target cell of a mammal comprising the steps of: (a) providing an isolated nucleic acid fragment of wild type or mutant cytoplasmic dynein heavy chain1 of the invention; (b) selecting a viral vector derived from a virus that has a low replicative efficiency in the target cell, wherein the vector has at least one insertion site for insertion of said isolated nucleic acid fragment operably linked to a promoter capable of expression in the target cells; (c) inserting the isolated nucleic acid fragment into the insertion site, and (d) introducing the vector into said target cell wherein said gene is expressed at detectable levels. In a preferred embodiment, the virus is selected from the group consisting of retrovirus, adenovirus, and pox virus. In another embodiment, the virus is a strain that has been genetically modified or selected to be non-virulent in a host. In another embodiment, the pox virus is vaccinia In one embodiment, the virus is selected from the group consisting of retrovirus, adenovirus, iridoviruses, coronaviruses, togaviruses, caliciviruses and picornaviruses.

In the context of the present invention it has been recognized that all members (or subunits) of the dynein-dynactin complex should be considered as suitable targets for screening for a predisposition in hereditary and sporadic forms of motor neuron degenerative diseases like ALS (Amyotrophic Lateral Sclerosis), SMA (Spinal Muscular Atrophy), SBMA (Bulbo-Spinal Muscular Atrophy), PBP (progressive Bulbar Palpsy), PMA (Progressive Muscular Atrophy), and PLS (Primary Lateral Sclerosis). It is also contemplated that not only diseases such as ALS but also other neurodegenerative disorders will be connected to mutations or alterations within the dynein-dynactin complex. This applies, e.g., to disorders which are connected to axonal transport dysfunction such as Huntington's disease (dynein-dynactin binds Huntington associated binding protein 1 (Hap1)), Parkinson's disease (demonstration of inclusion body formation similar to ALS indicated intracellular transport errors) or Alzheimer's disease (disturbance of glutamate homeostasis similar to ALS could be a result of altered retrograde axonal transport kinetics). Thus, such neurodegenerative diseases are likewise disease targets contemplated e.g. in connection with the methods of screening for a predisposition for a neurodegenerative disease according to the present invention.

Accordingly, the present invention provides in a further embodiment a method of identifying a protein or nucleic acid marker indicative of an increased risk of a mammalian subject, particularly a human subject, or a mouse, a rat, a rabbit, a cow or a hamster, preferably a mouse or a rat, of developing a neurodegenerative disease, said method comprising the step of analyzing a test sample derived from said subject for the presence of a difference compared to a similar test sample if derived from a subject of the same species unaffected by or known not to be at risk of developing said disease, wherein said difference is indicative of the presence of a mutation in an allele of a gene coding for a protein, which is a subunit of the dynactin/dynein complex.

In a further aspect, the present invention provides a method of identifying a protein or nucleic acid marker indicative of an association of a neurodegenerative disease in a mammalian subject, particularly a human subject, or a mouse, a rat, a rabbit, a cow or a hamster, preferably a mouse or a rat, with a mutation in an allele of a gene coding for a protein, which is a subunit of the dynactin/dynein complex.

In yet a further aspect of the present invention, a method is provided for identifying a predisposition of a mammalian subject, particularly a human subject, or a mouse, a rat, a rabbit, a cow or a hamster, preferably a mouse or a rat, for developing a neurodegenerative disease, said method comprising the step of determining whether a test sample derived from said subject indicates the presence of a mutation in an allele of a gene coding for a protein, which is a subunit of the dynactin/dynein complex, indicative of an increased risk of said subject of developing said neurodegenerative disease.

Also provided is a method for determining whether a neurodegenerative disease in a mammalian subject, particularly a human subject, or a mouse, a rat, a rabbit, a cow or a hamster, preferably a mouse or a rat, is associated with a mutation in an allele of a gene coding for a protein, which is a subunit of the dynactin/dynein complex, said method comprising the step of determining whether a test sample derived from said subject indicates the presence of a mutation in an allele of a gene coding for said protein.

In yet another aspect of the present invention, oligonucleotides suitable for identifying the above-mentioned mutations, kits containing such oligonucleotides; and solid supports, such as DNA chips, to which said oligonucleotides are bound, are provided.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the examples are illustrative only and are not intended to be limiting.

Features and advantages of the invention in addition to those explained above will be apparent from the following detailed description and claims.

### BRIEF DESCRIPTION OF THE DRAWINGS

FIG. 1 presents a heterozygous Cra1 animal (right) compared to a wildtype mouse (left) demonstrating cramping of the hindlimbs and forelimbs of the mutant animal.

FIG. 2 is a chart diagram, indicating movement as ambulatory activity expressed as beam breaks (average counts per hour) detected by a Cage Rack Photobeam Activity Cage System (San Diego Instruments, Inc., San Diego, Calif., USA) of heterozygous Cra1 (Cra1/+) animals and wildtype individuals during four 6-hour periods of an artificial 24 hour diurnal cycle, in which darkness lasted from 6 pm to 6 am, and the period from 6 am to 6 pm was light.

FIG. 3 is a listing of the latency to fall values (seconds) of heterozygous Cra1 mice (Cra1/+) and control individuals (+/+) in a semi-quantitative hanging wire assay. Muscle endurance of adult heterozygous Cra1 individuals (Cra1/+) was measured in comparison to wildtype individuals by measuring the mouse's latency to fall from an inverted wire grid, as described in Example 3.

FIG. 4 depicts photomicrographs of hematophilin and eosin (H&E) stained histological sections of a Cra1/+ and a wildtype mouse hippocampus. Neurons characterized by reversible excitatory neuronal damage can be identified by the dark staining (“dark neurons”; indicated by arrows). (See Example 5)

FIG. 5 depicts photomicrographs of cross sections of the lumbar area of spinal cords from a wild type (left panel) and a homozygous Cra1/Cra1 individual (right panel). The top row photomicrographs depicts a drastic reduction of the number of neurons in the anterior horn area of the Cra1/Cra1 individual (right), as compared to the wildtype (left). In the middle row, TUNEL stains of anterior horn are shown (see arrow), demonstrating significantly enhanced levels of apoptosis in the anterior horns of the Cra1/Cra1 individuals (right). In the upper row of photomicrographs a reduction is seen in the number of spinal ganglia neurons of Cra1/Cra1 individuals (right).

FIG. 5.2 shows that progressive impairment of muscle function and motor coordination is associated with decreasing numbers of α-motor neurons and altered composition of muscle fibre types. Motor neuron degeneration in 16 month old Cra1/+ heterozygotes (b, d, f) mice in comparison to wildtype (+/+) littermates (a, c, e) (a-d: immunhistochemistry using NeuN primary antibody; e, f: H&E histochemical staining). Loss of spinal α-motor neurons in 16 months old Cra1/+ (h) mice is accompanied by altered composition of muscle fibre types, with a predominance of large type I fibres in Periodic acid-Schiff (PAS) staining, compared to +/+ littermates (g).

FIG. 6 represents a list of selected genes expressed in fetal brain of individual Cra1/Cra1 mice, shown to be transcriptionally deregulated by comparison of transcript levels to those detected in wildtype individuals. The direction of deregulation (up or down) and the biological significance of those genes is indicated.

FIG. 7 schematically depicts an exon structure comparison of the human cytoplasmic dynein heavy chain1 (HsDNCH1) and the mouse cytoplasmic dynein heavy chain1 (MmDnchc1) genes. Exonic sequences were aligned with sequencher software (version 4.0.5, GeneCodes Corp., Ann Arbor Mi, USA). Assembly of the human exons and the mouse exons, respectively, results in a full-length cDNA of human DNCH1 and a full-length cDNA of mouse Dnchc1 (representing the protein coding sequence within the rMRNA, defined under GenBank Accession No. AY004877) as indicated by the arrows at the top of the figure. Equivalent exons are paired, with the human exon positioned above the corresponding mouse exon.

FIG. 8 is a comparative listing of all exons of the mouse (MmDnchc1) and human (Hs DNCH1) dynein heavy chain1 genes. The size of each exon is indicated by its length in basepairs (bp). The human gene consists of 78 exons, and the mouse gene consists of 77 exons. The difference in exon number between these two species is caused by an additional RNA splice event in human, separating exon 67 (61 bp) and exon 68 (124 bp); mouse exon 67 having 185 bp.

FIG. 9 depicts the amino acid sequence alignment between the human (Hs DNCH1) and the mouse (Mm Dnchc1) cytoplasmic dynein heavy chain1 proteins. Black boxes indicate amino acid identity, and grey boxes indicate conserved amino acid substitutions. The degree of identity is 97%.

FIG. 10 depicts the amino acid sequence alignment between the human (Hs DNCH1), the mouse (Mm Dnchc1), and the rat cytoplasmic dynein heavy chain1 proteins, indicating highly conserved amino acid residues between species. Black boxes indicate amino acid identity, and grey boxes indicate conserved amino acid substitutions. The degree of identity is 96%.

FIG. 11 depicts the amino acid sequence alignment between the human and the mouse cytoplasmic dynein intermediate chain 1 proteins. Black boxes indicate amino acid identity, and grey boxes indicate conserved amino acid substitutions. The degree of identity is 88%.

FIG. 12 depicts the amino acid sequence aligment between the human, the mouse, and the rat cytoplasmic dynein intermediate chain 1 proteins, indicating highly conserved amino acid residues between species. Black boxes indicate amino acid identity, and grey boxes indicate conserved amino acid substitutions. The degree of identity is 87%.

FIG. 13 depicts the amino acid sequence alignment between the human and the mouse cytoplasmic dynein intermediate chain 2 proteins. Black boxes indicate amino acid identity, and grey boxes indicate conserved amino acid substitutions. The degree of identity is 94%.

FIG. 14 depicts the amino acid sequence alignment between the human, the mouse, and the rat cytoplasmic dynein intermediate chain 2 proteins, indicating highly conserved amino acid residues between species. Black boxes indicate amino acid identity, and grey boxes indicate conserved amino acid substitutions. The degree of identity is 93.5%.

FIG. 15 depicts the amino acid sequence alignment between the human and the mouse DCTN1 proteins. Black boxes indicate amino acid identity, and grey boxes indicate conserved amino acid substitutions. The degree of identity is 97%.

FIG. 16 depicts the amino acid sequence alignment between the human, the mouse, and the rat DCTN 1 proteins, indicating highly conserved amino acid residues between species. Black boxes indicate amino acid identity, and grey boxes indicate conserved amino acid substitutions. The degree of identity is 95%.

### DETAILED DESCRIPTION OF THE INVENTION

This invention provides a non-human animal useful as a model of dynein heavy chain disorders in humans, particularly cytoplasmic dynein heavy chain1 disorders. The Cra1 mutation, disclosed herein, was generated in offspring of male C3HeB/FeJ mice (Cra1, Institute of Mammalian Genetics at the GSF—National Research Center for Environment and Health) mutagenized with N-ethyl-N-nitrosourea (ENU). The mutation was detected by Cra1 mice displaying an unusual twisting of the body and clenching of the hindlimbs when suspended by the tail. Subsequent breeding and phenotype analysis showed Cra1 as an autosomal dominant trait that gives rise to progressive loss of muscle tone and locomotor ability in heterozygous mice, although life-span appears normal. Furthermore, Cra1 heterozygotes were impaired in motor coordination tests and in muscle function tests. Homozygous Cra1 mice die shortly after birth, and exhibit increased neuronal apoptosis. It is predicted that these homozygous mice also have neuronal inclusion bodies that are positive for both neurofilaments and SOD1 protein. This result implicates modulation of dynein gene function in mechanisms of human motor neuron degeneration, and cell death in general. It also shows an unexpected link between the dynein complex and SOD1 function that may shed further light on why SOD1 mutations cause motor neuron degeneration and ALS.

Dyneins are implicated in a number of different diseases and disorders. Abnormalities in axonal transport are described in the human disease ALS (Sasaki and Iwata (1996) Neurology 47, 535-540). Such abnormalities are also observed in several mouse models with motor neuron degeneration, e.g. the wobbler mouse (Mitsumoto et al. (1986) Ann Neurol 19, 36-43) and transgenic mice overexpressing the heavy chain of neurofilament protein (Collard et al. (1995) Nature 375, 61-64), SOD-1 (Zhang et al. (1997) J Cell Biol 139, 1307-1315) and dynamitin (LaMonte et al. (2002) Neuron 34, 715-727). Studies in Drosophila have shown that mutations in the cytoplasmic dynein heavy chain (cDhc64c) lead to a disruption of axonal organelle transport in both directions and as a consequence to axonal swelling and posterior paralysis (Martin et al. (1999) Mol Biol Cell 10, 3717-3728).

In contrast to the dynactin-overexpressing transgenic mouse described by LaMonte et al. ((2002) Neuron 34, 715-727) which is characterized by an indirect mechanism of inhibition of dynein function in only the neurons, the mouse mutant of the present invention is suitable to investigate the role of cytoplasmic dynein heavy chain1 not only in transport processes in neurons but in all cell types expressing cytoplasmic dynein heavy chain1. Additionally, the mouse mutant of the present invention enables the analysis of cytoplasmic dynein malfunction during development whereas the transgenic mouse described by LaMonte et al. ((2002) Neuron 34, 715-727) does not express the transgene before birth and, therefore, it is characterized by a late onset of symptoms. It has been shown that dynein can associate with its cargo also through interactions with proteins other than dynactin (for review: Karcher et al. (2002) Trends in Cell Biology 12, 21-27). Since the mutation found in the cytoplasmic dynein heavy chain 1 gene of the mouse mutant of the present invention is located outside the binding site for the dynein intermediate chains which impart the binding of dynactin, the mouse mutant of the present invention will prove useful for studying dynactin-independant dynein functions.

Apart from diseases characterized by motomeuron degeneration other types of neurodegenerative diseases and age-related neurodegenerative processes may be related to disturbances of axonal transport. One example is the atrophy of basal forebrain cholinergic neurons in the rat which have been shown to be accompanied by an impairment of uptake or retrograde transport of tracer molecules (De Lacalle et al., (1996) Neuroscience 75, 19-27). It is predicted that dynein function may play a role in the development of such neurodegenerative diseases and age-related neurodegenerative processes, and embodiments of the invention include correlative diagnostic methods, therapeutic medicaments and treatments, as described inn greater detail below.

Another example is Huntington's disease. HAP1, a neuronal cytoplasmic protein, is associated with huntingtin, the protein which carries a polyglutamine repeat that is expanded in Huntington's disease (>36 units). It has been shown that the affinity of HAP1 for huntingtin is increased if the latter bears an expanded polyglutamine repeat (cf Li et al. (2002) J Biol Chem, in press). HAP1 has been found on synaptic vesicles (Gutekunst et al. (1998) J Neurosci 18, 7674-7686) and it binds to P150 (Li et al. (1998) J Biol Chem 273, 19220-19227), the largest subunit of the dynactin complex, which in turn, is required for cytoplasmic dynein mediated intracellular movement including retrograde axonal transport. Functional results obtained by Block-Galarza ((1997) Neuroreport 8, 2247-2251) are in agreement with this potential link between huntingtin and dynein-mediated axonal transport processes. Additionally, Li et al. ((2002) J Biol Chem, in press) provided evidence that HAP 1 is involved in the regulation of vesicular trafficking from early endosomes to the late endocytotic compartment as does dynein (Apodaca (2001) Traffic 2, 149-159). Recent work by Kamal et al. (2000, Neuron 28, 449459) suggests that the armyloid precursor protein (APP), which plays a major role in the development of Alzheimer's disease might function as a membrane bound cargo receptor for kinesin which is responsible for the anterograde transport of vesicles in neurons. Gunawardena and Goldstein ((2001) Neuron 32, 389-401) again found that the axonal transport phenotypes of certain AppI (the Drosophila homolog of APP) mutants are suppressed by a reduction of dynein expression. This result for the first time suggests a functional link between dynein and APP.

The mouse mutant of the present invention provides a means to analyze whether three proteins (huntingtin, HAP1 and APP) involved in the pathogenesis of the neurodegenerative diseases, Huntington's disease and Alzheimer's dementia are linked by the same intracellular transport pathways. This connection will help reveal the pathogenetic mechanisms of both diseases.

Lissencephaly syndromes are a family of diseases which involve incomplete development of the human brain (Dobyns et al. (1993) JAMA 270, 2838-2842) characterized by extensive disorganization of cortical neurons. This defect is thought to be due to a failure of the capability of neuronal cell body migration during the early stage of brain development. As a consequence of this, the convolutions characteristic of the normal brain surface are reduced in extent and number leading to a smooth appearence of wide portions of the brain.

Syndromes involving lissencephaly include Norman-Roberts Syndrome, Fukuyama Syndrome, Walker-Warberg Syndrome and Miller-Dieker Syndrome. Some cases of isolated lissencephaly have been shown to arise from mutations in the LIS-1 gene (e.g. Lo Nigro et al. (1997) Hum Mol Genet 6, 157-164).

Recent work has implicated the human LIS-1 gene in the regulation of function of cytoplasmic dyneins (for review see Vallee et al. (2000) Biochim Biophys Acta 1496, 89-98). Neurons in genetic mosaics of Drosophila which contain a mutated cytoplasmic dynein heavy chain (Dhc64C) display phenotypes similar to Lis 1 mutants (Liu et al. (2000) Nat Cell Biol 2, 776-783). We predict that deregulation of cytoplasmic dynein might be of causative relevance for the development of lissencephalic symptoms.

Homozygous null mice for the Lis1 gene die early in embryogenesis whereas mice bearing mutations which cause different degrees of reduction of the function of Lis1 exhibit corresponding degrees of brain disorganization as well as cerebellar defects (Hirotsune et al. (1998) Nature Genetics 19, 333-339). The mouse mutant of the present invention provides a mean to comparatively analyze the overlap and the differences of cytoplasmic dynein and Lis 1 function in a mammalian model organism.

The homozygous knockout of the gene encoding cytoplasmic dynein heavy chain1 in the mouse results in embryonic lethality before 8.5 days post coitus. Heterozygous individuals have no obvious phenotype (Harada et al. (1998) J Cell Biol 141, 51-59). The transgenic dynamitin-overexpressing mouse described by LaMonte et al. ((2002) Neuron 34, 715-727) is characterized by a motorneuron specific inhibition of the functions of cytoplasmic dynein, which is mediated by the accessory protein complex dynactin. Due to the developmental regulation of the transgene by the Thy2 promotor of the transgene construct, inhibition is restricted to postnatal stages.

There is no animal model available which reflects the consequences of malfunction of cytoplasmic dynein heavy chain1 in vivo in all types of cells and during embryonic and fetal development. The mutation leading to cytoplasmic dynein heavy chain1 dysfunction in the mouse model of the present invention is located within a region of cytoplasmic dynein heavy chain1 dimerization (Tynan et al. (2000) J Biol Chem 275, 32769-32774) which does not overlap with intermediate chain binding. Intermediate chain binding is required for dynactin binding although Vaughan and Vallee ((1995) J Cell Biol 131, 1507-1516) and Tynan et al. ((2000) J Biol Chem 275, 32769-32774) have provided evidence for the existence of non-dynactin based cargo binding mechanisms.

The characterization of such an animal model (i.e. one that reflects the consequences of malfunction of cytoplasmic dynein heavy chain1 in vivo in all types of cells and during embryonic and fetal development) would provide a model for determining the consequences of cytoplasmic dynein malfunction in humans. This in turn would help to evaluate the role of dynein malfunction in various disease processes such as those described above.

The present invention provides a non-human animal model which expresses a modified cytoplasmic dynein heavy chain1 protein compared to the amino acid sequence of the wild type protein. The expressed cytoplasmic dynein heavy chain1 may have similarity in sequence and secondary structure to a vertebrate cytoplasmic dynein heavy chain1. In a preferred embodiment, the vertebrate is preferably from a genus selected from the group consisting of Homo sapiens, Mus musculus (e.g. mice), Rattus (e.g. rats), Oryctologus (e.g. rabbits) and Mesocricetus (e.g. hamsters).

Animals carrying a mutated cytoplasmic dynein heavy chain1 allele expressing the modified cytoplasmic dynein heavy chain1, exhibit a variety of phenotypical features including: myoclonic cramps (which are especially pronounced in the hindlimbs), movement hyperactivity, reduced muscle endurance, excitatory neuronal damage (“dark neurons”) in the hippocampus (gyrus dentatus, CA4, CA3) as well as in the upper layer of the cortex and in the Purkinje cell layer of the cerebellum. These animals also exhibit decreasing numbers of α-motor neurons and altered composition of musculus vastus fibre types.

Compared with heterozygous animals, homozygous individuals exhibit an elevated incidence of perinatal lethality (100% in homozygous animals, versus approximately 20% amongst heterozygous animals), accelerated neurondegeneration in the anterior horns of the spinal cord, and accelerated neurodegeneration in the dorsal root ganglia. Neurodegeneration in heterozygous animals is apparent at a much later stage, i.e. late adulthood.

The observed neurological phenotype of myoclonic cramping and epilepsy in combination with the morphological correlate of reversible excitatory neuronal cell stress within the hippocampal layers CA4 and CA3 are indications for a hyperexcitability status in Cra1 animals. This in turn is consistent with an ambulatory locomotor hyperactivity of Cra1 animals. Hyperexcitation, if occuring repeatedly or continuously over longer periods of time, could result in excitotoxicity by mechanisms such as enhanced generation of reactive oxygen species (ROS) (see Haberny et al. (2002) Toxicol Sci, 68, 9-17; Bondy and LeBel, (1993) Free Radic Biol Med 14, 633-642; Tapia et al. (1993) Neurochem Int 34, 23-31; Niebroj-Dobosz et al. (2002) Acta Neurol Scand 106, 39-43; Shaw and Bains (2002) Cell Mol Biol (Noisy-le-grand) 48, 127-136). ROS are suggested to be involved in the development of several neurodegenerative disorders, e.g. ALS, Parkinson's disease and Alzheimer's disease as well as aging. Therefore there is a possible link between the hyperexcitation status in Cra1 animals and the development of neurodegeneration at a later age.

The term “modified” according to the present invention refers to an alteration compared to the wild type. The term “phenotype” according to the invention refers to a collection of morphological, physiological, behavioral and biochemical traits possessed by a cell or organism that results from the interaction of the genotype and the environment. As mentioned above, the animal model of the present invention displays readily observable abnormalities. In a preferred embodiment the animal of the invention shows at least 2, preferably at least 4, more preferably 6 and most preferably all of the above listed phenotypical features.

Nucleic Acids

The present invention provides nucleic acid sequences encoding wild type and mutated murine and human cytoplasmic dynein heavy chain1 proteins, i.e., muteins as described in an individualized manner in connection with the preferred muteins of the invention below. Specifically, this invention provides mutated nucleic acid sequences for wild type murine cytoplasmic dynein heavy chain1 mRNA (GenBank Accession Number AY004877.1, SEQ ID NO: 1). For example, a mutated version of SEQ ID NO: 1, containing a point mutation at position 3328 (which is position +3164 of the polypeptide coding sequence) is shown in SEQ ID NO:3. In addition, this invention provides a mutated coding nucleic acid sequence for human cytoplasmic dynein heavy chain1 containing the open reading frame for the human ortholog nucleic acid sequence, with a point mutation at the corresponding position (position +3170) in the sequence, as shown in SEQ ID NO:5. In addition, this invention provides the wild type coding nucleic acid sequence for human cytoplasmic dynein heavy chain1, as shown in SEQ ID NO:17.

TABLE 1
GenBank Accession Number AY004877.1 (SEQ ID NO:1)

gi|9717244|gb|AY004877.|Mus musculus cytoplasmic
dynein heavy chain 1 mRNA, complete cds
GCCGCGTCGACAGCCGGCTCCGCTGTCCGCAGTCGGCCGCGGGTTAGCGG
GCAGAGCGTCTCCTGTCGCCGGTTCCCCTCGCTTACTGCTCTCTCGTGCC
GGAGGCCGCTCCTCTCGGCTCTCGCTCTCTCTCTTTCTATCTCTCTCCCT
TCCGCGGATCCGCCATGTCGGAGCCGGGCGGCGGCGAGGACGGCTCGGCA
GGCCTGGAGGTGTCGGCGGTGCAGAATGTGGCGGACGTGGCGGTGCTGCA
GAAGCACCTGCGTAAGCTGGTGCCGCTGCTGCTGGAGGACGGCGGCGACG
CGCCGGCTGCGCTGGAGGCGGCGCTGGAGGAGAAGAGCGCCCTGGAGCAG
ATGCGCAAGTTCCTGTCAGACCCGCAGGTCCACACGGTCCTGGTGGAGCG
CTCCACCCTCAAAGAGGACGTTGGTGATGAAGGAGAAGAGGAGAAAGAAT
TCATTTCCTATAACATCAACATAGACATTCATTACGGGGTAAAGTCCAAC
AGCTTGGCGTTCATCAAGCGAGCTCCTGTGATTGATGCAGACAAGCCAGT
GTCGTCCCAGCTCCGAGTCCTCACTCTGAGTGAAGACTCGCCGTATGAGA
CACTGCACTCTTTCATCAGCAATGCAGTGGCTCCTTTCTTTAAGTCCTAC
ATCAGAGAGTCTGGCAAAGCAGACAGGGATGGTGATAAGATGGCTCCTTC
AGTTGAAAAAAAGATTGCAGAGCTTGAAATGGGACTCCTCCACCTGCAGC
AGAACATTGAGATCCCAGAGATCAGCTTGCCAATTCATCCCATCATCACG
AACGTTGCGAAGCAGTGCTATGAGCGCGGAGAAAAGCCCAAAGTTACAGA
TTTTGGTGATAAGGTTGAAGACCCAACTTTCCTTAACCAATTACAGTCTG
GAGTTAATCGGTGGATCCGAGAAATTCAAAAGGTGACCAAGCTTGACCGG
GATCCAGCATCAGGAACTGCCTTGCAGGAAATCAGCTTTTGGCTAAACTT
GGAACGTGCGTTGTACCGCATCCAAGAGAAGCGGGAGAGCCCCGAGGTGC
TCCTGACTCTGGACATCCTGAAGCATGGCAAACGTTTCCATGCCACTGTC
AGTTTTGACACTGACACAGGTCTGAAGCAGGCGTTGGAAACGGTGAATGA
CTACAACCCTCTGATGAAAGACTTCCCTCTGAATGACTTGCTGTCTGCCA
CGGAGCTGGACAAGATAAGGCAGGCCCTTGTTGCAATTTTCACACATCTG
AGAAAGATCCGCAACACGAAGTACCCGATTCAGCGGGCCCTGCGTCTCGT
GGAGGCCATCTCCAGAGACCTGAGTTCTCAGCTGCTCAAGGTCTTGGGCA
CTCGGAAGTTCATGCATGTAGCCTATGAAGAGTTTGAAAAGCTCATCGTG
GCTTGCTTCGAAGTCTTCCAGACGTGGGACGACGAGTACGAGAAGCTCCA
GGTGCTGCTGCGAGACATCGTCAAGAGGAAGCGGGAGGAGAACCTGAAGA
TGGTGTGGCGCATCAACCCCCCTCACAGGAAGCTTCAGGCCCCCCTCGAC
CAGATGAGGAAGTTCCGCCGCCAGCACGAGCAGCTGAGGGCTGTCATTGT
CAGGGTCCTGAGGCCACAGGTCACAGCAGTCGCACAACAGAACCAAGGAG
AAGCACCTGAACCCCAAGACATGAAAGTGGCCGAGGTGCTCTTTGATGCT
GCCGACGCCAACACCATTGAGGAGCTGAACCTGGCCTACGAGAATGTCAA
GGAAGTCGATGGTCTGGATGTTTCCAAAGAAGGGACGGAGGCCTGGGAGG
CCGCGATGAAGAGATACGATGAGAGGATCGACCGTGTGGAGACCCGCATC
ACCGCCCGCCTCCGAGATCAGCTCGGCACGGCCAAGAATGCCAATGAGAT
GTTCAGGATTTTCTCCAGGTTCAATGCACTGTTCCTCCGCCCACACATCC
GAGGGGCCATTCGTGAATACCAGACCCAGCTCATCCAACGTCTGAAAGAT
GACATCGAATCTCTCCACGACAAGTTCAAGGTCCAGTACCCGCAAAGCCA
AGCTTGTAAAATGAGTCATGTCCGTGACCTGCCCCCCGTGTCAGGGTCTA
TCATCTGGGCTAAACAGATCGACAGACAGCTCACTGCCTACATGAAGCGA
GTGGAGGATGTCCTGGGCAAAGGCTGGGAGAACCACGTGGAAGGGCAGAA
GCTGAAGCAGGATGGAGACAGCTTCCGAATGAAGCTCAACACCCAGGAGA
TCTTCGATGACTGGGCGAGGAAGGTTCAGCAGCCCAACCTTGGTCTCTCC
GGGCCCATCTTCACCATTGAGAGCGCTCGCGTGCGGGGCCGCACTGGCAA
TGTCCTCAAGCTAAAGGTGAACTTCCTTCCAGAGATCATAACACTCTCCA
AAGAAGTCCGGAACCTCAAGTGGCTTGGCTTCCGAGTCCCACTTGCAATT
GTGAACAAGGCTCACCAAGCCAACCAGCTCTACCCATTTGCCATCTCGCT
GATTGAAAGCGTCCGGACTTACGAGCGCACCTGTGAGAAGGTGGAGGAGC
GCAATACCATTTCCCTGCTAGTGGCCGGCCTAAAGAAGGAGGTGCAGGCC
TTAATCGCAGAAGGCATCGCGTTGGTGTGGGAGTCCTACAAGCTCGACCC
CTACGTGCAGCGCCTGGCAGAGACGGTTTTCAACTTCCAGGAGAAGGTGG
ACGACCTACTGATTATTGAGGAAAAGATAGACCTGGAGGTCCGCTCCTTG
GAAACCTGCATGTATGACCACAAGACATTCTCAGAGATCTTGAACAGAGT
GCAGAAAGCAGTGGACGACCTAAACCTGCACTCCTATTCCAATCTGCCCA
TCTGGGTCAACAAGCTGGACATGGAGATTGAAAGGATACTGGGCGTCCGC
CTGCAAGCTGGCCTTCGAGCTTGGACCCAGGTTCTTCTTGGACAAGCTGA
GGATAAAGCAGAAGTCGACATGGATACAGATGCTCCACAAGTCAGTCACA
AGCCTGGTGGAGAGCCCAAGATCAAAAACGTGGTTCACGAGCTGAGGATA
ACAAATCAGGTCATCTATCTGAACCCACCCATTGAGGAGTGCAGGTACAA
GCTGTACCAGGAGATGTTCGCGTGGAAGATGGTTGTGCTGTCCCTGCCCA
GGATCCAGAGCCAGAGGTACCAGGTGGGAGTGCATTACGAGTTGACGGAG
GAAGAGAAGTTCTATCGGAATGCACTGACAAGGATGCCCGACGGCCCTGT
CGCCCTGGAAGAGTCCTACTCTGCGGTCATGGGCATAGTGACTGAAGTTG
AGCAGTATGTTAAGGTTTGGCTTCAGTACCAGTGTTTGTGGGATATGCAG
GCAGAAAACATTTACAACAGGCTAGGGGAAGATCTCAACAAGTGGCAAGC
TCTCCTGGTCCAGATAAGGAAAGCCAGAGGAACCTTTGACAATGCGGAAA
CCAAGAAAGAGTTTGGTCCGGTGGTGATAGATTACGGCAAGGTCCAGTCC
AAGGTGAACTTGAAGTATGACTCCTGGCATAAGGAGGTTCTCAGCAAGTT
TGGGCAGATGCTGGGCTCAAACATGACAGAATTCCACTCCCAGATCTCAA
AGTCTCGCCAAGAGCTGGAGCAGCACTCCGTTGACACAGCCAGTACCTCT
GACGCGGTGACCTTCATCACCTATGTGCAGTCTCTGAAACGGAAGATCAA
ACAGTTTGAGAAGCAAGTTGAACTCTACCGCAATGGTCAGCGTCTGCTGG
AAAAACAAAGGTTCCAGTTCCCGCCTTCTTGGCTTTACATTGACAACATC
GAGGGTGAGTGGGGGGCCTTCAATGACATCATGCGGCGGAAGGACTCTGC
CATTCAGCAACAGGTGGCAAACCTGCAAATGAAGATCGTACAGGAGGACA
GAGCTGTGGAAAGCCGGACCACGGATCTGTTGACAGACTGGGAGAAGACC
AAACCTGTCACAGGCAATCTGCGTCCTGAGGAGGCTCTTCAGGCCCTCAC
CATATATGAAGGGAAGTTCGGCCGGCTGAAGGACGACAGAGAGAAATGTG
CAAAGGCTAAGGAAGCGTTGGAACTCACAGACACAGGCCTCCTCAGTGGC
AGTGAAGAGCGTGTACAGGTGGCCTTGGAGGAGTTACAAGACCTCAAAGG
TGTGTGGTCAGAGCTTTCTAAGGTCTGGGAACAAATTGATCAAATGAAGG
AGCAGCCGTGGGTCTCCGTTCAGCCTCGGAAGCTTCGACAAAACCTAGAC
GGCCTCTTGAACCAGCTGAAGAACTTCCCTGCGAGGCTTCGGCAGTATGC
TTCCTATGAGTTTGTTCAGAGGCTGCTGAAAGGCTACATGAAGATAAACA
TGTTGGTAATTGAACTGAAATCAGAAGCACTCAAAGACCGGCACTGGAAA
CAGCTGATGAAGAGGCTTCATGTTAACTGGGTGGTTTCTGAGCTGACGCT
GGGCCAGATCTGGGACGTGGACCTTCAGAAGAACGAAGCTGTCGTCAAGG
ATGTGCTGCTGGTGGCACAAGGCGAGATGGCCTTGGAAGAATTTTTGAAG
CAGATACGAGAAGTGTGGAACACATACGAACTAGACTTGGTCAACTATCA
GAACAAATGCCGCCTCATCCGAGGCTGGGATGACCTCTTCAACAAGGTCA
AGGAGCATATCAACAGCGTCTCTGCCATGAAACTCTCTCCCTATTACAAG
GTTTTTGAAGAAGATGCCCTGAGCTGGGAGGACAAGCTGAACCGAATCAT
GGCTCTCTTTGACGTGTGGATTGACGTGCAGAGGCGCTGGGTCTACTTGG
AAGGCATCTTCACAGGCAGTGCGGACATCAAACATCTGCTGCCTGTGGAA
ACCCAGCGCTTCCAGAGTATCAGCACGGAGTTTTTGGCTCTGATGAAAAA
AGTCTCCAAGTCTCCACTTGTGATGGATGTTCTGAACATACAGGGAGTCC
AGAGGTCTCTGGAAAGGTTGGCAGACCTGCTGGGGAAGATCCAGAAAGCA
TTGGGGGAGTACCTGGAACGGGAGCGCTCGTCTTTCCCCAGGTTCTACTT
TGTGGGTGATGAGGATTTGCTTGAGATCATTGGGAATAGCAAGAACGTGG
CTAAGTTGCAGAAGCACTTCAAGAAAATGTTCGCCGGCGTTTCGAGCATC
ATCTTAAATGAGGATAACTCTGTGGTCCTCGGCATCTCATCCCGAGAAGG
GGAGGAGGTTATGTTTAAAACTCCAGTGTCCATCACTGAACACGCCAAAA
TCAATGAGTGGCTCACTCTGGTGGAAAAGGAGATGAGAGTCACCTTGGCT
AAACTTCTGGCAGAGTCTGTTACAGAAGTGGAGATTTTTGGAAAAGCAAC
TTGTATTGACCCAAATACCTACATTACTTGGATTGATAAATACCAGGCCC
AGCTAGTGGTATTGTCAGCGCAGATAGCGTGGTCCGAGAACGTGGAGAAT
GCGCTGAGCAACGTTGGGGGAGGTGGTGATGTGGGGCCCTTGCAGTCTGT
GCTGAGCAATGTGGAGGTCACACTCAACGTCTTAGGGGACTGTGTCCTCA
TGGAGCAGCCTCGTGTCCGGAGAGGGAAGCTGGAGCACTTGATCACAGAG
CTGGTTGACCAAAGAGAGGTTACCAGGTCCTTGATGAAAAGCAAGATCGA
CAACGGCAAATCTTTCGAATGGCTCAGCCAGATGCGATTTTACTTTGAGC
CTAAGCAAACGGACGTGTTACAGCAGCTGTCCATTCAGATGGCCAATGCC
AAGTTTAACTACGGCTTTGAGTACCTGGGTGTTCAAGACAAGCTGGTTCA
GACGCCCCTCACTGACCGTTGCTATTTGACAATGACACAAGCTTTGGAGG
CCAGGCTTCGGGGCTCCCCATTTGGAGCTGCTGGAACCGGAAAGACGGAG
TCCGTCAAAGCCCTCGGCCATCAGCTGGGGCGGTTCGTCTTAGTTTTCAA
CTGTGAGGAAACCTTTGATTTCCAGCCGATGGGAGGTATCTTTGTGGGGC
TTTGCCAAGTGGGCGCGTGGGGCTGCTTTGATGAGTTTAACCGCCTGGAG
GAGCGGATGCTGTCAGCTGTATCCCAGCAGGTGCAGTGCATACAAGAGCC
ACTGCGGGAGCATTCCAACCCCAACTAGGAGAAGAGCTCAGGCCCCATTA
CTTGTGAGCTGCTGAACAAACAAGTCAAGGTTAGGCGAGAGATGGGTATC
TTCATCACCATGAACCCAGGCTATGCAGGTCGCTGAAACGTCCCCGACAA
CGTCAAGAAGCTCTTCCGGAGCTTGGCGATGACCAAGGCTGACGCGCAGC
TGATCGCGCAGGTCATGCTGTACTCGGAGGCCTTGCGGACTGGTGAGGTC
CTTCGCAACAAGATCGTCCCGTTCTTCAAACTGTCTGATGAGCAACTGTC
TTCTCAAAGCCATTATGACTTTGGGCTTCGGGCTTTGAAGAGTGTCCTTG
TAAGTGCAGGCAATGTGAACAGGGAGAGGATCCACAAGATAAAGAGGGAG
AAAGAAGAACGGGGGGAAGCAGTTGATGAAGGAGAGATCGCTGAGAACCT
ACCTGAGGAGGAGATTCTGATCCAGAGTGTGTGCGAGACAATGGTGCGCA
AGCTGGTGGCAGAAGATATCCCGGTGCTCTTCAGCCTCCTGTCTGAGGTG
TTGCCTGGAGTCCAGTACCACCGTGGGGAGATGAGACGCCTTCGAGAAGA
GCTGAAGAAAGTGTGTGAGGAGATGTACTTGACATATGCCGAGGGAGAAG
AAGTTGGGGGGATGTGGGTTGAAAAGGTTCTCCAGGTCTATGAGATCACG
CAGATTAACCACGGCCTCATGATCCTTGGGCCTTGAGGAAGTGGGAAGAG
CATGCGCTGGCGAGTGCTGCTGAACGCTCTAGAGAGGCTTGAGGGTGTCG
ACGGCGTGGCGCACATAATTGAGCCCAAGGCCATGAGCAAAGACCAGCTC
TACCGAACCTTCGATCCCAACACCAGGGAGTGGACAGAGGGACTCTTCAC
ACACCTCCTCACAAACATCATAGACAATGTGAGAGGTGAGCTGCAGAAAC
GCCAGTGGATTGTCTTTGATCGCGATGTCGACCCCGAGTGCGTGGAGAAC
CTGAACTCCGTGGTGGATGACAACAAGCTCGTGACGCTCCCTAATGCAGA
GCGCCTTAGCCTGCCTGCCAATGTCGCCATAATGTTTCAAGTCGAGGACT
TGAAATACGCGACCGTCGCGAGGGTGTCTCGCTGTGGCATCGTGTCGTTC
AGTGAGGACGTCCTGACCACAGACATGATGTTAAATAACTTCCTGCCGAG
CCTGCGGAGCATTCCCCTGCATCAACGCGAGGACCAGCGACAGCCTAGCC
GAAAACCCAAAGAAGAGGAGCCCCAACAGGCGCCTTCTCCAATCCTACAG
ATTCAAAGAGATCGAGCAACCATCATCCAGCCCTAGTTCACGTCCAACGG
CCTGGTGAGCAAGGCCCTGCAGCACGCCTTCAAGGTGGAGCACATCATCC
ACCTCACGCGGCTGCGCTGCCTGGGCTCGCTCTTCTCCATGCTGCACCAG
GCCTGCCGCAACGTGGCCCAGTACAATCCCAACCATCCTGATTTCCCCAT
GCAGATTGAGCAGCTGGAGCGCTACATTCAGCCCTATCTGGTTTACCCCA
TCCTCTGGTCCTTATCTGGAGACAGCCGGCTGAAGATGAGAGCAGAGCTG
GGCGAGTACATCCGGAGCATCACCACTGTGCCGCTGCCCACTGCGCCCAA
CGTCCCCATCATTGACTATGAGGTGTCCATCAGTGGAGACTGGTCCCCCT
CCCAGGCCAAGGTGCCTCAGATTGAAGTCCAGACACACAAGGTGGCAGCC
CCAGATGTCGTCGTGCCAACCTTAGATACAGTCCGCCATGAAGCTCTCCT
CTACACTTGGCTGGCTGAACACAAACCCCTGGTCTTATGTGGCCCTCCTG
CCTCTCCCAAGACCATGACACTCTTCACTGCTCTCCGGGCCTTCCCTCAC
ATGGACCTTCTGGGCCTAAACTTCTCAAGTGCCACTACACCACAGCTGTT
GCTGAAGACGTTTGACCACTACTGCGAATACAGGCGCACACCTAACGGTG
TTGTCTTGGCTCCTGTTCAGCTTGGAAAGTGGCTCCTCTTGTTCTGTGAT
GAAATCAACTTGCCCGATATGGATAAATACGGCACACAGAGAGTCATCTC
CTTCATCAGACACATCGTGGAGCATGGACCCTTCTATCGTACCTCAGACC
AAACGTCGGTAAACCTAGAGAGAATCCAGTTTGTTGGGGCTTGCAATCCA
CCCACTGACCCTGGGAGAAAGCCCCTCTCACACAGGTTCTTGCGCCACGT
GCCTGTTGTATATGTGGATTACCCAGGACCAGCCTCCCTGACGCAAATCT
ATGGCACCTTCAACCGCGCCATGCTGAGGCTCATTCCCTCTCTCCCTACC
TATCCAGAACCCCTCACTGCCCCCATGGTGGAGTTCTATACCATGTCACA
GGACCGGTTTACTCAGGACACACAGCCACACTATATCTACTCGCCCCGTC
AGATGACTCGGTGGGTAAGAGGAATCTTTGAAGCACTGAGACCCCTGGAG
ACATTGCCTCTTCAAGGTCTCATTCGTATTTGGGCACATGAAGCCCTCCG
TCTCTTCCAAGACAGCCTTGTGGAGGATGAGCAGCGGCGCTGGACTGACG
AAAACATTGACATGCTTGCCTTGAAGCACTTCCCTAACATAGACAAAGAG
AAAGCCATGAGCCGGCCTATCCTGTACAGCAACTGGCTGTCAAAGGATTA
TATCCCAGTTGACCAAGAAGAGCTAAGGGATTATGTCAAGGCTAGGCTGA
ACGTCTTCTATGAAGAAGAACTTGATGTTCCCTTGGTCTTGTTCAATGAA
GTTCTGGACCATGTGCTGAGGATAGACAGAATATTCCGACAACCTCAAGC
CCATCTGCTTCTGATTGGTGTTAGTGGAGCTCCAAAAACCACCCTGTCTC
GCTTTGTCGCCTCGATCAATGGCTTGAGTGTATACCAGATTAACGTCCAC
AGGAACTACACTGCGCAACACTTCGATCAAGATCTTAGGACACTCCTGAG
ACCGTCTGCCTCTAAAAATCAAAAGATAGCATTCATAATCCATGACTCCA
ACGTCTTAGATTCCGGATTCCTGGAACGAATGAATACCCTTCTGCCTAAT
CCCGAGGTTCCTCGCCTCTTTCAAGCCGATGAGTACCCCACGCTCATGAC
ACAGTGCAAAGAGGCGCCCCAGAAGGAGGGGCTCATGCTGGACTCCCACC
ACCAGCTCTACAACTCCTTCACCACCCACCTCATCCCCAACCTCCACCTC
CTCTTCACCATGAACCCCTCCTCACAGCCCCTCAAGGACACCCCCCCCAC
CTCACCACCCCTTTTCAACACGTCTCTGCTCAACTCCTTTGGTGACTCCT
CCACTCAACCACTCTATCAACTCCCCAAACAATTCACCAGTAAGATCCAC
CTCCACAACCCGAACTATATACTGCCTCACTACATCCCAGTTCTCTATGA
TAAACTCCCCCAGCCCCCAACACACCCCCAAGCCATTCTGAACACCTCTC
TGTTTGTCCATCACACTCTTCACCAGGCTAATGCCCCCCTCCCCAAGCGA
GCTCCCAGAACCATCCCCATCACACCTCCCCACTACCTCGACTTCATCAA
CCACTACCCCAACCTCTTCCATGACAAGCCCACCGACTTCCAGGAGCAGC
AGATCCACCTCAATCTTGGGCTCCCAAAAATCAAAGACACCCTCGACCAC
CTCGAAGAACTCCCCCCTCACCTGAGCATCAAGAGCCAAGACCTCGAGCT
CAACAACCCAGCCCCCAATGACAAGCTCAAGAAAATCCTGAAAGACCAGC
AACACCCTCACAACAAGAACGTCATGACCCAAGAAATCCACCACCACCTA
CACAAACAGCACGAACTGATCCCACACAACCAGATGACTCTCAAGGAGCA
CCTCGATAAACTGCAGCCTCCTCTCATCCACGCCCAGAACGCTGTGAAGT
CCATCAAGAAGCAGCACCTGGTGGAGGTGAGGTCCATGGCCAACCCTCCT
GCAGCTGTGAAGCTGGCTCTGGAGTCCATCTGCCTGCTGCTTGGTGAGAG
CACCACGGACTGGAAGCAGATCCGCTCCATCATCATGAGAGAGAACTTCA
TCCCCACCATCGTCAACTTCTCGGCTGAGGAGATCAGTGATGCCATACGA
GAGAAGATGAAGAAAAACTACATGTCCAACCCCAGTTACAACTATGAGAT
CGTCAACCGGGCTTCCCTGGCTTGTGGTCCTATGGTGAAGTGGGCGATTG
CACAGCTCAATTATGCAGACATGTTAAAGCGAGTGGAGCCCCTGAGGAAT
GAGCTGCAGAAGCTGGAAGATGACGCCAAGGACAACCAGCAGAAAGCCAA
TGAGGTGGAGCAGATGATCAGGGACCTGGAAGCCAGCATTGCCCGCTACA
AGGAGGAGTACGCTGTCCTCATCTCTGAGGCCCAGGCCATCAAGGCAGAC
CTGGCAGCTGTGGAAGCAAAGGTAAACCGGAGCACTGCACTTCTAAAGAG
TTTATCTGCCGAAAGGGAACGTTGGGAAAAGACGAGTGAAACTTTCAAAA
ACCAGATGTCTACCATTGCTGGGGACTGCCTCCTGTCGGCGGCCTTCATT
GCTTACGCTGGCTACTTTGACCAGCAAATGCGCCAGAACTTGTTCACCAC
CTGGTCTCATCACTTACAGCAAGCCAACATCCAGTTCCGCACAGACATTG
CAAGGACGGAGTACCTCTCCAATGCTGACGAACGCCTCCGCTGGCAGGCC
AGCTCTCTGCCTGCCGACGACCTGTGCACAGAGAATGCCATCATGCTAAA
ACGATTCAATAGGTACCCGCTGATCATCGACCCCTCCGGACAGGCCACAG
AATTCATCATGAATGAATATAAGGACCGGAAGATCACAAGGACCAGCTTC
TTAGATGATGCCTTCCGGAAGAATTTGGAGAGTGCTCTGAGATTTGGCAA
TCCCCTTCTTGTCCAGGATGTGGAAAGCTACGATCCAGTTTTGAATCCAG
TACTGAACCGTGAAGTTCGGCGAACAGGAGGGAGGGTGCTGATTACCCTC
GGTGACCAAGACATAGACCTGTCACCATCCTTTGTCATCTTTCTGTCTAC
CCGAGACCCAACTGTGGAGTTCCCACCAGACCTCTGCTCCCGGGTGACCT
TTGTGAACTTCACCGTCACCCGCAGCAGTTTACAGAGCCAGTGTCTCAAT
GAAGTGCTTAAAGCAGAAAGGCCAGACGTGGACGAGAAGCGCTCCGACCT
CCTGAAACTGCAAGGGGAGTTCCAACTCCGTTTACGGCAGCTTGAAAAGT
CTCTGCTCCAAGCTCTGAATGAGGTAAAAGGCCGCATTCTGGTTGACGAC
ACCATCATTACAACCCTGGAGAATCTGAAGCGGGAAGCTGCCGAGGTTAC
CAGAAAGGTGGAGGAGACAGATATTGTCATGCAGGAGGTGGAGACCGTGT
CCCAGCAGTACCTGCCGCTGTCCACTGCCTGCAGCAGCATCTACTTCACT
ATGGAGTCCTTGAAGCAGGTTCACTTCCTGTACCAGTACTCTCTGCAGTT
CTTCCTGGACATCTACCACAACGTGCTGTATGAGAACCCCAACCTGAAGG
GTGCCACCGATCACACACAGCGCCTCTCCGTCATAACCAAGGACCTCTTC
CAGGTGGCCTTTAACCGAGTGGCTCGAGGCATGCTGCACCAGGACCACAT
CACCTTCGCCATGCTGCTAGCGAGGATCAAGCTGAAAGGCACCGTGGGGG
AGCCCACCTATGATGCAGAGTTCCAGCACTTCCTCAGAGGGAAGGAGATT
GTGCTGAGTGCTGGCTCCACCCCCAAAATCCAGGGCCTGACCGTAGAGCA
GGCAGAAGCTGTCGTGAGGTTGAGCTGTCTGCCTGCATTTAAAGATCTGA
TTGCCAAGGTGCAGGCAGATGAGCAGTTTGGCATTTGGCTGGATAGCAGT
TCACCAGAGCAGACCGTGCCATACCTCTGGAGCGAGGAAACGCCTACAAC
TCCCATTGGGCAGGCCATCCACCGCCTGCTCCTGATTCAAGCCTTCCGAC
CCGACCGCCTGTTGGCCATGGCCCACATGTTTGTTTCCACAAACCTCGGA
GAGTCCTTCATGTCCATCATGGAGCAGCCTCTCGACCTCACCCACATTGT
GGGCACAGAGGTGAAGCCAAACACACCTGTCCTGATGTGCTCTGTGCCTG
GGTATGATGCCAGCGGACATGTTGAGGACCTCGCAGCAGAGCAGAACACG
CAGATCACGTCTATTGCCATCGGCTCAGCAGAAGGCTTTAACCAAGCAGA
TAAGGCCATCAACACCGCCGTGAAGTCAGGCAGGTGGGTGATGTTGAAGA
ACGTGCACCTGGCCCCGGGCTGGCTGATGCAGCTGGAGAAGAAGCTGCAC
TCCCTGCAGCCCCACGCCTGCTTCCGACTTTTCCTCACCATGGAGATCAA
CCCCAAGGTGCCCGTGAACCTGCTCCGTGCTGGTCGGATCTTTGTGTTTG
AGCCACCACCCGGAGTGAAAGCCAACATGCTGAGGACATTTAGCAGCATC
CCTGTCTCTAGGATATGCAAGTCTCCAAATGAGCGCGCCCGCTTGTATTT
CCTGCTGGCTTGGTTCCATGCCATCATCCAAGAGCGCCTGCGCTATGCTC
CACTGGGGTGGTCAAAGAAGTACGAATTTGGAGAATCTGACCTTCGATCA
GCTTGTGACACGGTGGACACGTGGCTGGACGACACAGCCAAGGGCCGGCA
GAACATCTCACCGGATAAGATCCCATGGTCTGCCCTGAAGACCCTGATGG
CGCAGTCCATCTATGGTGGGCGGGTGGACAATGAGTTCGACCAGCGTCTG
CTCAACACCTTCCTGGAGCGGCTGTTCACCACCCGCAGCTTCGACAGTGA
ATTCAAGCTGGCGTGCAAGGTTGACGGGCACAAGGACATCCAGATGCCTG
ATGGTATGAGGCGAGAGGAGTTTGTGCAGTGGGTGGAGCTGCTGCCTGAC
GCCCAGACACCCTCATGGCTGGGCCTCCCCAACAATGCTGAAAGGGTCCT
GCTTACCACCCAAGGCGTGGACATGATCAGCAAGATGCTGAAGATGCAGA
TGCTGGAGGACGAGGATGACCTGGCCTACGCAGAGACGGAGAAGAAGGCA
AGGACGGACTCCACCTCCGATGGCCGTCCAGCCTGGATGAGGACACTGCA
CACCACGGCGTCCAACTGGCTGCACCTCATCCCACAGACACTGAGCCCCC
TGAAGCGCACCGTGGAGAACATCAAGGATCCTTTGTTTAGGTTCTTTGAG
AGAGAAGTAAAGATGGGGGCCAAGTTACTCCAAGATGTGCGCCAAGACCT
TGCTGACGTCGTTCAAGTGTGCGAGGGGAAAAAGAAGCAGACAAACTACC
TGCGCACGCTCATCAATGAGCTGGTGAAAGGGATCTTGCCTCGGAGCTGG
TCCCATTACACAGTGCCTGCAGGCATGACGGTGATCCAGTGGGTGTCAGA
TTTCAGCGAGAGGATCAAACAGCTGCAGAACATCTCACAAGCAGCTGCAT
CTGGTGGTGCCAAGGAACTGAAGAACATCCACGTGTGCCTGGGTGGCCTG
TTTGTACCTGAGGCCTACATCACTGCCACCAGACAGTATGTGGCACAAGC
TAACAGCTGGTCCCTGGAGGAACTCTGCCTGGAAGTCAATGTCACCGCCT
CACAGAGTGCCACCCTTGATGCCTGCAGCTTTGGAGTCACAGGTCTGAAG
CTCCAAGGGGCCACGTGCAGCAACAACAAGCTATCACTCTCCAATGCCAT
CTCCACAGTCCTCCCCCTCACACAGCTGCGCTGGGTGAAACAAACAAGCG
CCGAGAAGAAGGCCAGCGTGGTGACCTTACCTGTCTACCTGAACTTCACC
CGGGCAGACCTCATCTTCACCGTGGACTTTGAAATTGCTACAAAGGAAGA
TCCTCGCAGCTTCTATGAGCGTGGAGTTGCCGTTCTGTGCACTGAGTAAA
GCTAGACTTTTCTCCAGTCCTCTTTCTGGAATAGGAAACTCCCAATATTT
AACCTTCATTCATCCTTAACGGTGTGGAAGGGTTGATGGACTGGAAAGCA
AGTGGTTGGTTCGCGTTGGCGGGTGACAGGTGCTGCGGTTGGAGACGGTG
GGATGGGAGAAGAGAAGAGTGCGCTGTCCGAGGGCTGACCGAATGGCTGG
GTTTAGTAAATAAAACACTAAGCATGACGGGCAAAAAAAAAAAAAAAAAA
A

TABLE 2

Mutated murine cytoplasmic dynein heavy chain 1,
mRNA(SEQ ID NO:3)

GCCGCGTCGACAGCCGGCTCCGCTGTCCGCAGTCGGCCGCGGGTTAGCGG
GCAGAGCGTCTCCTGTCGCCGGTTCCCCTCGCTTACTGCTCTCTCGTGCC
GGAGGCCGCTCCTCTCGGCTCTCGCTCTCTCTCTTTCTATCTCTCTCCCT
TCCGCGGATCCGCCATGTCGGAGCCGGGCGGdGGCGAGGACGGCTCGGCA
GGCCTGGAGGTGTCGGCGGTGCAGAATGTGGCGGACGTGGCGGTGCTGCA
GAAGCACCTGCGTAAGCTGGTGCCGCTGCTGCTGGAGGACGGCGGCGACG
CGCCGGCTGCGCTGGAGGCGGCGCTGGAGGAGAAGAGCGCCCTGGAGCAG
ATGCGCAAGTTCCTGTCAGACCCGCAGGTCCACACGGTCCTGGTGGAGCG
CTCCACCCTCAAAGAGGACGTTGGTGATGAAGGAGAAGAGGAGAAAGAAT
TCATTTCCTATAACATCAACATAGACATTCATTACGGGGTAAAGTCCAAC
AGCTTGGCGTTCATCAAGCGAGCTCCTGTGATTGATGCAGACAAGCCAGT
GTCGTCCCAGCTCCGAGTCCTCACTCTGAGTGAAGACTCGCCGTATGAGA
CACTGCACTCTTTCATCAGCAATGCAGTGGCTCCTTTCTTTAAGTCCTAC
ATCAGAGAGTCTGGCAAAGCAGACAGGGATGGTGATAAGATGGCTCCTTC
AGTTGAAAAAAAGATTGCAGAGCTTGAAATGGGACTCCTCCACCTGCAGC
AGAACATTGAGATCCCAGAGATCAGCTTGCCAATTCATCCCATCATCACG
AACGTTGCGAAGCAGTGCTATGAGCGCGGAGAAAAGCCCAAAGTTACAGA
TTTTGGTGATAAGGTTGAAGACCCAACTTTCCTTAACCAATTACAGTCTG
GAGTTAATCGGTGGATCCGAGAAATTCAAAAGGTGACCAAGCTTGACCGG
GATCCAGCATCAGGAACTGCCTTGCAGGAAATCAGCTTTTGGCTAAACTT
GGAACGTGCGTTGTACCGCATCCAAGAGAAGCGGGAGAGCCCCGAGGTGC
TCCTGACTCTGGACATCCTGAAGCATGGCAAACGTTTCCATGCCACTGTC
AGTTTTGACACTGACACAGGTCTGAAGCAGGCGTTGGAAACGGTGAATGA
CTACAACCCTCTGATGAAAGACTTCCCTCTGAATGACTTGCTGTCTGCCA
CGGAGCTGGACAAGATAAGGCAGGCCCTTGTTGCAATTTTCACACATCTG
AGAAAGATCCGCAACACGAAGTACCCGATTCAGCGGGCCCTGCGTCTCGT
GGAGGCCATCTCCAGAGACCTGAGTTCTCAGCTGCTCAAGGTCTTGGGCA
CTCGGAAGTTGATGCATGTAGCCTATGAAGAGTTTGAAAAGGTCATGGTG
GCTTGCTTCGAAGTCTTCCAGACGTGGGACGACGAGTACGAGAAGCTCCA
GGTGCTGCTGCGAGACATCGTCAAGAGGAAGCGGGAGGAGAACCTGAAGA
TGGTGTGGCGCATCAACCCCGCTCACAGGAAGCTTCAGGCCCGCCTCGAC
CAGATGAGGAAGTTCCGCCGCCAGCACGAGCAGCTGAGGGCTGTCATTGT
CAGGGTCCTGAGGCCACAGGTCACAGCAGTCGCACAACAGAACCAAGGAG
AAGCACCTGAACCCCAAGACATGAAAGTGGCCGAGGTGCTCTTTGATGCT
GCCGACGCCAACACCATTGAGGAGGTGAACCTGGCCTACGAGAATGTCAA
GGAAGTCGATGGTCTGGATGTTTCCAAAGAAGGGACGGAGGCCTGGGAGG
CCGCGATGAAGAGATACGATGAGAGGATCGACCGTGTGGAGACCCGCATC
ACCGCCCGCCTCCGAGATCAGCTCGGCACGGCCAAGAATGCCAATGAGAT
GTTCAGGATTTTCTCCAGGTTCAATGCACTGTTCGTCCGCCCACACATCC
GAGGGGCCATTCGTGAATACCAGACCCAGCTGATCCAACGTGTGAAAGAT
GACATCGAATCTCTGCACGACAAGTTCAAGGTCCAGTACCCGCAAAGCCA
AGCTTGTAAAATGAGTCATGTCCGTGACCTGCCCCCCGTGTCAGGGTCTA
TCATCTGGGCTAAACAGATCGACAGACAGCTCACTGCCTACATGAAGCGA
GTGGAGGATGTCCTGGGCAAAGGCTGGGAGAACCACGTGGAAGGGCAGAA
GCTGAAGCAGGATGGAGACAGCTTCCGAATGAAGCTCAACACCCAGGAGA
TCTTCGATGACTGGGCGAGGAAGGTTCAGCAGCGCAACCTTGGTGTCTCC
GGGCGCATCTTCACCATTGAGAGCGCTCGCGTGCGGGGCCGCACTGGGAA
TGTCCTCAAGCTAAAGGTGAACTTCCTTCCAGAGATCATAACACTCTCCA
AAGAAGTCCGGAACCTCAAGTGGCTTGGCTTCCGAGTGCCACTTGCAATT
GTGAACAAGGCTCACCAAGCCAACCAGCTCTACCCATTTGCCATCTCGCT
GATTGAAAGCGTCCGGACTTACGAGCGCACCTGTGAGAAGGTGGAGGAGC
GCAATACCATTTCCCTGCTAGTGGCCGGCCTAAAGAAGGAGGTGCAGGCC
TTAATCGCAGAAGGCATCGCGTTGGTGTGGGAGTCCTACAAGCTCGACCC
CTACGTGCAGCGCCTGGCAGAGACGGTTTTCAACTTCCAGGAGAAGGTGG
ACGACCTACTGATTATTGAGGAAAAGATAGACCTGGAGGTCCGCTCCTTG
GAAACCTGCATGTATGACCACAAGACATTCTCAGAGATCTTGAACAGAGT
GCAGAAAGCAGTGGACGACCTAAACCTGCACTCCTATTCCAATCTGCCCA
TCTGGGTCAACAAGCTGGACATGGAGATTGAAAGGATACTGGGCGTCCGC
CTGCAAGCTGGCCTTCGAGCTTGGACCCAGGTTCTTCTTGGACAAGCTGA
GGATAAAGCAGAAGTCGACATGGATACAGATGCTCCACAAGTCAGTCACA
AGCCTGGTGGAGAGCCCAAGATCAAAAACGTGGTTCACGAGCTGAGGATA
ACAAATCAGGTCATCTATCTGAACCCACCCATTGAGGAGTGCAGGTACAA
GCTGTACCAGGAGATGTTCGCGTGGAAGATGGTTGTGCTGTCCCTGCCCA
GGATCCAGAGCCAGAGGTACCAGGTGGGAGTGCATTACGAGTTGACGGAG
GAAGAGAAGTTCTATCGGAATGCACTGACAAGGATGCCCGACGGCCCTGT
CGCCCTGGAAGAGTCCTACTCTGCGGTCATGGGCATAGTGACTGAAGTTG
AGCAGTATGTTAAGGTTTGGCTTCAGTGCCAGTGTTTGTGGGATATGCAG
GCAGAAAACATTTACAACAGGCTAGGGGAAGATCTCAACAAGTGGCAAGC
TCTCCTGGTCCAGATAAGGAAAGCCAGAGGAACCTTTGACAATGCGGAAA
CCAAGAAAGAGTTTGGTCCGGTGGTGATAGATTACGGCAAGGTCCAGTCC
AAGGTGAACTTGAAGTATGACTCCTGGCATAAGGAGGTTCTCAGCAAGTT
TGGGCAGATGCTGGGCTCAAACATGACAGAATTCCACTCCCAGATCTCAA
AGTCTCGCCAAGAGCTGGAGCAGCACTCCGTTGACACAGCCAGTACCTCT
GACGCGGTGACCTTCATCACCTATGTGCAGTCTCTGAAACGGAAGATCAA
ACAGTTTGAGAAGCAAGTTGAACTCTACCGCAATGGTCAGCGTCTGCTGG
AAAAACAAAGGTTCCAGTTCCCGCCTTCTTGGCTTTACATTGACAACATC
GAGGGTGAGTGGGGGGCCTTCAATGACATCATGCGGCGGAAGGACTCTGC
CATTCAGCAACAGGTGGCAAACCTGCAAATGAAGATCGTACAGGAGGACA
GAGCTGTGGAAAGCCGGACCACGGATCTGTTGACAGACTGGGAGAAGACC
AAACCTGTCACAGGCAATCTGCGTCCTGAGGAGGCTCTTCAGGCCCTCAC
CATATATGAAGGGAAGTTCGGCCGGCTGAAGGACGACAGAGAGAAATGTG
CAAAGGCTAAGGAAGCGTTGGAACTCACAGACACAGGCCTCCTCAGTGGC
AGTGAAGAGCGTGTACAGGTGGCCTTGGAGGAGTTACAAGACCTCAAAGG
TGTGTGGTCAGAGCTTTCTAAGGTCTGGGAACAAATTGATCAAATGAAGG
AGCAGCCGTGGGTCTCCGTTCAGCCTCGGAAGCTTCGACAAAACCTAGAC
GGCCTCTTGAACCAGCTGAAGAACTTCCCTGCGAGGCTTCGGCAGTATGC
TTCCTATGAGTTTGTTCAGAGGCTGCTGAAAGGCTACATGAAGATAAACA
TGTTGGTAATTGAACTGAAATCAGAAGCACTCAAAGACCGGCACTGGAAA
CAGCTGATGAAGAGGCTTCATGTTAACTGGGTGGTTTCTGAGCTGACGCT
GGGCCAGATCTGGGACGTGGACCTTCAGAAGAACGAAGCTGTCGTCAAGG
ATGTGCTGCTGGTGGCACAAGGCGAGATGGCCTTGGAAGAATTTTTGAAG
CAGATACGAGAAGTGTGGAACACATACGAACTAGACTTGGTCAACTATCA
GAACAAATGCCGCCTCATCCGAGGCTGGGATGACCTCTTCAACAAGGTCA
AGGAGCATATCAACAGCGTCTCTGCCATGAAACTCTCTCCCTATTACAAG
GTTTTTGAAGAAGATGCCCTGAGCTGGGAGGACAAGCTGAACCGAATCAT
GGCTCTCTTTGACGTGTGGATTGACGTGCAGAGGCGCTGGGTCTACTTGG
AAGGCATCTTCACAGGCAGTGCGGACATCAAACATCTGCTGCCTGTGGAA
ACCCAGCGCTTCCAGAGTATCAGCACGGAGTTTTTGGCTCTGATGAAAAA
AGTCTCCAAGTCTCCACTTGTGATGGATGTTCTGAACATACAGGGAGTCC
AGAGGTCTCTGGAAAGGTTGGCAGACCTGCTGGGGAAGATCCAGAAAGCA
TTGGGGGAGTACGTGGAACGGGAGCGCTCGTCTTTCCCCAGGTTCTACTT
TGTGGGTGATGAGGATTTGCTTGAGATCATTGGGAATAGCAAGAACGTGG
CTAAGTTGCAGAAGCACTTCAAGAAAATGTTCGCCGGCGTTTCCAGCATC
ATCTTAAATGAGGATAACTCTGTGGTCCTCGGCATCTCATCCCGAGAAGG
GGAGGAGGTTATGTTTAAAACTCCAGTGTCCATCACTGAACACCCCAAAA
TCAATGAGTGGCTCACTCTGGTGGAAAAGGAGATGAGAGTCACCTTGGCT
AAACTTCTGGCAGAGTCTGTTACAGAAGTGGAGATTTTTGGAAAAGCAAC
TTCTATTGACCCAAATACCTACATTACTTGGATTGATAAATACCAGGCCC
AGCTAGTGGTATTGTCAGCCCAGATAGCCTGGTCCGAGAACGTGGAGAAT
GCGCTGAGCAACGTTGGGGGAGGTGGTGATGTGGGGCCCTTGCAGTCTGT
GCTGAGCAATGTGGAGGTCACACTCAACGTCTTAGCGGACTCTGTCCTCA
TGGAGCAGCCTCCTCTCCGGAGACGGAAGCTGGAGCACTTGATCACAGAG
CTGGTTCACCAAAGAGACGTTACCAGGTCCTTGATCAAAAGCAAGATCGA
CAACGCCAAATCTTTCGAATGGCTCAGCCAGATGCGATTTTACTTTGACC
CTAAGCAAACGGACGTGTTACAGCAGCTGTCCATTCAGATGGCCAATGCC
AAGTTTAACTACGGCTTTGAGTACCTGGGTGTTCAAGACAAGCTGGTTCA
GACGCCCCTCACTGACCGTTGCTATTTGACAATGACACAAGCTTTGGAGG
CCAGGCTTGGGGGCTCCCCATTTGGACCTGCTGGAACCGGAAAGACGGAG
TCCGTCAAAGCCCTCGGCCATCAGCTGGGGCGGTTCGTCTTAGTTTTCAA
CTGTGACGAAACCTTTGATTTCCAGGCGATGGGACGTATCTTTGTGGGGC
TTTGCCAAGTGGGCGCGTGGGGCTGCTTTGATGAGTTTAACCGCCTGGAG
GAGCGGATGCTCTCAGCTGTATCCCAGCAGGTGCAGTGCATACAAGAGGC
ACTGCGGGAGCATTCCAACCCCAACTACGACAAGACCTCAGCCCCCATTA
CTTGTGAGCTGCTGAACAAACAAGTCAAGGTTAGCCCAGACATGGCTATC
TTCATCACCATGAACCCAGGCTATGCAGGTCGCTCAAACCTCCCCGACAA
CCTCAAGAAGCTCTTCCGGAGCTTGGCGATGACCAAGCCTGACCGGCAGC
TGATCGCCCAGGTCATGCTGTACTCGCAGGGCTTCCGGACTGCTGAGGTC
CTTGCCAACAAGATCGTCCCGTTCTTCAAACTGTGTGATGAGCAACTGTC
TTCTCAAAGCCATTATGACTTTGGGCTTCGGGCTTTGAAGAGTGTGCTTG
TAAGTGCAGGCAATGTGAAGAGGGAGAGGATCCAGAAGATAAAGAGGGAG
AAAGAAGAACGGGGGGAAGCAGTTGATGAAGGAGAGATCGCTGAGAACCT
ACCTGAGCAGGAGATTCTGATCCAGAGTGTCTGCGAGACAATGGTGCCCA
AGCTGGTGGCAGAAGATATCCCGCTGCTCTTCAGCCTCCTGTCTGACGTG
TTCCCTGGAGTCCAGTACCACCGTGGGGAGATGACAGCCCTTCGAGAAGA
GCTGAAGAAAGTGTGTCAGGAGATGTACTTGACATATGGCGACGGAGAAG
AAGTTGGCGGGATGTGGGTTGAAAAGGTTCTCCAGCTCTATCAGATCACG
CAGATTAACCACGGCCTGATGATGGTTGGGCCTTCAGGAAGTGGGAAGAG
CATGGCCTGGCGAGTGCTGCTGAAGGCTCTAGAGAGGCTTGAGGGTGTGG
AGGGCGTGGCCCACATAATTGACCCCAAGGCCATCAGCAAAGACCACCTC
TACGGAACCTTGGATCCCAACACCAGGGAGTGGACAGACGGACTCTTCAC
ACACGTGCTGAGAAAGATCATAGACAATGTGAGAGGTGAGCTGCAGAAAC
GCCAGTGGATTGTCTTTGATGGCGATGTGGACCCCGAGTGGGTGGAGAAC
CTGAACTCCGTGCTGGATGACAACAAGCTCCTGACGCTGCCTAATGGAGA
GCGCCTTAGCCTGCCTCCCAATGTGCGGATAATGTTTGAAGTGCAGGACT
TGAAATACGCGACGCTGGCCACGGTGTCTCGCTGTGGCATGGTGTGGTTC
AGTGAGGACGTCCTGAGCACAGACATGATCTTAAATAACTTCCTGGCCAG
GCTGCGCAGCATTCCCCTGGATGAAGGGGAGGACGAGGCACAGCGTAGGC
GAAAAGGCAAAGAAGACGAGGGCGAAGAGGCGGCTTCTCCAATGCTACAG
ATTCAAAGAGATGCAGCAACGATCATGCAGCCGTACTTCACGTCCAACGG
CCTGGTCACCAAGGCGCTGGAGCACGCCTTCAAGCTGGAGCACATCATGG
ACCTGACGCGCCTGCGCTGCCTGGGCTCGCTCTTCTCCATGCTGCACCAG
GCCTGCCGCAACGTGGCGCAGTACAATGCCAACCATCCTGATTTCCCCAT
GCAGATTGAGCAGCTGGAGCGCTACATTCAGCGCTATCTGGTTTACGCCA
TCCTCTGGTCCTTATCTGGAGACAGCCGGCTGAAGATGAGAGCAGAGCTG
GGCGAGTACATCCGGAGGATCACCACTGTGCCGCTGCCCACTGCGCCCAA
CGTCCCCATCATTGACTATGAGGTGTCCATCAGTGGAGAGTGGTCGCCGT
GGCAGGCCAAGGTGCCTCAGATTGAAGTGGAGACACACAAGGTGGCAGCC
CCAGATGTCGTCGTGCCAACCTTAGATACAGTCCGCCATGAAGCTCTCCT
GTACACTTGGCTGGCTGAACACAAACCCCTGGTCTTATGTGGCCCTCCTG
GGTCTGGCAAGACCATGACACTCTTCAGTGCTCTCCGGGCCTTGCCTGAC
ATGGAGGTTGTGGGCCTAAACTTCTCAAGTGCCACTACACCAGAGCTGTT
GCTGAAGACGTTTGACCACTACTGCGAATACAGGCGCACACCTAACGGTG
TTGTCTTGGCTCCTGTTCAGCTTGGAAAGTGGCTGGTCTTGTTCTGTGAT
GAAATCAACTTGCCCGATATGGATAAATACGGCACACAGAGAGTCATCTC
CTTCATCAGACAGATGGTGGAGCATGGAGCCTTCTATCGTACCTCAGACC
AAACGTGGGTAAAGCTAGAGAGAATCCAGTTTGTTGGGGCTTGCAATCCA
CCCACTGACCCTGGGAGAAAGCCCCTCTCACACAGGTTCTTGCGCCACGT
GCCTGTTGTATATGTGGATTACCCAGGACCAGCCTCCCTGACGCAAATCT
ATGGCACCTTCAACCGCGCCATGCTGAGGCTCATTCCCTCTCTCCGTACC
TATGCAGAACCCCTCACTGCCGCCATGGTGGAGTTCTATACCATGTCACA
GGAGCGGTTTACTCAGGACACACAGCCACACTATATCTACTCGCCCCGTG
AGATGACTCGGTGGGTAAGAGGAATCTTTGAAGCACTGAGACCCCTGGAG
ACATTGCCTGTTGAAGGTCTCATTCGTATTTGGGCACATGAAGCCCTCCG
TCTCTTCCAAGACAGGCTTGTGGAGGATGAGGAGCGGCGCTGGACTGACG
AAAACATTGACATGGTTGCCTTGAAGCACTTCCCTAACATAGACAAAGAG
AAAGCCATGAGCCGGCCTATCCTGTACAGCAACTGGCTGTCAAAGGATTA
TATCCCAGTTGACCAAGAAGAGCTAAGGGATTATGTCAAGGCTAGGCTGA
AGGTCTTCTATGAAGAAGAACTTGATGTTCCCTTGGTCTTGTTCAATGAA
GTTCTGGACCATGTGCTGAGGATAGACAGAATATTCCGACAACCTCAAGG
CCATCTGCTTCTGATTGGTGTTAGTGGAGCTGGAAAAACCACCCTGTCTC
GCTTTGTCGCCTGGATGAATGGCTTGAGTGTATACCAGATTAAGGTCCAC
AGGAAGTACACTGGGGAAGACTTCGATGAAGATCTTAGGACAGTGCTGAG
ACGGTCTGGCTGTAAAAATGAAAAGATAGCATTCATAATGGATGAGTCCA
ACGTCTTAGATTCCGGATTCCTGGAACGAATGAATACCCTTCTGGCTAAT
GGCGAGGTTCCTGGGCTCTTTGAAGGGGATGAGTACGCCACGCTGATGAC
ACAGTGCAAAGAGGGGGCCCAGAAGGAGGGGCTCATGCTGGACTCCCACG
AGGAGCTCTACAAGTGGTTCACCAGCCAGGTGATCCGCAACCTGCACGTG
GTCTTCACCATGAACCCGTCCTCAGAGGGGCTCAAGGACAGGGCGGCCAC
CTCACCAGCCCTTTTCAACAGGTGTGTGCTGAACTGGTTTGGTGACTGGT
CCACTGAAGCACTGTATCAAGTCGGCAAAGAATTCACCAGTAAGATGGAC
CTGGAGAAGCCGAACTATATAGTGCCTGACTACATGCCAGTTGTGTATGA
TAAACTGCCGCAGCCCCCAACACACCGGGAAGCCATTGTGAACAGCTGTG
TGTTTGTCCATCAGACTCTTCACCAGGCTAATGCGCGCCTGGCGAAGCGA
GGTGGCAGAACGATGGCCATCACACCTCGGCACTACCTGGACTTCATCAA
CCACTACGCCAACCTCTTCCATGAGAAGCGCAGCGAGTTGGAGGAGCAGC
AGATGCACCTGAATGTTGGGCTCCGAAAAATCAAAGAGACCGTGGACCAG
GTGGAAGAACTGCGCCGTGACCTGAGGATCAAGAGCCAAGAGCTGGAGGT
CAAGAACGCAGCGGCCAATGACAAGCTGAAGAAAATGGTGAAAGACCAGC
AAGAGGCTGAGAAGAAGAAGGTCATGAGCCAAGAAATCCAGGAGCAGCTA
CACAAACAGCAGGAAGTGATCGCAGACAAGCAGATGAGTGTCAAGGAGGA
CCTGGATAAAGTGGAGCCTGCTGTCATCGAGGCCCAGAACGCTGTGAAGT
CCATCAAGAAGCAGCACCTGGTGGAGGTGAGGTCCATGGCCAACCCTCCT
GCAGCTGTGAAGCTGGCTCTGGAGTCCATCTGCCTGCTGCTTGGTGAGAG
CACCACGGACTGGAAGCAGATCCGCTCCATCATCATGAGAGAGAACTTCA
TCCCCACCATCGTCAACTTCTCGGCTGAGGAGATCAGTGATGCCATACGA
GAGAAGATGAAGAAAAACTACATGTCCAACCCCAGTTACAACTATGAGAT
CGTCAACCGGGCTTCCCTGGCTTGTGGTCCTATGGTGAAGTGGGCGATTG
CACAGCTCAATTATGCAGACATGTTAAAGCGAGTGGAGCCCCTGAGGAAT
GAGCTGCAGAAGCTGGAAGATGACGCCAAGGACAACCAGCAGAAAGCCAA
TGAGGTGGAGCAGATGATCAGGGACCTGGAAGCCAGCATTGCCCGCTACA
AGGAGGAGTACGCTGTCCTCATCTCTGAGGCCCAGGCCATCAAGGCAGAC
CTGGCAGCTGTGGAAGCAAAGGTAAACCGGAGCACTGCACTTCTAAAGAG
TTTATCTGCCGAAAGGGAACGTTGGGAAAAGACGAGTGAAACTTTCAAAA
ACCAGATGTCTACCATTGCTGGGGACTGCCTCCTGTCGGCGGCCTTCATT
GCTTACGCTGGCTACTTTGACCAGCAAATGCGCCAGAACTTGTTCACCAC
CTGGTCTCATCACTTACAGCAAGCCAACATCCAGTTCCGCACAGACATTG
CAAGGACGGAGTACCTCTCCAATGCTGACGAACGCCTCCGCTGGCAGGCC
AGCTCTCTGCCTGCCGACGACCTGTGCACAGAGAATGCCATCATGCTAAA
ACGATTCAATAGGTACCCGCTGATCATCGACCCCTCCGGACAGGCCACAG
AATTCATCATGAATGAATATAAGGACCGGAAGATCACAAGGACCAGCTTC
TTAGATGATGCCTTCCGGAAGAATTTGGAGAGTGCTCTGAGATTTGGCAA
TCCCCTTCTTGTCCAGGATGTGGAAAGCTACGATCCAGTTTTGAATCCAG
TACTGAACCGTGAAGTTCGGCGAACAGGAGGGAGGGTGCTGATTACCCTC
GGTGACCAAGACATAGACCTGTCACCATCCTTTGTCATCTTTCTGTCTAC
CCGAGACCCAACTGTGGAGTTCCCACCAGACCTCTGCTCCCGGGTGACCT
TTGTGAACTTCACCGTCACCCGCAGCAGTTTACAGAGCCAGTGTCTCAAT
GAAGTGCTTAAAGCAGAAAGGCCAGACGTGGACGAGAAGCGCTCCGACCT
CCTGAAACTGCAAGGGGAGTTCCAACTCCGTTTACGGCAGCTTGAAAAGT
CTCTGCTCCAAGCTCTGAATGAGGTAAAAGGCCGCATTCTGGTTGACGAC
ACCATCATTACAACCCTGGAGAATCTGAAGCGGGAAGCTGCCGAGGTTAC
CAGAAAGGTGGAGGAGACAGATATTGTCATGCAGGAGGTGGAGACCGTGT
CCCAGCAGTACCTGCCGCTGTCCACTGCCTGCAGCAGCATCTACTTCACT
ATGGAGTCCTTGAAGCAGGTTCACTTCCTGTACCAGTACTCTCTGCAGTT
CTTCCTGGACATCTACCACAACGTGCTGTATGAGAACCCCAACCTGAAGG
GTGCCACCGATCACACACAGCGCCTCTCCGTCATAACCAAGGACCTCTTC
CAGGTGGCCTTTAACCGAGTGGCTCGAGGCATGCTGCACCAGGACCACAT
CACCTTCGCCATGCTGCTAGCGAGGATCAAGCTGAAAGGCACCGTGGGGG
AGCCCACCTATGATGCAGAGTTCCAGCACTTCCTCAGAGGGAAGGAGATT
GTGCTGAGTGCTGGCTCCACCCCCAAAATCCAGGGCCTGACCGTAGAGCA
GGCAGAAGCTGTCGTGAGGTTGAGCTGTCTGCCTGCATTTAAAGATCTGA
TTGCCAAGGTGCAGGCAGATGAGCAGTTTGGCATTTGGCTGGATAGCAGT
TCACCAGAGCAGACCGTGCCATACCTCTGGAGCGAGGAAACGCCTACAAC
TCCCATTGGGCAGGCCATCCACCGCCTGCTCCTGATTCAAGCCTTCCGAC
CCGACCGCCTGTTGGCCATGGCCCACATGTTTGTTTCCACAAACCTCGGA
GAGTCCTTCATGTCCATCATGGAGCAGCCTCTCGACCTCACCCACATTGT
GGGCACAGAGGTGAAGCCAAACACACCTGTCCTGATGTGCTCTGTGCCTG
GGTATGATGCCAGCGGACATGTTGAGGACCTCGCAGCAGAGCAGAACACG
CAGATCACGTCTATTGCCATCGGCTCAGCAGAAGGCTTTAACCAAGCAGA
TAAGGCCATCAACACCGCCGTGAAGTCAGGCAGGTGGGTGATGTTGAAGA
ACGTGCACCTGGCCCCGGGCTGGCTGATGCAGCTGGAGAAGAAGCTGCAC
TCCCTGCAGCCCCACGCCTGCTTCCGACTTTTCCTCACCATGGAGATCAA
CCCCAAGGTGCCCGTGAACCTGCTCCGTGCTGGTCGGATCTTTGTGTTTG
AGCCACCACCCGGAGTGAAAGCCAACATGCTGAGGACATTTAGCAGCATC
CCTGTCTCTAGGATATGCAAGTCTCCAAATGAGCGCGCCCGCTTGTATTT
CCTGCTGGCTTGGTTCCATGCCATCATCCAAGAGCGCCTGCGCTATGCTC
CACTGGGGTGGTCAAAGAAGTACGAATTTGGAGAATCTGACCTTCGATCA
GCTTGTGACACGGTGGACACGTGGCTGGACGACACAGCCAAGGGCCGGCA
GAACATCTCACCGGATAAGATCCCATGGTCTGCCCTGAAGACCCTGATGG
CGCAGTCCATCTATGGTGGGCGGGTGGACAATGAGTTCGACCAGCGTCTG
CTCAACACCTTCCTGGAGCGGCTGTTCACCACCCGCAGCTTCGACAGTGA
ATTCAAGCTGGCGTGCAAGGTTGACGGGCACAAGGACATCCAGATGCCTG
ATGGTATCAGGCGAGAGGAGTTTGTGCAGTGGGTGGAGCTGCTGCCTGAC
GCCCAGACACCCTCATGGCTGGGCCTCCCCAACAATGCTGAAAGGGTCCT
GCTTACCACCCAAGGCGTGGACATGATCAGCAAGATGCTGAAGATGCAGA
TGCTGGAGGACGAGGATGACCTGGCCTACGCAGAGACGGAGAAGAAGGCA
AGGACGGACTCCACCTCCGATGGCCGTCCAGCCTGGATGAGGACACTGCA
CACCACGGCGTCCAACTGGCTGCACCTCATCCCACAGACACTGAGCCCCC
TGAAGCGCACCGTGGAGAACATCAAGGATCCTTTGTTTAGGTTCTTTGAG
AGAGAAGTAAAGATGGGGGCCAAGTTACTCCAAGATGTGCGCCAAGACCT
TGCTGACGTCGTTCAAGTGTGCGAGGGGAAAAAGAAGCAGACAAACTACC
TGCGCACGCTCATCAATGAGCTGGTGAAAGGGATCTTGCCTCGGAGCTGG
TCCCATTACACAGTGCCTGCAGGCATGACGGTGATCCAGTGGGTGTCAGA
TTTCAGCGAGAGGATCAAACAGCTGCAGAACATCTCACAAGCAGCTGCAT
CTGGTGGTGCCAAGGAACTGAAGAACATCCACGTGTGCCTGGGTGGCCTG
TTTGTACCTGAGGCCTACATCACTGCCACCAGACAGTATGTGGCACAAGC
TAACAGCTGGTCCCTGGAGGAACTCTGCCTGGAAGTCAATGTCACCGCCT
CACAGAGTGCCACCCTTGATGCCTGCAGCTTTGGAGTCACAGGTCTGAAG
CTCCAAGGGGCCACGTGCAGCAACAACAAGCTATCACTCTCCAATGCCAT
CTCCACAGTCCTCCCCCTCACACAGCTGCGCTGGGTGAAACAAACAAGCG
CCGAGAAGAAGGCCAGCGTGGTGACCTTACCTGTCTACCTGAACTTCACC
CGGGCAGACCTCATCTTCACCGTGGACTTTGAAATTGCTACAAAGGAAGA
TCCTCGCAGCTTCTATGAGCGTGGAGTTGCCGTTCTGTGCACTGAGTAAA
GCTAGACTTTTCTCCAGTCCTCTTTCTGGAATAGGAAACTCCCAATATTT
AACCTTCATTCATCCTTAACGGTGTGGAAGGGTTGATGGACTGGAAAGCA
AGTGGTTGGTTCGCGTTGGCGGGTGAcAGGTGCTGCGGTTGGAGACGGTG
GGATGGGAGAAGAGAAGAGTGCGCTGTcCGAGGGCTGACCGAATGGCTGG
GTTTAGTAAATAAACACTAAGCATGACGGGCAAAAAAAAAAAAAAAAAAA

TABLE 3

Mutated human cycloplasmic dynein heavy chain 1,
cDNA (SEQ ID NO:5)

ATGTCGGAGCCCGGGGGCGGCGGCGGCGAGGACGGCTCGGCCGGATTGGA
AGTGTCGGCCGTGCAGAATGTGGCGGACGTGTCGGTGCTGCAGAAGCACC
TGCGCAAGCTGGTGCCGCTGCTGCTGGAGGACGGCGGCGAGGCGCCGGCC
GCGCTGGAGGCGGCGCTGGAGGAGAAGAGCGCCCTGGAGCAGATGCGCAA
GTTCCTTTCGGACCCGCAGGTCCACACGGTGCTGGTGGAGCGCTCCACGC
TCAAAGAGGACGTCGGTGATGAAGGAGAAGAAGAAAAAGAATTCATTTCC
TATAACATCAACATAGACATTCATTATGGGGTTAAATCCAATAGCTTGGC
ATTCATTAAACGTACTCCCGTGATTGATGCAGATAAACCCGTGTCTTCTC
AGCTCCGGGTCCTTACACTCAGTGAAGACTCGCCCTACGAAACTTTGCAT
TCTTTCATTAGCAATGCAGTGGCTCCTTTTTTTAAGTCCTACATTAGAGA
GTCTGGCAAGGCAGACAGGGATGGTGATAAAATGGCTCCTTCAGTTGAAA
AGAAGATTGCAGAACTCGAAATGGGACTCCTTCACTTGCAGCAAAATATT
GAAATTCCGGAGATCAGCCTGCCGATTCATCCAATGATCACAAATGTTGC
AAAACAGTGTTATGAGCGTGGAGAAAAGCCAAAAGTTACAGACTTTGGTG
ATAAGGTTGAAGACCCAACATTTCTTAATCAGTTACAATCTGGAGTTAAC
CGCTGGATCCGAGAAATTCAAAAAGTGACCAAACTGGATCGAGATCCTGC
ATCAGGAACTGCCTTACAGGAAATTAGTTTTTGGCTAAACTTGGAACGTG
CGTTATACCGCATCCAGGAGAAACGGGAGAGCCCGGAAGTTCTCCTGACT
CTGGATATCTTGAAACATGGCAAGCGCTTCCATGCCACCGTCAGTTTTGA
CACTGACACAGGTCTAAAACAGGCTTTGGAAACTGTGAATGACTACAATC
CTCTGATGAAAGATTTCCCTCTGAATGATTTGCTGTCTGCCACGGAGCTG
GACAAAATAAGACAGGCGCTTGTTGCCATTTTCACACATTTGAGAAAGAT
CCGAAACACAAAATATCCTATTCAGAGGGCACTGCGTTTGGTGGAGGCAA
TTTCAAGAGACTTGAGTTCTCAATTACTCAAAGTATTGGGCACTAGGAAA
TTGATGCATGTTGCTTATGAAGAATTTGAAAAAGTTATGGTAGCATGCTT
TGAAGTTTTTCAGACTTGGGATGATGAGTATGAGAAACTTCAGGTATTGT
TGAGAGACATCGTCAAAAGAAAAAGGGAAGAAAATCTGAAGATGGTGTGG
CGTATCAACCCTGCCCACAGGAAGCTGCAGGCCCGCCTTGAGCAGATGAG
AAAATTTAGACGCCAGCATGAACAGCTAAGAGCTGTTATCGTCAGGGTCC
TGAGGCCACAGGTGAGGGCAGTTGCACAACAGAATCAAGGAGAGGTCCCT
GAACCCCAAGATATGAAAGTGGCTGAGGTTCTCTTTGATGCTGCAGATGC
AAATGCCATTGAGGAAGTAAACCTTGCTTATGAGAACGTCAAGGAAGTGG
ATGGACTGGATGTTTCCAAAGAGGGCACGGAAGCCTGGGAGGCTGCTATG
AAGAGGTACGATGAGAGGATCGACAGAGTGGAGACCCGGATCACCGCTCG
CCTTCGGGATCAGCTTGGCACAGCCAAGAATGCCAACGAGATGTTTAGGA
TTTTCTCCAGGTTTAATGCACTGTTTGTCAGGCCTCACATCCGTGGGGCC
ATTCGCGAATACCAGACCCAGCTGATCCAGCGCGTGAAAGATGACATTGA
GTCTCTTCACGACAAGTTCAAGGTCCAGTACCCACAGAGTCAGGCTTGTA
AGATGAGTCACGTTCGTGACTTGCCCCCTGTGTCAGGGTCTATCATCTGG
GCTAAACAGATCGACAGGCAGCTGAGGGCCTACATGAAGCGGGTGGAAGA
TGTCCTTGGCAAGGGCTGGGAGAATCACGTGGAGGGGCAGAAGCTGAAGC
AGGATGGAGACAGCTTCCGCATGAAGCTCAACACGCAGGAGATCTTTGAT
GACTGGGCAAGGAAGGTGGAGCAGCGCAACCTCGGTGTCTCGGGGCGCAT
TTTCACCATCGAAAGTACTCGGGTTCGGGGCCGAACTGGAAATGTGCTTA
AGCTGAAAGTTAACTTTCTTCCTGAGATTATCACACTATCCAAAGAAGTC
CGGAACCTCAAATGGCTTGGTTTCCGCGTCCCACTGGCGATTGTGAACAA
AGCCCATCAAGCAAACCAGCTTTACCCGTTTGCCATCTCACTGATCGAGA
GCGTTCGTACCTATGAACGGACCTGCGAGAAGGTGGAGGAGCGGAACACC
ATTTCCCTTTTGGTGGCTGGCTTGAAAAAGGAAGTGCAGGCCCTGATCGC
AGAAGGCATTGCGTTGGTGTGGGAGTCCTACAAACTTGACCCATATGTAC
AGCGCTTAGCAGAGACTGTCTTCAACTTCCAAGAAAAGGTGGATGATCTG
CTGATCATTGAAGAAAAAATAGACCTAGAAGTCCGTTCCTTGGAAACTTG
TATGTATGACCATAAGACATTCTCGGAAATCTTGAACAGAGTCCAGAAAG
CAGTGGATGACTTAAATCTGCACTCCTATTCCAATTTGCCCATCTGGGTC
AACAAGCTTGACATGGAGATTGAAAGAATATTGGGCGTCCGTCTGCAAGC
TGGCCTGAGAGCTTGGAGGCAGGTTCTTCTTGGACAAGCTGAAGATAAAG
CAGAAGTTGACATGGACACAGATGCTCCACAAGTTAGTCACAAGCCTGGT
GGAGAGCCAAAGATCAAAAATGTCGTTCATGAGCTAAGAATAACCAATCA
GGTAATCTACTTGAATCCACCAATTGAAGAGTGCAGATACAAGCTGTATC
AGGAAATGTTTGCCTGGAAGATGGTTGTAGTGTCTCTCCCCAGGATCCAG
AGCAGAGGTAGCAGGTGGGTGTACATTACGAATTGACTGAGGAAGAGAA
ATTCTATCGGAATGCTTTAACACGGATGCGTGATGGCCCTGTTGCGCTGG
AAGAGTCGTATTCTGCTGTCATGGGCATTGTATCTGAAGTTGAACAGTAT
GTCAAGGTTTGGCTTCAGTGTCAGTGTTTATGGGATATGCAAGCTGAAAA
CATCTATAACAGACTTGGAGAAGATCTGAACAAATGGCAGGCTCTCCTGG
TCCAAATAAGGAAGGCCAGAGGAAGCTTTGACAATGCAGAAACCAAGAAA
GAGTTTGGACCAGTAGTTATAGATTATGGCAAGGTACAATCTAAGGTGAA
CTTGAAATATGACTGTTGGCATAAGGAGGTTCTTAGCAAATTTGGGCAGA
TGGTAGGATCAAAGATGACGGAATTCCATTCCCAGATCTCAAAGTCCCGC
CAAGAGTTGGAGGAGCACTCAGTAGACACGGCCAGCACCTCCGATGCAGT
GACCTTGATGACCTATGTGCAGTCTTTGAAACGGAAGATCAAGCAGTTTG
AGAAGCAAGTTGAGCTCTACCGCAATGGCCAGCGCTTACTGGAAAAGCAA
AGGTTCCAGTTCCGAGCTTCCTGGCTTTATATTGACAAGATCGAGGGAGA
GTGGGGAGGGTTGAATGAGATCATGCGGCGAAAGGACTCTGCCATTCAGG
AGGAGGTGGCAAACGTGCAAATGAAGATTGTCCAGGAGGATCGGGCCGTG
GAAAGGCGGAGCACCGAGCTGCTGACTGAGTGGGAGAAGACCAAGCCTGT
GAGGGGCAACCTTCGCCCAGAAGAGGGAGTTCAGGCTCTGAGGATATATG
AGGGGAAGTTTGGTAGGCTGAAGGACGACAGAGAGAAGTGTGCAAAGGCC
AAGGAGGCGCTGGAATTGACAGATACTGGGCTTCTCAGTGGCAGTGAAGA
GCGCGTGCAGGTGGCCTTAGAAGAATTACAGGACCTCAAAGGCGTTTGGT
CAGAACTTTCTAAGGTTTGGGAGCAAATCGATGAGATGAAGGAGCAACCC
TGGGTTTCAGTACAGCCTCGAAAGCTTCGACAAAATTTGGATGCCCTCCT
GAACCAGCTGAAAAGCTTCCCTGCCCGGTTGCGACAGTATGCGTCCTATG
AGTTTGTTCAGAGGCTTCTGAAAGGTTAGATGAAGATAAATATGCTGGTG
ATTGAACTGAAATCCGAAGCACTTAAAGACCGCCATTGGAAACAGCTCAT
GAAAAGGCTTGAGGTTAATTGGGTTGTTTCTGAGCTAACCCTTGGCCAAA
TCTGGGATGTTGACTTGGAGAAAAATGAAGCGATTGTCAAGGATGTACTG
CTTGTGGCACAAGGGGAGATGGCTTTGGAAGAATTTTTGAAGCAGATAAG
AGAAGTGTGGAATACTTATGAACTAGACTTGGTTAATTATCAGAACAAGT
GCCGGTTGATCCGTGGGTGGGATGACCTCTTCAACAAGGTCAAAGAACAC
ATCAACAGCGTCTCGGCCATGAAGGTCTCTCCGTATTACAAGGTTTTTGA
AGAGGATGGTCTCAGCTGGGAAGATAAGCTGAAGAGGATCATGGCTCTCT
TTGATGTGTGGATTGATGTGCAGAGGCGGTGGGTCTACCTGGAAGGTATC
TTCACAGGCAGTGCAGATATCAAGCACCTGCTGGCAGTGGAAAGCCAGCG
GTTTCAGAGGATGAGCACTGAGTTTTTGGCTCTAATGAAAAAAGTGTCCA
AGTCTCCCCTTGTTATGGATGTTCTGAACATCCAGGGAGTAGAGAGGTCT
CTGGAAAGATTGGCAGACCTGCTAGGAAAGATCCAGAAAGCATTGGGAGA
ATATCTGGAAAGAGAGGGGTCATCTTTCCCGAGGTTCTATTTTGTGGGTG
ATGAAGATTTGGTTGAAATCATTGGAAACAGCAAGAATGTGGCTAAATTA
CAGAAACACTTCAAGAAGATGTTTGCTGGAGTTTCGAGCATCATCCTGAA
CGAGGATAACTCTGTTGTTTTGGGTATTTCATCTCGGGAAGGAGAGGAGG
TTATGTTTAAAACTCCTGTGTCAATTACTGAAGATCCCAAAATCAATGAG
TGGCTCACATTGGTAGAAAAGGAGATGAGAGTCACCCTGGCCAAACTGCT
TGCTGAGTCTGTTACGGAAGTTGAGATTTTTGGTAAAGCAACTTCAATTG
ACCCAAATACCTACATCACTTGGATTGATAAATACGAGGCCCAGCTTGTG
GTTTTGTCAGCCCAGATAGCCTGGTCTGAGAACGTGGAGACCGGAGTGAG
GAGGATGGGCGGAGGTGGAGATGGCGCGCCCTTGCACTCTGTGCTGAGCA
ATGTGGAGGTGACCCTCAATGTGTTAGCAGACTCTGTCCTCATGGAGGAG
CCCCCACTCCGAAGGCGGAAGCTAGAACACTTGATTACAGAGTTGGTTCA
CCAGAGAGATGTTACAAGGTCCTTGATCAAAAGCAAGATTGACAACGCCA
AATCTTTTGAATGGCTCAGCCAGATGCGATTTTACTTTGACCCTAAGCAA
ACTGATGTGTTACAGCAGTTGTCAATTCAAATGGCAAATGCCAAATTTAA
CTATGGCTTTGAGTACCTGGGTGTTCAGGACAAACTGGTCCAGACCCCCC
TCACTGACCGCTGCTATTTGACAATGACACAAGCCTTGGAGGCCAGGCTG
GGGGGTTCCCCATTTGGACCTGCTGGAACTGGGAAAACAGAGTCTGTCAA
AGCTCTTGGCCATCAGCTTGGACGGTTTGTTTTAGTTTTCAACTGTGATG
AAACCTTTGATTTCCAGGCAATGGGCCGGATCTTTGTGGGCCTTTGCCAG
GTGGGTGCCTGGGGCTGCTTTGACGAGTTCAACCGCCTGGAGGAGCGGAT
GCTCTCGGCTGTGTCCCAGCAGGTGCAGTGCATACAGGAAGCACTGCGTG
AACATTCCAACCCCAACTACGACAAGACCTCTGCCCCCATTACTTGTGAG
CTGCTGAACAAACAAGTCAAGGTGAGCCCGGACATGGCCATCTTCATCAC
CATGAACCCTGGCTACGCGGGCCGGTCTAACCTTCCTGACAACTTGAAGA
AGCTGTTCCGGAGCTTGGCCATGACCAAGCCCGACCGGCAGTTAATCGCC
CAGGTCATGCTGTACTCACAGGGTTTCCGCACTGCTGAAGTGCTTGCCAA
CAAAATCGTCCCGTTTTTTAAACTATGCGATGAGCAGCTCTCTTCCCAAA
GCCATTATGACTTCGGTCTTCGGGCTTTGAAGAGTGTGCTGGTGAGTGCA
GGCAATGTGAAGAGAGAGAGAATCCAGAAGATAAAGAGGGAGAAAGAGGA
ACGAGGGGAAGCAGTTGATGAAGGAGAAATTGCTGAAAATCTCCCTGAAC
AAGAGATTCTGATACAGAGCGTCTGTGAGACGATGGTGCCAAAGCTGGTG
GCAGAGGACATCCCGCTGCTCTTCAGCCTCCTGTCGGACGTGTTCCCTGG
AGTCCAGTATCACAGGGGTGAGATGACTGCCCTTCGAGAGGAGCTGAAGA
AAGTGTGTCAGGAGATGTATTTGACATATGGAGATGGAGAAGAAGTTGGT
GGAATGTGGGTTGAAAAGGTTCTCCAGCTCTATCAGATCACCCAGATCAA
TCATGGCCTGATGATGGTGGGGCCCTCGGGAAGTGGGAAGAGCATGGCCT
GGCGTGTCCTGCTGAAGGCATTGGAGAGACTCGAGGGTGTGGAAGGTGTG
GCCCATATCATCGACCCCAAGGCCATCAGCAAAGACCACCTCTACGGAAC
CCTGGACCCCAACACCAGGGAATGGACAGATGGGCTCTTCACACACGTGC
TGAGAAAGATCATCGACAGCGTGAGAGGCGAGCTGCAGAAGCGCCAGTGG
ATCGTCTTCGATGGCGATGTGGATCCAGAGTGGGTTGAGAACTTGAACTC
AGTGCTGGATGACAATAAGCTCCTAACTTTGCCCAATGGAGAGCGCCTCA
GTCTTCCACCCAATGTGAGAATAATGTTTGAGGTACAGGACTTGAAATAC
GCGACCTTGGCCACAGTGTCGCGCTGCGGCATGGTCTGGTTCAGTGAGGA
TGTGCTGAGCACCGACATCATCTTCAACAACTTCCTGGCCAGGCTGCGCA
GCATCCCGCTGCATGAAGGGGAGCATGAGCCACAGCGGCGGCGTAACGCC
AAAGAGGATGAGGGCGAGGAGGCCGCTTCCCCCATGCTGCAGATCCAAAG
AGATGCAGCTACGATCATGCAACCGTACTTCACGTCCAACGGCCTGGTCA
CCAAGGCGCTAGAGCACGCCTTCCAGCTGGAGCACATCATGGACCTAACA
CGCCTGCGCTGCCTGCGCTCGCTCTTCTCCATGCTGCACCAGGCCTGCCG
CAACCTCGCGCAGTATAACGCCAACCATCCCGACTTCCCCATGCACATCG
AGCAGCTCGAGCGCTACATTCAGCGATATCTGGTTTATGCCATACTCTCG
TCCCTGTCTGGAGACACCCGGCTAAAAATGAGACCAGAGCTGCGTCAATA
CATCAGAAGAATCACGACCGTGCCTCTCCCCACTGCGCCCAACATACCCA
TTATCCATTATGAGGTCTCCATCAGCGGAGAATGGTCTCCGTGCCAGCCC
AAGGTCCCTCACATTCAAGTCCAGACGCACAAGGTGGCAGCCCCTCATGT
CGTCCTCCCAACGCTCGACACAGTCCCCCACGAACCCCTCTTCTACACTT
CGCTGGCCCAACACAAGCCCCTGGTCTTGTGTGGCCCTCCTGGGTCTCGC
AAGACCATGACACTCTTCAGCGCCCTCCGGCCCTTGCCTGACATCGAGGT
GCTCGCTCTCAACTTCTCCAGTGCTACTACTCCAGAGCTCCTTCTCAAGA
CTTTTGATCACTACTGCGACTACAGGCGCACACCTAATGGGGTGGTTTTG
GCTCCTGTTCAACTTGGAAAGTGGCTGGTGTTGTTCTGTGATGAAATCAA
CTTGCCAGATATGGATAAATATGGGACCCAGAGGGTCATATCCTTCATCA
GACAGATGGTGGAGCACGGAGGCTTTTACCGTACCTCAGATCAAACATGG
GTGAAGCTGGAGAGAATCCAGTTTGTTGGGGCTTGTAATCCCCCCACAGA
CCCTGGAAGAAAGCCCCTCTCACACAGGTTCCTGCGCCACGTGCCTGTCG
TGTATGTGGATTACCCGGGCCCCGCCTCCCTCACACAGATCTACGGCACC
TTCAACCGCGCCATGCTGAGGCTCATTCCATCCCTGCGGACGTATGCAGA
GCCGCTCACTGCTGCCATGGTGGAGTTCTACACCATGTCTCAGGAGAGAT
TCACCCAGGATACACAACCTCACTATATCTATTCACCCCGTGAAATGACT
AGGTGGGTGAGAGGCATCTTTGAAGCGCTGAGACCTCTGGAGACCCTGCC
TGTTGAAGGCCTCATTCGGATTTGGGCACATGAAGCTCTGCGTCTCTTCC
AAGATAGACTCGTAGAGGATGAGGAGAGGCGTTGGACTGATGAGAACATC
GACACGGTTGCTCTGAAGCACTTCCCTAACATCGACAGAGAGAAGGCAAT
GAGCCGACCCATCTTGTACAGCAACTGGCTGTCAAAGGATTACATCCCAG
TAGACCAAGAAGAGTTAAGAGATTATGTCAAAGCTAGGCTGAAGGTCTTT
TATGAAGAAGAACTTGATGTTCCGCTGGTGCTGTTTAATGAAGTCCTAGA
CCACGTGCTGAGGATTGACAGAATATTCCGTCAACCTCAAGGCCACTTGC
TTCTGATTGGTGTTAGTGGAGCAGGAAAAACTACCCTGTCTCGTTTCGTC
GCCTGGATGAACGGTTTGAGTGTGTACCAGATTAAGGTCCATAGGAAGTA
CACAGGGGAAGACTTTGATGAAGATCTACGGACAGTGTTGAGACGTTCTG
GCTGTAAAAATGAAAAGATAGCATTTATAATGGATGAATCTAATGTGTTA
GATTCTGGATTCCTGGAGCGAATGAATACCCTTCTGGCCAATGGAGAGGT
GCCTGGTCTCTTTGAAGGAGACGAGTATGCCACCTTGATGACGCAGTGCA
AAGAGGGGGCACAGAAGGAAGGCCTGATGCTGGACTCGCACGAGGAGCTC
TACAAGTGGTTCACTAGCCAGGTTATCCGCAACCTCCACGTCGTGTTCAC
CATGAACCCGTCCTCGGAGGGACTCAAGGACCGGGCAGCTACATCACCAG
CACTTTTCAACAGGTGTGTGTTGAATTGGTTTGGAGACTGGTCCACCGAA
GCACTGTATCAGGTTGGCAAAGAATTCACAAGTAAGATGGATCTGGAGAA
GCCAAATTACATCGTGCCTGATTACATGCCAGTTGTGTATGATAAGCTGC
CGCAGCCACCATCCCATCGGGAAGCCATTGTGAACAGCTGTGTGTTTGTT
CATCAGACTCTTCACCAGGCGAATGCTCGGCTAGCAAAGCGAGGCGGCAG
AACGATGGCCATCACCCCTCGCCACTACCTGGACTTCATCAATCACTATG
CCAACCTGTTCCACGAGAAGCGGAGCGAGCTGGAGGAGCAGCAGATGCAC
TTGAACGTGGGGCTCAGGAAGATCAAAGAGACAGTCGACCAGGTAGAAGA
ACTGCGTCGTGACTTGAGGATAAAGAGCCAAGAGCTGGAGGTGAAGAATG
CAGCAGCCAATGACAAGCTGAAAAAGATGGTGAAAGACCAGCAGGAGGCT
GAAAAGAAGAAGGTTATGAGCCAAGAAATCCAGGAACAGCTGCATAAGCA
GCAGGAGGTAATTGCAGACAAACAGATGAGTGTCAAAGAAGATCTTGATA
AGGTGGAACCTGCCGTCATTGAGGCoCAGAATGCTGTGAAGTCGATCAAG
AAGCAGCACCTGGTGGAGGTGAGGTCCATGGCCAACCCTCCTGCTGCTGT
GAAGCTGGCGCTGGAGTCCATCTGCCTGCTGCTGGGGGAAAGCACCACAG
ACTGGAAGCAGATCCGCTCCATCATCATGCGGGAGAACTTCATCCCCACC
ATCGTCAACTTCTCTGCAGAGGAGATCAGTGACGCCATAAGGGAGAAGAT
GAAGAAAAATTACATGTCCAATCCAAGTTACAATTATGAAATTGTGAATC
GGGCTTCCCTGGCTTGCGGCCCTATGGTGAAATGGGCAATTGCACAGCTT
AACTATGCAGACATGTTAAAGAGAGTGGAGCCCCTACGCAATGAGCTGCA
GAAGCTGGAAGATGACGCCAAGGACAACCAGCAGAAGGCCAACGAGGTGG
AGCAGATGATCCGAGACCTGGAAGCCAGCATCGCCCGCTACAAGGAGGAA
TACGCCGTCCTGATCTCAGAGGCCCAGGCCATCAAGGCAGACCTGGCAGC
TGTCGAGGCAAAAGTAAACCGGAGCACTGCTCTTCTGAAGAGCTTGTCTG
CTGAACGTGAACGATGGGAAAAAACAAGTGAAACTTTCAAAAACCAGATG
TCCACCATTGCTGGGGACTGTCTCTTGTCAGCTGCGTTCATTGCCTACGC
GGCTTACTTTGACCAGCAGATGCGTCAGAACTTCTTCACTACCTGGTCCC
ATCACCTACAGCAACCCAACATCCAGTTCCCTACAGATATTGCCAGGACG
GAATACCTTTCCAATGCTGATGAGCCTCTTCGCTGGCAGGCCAGCTCCTT
GCCTGCTGATGACCTTTGCACAGAAAATCCCATCATGCTCAAACGATTCA
ATACGTATCCCCTCATCATTGACCCCTCTGGACAGCCCACAGAATTCATT
ATGAATGAATATAAGGATCCTAACATCACACCCACCACCTTCCTCGATGA
CGCCTTCAGAAAGAACTTAGAGAGTGCACTGAGATTCGCTAACCCCCTTC
TCGTCCAGGATGTGGAAAGCTACGATCCAGTTTTGAACCCGCTCCTGAAC
CCTGAACTCCCCCCAACACGCCCCACAGTCCTGATCACTCTCGGGGACCA
GGACATAGACCTGTCGCCATCGTTTCTCATCTTCCTGTCCACCCGGGATC
CAACTGTCGAGTTCCCACCAGATCTCTCTTCCCCGCTTACTTTTGTAAAC
TTCACACTTACCCGTAGCACTTTACAAAGCCAGTGTCTAAATGAAGTACT
TAAAGCACAAAGACCTCATCTGGACGAGAAACGATCTCATCTTCTTAAAC
TTCAAGGGCAATTTCACCTCCGTTTGCGTCAGCTGGAAAAATCTCTACTA
CAAGCTCTGAACCACCTCAAAGGGCGCATTTTGGATGACCACACGATCAT
AACCACTCTGGACAACCTGAACACACAGGCTCCAGACCTCACCACCAAAC
TTCAGCAGACGGACATTCTCATCCACCAGGTGGAGACCGTGTCCCAGCAC
TACCTCCCGCTCTCCACCGCCTCCACCAGCATCTACTTCACCATCGAGTC
CCTCAACCAGATACACTTCTTCTACCAGTACTCCCTCCAGTTTTTCCTGC
ACATTTATCACAACCTCCTATACCACAACCCCAACCTCAAGCCTCTCACC
GACCACACACACCCCCTCTCCATTATAACAAACCACCTCTTCCACCTGGC
CTTTAACCGAGTCCCTCCACCCATCCTCCATCACCACCACATTACCTTTC
CCATCCTCCTCGCAACAATCAAACTCAACGGCACCCTCCCGGAGCCCACC
TACCATCCACAATTCCAGCACTTCTTGAGACCAAATCAGATTCTCCTCAG
TCCTCCCTCCACCCCCACCATCCACCCCCTGACTCTCCACCAGCCCGAGC
CCCTCCTGAGCCTGAGCTCCCTTCCCCCCTTTAACGACTTCATTCCAAAG
CTTGAGCCAGACGAGCAATTTCCCATCTCCCTCCACACCACCTCCCCGGA
GCAGACTCTCCCCTACCTCTCCACTGAACAAACACCTCCAACACCCATTC
CCCACGCCATCCACCCCCTCCTCCTCATCCACCCTTTCCCCCCCCATCCC
CTCTTCCCCATCCCCCACATCTTTCTTTCAACAAACCTTCCCCACTCTTT
CATCTCCATCATCGAGCACCCCCTCGACCTCACCCACATTCTCCCCACAC
AGCTGAACCCCAACACTCCTCTCTTAATCTCCTCTCTGCCTCCTTATCAT
CCCACTCCACATCTCGACGACCTTCCAGCCCAGCACAACACCCACATCAC
TTCAATTCCAATCCCCTCTCCACAACGCTTTAACCAACCACATAACCCAA
TAAACACCCCTCTAAACTCCCCCAGCTCCCTCATCCTCAACAATCTCCAT
CTCGCCCCACCCTCCCTCATCCACCTCGAGAAGAACTTCCATTCCCTCCA
CCCCCATCCCTCCTTCCCACTCTTCCTCACCATCCACATCAACCCCAAGC
TCCCTCTCAATCTCCTCCCTCCCCCCCCCATCTTTCTCTTCCACCCACCC
CCACCCCTCAACCCCAACATGCTGAGCACCTTCACCACCATTCCCCTCTC
ACCCATATCCAAGTCTCCCAACCACCGTCCCCCCTTCTACTTCCTGCTCC
CCTCCTTTCATCCCATCATCCAACAACCCTTACCATACCCACCACTCCCC
TCCTCAAACAACTATGAATTTCCAGACTCTCACCTCCGCTCACCTTCCCA
TACGCTCCACACCTCCCTCCATCACACCCCCAACCCCACCCACAACATCT
CACCCCATAACATCCCCTCCTCTCCACTAAACACCTTAATCCCCCACTCC
ATTTATCCCGCCCCCCTCCACAACCAGTTTGACCACCCTCTCCTCAACAC
CTTCCTGCACCCCCTCTTCACAACCACCACTTTCCACACTCAGTTTAAGC
TCGCATGCAACCTCCACCGACATAAACACATTCAAATCCCACATCCCATC
ACCCCACACCACTTTCTCCACTCCCTCCACTTCCTCCCCCACACCCACAC
CCCCTCCTCCCTGGGCCTGCCCAACAACGCCGAGAGAGTCCTCCTTACCA
CACAGGGTGTGGACATGATCAGTAAAATGCTGAAGATGCAGATGTTGGAG
GATGAGGACGACCTGGCCTACGCAGAGACTGAGAAGAAGACGAGGACAGA
CTCCACGTCCGACGGGCGCCCTGCCTGGATGCGGACACTGCACACCACCG
CGTCCAACTGGCTGCACCTCATCCCCCAGACGCTGAGCCACCTCAAGCGC
ACCGTGGAGAATATCAAGGATCCTTTGTTCAGGTTCTTTGAGAGAGAAGT
GAAGATGGGCGCAAAGCTGCTTCAGGACGTTCGCCAGGACCTTGCAGATG
TCGTCCAGGTGTGCGAAGGAAAGAAGAAGCAGACCAACTACTTGCGCACG
CTGATCAACGAGCTAGTGAAAGGGATCTTGCCTCGGAGCTGGTCCCACTA
CACGGTGCCTGCCGGCATGACCGTCATCCAGTGGGTGTCCGACTTCAGCG
AGAGGATCAAACAGCTGCAGAACATCTCACTGGCAGCTGCATCTGGTGGC
GCCAAGGAGCTAAAGAACATCCACGTGTGCCTGGGTGGCCTGTTCGTGCC
TGAGGCGTACATCACTGCCACCAGGCAGTATGTGGCCCAGGCCAACAGCT
GGTCCCTGGAGGAGCTCTGCCTGGAAGTCAACGTCACCACCTCACAGGGC
GCCACCCTTGACGCTTGCAGCTTCGGAGTCACGGGTTTGAAACTTCAAGG
GGCCACGTGCAACAACAACAAGCTGTCACTGTCCAATGCCATCTCAACCG
CCCTTCCCCTGACGCAGCTGCGCTGGGTCAAGCAGACAAACACCGAGAAG
AAGGCCAGTGTGGTAACCTTACCTGTCTACCTGAACTTCACCCGTGCAGA
CCTCATCTTCACCGTGGACTTCGAAATTGCTACAAAGGAGGATCCTCGCA
GCTTCTACGAGCGGGGTGTCGCAGTCTTGTGCACAGAGTAA

TABLE 4

Wild type human cytoplasmic dynein heavy chain 1,
cDNA (SEQ ID NO:17)

ATGTCGGAGCCCGGGGGCGGCGGCGGCGAGGACGGCTCGGCCGGATTGGA
AGTGTCGGCCGTGCAGAATGTGGCGGACGTGTCGGTGCTGCAGAAGCACC
TGCGCAAGCTGGTGCCGCTGCTGCTGGAGGACGGCGGCGAGGCGCCGGCC
GCGCTGGAGGCGGCGCTGGAGGAGAAGAGCGCCCTGGAGCAGATGCGCAA
GTTCCTTTCGGACCCGCAGGTCCACACGGTGCTGGTGGAGCGCTCCACGC
TCAAAGAGGACGTCGGTGATGAAGGAGAAGAAGAAAAAGAATTCATTTCC
TATAACATCAACATAGACATTCATTATGGGGTTAAATCCAATAGCTTGGC
ATTCATTAAACGTACTCCCGTGATTGATGCAGATAAACCCGTGTCTTCTC
AGCTCCGGGTCCTTACACTCAGTGAAGACTCGCCCTACGAAACTTTGCAT
TCTTTCATTAGCAATGCAGTGGCTCCTTTTTTTAAGTCCTACATTAGAGA
GTCTGGCAAGGCAGACAGGGATGGTGATAAAATGGCTCCTTCAGTTGAAA
AGAAGATTGCAGAACTCGAAATGGGACTCCTTCACTTGCAGCAAAATATT
GAAATTCCGGAGATCAGCCTGCCGATTCATCCAATGATCACAAATGTTGC
AAAACAGTGTTATGAGCGTGGAGAAAAGCCAAAAGTTACAGACTTTGGTG
ATAAGGTTGAAGACCCAACATTTCTTAATCAGTTACAATCTGGAGTTAAC
CGCTGGATCCGAGAAATTCAAAAAGTGACCAAACTGGATCGAGATCCTGC
ATCAGGAACTGCCTTACAGGAAATTAGTTTTTGGCTAAACTTGGAACGTG
CGTTATACCGCATCCAGGAGAAACGGGAGAGCCCGGAAGTTCTCCTGACT
CTGGATATCTTGAAACATGGCAAGCGCTTCCATGCCACCGTCAGTTTTGA
CACTGACACAGGTCTAAAACAGGCTTTGGAAACTGTGAATGACTACAATC
CTCTGATGAAAGATTTCCCTCTGAATGATTTGCTGTCTGCCACGGAGCTG
GACAAAATAAGACAGGCGCTTGTTGCCATTTTCACACATTTGAGAAAGAT
CCGAAACACAAAATATCCTATTCAGAGGGCACTGCGTTTGGTGGAGGCAA
TTTCAAGAGACTTGAGTTCTCAATTACTCAAAGTATTGGGCACTAGGAAA
TTGATGCATGTTGCTTATGAAGAATTTGAAAAAGTTATGGTAGCATGCTT
TGAAGTTTTTCAGACTTGGGATGATGAGTATGAGAAACTTCAGGTATTGT
TGAGAGACATCGTCAAAAGAAAAAGGGAAGAAAATCTGAAGATGGTGTGG
CGTATCAACCCTGCCCACAGGAAGCTGCAGGCCCGCCTTGAGCAGATGAG
AAAATTTAGACGCCAGGATGAACAGCTAAGAGGTGTTATCGTCAGGGTCC
TGAGGCCACAGGTCACGGCAGTTGCACAACAGAATGAAGGAGAGGTCCCT
GAACCCCAAGATATGAAAGTGGCTGAGGTTCTCTTTGATGCTGCAGATGC
AAATGCCATTGAGGAAGTAAACCTTGCTTATGAGAACGTCAAGGAAGTGG
ATGGACTGGATGTTTCCAAAGAGGGCACGGAAGCCTGGGAGGCTGCTATG
AAGAGGTACGATGAGAGGATCGACAGAGTGGAGACCGGGATCACCGCTCG
CGTTCGGGATCAGCTTGGCACAGCCAAGAATGCGAACGAGATGTTTAGGA
TTTTCTCCAGGTTTAATGCACTGTTTGTCAGGCCTCACATCCGTGGGGCC
ATTCGCGAATACGAGACCCAGCTGATCCAGCGCGTGAAAGATGAGATTGA
GTCTCTTGAGGACAAGTTCAAGGTCCAGTAGCGACAGAGTCAGGCTTGTA
AGATGAGTCACGTTCGTGACTTGCCGCCTGTGTCAGGGTCTATCATCTGG
GCTAAACAGATCGACAGGCAGCTGAGGGCCTACATGAAGCGGGTGGAAGA
TGTCCTTGGCAAGGGCTGGGAGAATCAGGTGGAGGGGGAGAAGCTGAAGC
AGGATGGAGACAGCTTGGGGATGAAGCTCAACACGCAGGAGATCTTTGAT
GACTGGGCAAGGAAGGTGCAGCAGCGCAACCTCGGTGTCTGGGGGGGGAT
TTTCACCATCGAAAGTAGTCGGGTTCGGGGCCGAACTGGAAATGTGCTTA
AGCTGAAAGTTAACTTTCTTCGTGAGATTATCAGACTATCCAAAGAAGTC
CGGAACCTCAAATGGCTTGGTTTCCGCGTCCCACTGGCGATTGTGAACAA
AGCCCATCAAGCAAACGAGGTTTACCCGTTTGCGATCTCACTGATCGAGA
GCGTTCGTACCTATGAAGGGAGGTGCGAGAAGGTGGAGGAGCGGAAGACC
ATTTCCGTTTTGGTGGCTGGCTTGAAAAAGGAAGTGGAGGCCCTGATCGC
AGAAGGCATTGCGTTGGTGTGGGAGTCCTACAAACTTGACCCATATGTAC
AGCGGTTAGCAGAGAGTGTCTTCAACTTCCAAGAAAAGGTGGATGATCTG
CTGATCATTGAAGAAAAAATAGACCTAGAAGTCCGTTCCTTGGAAACTTG
TATGTATGAGCATAAGACATTCTCGGAAATCTTGAACAGAGTCCAGAAAG
CAGTGGATGAGTTAAATCTGGACTCCTATTCGAATTTGCCCATCTGGGTC
AACAAGCTTGACATGGAGATTGAAAGAATATTGGGCGTCCGTCTGCAAGC
TGGCGTGAGAGCTTGGACGCAGGTTGTTCTTGGACAAGCTGAAGATAAAG
CAGAAGTTGACATGGACACAGATGCTCCACAAGTTAGTGACAAGCCTGGT
GGAGAGCCAAAGATCAAAAATGTCGTTCATGAGCTAAGAATAACCAATCA
GGTAATCTACTTGAATCCACCAATTGAAGAGTGCAGATACAAGCTGTATC
AGGAAATGTTTGCCTGGAAGATGGTTGTACTGTCTGTGCCCAGGATGCAG
AGTCAGAGGTACCAGGTGGGTGTACATTACGAATTGAGTGAGGAAGAGAA
ATTCTATCGGAATGCTTTAACAGGGATGCCTGATGGCCCTGTTGCCCTGG
AAGAGTCGTATTCTGGTGTCATGGGCATTGTATGTGAAGTTGAACAGTAT
GTCAAGGTTTGGCTTCAGTATCAGTGTTTATGGGATATGCAAGCTGAAAA
CATCTATAACAGACTTGGAGAAGATCTCAACAAATGGCAGGCTCTCCTGG
TCGAAATAAGGAAGGCCAGAGGAACCTTTGACAATGCAGAAACCAAGAAA
GAGTTTGGACCAGTAGTTATAGATTATGGCAAGGTACAATGTAAGGTGAA
CTTGAAATATGACTCTTGGCATAAGGAGGTTCTTAGCAAATTTGGGGAGA
TGGTAGGATCAAAGATGACGGAATTCCATTCCCAGATCTCAAAGTCCCGC
CAAGAGTTGGAGGAGGAGTGAGTAGAGACGGCGAGGAGCTCCGATGCAGT
GAGCTTCATCACCTATGTGGAGTCTTTGAAACGGAAGATCAAGCAGTTTG
AGAAGGAAGTTGAGCTCTAGCGGAATGGCCAGCGCTTACTGGAAAAGGAA
AGGTTGCAGTTCCCAGCTTCCTGGCTTTATATTGACAACATCGAGGGAGA
GTGGGGAGCCTTCAATGACATGATGCGGCGAAAGGACTGTGCCATTGAGC
AGCAGGTGGCAAACGTGCAAATGAAGATTGTCGAGGACGATCCGCCCGTG
GAAACCCGCACCACCCACCTCCTGACTGACTGGCAGAAGACCAAGCCTGT
CACGGGCAACCTTCGCCCAGAACAGGCACTTCAGGCTCTCACCATATATG
AGGGGAACTTTGGTAGCCTCAAGGACGACAGAGAGAAGTGTGCAAAGGCC
AACGAGGCCCTGGAATTGACAGATACTCGGCTTCTCAGTCCCAGTGAAGA
GCCCGTGCAGCTCCCCTTACAACAATTACAGCACCTCAAAGGCGTTTGGT
CAGAACTTTCTAAGCTTTGGGAGCAAATCGATCAGATGAAGGAGCAACCC
TGGGTTTCAGTACAGCCTCGAAAGCTTCCACAAAATTTGGATCCCCTCCT
GAACCAGCTGAAAAGCTTCCCTCCCCGGTTGCGACAGTATGCGTCCTATG
AGTTTGTTCAGAGGCTTCTGAAAGGTTACATGAAGATAAATATGCTGGTG
ATTGAACTGAAATCCGAAGCACTTAAAGACCCCCATTGGAAACAGCTCAT
GAAAAGGCTTCACGTTAATTGGGTTGTTTCTGAGCTAACCCTTCGCCAAA
TCTGGGATGTTGACTTGCACAAAAATGAACCGATTGTCAAGGATGTACTG
CTTCTCGCACAAGGGCAGATGGCTTTGGAAGAATTTTTGAACCACATAAC
AGAAGTGTGCAATACTTATGAACTAGACTTGGTTAATTATCAGAACAAGT
CCCGCTTCATCCGTGGCTGGGATGACCTCTTCAACAACGTCAAAGAACAC
ATCAACAGCGTCTCGGCCATGAAGCTCTCTCCGTATTACAAGCTTTTTGA
AGAGGATGCTCTCACCTGCGAAGATAAGCTGAACAGCATCATGGCTCTCT
TTGATGTGTCCATTCATGTCCACACGCGGTCCGTCTACCTCCAACGTATC
TTCACAGGCAGTCCACATATCAAGCACCTGCTCCCACTGGAAACCCAGCC
GTTTCACAGCATCAGCACTGAGTTTTTCCCTCTAATGAAAAAACTGTCCA
AGTCTCCCCTTCTTATGGATGTTCTGAACATCCACCCAGTACAGACGTCT
CTGGAAAGATTGCCAGACCTCCTACCAAACATCCACAAACCATTCCCAGA
ATATCTCCAAACACACCCCTCATCTTTCCCCACCTTCTATTTTCTCCCTC
ATCAACATTTCCTTCAAATCATTCGAAACACCAACAATGTCCCTAAATTA
CACAAACACTTCAAGAACATCTTTCCTGGACTTTCGAGCATCATCCTGAA
CCACCATAACTCTCTTGTTTTCGGTATTTCATCTCGCGAACCACACCAGG
TTATCTTTAAAACTCCTCTCTCAATTACTCAACATCCCAAAATCAATCAG
TCCCTCACATTCCTAGAAAACCACATGAGACTCACCCTCCCCAAACTGCT
TGCTGAGTCTCTTACCCAACTTGAGATTTTTCGTAAACCAACTTCAATTC
ACCCAAATACCTACATCACTTCCATTCATAAATACCAGCCCCACCTTCTC
CTTTTCTCACCCCACATACCCTCCTCTGACAACCTCCACACCCCACTCAC
CACCATGGGCCGACCTCCACATCCCGCCCCCTTGCACTCTCTCCTGAGCA
ATGTCGACCTCACCCTCAATCTCTTAGCACACTCTCTCCTCATCGAGCAG
CCCCCACTCCGAACGCCCAAGCTACAACACTTCATTACACACTTCCTTCA
CCAGACACATCTTACAACGTCCTTCATCAAAACCAACATTCACAACCCCA
AATCTTTTCAATCCCTCAGCCACATCCCATTTTACTTTCACCCTAACCAA
ACTCATCTCTTACAGCAGTTCTCAATTCAAATCCCAAATCCCAAATTTAA
CTATCCCTTTCACTACCTCCCTCTTCACCACAAACTCCTCCAGACCCCCC
TCACTGACCCCTCCTATTTCACAATCACACAACCCTTCCACCCCACCCTC
CCCCGTTCCCCATTTCCACCTCCTCCAACTCCCAAAACACACTCTCTCAA
AGCTCTTCCCCATCACCTTCCACCGTTTGTTTTACTTTTCAACTCTCATC
AAACCTTTCATTTCCACCCAATCCGCCGCATCTTTCTGCCCCTTTCCCAG
CTCCCTCCCTCCCCCTCCTTTGACCACTTCAACCCCCTCGAGGAGCCCAT
GCTCTCCCCTCTCTCCCACCACCTCCACTCCATACACCAACCACTCCCTC
AACATTCCAACCCCAACTACCACAACACCTCTCCCCCCATTACTTCTGAC
CTCCTCAACAAACAACTCAACCTCACCCCCGACATCCCCATCTTCATCAC
CATCAACCCTCCCTACGCCCCCCCCTCTAACCTTCCTGACAACTTGAACA
ACCTCTTCCCGACCTTCGCCATCACCAACCCCCACCCGCACTTAATCCCC
CACCTCATCCTCTACTCACAGCCTTTCCCCACTCCTCAACTCCTTGCCAA
CAAAATCCTCCCCTTTTTTAAACTATCCCATGAGCAGCTCTCTTCCCAAA
CCCATTATGACTTCGGTCTTCGGGCTTTGAAGAGTGTGCTGGTGAGTGCA
GGCAATGTGAAGAGAGAGAGAATCCAGAAGATAAAGAGGGAGAAAGAGGA
ACGAGGGGAAGCAGTTGATGAAGGAGAAATTGCTGAAAATCTCCCTGAAC
AAGAGATTCTGATACAGAGCGTCTGTGAGACGATGGTGCCAAAGCTGGTG
GCAGAGGACATCCCGCTGCTCTTCAGCCTCCTGTCGGACGTGTTCCCTGG
AGTCCAGTATCACAGGGGTGAGATGACTGCCCTTCGAGAGGAGCTGAAGA
AAGTGTGTCAGGAGATGTATTTGACATATGGAGATGGAGAAGAAGTTGGT
GGAATGTGGGTTGAAAAGGTTCTCCAGCTCTATCAGATCACCCAGATCAA
TCATGGCCTGATGATGGTGGGGCCCTCGGGAAGTGGGAAGAGCATGGCCT
GGCGTGTCCTGCTGAAGGCATTGGAGAGACTCGAGGGTGTGGAAGGTGTG
GCCCATATCATCGACCCCAAGGCCATCAGCAAAGACCACCTCTACGGAAC
CCTGGACCCCAACACCAGGGAATGGACAGATGGGCTCTTCACACACGTGC
TGAGAAAGATCATCGACAGCGTGAGAGGCGAGCTGCAGAAGCGCCAGTGG
ATCGTCTTCGATGGCGATGTGGATCCAGAGTGGGTTGAGAACTTGAACTC
AGTGCTGGATGACAATAAGCTCCTAACTTTGCCCAATGGAGAGCGCCTCA
GTCTTCCACCCAATGTGAGAATAATGTTTGAGGTACAGGACTTGAAATAC
GCGACCTTGGCCACAGTGTCGCGCTGCGGCATGGTCTGGTTCAGTGAGGA
TGTGCTGAGCACCGACATGATCTTCAACAACTTCCTGGCCAGGCTGCGCA
GCATCCCGCTCGATGAAGGGGAGGATGAGGCACAGCGGCGGCGTAAGGGC
AAAGAGCATGAGGGGGAGGAGGCCGCTTCCCCCATGCTCCAGATCCAAAG
AGATGCAGCTACGATCATGCAACCGTACTTCACGTCCAACGGCCTGGTCA
CCAAGGCGCTAGAGCACGCCTTCCAGCTGGAGCACATCATGGACCTAACA
CGCCTGCGCTGCCTGGGCTCGCTCTTCTCCATGCTGCACCAGGCCTGCCG
CAACGTGGCGCAGTATAACGCCAACCATCCCGACTTCCCCATGCAGATCG
AGCAGCTGGAGCGCTACATTCAGCGATATCTGGTTTATGCCATACTCTGG
TCCCTGTCTGGAGACAGCCGGCTAAAAATGAGAGCAGAGCTGGGTCAATA
CATCAGAACAATCACGACCGTGCCTCTCCCCACTCCGCCCAACATACCCA
TTATCGATTATGAGGTGTCCATCAGCGGAGAATGCTCTCCGTGGCAGGCC
AAGGTGCCTCAGATTGAAGTGGAGACGCACAAGGTGGCAGCCCCTGATGT
CGTCGTGCCAACGCTGGACACAGTCCGCCACGAAGCCCTCTTGTACACTT
GGCTGGCCGAACACAAGCCCCTGGTCTTGTGTGGCCCTCCTGGGTCTGGC
AAGACCATGACACTCTTCAGCGCCCTCCGGGCCTTGCCTGACATGGAGGT
GGTCGGTCTCAACTTCTCCAGTGCTACTACTCCAGAGCTGCTTCTGAAGA
CTTTTGATCACTACTGCGAGTACAGGCGCACACCTAATGGGGTGGTTTTG
GCTCCTGTTCAACTTGGAAAGTGGCTGGTGTTGTTCTGTGATGAAATCAA
CTTGCCAGATATGGATAAATATGGCACCCAGAGGGTCATATCCTTCATCA
GACAGATGGTGGAGCACGGAGGCTTTTACCCTACCTCAGATCAAACATGG
GTGAAGCTGGAGAGAATCCAGTTTGTTCGGGCTTGTAATCCCCCCACAGA
CCCTGGAAGAAAGCCCCTCTCACACAGGTTCCTGCGCCACGTGCCTGTCG
TGTATCTGGATTACCCGGGCCCCGCCTCCCTCACACAGATCTACGGCACC
TTCAACCGCGCCATGCTGAGGCTCATTCCATCCCTGCGGACGTATGCAGA
GCCGCTCACTGCTGCCATGCTGGAGTTCTACACCATGTCTCAGGAGAGAT
TCACCCAGGATACACAACCTCACTATATCTATTCACCCCGTGAAATGACT
ACGTGCCTGAGAGGCATCTTTGAAGCGCTGAGACCTCTGGAGACCCTGCC
TGTTGAAGGCCTCATTCGGATTTCCCCACATGAAGCTCTGCGTCTCTTCC
AAGATAGACTCGTAGAGGATGACCAGAGCCGTTGGACTGATGAGAACATC
GACACGGTTGCTCTCAAGCACTTCCCTAACATCGACAGAGAGAAGCCAAT
GAGCCGACCCATCTTCTACAGCAACTGGCTGTCAAAGCATTACATCCCAG
TAGACCAAGAAGAGTTAACAGATTATGTCAAACCTAGGCTGAAGGTCTTT
TATGAAGAAGAACTTCATCTTCCGCTGGTCCTGTTTAATGAAGTCCTAGA
CCACGTGCTGAGGATTGACAGAATATTCCGTCAACCTCAAGGCCACTTGC
TTCTGATTGGTGTTAGTGGAGCAGGAAAAACTACCCTGTCTCGTTTCGTC
GCCTGGATGAACGGTTTGAGTGTGTACCAGATTAAGGTCCATAGGAAGTA
CACAGGGGAAGACTTTGATGAAGATCTACGGACAGTGTTGAGACGTTCTG
GCTGTAAAAATGAAAAGATAGCATTTATAATGGATGAATCTAATGTGTTA
GATTCTGGATTCCTGGAGCGAATGAATACCCTTCTGGCCAATGGAGAGGT
GCCTGGTCTCTTTGAAGGAGACGAGTATGCCACCTTGATGACGCAGTGCA
AAGAGGGGGCACAGAAGGAAGGCCTGATGCTGGACTCGCACGAGGAGCTC
TACAAGTGGTTCACTAGCCAGGTTATCCGCAACCTCCACGTCGTGTTCAC
CATGAACCCGTCCTCGGAGGGACTCAAGGACCGGGCAGCTACATCACCAG
CACTTTTCAACAGGTGTGTGTTGAATTGGTTTGGAGACTGGTCCACCGAA
GCACTGTATCAGGTTGGCAAAGAATTCACAAGTAAGATGGATCTGGAGAA
GCCAAATTACATCGTGCCTGATTACATGCCAGTTGTGTATGATAAGCTGC
CGCAGCCACCATCCCATCGGGAAGCCATTGTGAACAGCTGTGTGTTTGTT
CATCAGACTCTTCACCAGGCGAATGCTCGGCTAGCAAAGCGAGGCGGCAG
AACGATGGCCATCACCCCTCGCCACTACCTGGACTTCATCAATCACTATG
CCAACCTGTTCCACGAGAAGCGGAGCGAGCTGGAGGAGCAGCAGATGCAC
TTGAACGTGGGGCTCAGGAAGATCAAAGAGACAGTCGACCAGGTAGAAGA
ACTGCGTCGTGACTTGAGGATAAAGAGCCAAGAGCTGGAGGTGAAGAATG
CAGCAGCCAATGACAAGCTGAAAAAGATGGTGAAAGACCAGCAGGAGGCT
GAAAAGAAGAAGGTTATGAGCCAAGAAATCCAGGAACAGCTGCATAAGCA
GCAGGAGGTAATTGCAGACAAACAGATGAGTGTCAAAGAAGATCTTGATA
AGGTGGAACCTGCCGTCATTGAGGCCCAGAATGCTGTGAAGTCGATCAAG
AAGCAGCACCTGGTGGAGGTGAGGTCCATGGCCAACCCTCCTGCTGCTGT
GAAGCTGGCGCTGGAGTCCATCTGCCTGCTGCTGGGGGAAAGCACCACAG
ACTGGAAGCAGATCCGCTCCATCATCATGCGGGAGAACTTCATCCCCACC
ATCGTCAACTTCTCTGCAGAGGAGATCAGTGACGCCATAAGGGAGAAGAT
GAAGAAAAATTACATGTCCAATCCAAGTTACAATTATGAAATTGTGAATC
GGGCTTCCCTGGCTTGCGGCCCTATGGTGAAATGGGCAATTGCACAGCTT
AACTATGCAGACATGTTAAAGAGAGTGGAGCCCCTACGCAATGAGCTGCA
GAAGCTGGAAGATGACGCCAAGGACAACCAGCAGAAGGCCAACGAGGTGG
AGCAGATGATCCGAGACCTGGAAGCCAGCATCGCCCGCTACAAGGAGGAA
TACGCCGTCCTGATCTCAGAGGCCCAGGCCATCAAGGCAGACCTGGCAGC
TGTCGAGGCAAAAGTAAACCGGAGCACTGCTCTTCTGAAGAGCTTGTCTG
CTGAACGTGAACGATGGGAAAAAACAAGTGAAACTTTCAAAAACCAGATG
TCCACCATTGCTGGGGACTGTCTCTTGTCAGCTGCGTTCATTGCCTACGC
GGGTTACTTTGACCAGCAGATGCGTCAGAACTTGTTCACTACCTGGTCCC
ATCACCTACAGCAAGCCAACATCCAGTTCCGTACAGATATTGCCAGGACG
GAATACCTTTCCAATGCTGATGAGCGTCTTCGCTGGCAGGCCAGCTCCTT
GCCTGCTGATGACCTTTGCACAGAAAATGCCATCATGCTGAAACGATTCA
ATAGGTATCCGCTGATCATTGACCCCTCTGGACAGGCCACAGAATTCATT
ATGAATGAATATAAGGATCGTAAGATCACACGGACCAGCTTCCTGGATGA
CGCCTTCAGAAAGAACTTAGAGAGTGCACTGAGATTCGGTAACCCCCTTC
TGGTCCAGGATGTGGAAAGCTACGATCCAGTTTTGAACCCGGTGCTGAAC
CGTGAAGTGCGGCGAACAGGGGGGAGAGTGCTGATCACTCTCGGGGACCA
GGACATAGACCTGTCGCCATCGTTTGTCATCTTCCTGTCCACCCGGGATC
CAACTGTCGAGTTCCCACCAGATCTCTGTTCCCGGGTTACTTTTGTAAAC
TTCACAGTTACCCGTAGCAGTTTACAAAGCCAGTGTCTAAATGAAGTACT
TAAAGCAGAAAGACCTGATGTGGACGAGAAACGATCTGATCTTCTTAAAC
TTCAAGGGGAATTTCAGCTCCGTTTGCGTCAGCTGGAAAAATCTCTACTA
CAAGCTCTGAACGAGGTGAAAGGGCGCATTTTGGATGACGACACGATCAT
AACCACTCTGGAGAACCTGAAGAGAGAGGCTGCAGAGGTCACCAGGAAAG
TTGAGGAGACGGACATTGTCATGCAGGAGGTGGAGACCGTGTCCGAGCAG
TACCTCCCGCTCTCCACCGCCTGCAGCAGCATCTAGTTCACCATGGAGTC
CCTCAAGCAGATACACTTCTTGTACCAGTACTCCCTCCAGTTTTTCCTGG
ACATTTATCACAACGTCCTATACGAGAACCCGAACCTGAAGGGTGTCACC
GACCACACACAGCGCCTGTCCATTATAACAAAGGACCTCTTCCAGGTGGC
GTTTAACCGAGTGGCTCGAGGCATGCTGCATCAGGACCACATTACCTTTG
CCATGCTGCTGGCAAGAATCAAACTGAAGGGCACCGTGGGGGAGCCCACC
TAGGATGCAGAATTCCAGCACTTCTTGAGAGGAAATGAGATTGTCCTGAG
TGCTGGCTCGACCCCGAGGATCCAGGGGGTGACTGTGGAGCAGGCGGAGG
CGGTGGTGAGGCTGAGCTGCCTTCCCGCGTTTAAGGACTTGATTGCAAAG
GTTCAGGCAGACGAGCAATTTGGCATCTGGCTGGAGAGCAGCTCCCCGGA
GCAGACTGTGCCCTAGCTGTGGAGTGAAGAAACACCTGCAACAGCCATTG
GCCAGGCCATCGAGCGCCTGCTGGTGATGGAGGCTTTCCGGCCCGATGGC
GTGTTGGCCATGGCCGACATGTTTGTTTGAAGAAACGTTGGGGAGTGTTT
CATGTCCATCATGGAGCAGCCGCTCGACCTGAGCCACATTGTGGGGACAG
AGGTGAAGGGGAACACTGCTGTCTTAATGTGCTGTGTGGGTGGTTATGAT
GGGAGTGGACATGTGGAGGACCTTGGAGCCGAGCAGAACACGGAGATGAC
TTCAATTGCAATGGGGTCTGCAGAAGGCTTTAACCAAGCAGATAAGGCAA
TAAACACCGCTGTAAAGTCGGGGAGGTGGGTGATGCTGAAGAATGTGCAT
CTGGGCCGAGGGTGGGTGATGCAGGTGGAGAAGAAGTTGCATTCCCTGCA
GCCGCATGGCTGCTTGCGACTGTTCCTGAGCATGGAGATCAACCCCAAGG
TGCCTGTGAATCTGCTGCGTGCGGGCCGCATCTTTGTGTTCGAGCGAGCC
CGAGGGGTGAAGGCCAACATGGTGAGGAGGTTGAGGAGGATTCCCGTCTC
AGGGATATCCAAGTCTCCCAACGAGGGTGCCCGGTTGTACTTCGTGCTGG
CGTGGTTTCATGCGATCATCCAAGAACGCTTAGGATAGGCAGCACTGGGG
TGGTCAAAGAAGTATGAATTTGGAGAGTCTGACCTGCGGTCAGCTTCCGA
TACGGTGGAGACGTGGCTGGATGACACGCCCAAGGGCAGGCAGAACATCT
GACCGGATAAGATCCCGTGGTCTGCACTAAAGACCTTAATGGCGCACTCC
ATTTATGGCGGGCCCGTGGAGAACGAGTTTGAGGAGCGTCTGCTCAACAC
CTTCCTGGAGCGGGTGTTCACAACCAGGAGTTTCGACAGTCACTTTAAGC
TGGCATGCAAGGTCGACGGACATAAAGACATTCAAATGCCAGATGGCATG
AGGCCACAGGAGTTTCTGCAGTCCGTGGAGTTGCTCGCCGACACCCACAC
CCCCTCCTGGCTGGGCCTCCCCAACAAGGCCGAGAGAGTCCTCCTTAGGA
CACAGGCTGTGGACATGATCAGTAAAATGCTGAAGATGCAGATCTTGCAG
GATCAGGAGCACGTGCGCTACGCAGAGACTGAGAAGAAGACGAGGACAGA
CTCGACGTCGGACGCGCGCCCTGGGTGGATGCGGACACTGCAGAGGAGCG
CGTCCAACTCCCTCCACCTCATCCCCCAGACGCTGAGCGAGCTCAAGCGC
ACCGTGGAGAATATCAAGGATCCTTTGTTCAGGTTCTTTGAGAGAGAAGT
GAAGATGGGGGCAAAGCTGCTTGACGACCTTCGGCACCAGCTTCCAGATG
TGGTCCAGGTGTGCGAACGAAAGAAGAAGCACACCAACTACTTGCCCAGG
GTGATCAACCAGCTAGTGAAAGGGATCTTGCCTCGGAGCTGCTCCGAGTA
CACCGTGCCTGCGCGCATGACCGTGATCCAGTGGGTGTCCGACTTCAGCG
AGAGGATCAAACAGCTCCAGAAGATCTCAGTGGCAGCTGCATCTGGTGGC
CCCAAGGAGCTAAAGAACATCGAGGTGTCCCTGCGTGCCGTCTTGGTGCG
TGAGGCGTACATCACTGCCAGCAGGCAGTATGTGGCCCAGCCCAACAGCT
GGTCCCTGGAGGAGCTGTGGCTGGAAGTCAACGTGACGAGCTGACAGGGC
CCGAGCCTTGACGCTTGCAGGTTGGGAGTCACGGGTTTGAAACTTCAAGG
GGCCACGTGCAACAACAACAAGCTCTGAGTGTCGAATGCCATCTCAACCG
CCGTTCCCCTGAGCCAGCTGCGCTCCGTCAAGCAGACAAACACCCAGAAC
AAGGCCACTGTGGTAACCTTACCTGTCTACCTGAACTTCACCCGTGCAGA
CCTCATCTTCACCGTGGACTTCGAAATTGCTAGAAAGGAGGATCCTCGCA
GCTTCTACGAGCGGGGTGTCGCAGTCTTGTGCACAGAGTAA

Other nucleic acid sequences that are contemplated as within the scope of this invention include sequences that hybridize to the nucleic acid sequences encoding cytoplasmic dynein heavy chain1 shown in SEQ ID NOS:1, 3, 5, and 17, other than those known in the prior art (as described above), with at least 75%, preferably at least 80%, more preferably at least 90%, even more preferably 95% and most preferably at least 99% sequence homology to the sequence of SEQ ID NOS: 1, 3, 5, or 17. Additionally, nucleic acid sequences that are degenerate with respect to the foregoing sequences are contemplated.

The nucleic acid sequences encoding cytoplasmic dynein heavy chain1 or mutant cytoplasmic dynein heavy chain1 of the invention may exist alone or in combination with other nucleic acids as, for example, vector molecules, such as plasmids, including expression or cloning vectors.

The term “nucleic acid sequence” as used herein refers to any contiguous sequence series of nucleotide bases, e.g., a polynucleotide, and may be ribonucleic acid (RNA) or deoxy-ribonucleic acid (DNA). Preferably the nucleic acid sequence is cDNA.

The term “isolated nucleic acid molecule”, as utilized herein, refers to a nucleic acid molecule that is separated from other nucleic acid molecules ordinarily present in the natural source of the nucleic acid. Preferably, an “isolated” nucleic acid is free of sequences which naturally flank the nucleic acid (e.g., sequences located at the 5′- and 3′-termini of the nucleic acid) in the genomic DNA of the organism that is the natural (wild type) source of the DNA.

Cytoplasmic dynein heavy chain1 molecules can be isolated using standard hybridization and cloning techniques, as described, for instance, in Sambrook et al. (eds.), Molecular Cloning: A Laboratory Manual (2nd Ed.) (Cold Spring Harbor Laboratory Press, Cold Spring Harbor, N.Y., USA, 1989); and Ausubel et al. (eds.), Current Protocols in Molecular Biology (John Wiley & Sons, New York, N.Y., USA, 1993).

A nucleic acid of the invention can be amplified using cDNA, mRNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to cytoplasmic dynein heavy chain1 nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Generally, the term “oligonucleotide” is used to refer to a series of continguous nucleotides (a polynucleotide) of about 100 nucleotdies (nt) or less, e.g., portions of a nucleic acid sequence of about 100 nt, 50 nt, or 20 nt in length, preferably nucleotide sequences of about 15 nt to 30 nt in length.

As used herein, the term “complementary” refers to Watson-Crick or Hoogsteen base pairing between nucleotide units of a nucleic acid molecule, and the term “binding” means the physical or chemical interaction between two polypeptides or compounds or associated polypeptides or compounds or combinations thereof.

The terms “homologous nucleic acid sequence” or “homologous amino acid sequence,” or variations thereof, refer to sequences characterized by a homology at the nucleotide level or amino acid level, respectively. Homologous nucleotide sequences encode those sequences coding for isoforms of cytoplasmic dynein heavy chain1 polypeptides. Isoforms can be expressed in different tissues of the same organism as a result of, for example, alternative splicing of RNA. Alternatively, isoforms can be encoded by different genes.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3, and the temperature is at least about 30° C. for short probes, primers or oligonucleotides (e.g. 10 nt to 50 nt) and at least about 60° C. for longer probes, primers and oligonucleotides. Stringent conditions may also be achieved with the addition of destabilizing agents, such as formamide. Stringent conditions are known to those skilled in the art and can be found in Ausubel et al. (eds.), Current Protocols in Molecular Biology (John Wiley & Sons, New York, N.Y., USA, 1989) at 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C.

As used herein, the term “ortholog” denotes a polypeptide or protein obtained from one species that is the functional counterpart of a polypeptide or protein from a different species. Sequence differences among orthologs are the result of speciation.

Amino Acids

The present invention also provides murine and human cytoplasmic dynein heavy chain1 anino acid sequences (wild type polypeptides and muteins). As used herein, the term “mutein” applies to a protein arising as a result of a mutation. The wild type murine cytoplasmic dynein heavy chain1 amino acid sequence is shown in SEQ ID NO:2. A mutated version wherein Tyr at position 1055 has been mutated to a Cys is shown in SEQ ID NO:4. A preferred embodiment is of a human mutein with the same amino acid change at the corresponding position (native Tyr residue to mutant Cys residue at position 1057) in the sequence is shown in SEQ ID NO:6. Another preferred embodiment is the wild type human dynein heavy chein 1 amino acid sequence, as shown in SEQ ID NO: 18.

TABLE 5
Accession Number AAF91078 (SEQ ID NO:2)

MSEPGGGEDGSAGLEVSAVQNVADVAVLQKHLRKLVPLLLEDGGDAPAAL
EAALEEKSALEQMRKFLSDPQVHTVLVERSTLKEDVGDEGEEEKEFISYN
INIDIHYGVKSNSLAFIKRAPVIDADKPVSSQLRVLTLSEDSPYETLHSF
ISNAVAPFFKSYIRESGKADRDGDKMAPSVEKKIAELEMGLLHLQQNIEI
PEISLPIHPIITNVAKQCYERGEKPKVTDFGDKVEDPTFLNQLQSGVNRW
IREIQKVTKLDRDPASGTALQEISFWLNLERALYRIQEKRESPEVLLTLD
ILKHGKRFHATVSFDTDTGLKQALETVNDYNPLMKDFPLNDLLSATELDK
IRQALVAIFTHLRKIRNTKYPIQRALRLVEAISRDLSSQLLKVLGTRKLM
HVAYEEFEKVMVACFEVFQTWDDEYEKLQVLLRDIVKRKREENLKMVWRI
NPAHRKLQARLDQMRKFRRQHEQLRAVIVRVLRPQVTAVAQQNQGEAPEP
QDNKVAEVLFDAADANTIEEVNLAYENVKEVDGLDVSKEGTEAWEMMKRY
DERIDRVETRITARLRDQLGTAKNANEMFRIFSRFNALFVRPHIRGAIRE
YQTQLIQRVKDDIESLHDKFKVQYPQSQACKMSHVRDLPPVSGSIIWAKQ
IDRQLTAYMKRVEDVLGKGWENHVEGQKLKQDGDSFRMKLNTQEIFDDWA
RKVQQRNLGVSGRIFTIESARVRGRTGNVLKLKVNFLPEIITLSKEVRNL
KWLGFRVPLAIVNKAHQANQLYPFAISLIESVRTYERTCEKVEERNTISL
LVAGLKKEVQALIAEGIALVWESYKLDPYVQRLAETVFNFQEKVDDLLLE
EKIDLEVRSLETCMYDHKTFSEILNRVQKAVDDLNLHSYSNLPIWVNKLD
MEIERILGVRLQAGLRAWTQVLLGQAEDKAEVDMDTDAPQVSHKPGGEPK
IKNVVHELRITNQVIYLNPPIEECRYKLYQEMFAWKMVVLSLPRIQSQRY
QVGVHYELTEEEKFYRNALTRMPDGPVALEESYSAVMGIVTEVEQYVKVW
LQYQCLWDMQAENIYNRLGEDLNKWQALLVQIRKARGTFDNAETKKEFGP
VVIDYGKVQSKVNLKYDSWHKEVLSKFGQMLGSNMTEFHSQISKSRQELE
QHSVDTASTSDAVTFITYVQSLKRKIKQFEKQVELYRNGQRLLEKQRFQF
PPSWLYIDNIEGEWGAFNDIMRRKDSAIQQQVANLQAAKIVQEDRAVESR
TTDLLTDWEKTKPVTGNLRPEEALQALTIYEGKFGRLKDDREKCAKAKEA
LELTDTGLLSGSEERVQVALEELQDLKGVWSELSKVWEQIDQMKEQPWVS
VQPRKLRQNLDGLLNQLKNFPARLRQYASYEFVQRLLKGYMKINMLVIEL
KSEALKDRHWKQLAAKRLHVNWVVSELTLGQIWDVDLQKNEAVVKDVLLV
AQGEAAALEEFLKQIREVWNTYELDLVNYQNKCRLIRGWDDLFNKVKEHI
NSVSAMKLSPYYKVFEEDALSWEDKLNRIMALFDVWIDVQRRWVYLEGIF
TGSADIKHLLPVETQRFQSISTEFLALMKKVSKSPLVMDVLNIQGVQRSL
ERLADLLGKIQKALGEYLERERSSFPRFYFVGDEDLLEIIGNSKNVAKLQ
KHFKKMFAGVSSIILNEDNSVVLGISSREGEEVMFKTPVSITEHPKINEW
LTLVEKEMRVTLAKLLAESVTEVEIFGKATSIDPNTYITWIDKYQAQLVV
LSAQIAWSENYENALSNVGGGGDVGPLQSVLSNVEVTLNVLADSVLMEQP
PLRRRKLEHLITELVHQRDVTRSLIKSKIDNAKSFEWLSQAARFYFDPKQ
TDVLQQLSIQMANAKFNYGFEYLGVQDKLVQTPLTDRCYLTMTQALEARL
GGSPFGPAGTGKTESVKALGHQLGRFVLVFNCDETFDFQMAGRIFVGLCQ
VGAWGCFDEFNRLEERMLSAVSQQVQCIQEALREHSNPNYDKTSAPITCE
LLNKQVKVSPDMAIFITMNPGYAGRSNLPDNLKKLFRSLAMTKPDRQLIA
QVMLYSQGFRTAEVLANKIVPFFKLCDEQLSSQSHYDFGLRALKSVLVSA
GNVKRERIQKIKREKEERGEAVDEGEIAENLPEQEILIQSVCETMVPKLV
AEDIPLLFSLLSDVFPGVQYHRGEAATALREELKKVCQEMYLTYGDGEEV
GGMWVEKVLQLYQITQINHGLMMVGPSGSGKSMAWRVLLKALERLEGVEG
VAHIIDPKAISKDHLYGTLDPNTREWTDGLFTHVLRKIIDNVRGEIQKRQ
WIVFDGDVDPEWVENLNSVLDDNKLLTLPNGERLSLPPNVRIAAFEVQDL
KYATLATVSRCGMVWFSEDVLSTDMILNNFLARLRSIPLDEGEDEAQRRR
KGKEDEGEEAASPMLQIQRDAATIMQPYFTSNGLVTKALEHAFKLEHIMD
LTRLRCLGSLFSMLHQACRNVAQYNANHPDFPMQIEQLERYIQRYLVYAI
LWSLSGDSRLKMRAELGEYIRRITTVPLPTAPNVPIIDYEVSISGEWSPW
QAKVPQIEVETHKVAAPDVVVPTLDTVRHEALLYTWLAEHKPLVLCGPPG
SGKTMTLFSALRALPDMEVVGLNFSSATTPELLLKTFDHYCEYRRTPNGV
VLAPVQLGKWLVLFCDEINLPDMDKYGTQRVISFIRQMVEHGAFYRTSDQ
TWVKLERIQFVGACNPPTDPGRKPLSHRFLRHVPVVYVDYPGPASLTQIY
GTFNRMALRLIPSLRTYAEPLTAAMVEFYTMSQERFTQDTQPHYIYSPRE
AATRWVRGIFEALRPLETLPVEGLIRIWAHEALRLFQDRLVEDEERRWTD
ENIDMVALKHFPNIDKEKMASRPILYSNWLSKDYIPVDQEELRDYVKARL
KVFYEEELDVPLVLFNEVLDHVLRIDRIFRQPQGHLLLIGVSGAGKTTLS
RFVAWMNGLSVYQIKVHRKYTGEDFDEDLRTVLRRSGCKNEKIAFIMDES
NVLDSGFLERAANTLLANGEVPGLFEGDEYATLMTQCKEGAQKEGLMLDS
HEELYKWFTSQVIRNLHVVFTAANPSSEGLKDRAATSPALFNRCVLNWFG
DWSTEALYQVGKEFTSKMDLEKPNYIVPDYMPVVYDKLPQPPTHREAIVN
SCVFVHQTLHQANARLAKRGGRTMAITPRHYLDFINHYANLFHEKRSELE
EQQMHLNVGLRKIKETVDQVEELRRDLRIKSQELEVKNAAANDKLKKMVK
DQQEAEKKKVMSQEIQEQLHKQQEVIADKQMSVKEDLDKVEPAVIEAQNA
VKSIKKQHLVEVRSMANPPMVKLALESICLLLGESTTDWKQIRSIIMREN
FIPTIVNFSAEEISDAIREKMKKNYAASNPSYNYEIVNRASLACGPMVKW
AIAQLNYADMLKRVEPLRNELQKLEDDAKDNQQKANEVEQMIRDLEASIA
RYKEEYAVLISEAQAIKADLAAVEAKVNRSTALLKSLSAERERWEKTSET
FKNQMSTIAGDCLLSAAFIAYAGYFDQQMRQNLFTTWSHHLQQANIQFRT
DIARTEYLSNADERLRWQASSLPADDLCTENAIAALKRFURYPLIIDPSG
QATEFIMNEYKDRKITRTSFLDDAFRKNLESALRFGNPLLVQDVESYDPV
LNPVLNREVRRTGGRVLITLGDQDIDLSPSFVIFLSTRDPTVEFPPDLCS
RVTFVNFTVTRSSLQSQCLNEVLKAERPDVDEKRSDLLKLQGEFQLRLRQ
LEKSLLQALNEVKGRILVDDTIITTLENLKREAAEVTRKVEETDIVMQEV
ETVSQQYLPLSTACSSIYFTMESLKQVHFLYQYSLQFFLDIYHNVLYENP
NLKGATDHTQRLSVITKDLFQVAFNRVARGMLHQDHITFMALLARIKLKG
TVGEPTYDAEFQHFLRGKEIVLSAGSTPKIQGLTVEQAEAVVRLSCLPAF
KDLIAKVQADEQFGIWLDSSSPEQTVPYLWSEETPTTPIGQAIHRLLLIQ
AFRPDRLLAMAHMFVSTNLGESFMSIMEQPLDLTHIVGTEVKPNTPVLAA
CSVPGYDASGHVEDLAAEQUTQITSIAIGSAEGFNQADKAINTAVKSGRW
VMLKNVHLAPGWLMQLEKKLHSLQPHACFRLFLTMEINPKVPVNLLRAGR
IFVFEPPPGVKANMLRTFSSIPVSRICKSPNERARLYFLLAWFHAIIQER
LRYAPLGWSKKYEFGESDLRSACDTVDTWLDDTAKGRQNISPDKIPWSAL
KTLMAQSIYGGRVDDDEFDQRLLNTFLERLFTTRSFDSEFKLACKVDGHK
DIQAAPDGIRREEFVQWVELLPDAQTPSWLGLPNNAERVLLTTQGVDMIS
KMLKMQMLEDEDDLAYAETEKKARTDSTSDGRPAWMRTIHTTASNWLHLI
PQTLSPLKRTVENIKDPLFRFFEREVKMGAKLLQDVRQDLADVVQVCEGK
KKQTNYIRTLINELVKGILPRSWSHYTVPAGMTVIQWVSDFSERIKQLQN
ISQAAASGGAKELKNTHVGLGGLFVPEAYITATRQYVAQANSWSLEELCL
EVNVTASQSATLDACSFGVTGLKLQGATCSNNKLSLSNAISTVLPLTQLR
WVKQTSAEKKASVVTLPVYLNFTRADLIFTVDFEIATKEDPRSFYERGVA
VLCTE

TABLE 6

Mutated murine cytoplasinic dynein heavy chain 1
amino acid sequence (SEQ ID NO:4)

MSEPGGGEDGSAGLEVSAVQNVADVAVLQKHLRKLVPLLLEDGGDAPAAL
EAALEEKSALEQMRKFLSDPQVHTVLVERSTLKEDVGDEGEEEKEFISYN
INIDIHYGVKSNSLAFIKRAPVIDADKPVSSQLRVLTLSEDSPYETLHSF
ISNAVAPFFKSYIRESGKADRDGDKMAPSVEKKIAELEMGLLHLQQNIEI
PEISLPIHPIITNVAKQCYERGEKPKVTDFGDKVEDPTFLNQLQSGVNRW
IREIQKVTKLDRDPASGTALQEISFWLNLERALYRIQEKRESPEVLLTLD
ILKHGKRFHATVSFDTDTGLKQALETVNDYNPLMKDFPLNDLLSATELDK
IRQALVAIFTHLRKIRNTKYPIQRALRLVEAISRDLSSQLLKVLGTRKLM
HVAYEEFEKVMVACFEVFQTWDDEYEKLQVLLRDIVKRKREENLKMVWRI
NPAHRKLQARLDQMRKFRRQHEQLRAVIVRVLRPQVTAVAQQNQGEAPEP
QDMKVAEVLFDAADANTIEEVNLAYENVKEVDGLDVSKEGTEAWEAAMKR
YDERIDRVETRITARLRDQLGTAKNANEMFRIFSRFNALFVRPHIRGAIR
EYQTQLIQRVKDDIESLHDKFKVQYPQSQACKMSHVRDLPPVSGSIIWAK
QIDRQLTAYMKRVEDVLGKGWENHVEGQKLKQDGDSFRMKLNTQEIFDDW
ARKVQQRNLGVSGRIFTIESARVRGRTGNVLKLKVNFLPEIITLSKEVRN
LKWLGFRVPLAIVNKAHQANQLYPFAISLIESVRTYERTCEKVEERNTIS
LLVAGLKKEVQALIAEGIALVWESYKLDPYVQRLAETVFNFQEKVDDLLI
IEEKIDLEVRSLETCMYDHKTFSEILNPVQKAVDDLNLHSYSNLPIWVNK
LDMEIERILGVRLQAGLRAWTQVLLGQAEDKAEVDMDTDAPQVSHKPGGE
PKIKNVVHELRITNQVIYLNPPIEECRYKLYQEMFAWKMVVLSLPRIQSQ
RYQVGVHYELTEEEKFYRNALTRMPDGPVALEESYSAVMGIVTEVEQYVK
VWLQCQCLWDMQAENIYNRLGEDLNKWQALLVQIRKARGTFDNAETKKEF
GPVVIDYGKVQSKVNLKYDSWHKEVLSKFGQMLGSNMTEFHSQISKSRQE
LEQHSVDTASTSDAVTFITYVQSLKRKIKQFEKQVELYRNGQRLLEKQRF
QFPPSWLYIDNIEGEWGAFNDIMRRKDSAIQQQVANLQMKIVQEDRAVES
RTTDLLTDWEKTKPVTGNLRPEEALQALTIYEGKFGRLKDDREKCAKAKE
ALELTDTGLLSGSEERVQVALEELQDLKGVWSELSKVWEQIDQMKEQPWV
SVQPRKLRQNLDGLLNQLKNFPARLRQYASYEFVQRLLKGYMKINMLVIE
LKSEALKDRHWKQLMKRLHVNWVVSELTLGQIWDVDLQKNEAVVKDVLLV
AQGEMALEEFLKQIREVWNTYELDLVNYQNKCRLIRGWDDIFNKVKEHIN
SVSAMKLSPYYKVFEEDALSWEDKLNRIMALFDVWIDVQRRWVYLEGIFT
GSADIKHLLPVETQRFQSISTEFLALMKKVSKSPLVMDVLNIQGVQRSLE
RLADLLGKIQKALGEYLERERSSFPRFYFVGDEDLLEIIGNSKNVANLQK
HFKKMFAGVSSIILNEDNSVVLGISSREGEEVMFKTPVSITEHPKINEWL
TLVEKEMRVTLAKLLAESVTEVEIFGKATSIDPNTYITWIDKYQAQLVVI
SAQIAWSENVENALSNVGGGGDVGPLQSVLSNVEVTLNVLADSVLMEQPP
LRRRKLEHLITELVHQRDVTRSLIKSKIDNAKSFEWLSQMRFYFDPKQTD
VLQQLSIQMANAKFNYGFEYLGVQDKLVQTPLTDRCYLTMTQALEARLGG
SPFGPAGTGKTESVKALGHQIGRFVLVFNCDETFDFQAMGRIFVGLCQVG
AWGCFDEFNRLEERMLSAVSQQVQCIQEALREHSNPNYDKTSAPITCELL
NKQVKVSPDMAIFITMNPGYAGRSNLPDNLKKLFRSLAMTKPDRQLIAQV
MLYSQGFRTAEVLANKIVPFFKLCDEQLSSQSHYDFGLRALKSVLVSAGN
VKRERIQKIKREKEERGEAVDEGEIAENLPEQEILIQSVCETMVPKLVAE
DIPLLFSLLSDVFPGVQYHRGEMTALREELKRVCQEMYLTYGDGEEVGGM
WVEKVLQLYQITQINHGLMMVGPSGSGKSMAWRVLLKALERLEGVEGVAH
IIDPKAISKDHLYGTLDPNTREWTDGLFTHVLRKIIDNVRGELQKRQWIV
FDGDVDPEWVENLNSVLDDNKLLTLPNGERLSLPPNVRIMFEVQDLKYAT
LATVSRCGMVWFSEDVLSTDMILNNFLARLRSIPLDEGEDEAQRRRKGKE
DEGEEAASPMLQIQRDAATIMQPYFTSNGLVTKALEHAFKLEHIMDLTRL
RCLGSLFSMLHQACRNVAQYNANIIPDFPMQIEQLERYIQRYLVYAILWS
LSGDSRLKMRAELGEYIRRITTVPLPTAPNVPIIDYEVSISGEWSPWQAK
VPQIEVETHKVAAPDVVVPTLDTVRHEALLYTWLAEHKPLVLCGPPGSGK
TMTLFSALRALPDMEVVGLNFSSATTPELLLKTFDHYCEYRRTPNGVVLA
PVQLGKWLVLFCDEINLPDMDKYGTQRVISFIRQMVEHGAFYRTSDQTWV
KLERIQFVGACNPPTDPGRKPLSHRFLRHVPVVYVDYPGPASLTQIYGTF
NRAMLRLIPSLRTYAEPLTAAMVEFYTMSQERFTQDTQPHYIYSPREMTR
WVRGIFEALRPLETLPVEGLIRIWAHEALRLFQDRLVEDEERRWTDENI
DMVALKHFPNIDKEKAMSRPILYSNWLSKDYIPVDQEELRDYVKARLKVF
YEEELDVPLVLFNEVLDIIVLRIDRIFRQPQGHLLLIGVSGAGKTTLSRF
VAWMNGLSVYQIKVHRKYTGEDFDEDLRTVLRRSGCKNEKIAFIMDESNV
LDSGFLERMNTLLANGEVPGLFEGDEYATLMTQCKEGAQKEGLMLDSHEE
LYKWFTSQVIRNLHVVFTMNPSSEGLKDRAATSPALFNRCVLNWFGDWST
EALYQVGKEFTSKMDLEKPNYIVPDYMPVVYDKLPQPPTHREAIVNSCVF
VHQTLHQANARLAKRGGRTMAITPRHYLDFINHYANLFHEKRSELEEQQM
HLNVGLRKIKETVDQVEELRRDLRIKSQELEVKNAAANDKLKKMVKDQQE
AEKKKVMSQEIQEQLHKQQEVIADKQMSVKEDLDKVEPAVIEAQNAVKSI
KKQHLVEVRSMANPPAAVKLALESICLLLGESTTDWKQIRSIIMRENFIP
TIVNFSAEEISDAIREKMKKNYMSNPSYNYEIVNRASLACGPMVKWAIAQ
LNYADMLKRVEPLRNELQKLEDDAKDNQQKANEVEQMIRDLEASIARYKE
EYAVLISEAQAIKADLAAVEAKVNRSTALLKSLSAERERWEKTSETFKNQ
MSTIAGDCLLSAAFIAYAGYFDQQMRQNLFTTWSHHLQQANIQFRTDIAR
TEYLSNADERLRWQASSLPADDLCTENAIMLKRFNRYPLIIDPSGQATEF
IMNEYKDRKITRTSFLDDAFRKNLESALRFGNPLLVQDVESYDPVLNPVL
NREVRRTGGRVLITLGDQDIDLSPSFVIFLSTRDPTVEFPPDLCSRVTFV
NFTVTRSSLQSQCLNEVLKAERPDVDEKRSDLLKLQGEFQLRLRQLEKSL
LQALNEVKGRILVDDTIITTLENLKREAAEVTRKVEETDIVMQEVETVSQ
QYLPLSTACSSIYFTMESLKQVHFLYQYSLQFFLDIYHNVLYENPNLKGA
TDHTQRLSVITKDLFQVAFNRVARGMLHQDHITFAMLLARIKLKGTVGEP
TYDAEFQHFLRGKEIVLSAGSTPKIQGLTVEQAEAVVRLSCLPAFKDLIA
KVQADEQFGIWLDSSSPEQTVPYLWSEETPTTPIGQAIIIRLLLIQAFRP
DRLLAMAHMFVSTNLGESFMSIMEQPLDLTHIVGTEVKPNTPVLMCSVPG
YDASGHVEDLAAEQNTQITSIAIGSAEGFNQADKAINTAVKSGRWVMLKN
VHLAPGWLMQLEKKLHSLQPHACFRLFLTMEINPKVPVNLLRAGRIFVFE
PPPGVKANMLRTFSSIPVSRICKSPNERARLYFLLAWFHAIIQERLRYAP
LGWSKKYEFGESDLRSACDTVDTWLDDTAKGRQNISPDKIPWSALKTLMA
QSIYGGRVDNEFDQRLLNTFLERLFTTRSFDSEFKLACKVDGHKDIQMPD
GIRREEFVQWVELLPDAQTPSWLGLPNNAERVLLTTQGVDMISKMLKMQM
LEDEDDLAYAETEKKARTDSTSDGRPAWMRTLHTTASNWLHLIPQTLSPL
KRTVENIKDPLFRFFEREVKMGAKLLQDVRQDLADVVQVCEGKKKQTNYL
RTLINELVKGILPRSWSHYTVPAGMTVIQWVSDFSERIKQLQNISQAAAS
GGAKELKNIHVCLGGLFVPEAYITATRQYVAQANSWSLEELCLEVNVTSQ
SATLDACSFGVTGLKLQGATCSNNKLSLSNAISTVLPLTQLRWVKQTSAE
KKASVVTLPVYLNFTRADLIFTVDFEIATKEDPRSFYERGVAVLCTE

TABLE 7

Mutated human cytoplasmic dyenin heavy chain 1
amino acid sequence (SEQ ID NO:6)

MSEPGGGGGEDGSAGLEVSAVQNVADVSVLQKHLRKLVPLLLEDGGEAPA
ALEAALEEKSALEQMRKFLSDPQVHTVLVERSTLKEDVGDEGEEEKEFIS
YNINIDIHYGVKSNSLAFIKRTPVIDADKPVSSQLRVLTLSEDSPYETLH
SFISNAVAPFFKSYIRESGKADRDGDKMAPSVEKKIAELEMGLLHLQQNI
EIPEISLPIHPMITNVAKQCYERGEKPKVTDFGDKVEDPTFLNQLQSGVN
RWIREIQKVTKLDRDPASGTALQEISFWLNLERALYRIQEKRESPEVLLT
LDILKHGKRFHATVSFDTDTGLKQALETVNDYNPLMKDFPLNDLLSATEL
DKIRQALVAIFTHLRKIRNTKYPIQRALRLVEAISRDLSSQLLKVLGTRK
LMHVAYEEFEKVMVACFEVFQTWDDEYEKLQVLLRDIVKRKREENLKMVW
RINPAHRKLQARLDQMRKFRRQHEQLRAVIVRVLRPQVTAVAQQNQGEVP
EPQDMKVAEVLFDAADANAIEEVNLAYENVKEVDGLDVSKEGTEAWEAAM
KRYDERIDRVETRITARLRDQLGTAKNANEMFRIFSRFNALFVRPHIRGA
IREYQTQLIQRVKDDIESLHDKFIVQYPQSQACKMSHVRDLPPVSGSIIW
AKQIDRQLTAYMKRVEDVLGKGWENHVEGQKLKQDGDSFRMKLNTQEIFD
DWARKVQQRNLGVSGRIFTIESTRVRGRTGNVLKLKVNFLPEIITLSKEV
RNLKWLGFRVPLAIVNKAHQANQLYPFAISLIESVRTYERTCEKVEERNT
ISLLVAGLKKEVQALIAEGIALVWESYKLDPYVQRLAETVFNFQEKVDDL
LIIEEKIDLEVRSLETCMYDHKTFSEILNRVQKAVDDLNLHSYSNLPIWV
NKLDMEIERILGVRLQAGLRAWTQVLLGQAEDKAEVDMDTDAPQVSHKPG
GEPKIKNVVHELRITNQVIYLNFPIEECRYKLYQEMFAWKMVVLSLPRIQ
SQRYQVGVHYELTEEEKFYRNALTRMPDGPVALEESYSAVMGIVSEVEQY
VKVWLQCQCLWDMQAENIYNRLGEDLNKWQALLVQIRKARGTFDNAETKKE
FGPVVIDYGKVQSKVNLKYDSWHKEVLSKFGQMLGSNMTEFHSQISKSRQ
ELEQHSVDTASTSDAVTFITYVQSLKRKIKQFEKQVELYRNGQRLLEKQR
FQFPPSWLYIDNIEGEWGAFNDIMRRKDSAIQQQVANLQMKIVQEDRAVE
SRTTDLLTDWEKTKPVTGNLRPEEALQALTIYEGKFGRLKDDREKCAKAK
EALELTDTGLLSGSEERVQVALEELQDLKGVWSELSKVWEQIDQMKEQPW
VSVQPRKLRQNLDALLNQLKSFPARLRQYASYEFVQRLLKGYMKINMLVI
ELKSEALKDRHWKQLMKRLHVNWVVSELTLGQIWDVULQKNEAIVKDVLL
VAQGEMALEEFLKQIREVWNTYELDLVNYQNKCRLIRGWDDLFNKVKEHI
NSVSAMKLSPYYKVFEEDALSWEDKLNRIMALFDVWIDVQRRWVYLEGIF
TGSADIKHLLPVETQRFQSISTEFLALNKKVSKSPLVMDVLNIQGVQRSL
ERLADLLGKIQKALGEYLERERSSFPRFYFVGDEDLLEIIGNSKNVAKLQ
KHFKKMFAGVSSIILNEDNSVVLGISSREGEEVMFKTPVSITEHPKINEW
LTLVEKEMRVTLAKLLAESVTEVEIFGKATSIDPNTYITWIDKYQAQLVV
LSAQIAWSENVETALSSMGGGGDAAPLHSVLSNVEVTLNVLADSVLMEQP
PLRRRKLEHLITELVHQRDVTRSLIKSKIDMAKSFEWLSQMRFYFDPKQT
DVLQQLSIQMAIAKFNYGFEYLGVQDKLVQTPLTDRCYLTMTQALEARLG
GSPFGPAGTGKTESVKALGHQLGRFVLVFNCDETFDFQAMGRIFVGLCQV
GAWGCFDEFNRLEERMLSAVSQQVQCIQEALREHSIIPNYDKTSAIITCE
LLNKQVKVSPDMAIFITMNPGYAGRSNLPDNLKKLFRSLAMTKPDRQLIA
QVMLYSQGFRTAEVLANKIVPFFKLCDEQLSSQSHYDFGLRALKSVLVSA
GNVKRERIQKIKREKEERGEAVDEGEIAENLPEQEILIQSVCETMVPKLV
AEDIPLLFSLLSDVFPGVQYHRGEMTALREELKKVCQEMYLTYGDGEEVG
GMWVEKVLQLYQITQINHGLMMVGPSGSGKSMAWRVLLKALERLEGVEGV
AHIIDPKAISKDHLYGTLDPNTREWTDGLFTHVLRKIIDSVRGELQKRQW
IVFDGDVDPEWVENLNSVLDDNKLLTLPNGERLSLPPNVRIMFEVQDLKY
ATLATVSRCGMVWFSEDVLSTDMIFNNFLARLRSIPLDEGEDEAQRRRKG
KEDEGEEAASPMLQIQRDAATIMQPYFTSNGLVTKALEHAFQLEHIMDLT
RLRCLGSLFSMLHQACRNVAQYNANHPDFPMQIEQLERYIQRYLVYAILW
SLSGDSRLKMRAELGEYIRRITTVPLPTAPNIPIIDYEVSISGEWSPWQA
KVPQIEVETHKVAAPDVVVPTLDTVRHEALLYTWLAEHKPLVLCGPPGSG
KTMTLFSALRALPDMEVVGLNFSSATTPELLLKTFDHYCEYRRTPNGVVL
APVQLGKWLVLFCDEINLFDMDKYGTQRVISFIRQMVEHGGFYRTSDQTW
VKLERIQFVGACNFPTDFGRKPLSHRFLRHVPVVYVDYPGPASLTQIYGT
FNRAMLRLIPSLRTYAEPLTAAMVEFYTMSQERFTQDTQPHYIYSPREMT
RWVRGIFEALRPLETLFVEGLIRIWAHEALRLFQDRLVEDEERRWTDEN
IDTVALKHFPNIDREKAMSRPILYSNWLSKDYIFVDQEELRDYVKARLKV
FYEEELDVPLVLFNEVLDHVLRIDRIFRQPQGHLLLIGVSGAGKTTLSRF
VAWMNGLSVYQIKVHRKYTGEDFDEDLRTVLRRSGCKNEKIAFIMDESNV
LDSGFLERMNTLLANGEVPGLFEGDEYATLMTQCKEGAQKEGLMLDSHEE
LYKWFTSQVIRNLHVVFTMNPSSEGLKDRAATSPALFNRCVLNWFGDWST
EALYQVGKEFTSKMDLEKFNYIVPDYMPVVYDDLPQPPSHREAIVNSCVF
VHQTLHQANARLAKRGGRTMAITPRHYLDFINHYANLFHEKRSELEEQQM
HLNVGLRKIKETVDQVEELRRDLRIKSQELEVKNAAANDKLKKMVICDQQ
EAEKKKVMSQEIQEQLHECQQEVIADKQMSVKEDLDKVEPAVIEAQNAVK
SIKKQHLVEVRSMANFFAAVKLALESICLLLGESTTDWKQIRSIIMRENF
IPTIVNFSAEEISDAIREKMKKNYMSNFSYNYEIVNRASLACGPMVKWAI
AQLNYADMLKRVEPLRNELQKLEDDAKDNQQKANEVEQMIRDLEASIARY
KEEYAVLISEAQAIKADLAAVEAKVNRSTALLKSLSAERERWEKTSETFK
FKNQMSTIAGDCLLSAAFIAYAGYFDQQMRQNLFTTWSHHLQQANIQFRT
DIARTEYLSNADERLRWQASSLPADDLCTENAIMLKRFNRYPLIIDPSGQ
ATEFIMNEYKDRKITRTSFLDDAFRKNLESALRFGNFLLVQDVESYDPVL
NFVLNREVRRTGGRVLITLGDQDIDLSPSFVIFLSTRDPTVEFPPDLCSR
VTFVNFTVTRSSLQSQCLIIEVLKAERPDVDEKRSDLLKLQGEFQLRLRQ
LEKSLLQALNEVKGRILDDDTIITTLENLKREAAEVTRKVEETDIVMQEV
ETVSQQYLPLSTACSSIYFTMESLKQIHFLYQYSLQFFLDIYHNVLYENP
NLKGVTDHTQRLSIITKDLFQVAFNRVARGMLHQDHITFAMLLARIKLKG
TVGEPTYDAEFQHFLRGNEIVLSAGSTPRIQGLTVEQAEAVVRLSCLPAF
KDLIAKVQADEQFGIWLDSSSPEQTVPYLWSEETFATLIGQAIHRLLLIQ
AFRPDRLLAMAHMFVSTNLGESFMSIMEQPLDLTHIVGTEVKPNTFVLMC
SVPGYDASGHVEDLAAEQNTQITSIAIGSAEGFNQADKAINTAVKSGRWV
MLKNVHLAPGWLMQLEKKLHSLQPHACFRLFLTMEINPKVFVNLLRAGRI
FYFEPPPGVKANMLRTFSSIPVSRICKSPNERARLYFLLAWFHAIIQERL
RYAPLGWSKKYEFGESDLRSACDTVDTWLDDTAKGRQNISPDKIPWSALK
TLMAQSIYGGRVDNEFDQRLLNTFLERLFTTRSFDSEFKLACKVDGHKDI
QMPDGIRREEFVQWVELLPDTQTPSWLGLPNNAERVLLTTQGVDMISKML
KMQMLEDEDDLAYAETEKICTRTDSTSDGRPAWMRTLHTTASNWLHLIPQ
TLSHLKRTVENIKDPLFRFFEREVKMGAKLLQDVRQDLADVVQVCEGKKK
QTNYLRTLINELVKGILPRSWSHYTVPAGMTVIQWVSDFSERIKQLQNIS
LAAASGGAELKNIHVCLGGLFVPEAYITATRQYVAQANSWSLEELCLEVN
VTTSQGATLDACSFGVTGLKLQGATCNNNKLSLSNAISTALFLTQLRWVK
QTNTEKKASVVTLPVYLNFTRADLIFTVDFEIATKEDPRSFYERGVAVLC
TE

TABLE 8

Wild type human cytoplasmic dynein heavy chain 1
amino acid sequence (SEQ ID NO:18)

NSEPGGGGGEDGSAGLEVSAVQNVADVSVLQKHLRKLVPLLLEDGGEAPA
ALEAALEEKSALEQMRKFLSDPQVHTVLVERSTLKEDVGDEGEEEKEFIS
YNINIDIHYGVKSNSLAFRKRTPVIDADKPVSSQLRVLTLSEDSPYETLH
SFISNAVAPFFKSYIRESGKADRDGDKMAPSVEKKIAELEMGLLHLQQNI
EIPEISLPIHPMITNVAKQCYERGEKPKVTDFGDKVEDPTFLNQLQSGVN
RWIREIQKVTKLDRDPASGTALOEISFWLNLERALYRIQEKRESPEVLLT
LDILKHGKRFHATVSFDTDTGLKQALETVNDYNFLMKDFPLNDLLSATEL
DKIRQALVAIFTHLRKIRNTKYPIQRALRLVEAISRDLSSQLLKVLGTRK
LMHVAYEEFEKVMVACFEVFQTWDDEYEKLQVLLRDIVKRKREENLKMVW
RINPAHRKLQARLDQMRKFRRQHEQLRAVIVRVLRPQVTAVAQQNQGEVP
EPQDMKVAEVLFDAADANAIEEVNLAYENVKEVDGLDVSKEGTEAWEAAM
KRYDERIDRVETRITARLRDQTGTAKNANEMFRIFSRFNALFVRPHIRGA
IREYQTQLIQRVKDDIESLHDKFKVQYPQSQACKMSHVRDLPPVSGSIIW
AKQIDRQLTAYMKRVEDVLGKGWENHVEGQKLKQDGDSFRMKLUTQEIFD
DWARKVQQRNLGVSGRIFTIESTRVRGRTGNVLKLKVNFLPEIITLSKEV
RNLKWLGFRVPLAIVNKAHQANQLYPFAISLIESVRTYERTCEKVEERNT
ISLLVAGLKKEVQALIAEGIALVWESYKLDPYVQRLAETVFNFQEKVDDL
LIlEEKIDLEVRSLETCMYDHKTFSEILNRVQKAVDDLNLHSYSNLPIWV
NKLDMEIERILGVRLQAGLRAWTQVLLGQAEDKAEVDMDTDAPQVSHKPG
GEPKIKNVVHELRITNQVIYLNPPIEECRYKLYQEMFAWKMVVLSLPRIQ
SQRYQVGVHYELTEEEKFYRNALTRMPDGPVALEESYSAVMGIVSEVEQY
VKVWLQYQCLWDMQAENIYNRLGEDLNKWQALLVQIRKARGTFDNAETKK
EFGPVVIDYGKVQSKVNLKYDSWHKEVLSKFGQMLGSNMTEFHSQISKSR
QELEQHSVDTASTSDAVTFITYVQSLKRKIKQFEKQVELYRNGQRLLEKQ
RFQFPPSWLYIDNIEGEWGAFNDIMRRKDSAIQQQVANLQMKIVQEDRAV
ESRTTDLLTDWEKTKPVTGNLRPEEALQALTIYEGKFGRLKDDREKCAKA
KEALELTDTGLLSGSEERVQVALEELQDLKGVWSELSKVWEQIDQMKEQP
WVSVQPRKLRQNLDALLNQLKSFPARLRQYASYEFVQRLLKGYMKINMLV
IELKSEALKDRHWKQLMKRLHVNWVVSELTLGQIWDVDLQKNEAIVKDVL
LVAQGEMALEEFLKQIREVWNTYELDLVNYQNKCRLIRGWDDLFNKVKEH
INSVSAMKLSPYYKVFEEDALSWEDKLNRIMALFDVWIDVQRRWVYKEGI
FTGSADIKHLLPVETQRFQSISTEFLALMKKVSKSPLVMDVLNIQGVQRS
LERLADLLGKIQKALGEYLERERSSFPRFYFVGDEDLLEIIGNSKNVAKL
QKHFKKMFAGVSSIILNEDNSVVLGISSREGEEVMFKTPVSITEHPKINE
WLTLVEKEMRVTLAKLLAESVTEVEIFGKATSIDPNTYITWIDKYQAQLV
VLSAQlAWSENVETALSSMGGGGDAAPLHSVLSNVEVTLNVLADSVLMEQ
PPLRRRKLEHLITELVHQRDVTRSLIKSKIDNAKSFEWLSQMRFYFDPKQ
TDVLQQLSIQMANAKFNYGFEYLGVQDKLVQTPLTDRCYLTMTQALEARL
GGSPFGPAGTGKTESVKALGHQLGRFVLVFNCDETFDFQAMGRIFVGLCQ
VGAWGCFDEFNRLEERMLSAVSQQVQCIQEALREHSNPNYDKTSAPITCE
LLNKQVKVSPDMAIFITMNPGYAGRSNLPDNLKKLFRSLAMTKPDRQLIA
QVMLYSQGFRTAEVLANKIVPFFKLCDEQLSSQSHYDFGLRALKSVLVSA
GNVKRERIQKIKREKEERGEAVDEGEIAENLPEQEILIQSVCETMVPKLV
AEDIPLLFSLLSDVFPGVQYHRGEMTALREELKKVCQEMYLTYGDGEEVG
GMWVEKVLQLYQITQINIIGLMMVGPSGSGKSMAWRVLLKALERLEGVEG
VAHIIDPKAISKDHLYGTLDPNTREWTDGLFTHVLRKITDSVRGELQKRQ
WIVFDGDVDPEWVENLNSVLDDNKLLTLPNGERLSLPPNVRIMFEVQDLK
YATLATVSRCGMVWFsEDVLSTDMIFNNFLARLRSIPLDEGEDEAQRRRK
GKEDEGEEAAsPMLQIQRDAATIMQPYFTSNGLVTKALEHAFQLEHIMDL
TRLRCLGSLFSMLHQACRNVAQYNANHPDFPMQIEQLERYIQRYLVYAIL
WSLSGDSRLKMRAELGEYIRRITTVPLPTAPNIPIIDYEVSISGEWSPWQ
AKVPQIEVETHKVAAPDVVVPTLDTVRHEALLYTWLAEHKPLVLCGPPGS
GKTMTLFSALRALPDMEVVGLNFSSATTPELLLKTFDHYCEYRRTPNGVV
LAPVQLGKWLVLFCDEINLPDMDKYGTQRVISFIRQMVEHGGFYRTSDQT
WVKLERIQFVGACNPPTDPGRKPLSHRFLRHVPVVYVDYPGPASLTQIYG
TFNRAMLRLIPSLRTYAEPLTAAMVEFYTMSQERFTQDTQPHYIYSPREM
TRWVRGIFEALRPLETLPVEGLIRIWAHEALRLFQDRLVELEERRWTDEN
IDTVALKHFPNIDREKAMSRPILYSNWLSKDYIPVDQEELRDYVKARLKV
FYEEELDVPLVLFNEVLDHVLRIDRIFRQPQGHLLLIGVSGAGKTTLSRF
VAWMNGLSVYQIKVHRKYTGEDFDEDLRTVLRRSGCKNEKIAFIMDESNV
LDSGFLERMNTLLANGEVPGLFEGDEYATLMTQCKEGAQKEGLMLDSHEE
LYKWFTSQVIRNLHVVFTMNPSSEGLKDRAATSPALFNRCVLNWFGDWST
EALYQVGKEFTSKMDLEKPNYIVPDYMPVVYDKLPQPPSHREAIVNSCVF
VHQTLHQANARLAKRGGRTMAITPRHYLDFINHYANLFHEKRSELEEQQM
HLNVGLRKIKETVDQVEETJRRDLRIKSQELEVKNAAANDKLKKMVKDQQ
EAEKKKVMSQEIQEQLHKQQEVIADKQMSVKEDLDKVEPAVIEAQNAVKS
IKKQHIIVEVRSMANPPAAVKLALESICLLLGESTTDWKQIRSIIMRENF
IPTIVNFSAEEISDAIREKMKKNYMSNPSYNYEIVNRASLACGPMVKWAI
AQLNYADMLKRVEPLRNELQKLEDDAKDNQQKANEVEQMIRDLEASIARY
KEEYAVLISEAQAIKADLAAVEAKVNRSTALLKSLSAERERWEKTSETFK
NQMSTIAGDCLLSAAFIAYAGYFDQQMRQNLFTTWSIIHLQQANIQFRTD
IARTEYLSNADERLRWQASSLPADDLCTENAIMLKRFNRYPITIDPSGQA
TEFIMNEYKDRKITRTSFLDDAFRKNLESALRFGNPLLVQDVESYDPVLN
PVLNREVRRTGGRVLITLGDQDIDLSPSFVIFLSTRDPTVEFPPDLCSRV
TFVNFTVTRSSLQSQCLNEVLKAERPDVDEKRSDLLKLQGEFQLRLRQLE
KSLLQATiNEVKGRILDDDTIITTLENIKREAAEVTRKVEETDIVMQEVE
TVSQQYLPLSTACSSIYFTMESLKQIHFLYQYSLQFFLDIYHNVLYENPN
LKGVTDHTQRLSIETKDLFQVAFNRVARGMLHQDHITFABMLLARIKLKG
TVGEPTYDAEFQHFLRGNEIVLSAGSTPRIQGLTVEQAEAVVRLSCLPAF
KDLIAKVQADEQFGIWLDSSSPEQTVPYLWSEETPATPIGQAIHRLLLIQ
AFRPDRLLAMAHMFVSTNLGESFMSIMEQPLDLTHIVGTEVKPNTPVLMC
SVPGYDASGHVEDLAAEQNTQITSIAIGSAEGFNQADKAINTAVKSGRWV
MLKNVHLAPGWLMQLEKKLHSLQPHACFRLFLTMEINPKVPVNLLRAGRI
FVFEPPPGVKANMLRTFSSIPVSRICKSPNERARLYFLIAWFHAIIQERL
RYAPLGWSKKYEFGESDLRSACDTVDTWLDDTAKGRQNISPDKIPWSALK
TLMAQSIYGGRVDNEFDQRLLLTFLERLFTTRSFDSEFKLACKVDGHKDI
QMPDGIRREEFVQWVELLPDTQTPSWLGLPNNAERVLLTTQGVDMISKML
KMQMLEDEDDLAYAETEKKTRTDSTSDGRPAWMRTLHTTASNWLHLIPQT
LSHLKRTVENIKDPLFRFFEREVKMGAKLLQDVRQDLADVVQVCEGKKKQ
TNYLRTLINELVKGILPRSWSHYTVPAGMTVIQWVSDFSERIKQLQNISL
AAASGGAKELKNIHVCLGGLFVPEAYITATRQYVAQANSWSLEELCLEVN
VTTSQGATLDACSFGVTGLKLQGATCNNNKLSLSNAISTALPLTQLRWVK
QTNTEKKASVVTLPVYLNFTRADLIFTVDFEIATKEDPRSFYERGVAVLC
TE

Preferably, the wild type residue of the modified human cytoplasmic dynein heavy chain1 protein is replaced by an amino acid with different size and/or polarity, e.g., a non-conservative amino acid substitution.

Preferably, residue 1055 of murine cytoplasmic dynein heavy chain1 and residue 1057 of human cytoplasmic dynein heavy chain1 according to the present invention is replaced by an amino acid other than Trp or Phe and preferably is replaced by an amino acid selected from Ala, Ser, Thr, Pro, Gly, Met, Leu, Ile, Val, or Cys, more preferably by Met, Leu, Ble, Val, or Cys, and most preferably by Cys.

In a most preferred embodiment the murine cytoplasmic dynein heavy chain1 mutein of the present invention has the amino acid sequence shown in SEQ ID NO:4. In another preferred embodiment the human cytoplasmic dynein heavy chain1 mutein of the present invention has the amino acid sequence shown in SEQ ID NO:6.

The present invention is not limited to muteins with a mutation of the residue at, e.g., position 1055 of the amino acid sequence shown in SEQ ID NO: 4, and it is not limited to the mutation of the residue at, e.g., position 1057 of the amino acid sequence shown in SEQ ID NO: 6 or at a corresponding position in other dynein heavy chain proteins (especially cytoplasmic dynein heavy chain1 proteins from different species). Rather it encompasses additional muteins with modifications in the amino acid sequence of the mouse or human cytoplasmic dynein heavy chain1 proteins as long as they impart a phenotype as described herein in connection with the animals. Such muteins, may comprise mutations, such as single or multiple further amino acid substitutions, deletions and insertions.

One embodiment of the invention relates to muteins wherein the amino acid residue affected by the mutation, e.g., a substitution, deletion, or insertion, corresponds to a residue selected from the group of conserved amino acid residues between Leu302 (302L) and Phe1140 (1140F) of the cytoplasmic dynein heavy chain1 amino acid sequence, e.g., SEQ ID NO:2 (Mus musculus) specified in Table 19.

A preferred mutein of the invention is one wherein the conserved amino acid affected by said mutation is the amino acid 302L.

A preferred mutein of the invention is one wherein the conserved amino acid affected by said mutation is the amino acid 303K.

A preferred mutein of the invention is one wherein the conserved amino acid affected by said mutation is the amino acid 305G.

A preferred mutein of the invention is one wherein the conserved amino acid affected by said mutation is the amino acid 306K.

A preferred mutein of the invention is one wherein the conserved amino acid affected by said mutation is the amino acid 307R.

A preferred mutein of the invention is one wherein the conserved amino acid affected by said mutation is the amino acid 308F.

A preferred mutein of the invention is one wherein the conserved amino acid affected by said mutation is the amino acid 309H.

A preferred mutein of the invention is one wherein the conserved amino acid affected by said mutation is the amino acid 310A.

A preferred mutein of the invention is one wherein the conserved amino acid affected by said mutation is the amino acid 311T.

A preferred mutein of the invention is one wherein the conserved amino acid affected by said mutation is the amino acid 312V.

A preferred mutein of the invention is one wherein the conserved amino acid affected by said mutation is the amino acid 314F.

A preferred mutein of the invention is one wherein the conserved amino acid affected by said mutation is the amino acid 315D.

A preferred mutein of the invention is one wherein the conserved amino acid affected by said mutation is the amino acid 317D.

A preferred mutein of the invention is one wherein the conserved amino acid affected by said mutation is the amino acid 319G.

A preferred mutein of the invention is one wherein the conserved amino acid affected by said mutation is the amino acid 320L.

A preferred mutein of the invention is one wherein the conserved amino acid affected by said mutation is the amino acid 321K.

A preferred mutein of the invention is one wherein the conserved amino acid affected by said mutation is the amino acid 322Q.

A preferred mutein of the invention is one wherein the conserved amino acid affected by said mutation is the amino acid 324L.

A preferred mutein of the invention is one wherein the conserved amino acid affected by said mutation is the amino acid 327V.

A preferred mutein of the invention is one wherein the conserved amino acid affected by said mutation is the amino acid 329D.

A preferred mutein of the invention is one wherein the conserved amino acid affected by said mutation is the amino acid 330Y.

A preferred mutein of the invention is one wherein the conserved amino acid affected by said mutation is the amino acid 331N.

A preferred mutein of the invention is one wherein the conserved amino acid affected by said mutation is the amino acid 333L.

A preferred mutein of the invention is one wherein the conserved amino acid affected by said mutation is the amino acid 334M.

A preferred mutein of the invention is one wherein the conserved amino acid affected by said mutation is the amino acid 335K.

A preferred mutein of the invention is one wherein the conserved amino acid affected by said mutation is the amino acid 337F.

A preferred mutein of the invention is one wherein the conserved amino acid affected by said mutation is the amino acid 338P.

A preferred mutein of the invention is one wherein the conserved amino acid affected by said mutation is the amino acid 342L.

A preferred mutein of the invention is one wherein the conserved amino acid affected by said mutation is the amino acid 344S.

A preferred mutein of the invention is one wherein the conserved amino acid affected by said mutation is the amino acid 345A.

A preferred mutein of the invention is one wherein the conserved amino acid affected by said mutation is the amino acid 346T.

A preferred mutein of the invention is one wherein the conserved amino acid affected by said mutation is the amino acid 350K.

A preferred mutein of the invention is one wherein the conserved amino acid affected by said mutation is the amino acid 354A.

A preferred mutein of the invention is one wherein the conserved amino acid affected by said mutation is the amino acid 358I.

A preferred mutein of the invention is one wherein the conserved amino acid affected by said mutation is the amino acid 359F.

A preferred mutein of the invention is one wherein the conserved amino acid affected by said mutation is the amino acid 361H.

A preferred mutein of the invention is one wherein the conserved amino acid affected by said mutation is the amino acid 362L.

A preferred mutein of the invention is one wherein the conserved amino acid affected by said mutation is the amino acid 363%

A preferred mutein of the invention is one wherein the conserved amino acid affected by said mutation is the amino acid 364K.

A preferred mutein of the invention is one wherein the conserved amino acid affected by said mutation is the amino acid 366R.

A preferred mutein of the invention is one wherein the conserved amino acid affected by said mutation is the amino acid 368T.

A preferred mutein of the invention is one wherein the conserved amino acid affected by said mutation is the amino acid 369K.

A preferred mutein of the invention is one wherein the conserved amino acid affected by said mutation is the amino acid 370Y.

A preferred mutein of the invention is one wherein the conserved amino acid affected by said mutation is the amino acid 371P.

A preferred mutein of the invention is one wherein the conserved amino acid affected by said mutation is the amino acid 373Q.

A preferred mutein of the invention is one wherein the conserved amino acid affected by said mutation is the amino acid 374R.

A preferred mutein of the invention is one wherein the conserved amino acid affected by said mutation is the amino acid 376L.

A preferred mutein of the invention is one wherein the conserved amino acid affected by said mutation is the amino acid 378L.

A preferred mutein of the invention is one wherein the conserved amino acid affected by said mutation is the amino acid 380E.

A preferred mutein of the invention is one wherein the conserved amino acid affected by said mutation is the amino acid 381A.

A preferred mutein of the invention is one wherein the conserved amino acid affected by said mutation is the amino acid 382I.

A preferred mutein of the invention is one wherein the conserved amino acid affected by said mutation is the amino acid 383S.

A preferred mutein of the invention is one wherein the conserved amino acid affected by said mutation is the amino acid 384R.

A preferred mutein of the invention is one wherein the conserved amino acid affected by said mutation is the amino acid 385D.

A preferred mutein of the invention is one wherein the conserved amino acid affected by said mutation is the amino acid 386L.

A preferred mutein of the invention is one wherein the conserved amino acid affected by said mutation is the amino acid 389Q.

A preferred mutein of the invention is one wherein the conserved amino acid affected by said mutation is the amino acid 390L.

A preferred mutein of the invention is one wherein the conserved amino acid affected by said mutation is the amino acid 391L.

A preferred mutein of the invention is one wherein the conserved amino acid affected by said mutation is the amino acid 392K.

A preferred mutein of the invention is one wherein the conserved amino acid affected by said mutation is the amino acid 393V.

A preferred mutein of the invention is one wherein the conserved amino acid affected by said mutation is the amino acid 394L.

A preferred mutein of the invention is one wherein the conserved amino acid affected by said mutation is the amino acid 399L.

A preferred mutein of the invention is one wherein the conserved amino acid affected by said mutation is the amino acid 400M.

A preferred mutein of the invention is one wherein the conserved amino acid affected by said mutation is the amino acid 406E.

A preferred mutein of the invention is one wherein the conserved amino acid affected by said mutation is the amino acid 407F.

A preferred mutein of the invention is one wherein the conserved amino acid affected by said mutation is the amino acid 411M.

A preferred mutein of the invention is one wherein the conserved amino acid affected by said mutation is the amino acid 414C.

A preferred mutein of the invention is one wherein the conserved amino acid affected by said mutation is the amino acid 418F.

A preferred mutein of the invention is one wherein the conserved amino acid affected by said mutation is the amino acid 421W.

A preferred mutein of the invention is one wherein the conserved amino acid affected by said mutation is the amino acid 422D.

A preferred mutein of the invention is one wherein the conserved amino acid affected by said mutation is the amino acid 423D.

A preferred mutein of the invention is one wherein the conserved amino acid affected by said mutation is the amino acid 424E.

A preferred mutein of the invention is one wherein the conserved amino acid affected by said mutation is the amino acid 425Y.

A preferred mutein of the invention is one wherein the conserved amino acid affected by said mutation is the amino acid 427K.

A preferred mutein of the invention is one wherein the conserved amino acid affected by said mutation is the amino acid 431L.

A preferred mutein of the invention is one wherein the conserved amino acid affected by said mutation is the amino acid 432L.

A preferred mutein of the invention is one wherein the conserved amino acid affected by said mutation is the amino acid 433R.

A preferred mutein of the invention is one wherein the conserved amino acid affected by said mutation is the amino acid 434D.

A preferred mutein of the invention is one wherein the conserved amino acid affected by said mutation is the amino acid 435I.

A preferred mutein of the invention is one wherein the conserved amino acid affected by said mutation is the amino acid 437K.

A preferred mutein of the invention is one wherein the conserved amino acid affected by said mutation is the amino acid 439K.

A preferred mutein of the invention is one wherein the conserved amino acid affected by said mutation is the amino acid 440R.

A preferred mutein of the invention is one wherein the conserved amino acid affected by said mutation is the amino acid 445K.

A preferred mutein of the invention is one wherein the conserved amino acid affected by said mutation is the amino acid 448W.

A preferred mutein of the invention is one wherein the conserved amino acid affected by said mutation is the amino acid 454H.

A preferred mutein of the invention is one wherein the conserved amino acid affected by said mutation is the amino acid 457L.

A preferred mutein of the invention is one wherein the conserved amino acid affected by said mutation is the amino acid 460R.

A preferred mutein of the invention is one wherein the conserved amino acid affected by said mutation is the amino acid 467F.

A preferred mutein of the invention is one wherein the conserved amino acid affected by said mutation is the amino acid 468R.

A preferred mutein of the invention is one wherein the conserved amino acid affected by said mutation is the amino acid 470Q.

A preferred mutein of the invention is one wherein the conserved amino acid affected by said mutation is the amino acid 471H.

A preferred mutein of the invention is one wherein the conserved amino acid affected by said mutation is the amino acid 472E.

A preferred mutein of the invention is one wherein the conserved amino acid affected by said mutation is the amino acid 473Q.

A preferred mutein of the invention is one wherein the conserved amino acid affected by said mutation is the amino acid 475R.

A preferred mutein of the invention is one wherein the conserved amino acid affected by said mutation is the amino acid 477V.

A preferred mutein of the invention is one wherein the conserved amino acid affected by said mutation is the amino acid 478I.

A preferred mutein of the invention is one wherein the conserved amino acid affected by said mutation is the amino acid 480R.

A preferred mutein of the invention is one wherein the conserved amino acid affected by said mutation is the amino acid 481V.

A preferred mutein of the invention is one wherein the conserved amino acid affected by said mutation is the amino acid 482L.

A preferred mutein of the invention is one wherein the conserved amino acid affected by said mutation is the amino acid 483R.

A preferred mutein of the invention is one wherein the conserved amino acid affected by said mutation is the amino acid 484P.

A preferred mutein of the invention is one wherein the conserved amino acid affected by said mutation is the amino acid 519E.

A preferred mutein of the invention is one wherein the conserved amino acid affected by said mutation is the amino acid 521V.

A preferred mutein of the invention is one wherein the conserved amino acid affected by said mutation is the amino acid 524A.

A preferred mutein of the invention is one wherein the conserved amino acid affected by said mutation is the amino acid 525Y.

A preferred mutein of the invention is one wherein the conserved amino acid affected by said mutation is the amino acid 526E.

A preferred mutein of the invention is one wherein the conserved amino acid affected by said mutation is the amino acid 529K.

A preferred mutein of the invention is one wherein the conserved amino acid affected by said mutation is the amino acid 531V.

A preferred mutein of the invention is one wherein the conserved amino acid affected by said mutation is the amino acid 532D.

A preferred mutein of the invention is one wherein the conserved amino acid affected by said mutation is the amino acid 534L.

A preferred mutein of the invention is one wherein the conserved amino acid affected by said mutation is the amino acid 535D.

A preferred mutein of the invention is one wherein the conserved amino acid affected by said mutation is the amino acid 543A.

A preferred mutein of the invention is one wherein the conserved amino acid affected by said mutation is the amino acid 544W.

A preferred mutein of the invention is one wherein the conserved amino acid affected by said mutation is the amino acid 545E.

A preferred mutein of the invention is one wherein the conserved amino acid affected by said mutation is the amino acid 547A.

A preferred mutein of the invention is one wherein the conserved amino acid affected by said mutation is the amino acid 549K.

A preferred mutein of the invention is one wherein the conserved amino acid affected by said mutation is the amino acid 550P.

A preferred mutein of the invention is one wherein the conserved amino acid affected by said mutation is the amino acid 551 Y.

A preferred mutein of the invention is one wherein the conserved amino acid affected by said mutation is the amino acid 555I.

A preferred mutein of the invention is one wherein the conserved amino acid affected by said mutation is the amino acid 558V.

A preferred mutein of the invention is one wherein the conserved amino acid affected by said mutation is the amino acid 559E.

A preferred mutein of the invention is one wherein the conserved amino acid affected by said mutation is the amino acid 560T.

A preferred mutein of the invention is one wherein the conserved amino acid affected by said mutation is the amino acid 562I.

A preferred mutein of the invention is one wherein the conserved amino acid affected by said mutation is the amino acid 563T.

A preferred mutein of the invention is one wherein the conserved amino acid affected by said mutation is the amino acid 566L.

A preferred mutein of the invention is one wherein the conserved amino acid affected by said mutation is the amino acid 569Q.

A preferred mutein of the invention is one wherein the conserved amino acid affected by said mutation is the amino acid 570L.

A preferred mutein of the invention is one wherein the conserved amino acid affected by said mutation is the amino acid 575N.

A preferred mutein of the invention is one wherein the conserved amino acid affected by said mutation is the amino acid 577N.

A preferred mutein of the invention is one wherein the conserved amino acid affected by said mutation is the amino acid 578E.

A preferred mutein of the invention is one wherein the conserved amino acid affected by said mutation is the amino acid 579M.

A preferred mutein of the invention is one wherein the conserved amino acid affected by said mutation is the amino acid 580F.

A preferred mutein of the invention is one wherein the conserved amino acid affected by said mutation is the amino acid 5821.

A preferred mutein of the invention is one wherein the conserved amino acid affected by said mutation is the amino acid 583F.

A preferred mutein of the invention is one wherein the conserved amino acid affected by said mutation is the amino acid 584S.

A preferred mutein of the invention is one wherein the conserved amino acid affected by said mutation is the amino acid 585R.

A preferred mutein of the invention is one wherein the conserved amino acid affected by said mutation is the amino acid 587N.

A preferred mutein of the invention is one wherein the conserved amino acid affected by said mutation is the amino acid 588A.

A preferred mutein of the invention is one wherein the conserved amino acid affected by said mutation is the amino acid 589L.

A preferred mutein of the invention is one wherein the conserved amino acid affected by said mutation is the amino acid 590F.

A preferred mutein of the invention is one wherein the conserved amino acid affected by said mutation is the amino acid 592R

A preferred mutein of the invention is one wherein the conserved amino acid affected by said mutation is the amino acid 593P.

A preferred mutein of the invention is one wherein the conserved amino acid affected by said mutation is the amino acid 595I.

A preferred mutein of the invention is one wherein the conserved amino acid affected by said mutation is the amino acid 596R.

A preferred mutein of the invention is one wherein the conserved amino acid affected by said mutation is the amino acid 597G.

A preferred mutein of the invention is one wherein the conserved amino acid affected by said mutation is the amino acid 598A.

A preferred mutein of the invention is one wherein the conserved amino acid affected by said mutation is the amino acid 599I.

A preferred mutein of the invention is one wherein the conserved amino acid affected by said mutation is the amino acid 601E.

A preferred mutein of the invention is one wherein the conserved amino acid affected by said mutation is the amino acid 602Y.

A preferred mutein of the invention is one wherein the conserved amino acid affected by said mutation is the amino acid 603Q.

A preferred mutein of the invention is one wherein the conserved amino acid affected by said mutation is the amino acid 604T.

A preferred mutein of the invention is one wherein the conserved amino acid affected by said mutation is the amino acid 606L.

A preferred mutein of the invention is one wherein the conserved amino acid affected by said mutation is the amino acid 607I.

A preferred mutein of the invention is one wherein the conserved amino acid affected by said mutation is the amino acid 609R.

A preferred mutein of the invention is one wherein the conserved amino acid affected by said mutation is the amino acid 610V.

A preferred mutein of the invention is one wherein the conserved amino acid affected by said mutation is the amino acid 611K.

A preferred mutein of the invention is one wherein the conserved amino acid affected by said mutation is the amino acid 613D.

A preferred mutein of the invention is one wherein the conserved amino acid affected by said mutation is the amino acid 614I.

A preferred mutein of the invention is one wherein the conserved amino acid affected by said mutation is the amino acid 617L.

A preferred mutein of the invention is one wherein the conserved amino acid affected by said mutation is the amino acid 621F.

A preferred mutein of the invention is one wherein the conserved amino acid affected by said mutation is the amino acid 639L.

A preferred mutein of the invention is one wherein the conserved amino acid affected by said mutation is the amino acid 640P.

A preferred mutein of the invention is one wherein the conserved amino acid affected by said mutation is the amino acid 641P.

A preferred mutein of the invention is one wherein the conserved amino acid affected by said mutation is the amino acid 646W.

A preferred mutein of the invention is one wherein the conserved amino acid affected by said mutation is the amino acid 655Q.

A preferred mutein of the invention is one wherein the conserved amino acid affected by said mutation is the amino acid 656L.

A preferred mutein of the invention is one wherein the conserved amino acid affected by said mutation is the amino acid 659Y.

A preferred mutein of the invention is one wherein the conserved amino acid affected by said mutation is the amino acid 661K.

A preferred mutein of the invention is one wherein the conserved amino acid affected by said mutation is the amino acid 662R.

A preferred mutein of the invention is one wherein the conserved amino acid affected by said mutation is the amino acid 663V.

A preferred mutein of the invention is one wherein the conserved amino acid affected by said mutation is the amino acid 664E.

A preferred mutein of the invention is one wherein the conserved amino acid affected by said mutation is the amino acid 665D.

A preferred mutein of the invention is one wherein the conserved amino acid affected by said mutation is the amino acid 666V.

A preferred mutein of the invention is one wherein the conserved amino acid affected by said mutation is the amino acid 667L.

A preferred mutein of the invention is one wherein the conserved amino acid affected by said mutation is the amino acid 668G.

A preferred mutein of the invention is one wherein the conserved amino acid affected by said mutation is the amino acid 669K.

A preferred mutein of the invention is one wherein the conserved amino acid affected by said mutation is the amino acid 671W.

A preferred mutein of the invention is one wherein the conserved amino acid affected by said mutation is the amino acid 672E.

A preferred mutein of the invention is one wherein the conserved amino acid affected by said mutation is the amino acid 674H.

A preferred mutein of the invention is one wherein the conserved amino acid affected by said mutation is the amino acid 677G.

A preferred mutein of the invention is one wherein the conserved amino acid affected by said mutation is the amino acid 680L.

A preferred mutein of the invention is one wherein the conserved amino acid affected by said mutation is the amino acid 681K.

A preferred mutein of the invention is one wherein the conserved amino acid affected by said mutation is the amino acid 683D.

A preferred mutein of the invention is one wherein the conserved amino acid affected by said mutation is the amino acid 684G.

A preferred mutein of the invention is one wherein the conserved amino acid affected by said mutation is the amino acid 685D.

A preferred mutein of the invention is one wherein the conserved amino acid affected by said mutation is the amino acid 687F.

A preferred mutein of the invention is one wherein the conserved amino acid affected by said mutation is the amino acid 690F.

A preferred mutein of the invention is one wherein the conserved amino acid affected by said mutation is the amino acid 691L.

A preferred mutein of the invention is one wherein the conserved amino acid affected by said mutation is the amino acid 697F.

A preferred mutein of the invention is one wherein the conserved amino acid affected by said mutation is the amino acid 700W.

A preferred mutein of the invention is one wherein the conserved amino acid affected by said mutation is the amino acid 704V.

A preferred mutein of the invention is one wherein the conserved amino acid affected by said mutation is the amino acid 705N.

A preferred mutein of the invention is one wherein the conserved amino acid affected by said mutation is the amino acid 715I.

A preferred mutein of the invention is one wherein the conserved amino acid affected by said mutation is the amino acid 717T.

A preferred mutein of the invention is one wherein the conserved amino acid affected by said mutation is the amino acid 731L.

A preferred mutein of the invention is one wherein the conserved amino acid affected by said mutation is the amino acid 733L.

A preferred mutein of the invention is one wherein the conserved amino acid affected by said mutation is the amino acid 736N.

A preferred mutein of the invention is one wherein the conserved amino acid affected by said mutation is the amino acid 744L.

A preferred mutein of the invention is one wherein the conserved amino acid affected by said mutation is the amino acid 746K.

A preferred mutein of the invention is one wherein the conserved amino acid affected by said mutation is the amino acid 747E.

A preferred mutein of the invention is one wherein the conserved amino acid affected by said mutation is the amino acid 748V.

A preferred mutein of the invention is one wherein the conserved amino acid affected by said mutation is the amino acid 752K.

A preferred mutein of the invention is one wherein the conserved amino acid affected by said mutation is the amino acid 755G.

A preferred mutein of the invention is one wherein the conserved amino acid affected by said mutation is the amino acid 756F.

A preferred mutein of the invention is one wherein the conserved amino acid affected by said mutation is the amino acid 757R.

A preferred mutein of the invention is one wherein the conserved amino acid affected by said mutation is the amino acid 758V.

A preferred mutein of the invention is one wherein the conserved amino acid affected by said mutation is the amino acid 759P.

A preferred mutein of the invention is one wherein the conserved amino acid affected by said mutation is the amino acid 760L.

A preferred mutein of the invention is one wherein the conserved amino acid affected by said mutation is the amino acid 762I.

A preferred mutein of the invention is one wherein the conserved amino acid affected by said mutation is the amino acid 763V.

A preferred mutein of the invention is one wherein the conserved amino acid affected by said mutation is the amino acid 764N.

A preferred mutein of the invention is one wherein the conserved amino acid affected by said mutation is the amino acid 766A.

A preferred mutein of the invention is one wherein the conserved amino acid affected by said mutation is the amino acid 767H.

A preferred mutein of the invention is one wherein the conserved amino acid affected by said mutation is the amino acid 768Q.

A preferred mutein of the invention is one wherein the conserved amino acid affected by said mutation is the amino acid 769A.

A preferred mutein of the invention is one wherein the conserved amino acid affected by said mutation is the amino acid 770N.

A preferred mutein of the invention is one wherein the conserved amino acid affected by said mutation is the amino acid 771Q.

A preferred mutein of the invention is one wherein the conserved amino acid affected by said mutation is the amino acid 774P.

A preferred mutein of the invention is one wherein the conserved amino acid affected by said mutation is the amino acid 776A.

A preferred mutein of the invention is one wherein the conserved amino acid affected by said mutation is the amino acid 778S.

A preferred mutein of the invention is one wherein the conserved amino acid affected by said mutation is the amino acid 779L.

A preferred mutein of the invention is one wherein the conserved amino acid affected by said mutation is the amino acid 780I.

A preferred mutein of the invention is one wherein the conserved amino acid affected by said mutation is the amino acid 781E.

A preferred mutein of the invention is one wherein the conserved amino acid affected by said mutation is the amino acid 784R.

A preferred mutein of the invention is one wherein the conserved amino acid affected by said mutation is the amino acid 785T.

A preferred mutein of the invention is one wherein the conserved amino acid affected by said mutation is the amino acid 802L.

A preferred mutein of the invention is one wherein the conserved amino acid affected by said mutation is the amino acid 804A.

A preferred mutein of the invention is one wherein the conserved amino acid affected by said mutation is the amino acid 808K.

A preferred mutein of the invention is one wherein the conserved amino acid affected by said mutation is the amino acid 816E.

A preferred mutein of the invention is one wherein the conserved amino acid affected by said mutation is the amino acid 817G.

A preferred mutein of the invention is one wherein the conserved amino acid affected by said mutation is the amino acid 820L.

A preferred mutein of the invention is one wherein the conserved amino acid affected by said mutation is the amino acid 822W.

A preferred mutein of the invention is one wherein the conserved amino acid affected by said mutation is the amino acid 824S.

A preferred mutein of the invention is one wherein the conserved amino acid affected by said mutation is the amino acid 825Y.

A preferred mutein of the invention is one wherein the conserved amino acid affected by said mutation is the amino acid 826K.

A preferred mutein of the invention is one wherein the conserved amino acid affected by said mutation is the amino acid 828D.

A preferred mutein of the invention is one wherein the conserved amino acid affected by said mutation is the amino acid 830Y.

A preferred mutein of the invention is one wherein the conserved amino acid affected by said mutation is the amino acid 834L.

A preferred mutein of the invention is one wherein the conserved amino acid affected by said mutation is the amino acid 836E.

A preferred mutein of the invention is one wherein the conserved amino acid affected by said mutation is the amino acid 838V.

A preferred mutein of the invention is one wherein the conserved amino acid affected by said mutation is the amino acid 842Q.

A preferred mutein of the invention is one wherein the conserved amino acid affected by said mutation is the amino acid 843E.

A preferred mutein of the invention is one wherein the conserved amino acid affected by said mutation is the amino acid 848L.

A preferred mutein of the invention is one wherein the conserved amino acid affected by said mutation is the amino acid 849L.

A preferred mutein of the invention is one wherein the conserved amino acid affected by said mutation is the amino acid 862L.

A preferred mutein of the invention is one wherein the conserved amino acid affected by said mutation is the amino acid 865C.

A preferred mutein of the invention is one wherein the conserved amino acid affected by said mutation is the amino acid 867Y.

A preferred mutein of the invention is one wherein the conserved amino acid affected by said mutation is the amino acid 871T.

A preferred mutein of the invention is one wherein the conserved amino acid affected by said mutation is the amino acid 876L.

A preferred mutein of the invention is one wherein the conserved amino acid affected by said mutation is the amino acid 880Q.

A preferred mutein of the invention is one wherein the conserved amino acid affected by said mutation is the amino acid 883V.

A preferred mutein of the invention is one wherein the conserved amino acid affected by said mutation is the amino acid 884D.

A preferred mutein of the invention is one wherein the conserved amino acid affected by said mutation is the amino acid 886L.

A preferred mutein of the invention is one wherein the conserved amino acid affected by said mutation is the amino acid 888L.

A preferred mutein of the invention is one wherein the conserved amino acid affected by said mutation is the amino acid 891Y.

A preferred mutein of the invention is one wherein the conserved amino acid affected by said mutation is the amino acid 892S.

A preferred mutein of the invention is one wherein the conserved amino acid affected by said mutation is the amino acid 893N.

A preferred mutein of the invention is one wherein the conserved amino acid affected by said mutation is the amino acid 894L.

A preferred mutein of the invention is one wherein the conserved amino acid affected by said mutation is the amino acid 897W.

A preferred mutein of the invention is one wherein the conserved amino acid affected by said mutation is the amino acid 898V.

A preferred mutein of the invention is one wherein the conserved amino acid affected by said mutation is the amino acid 899N.

A preferred mutein of the invention is one wherein the conserved amino acid affected by said mutation is the amino acid 901L.

A preferred mutein of the invention is one wherein the conserved amino acid affected by said mutation is the amino acid 902D.

A preferred mutein of the invention is one wherein the conserved amino acid affected by said mutation is the amino acid 906E.

A preferred mutein of the invention is one wherein the conserved amino acid affected by said mutation is the amino acid 909L.

A preferred mutein of the invention is one wherein the conserved amino acid affected by said mutation is the amino acid 912R.

A preferred mutein of the invention is one wherein the conserved amino acid affected by said mutation is the amino acid 920W.

A preferred mutein of the invention is one wherein the conserved amino acid affected by said mutation is the amino acid 921T.

A preferred mutein of the invention is one wherein the conserved amino acid affected by said mutation is the amino acid 951P.

A preferred mutein of the invention is one wherein the conserved amino acid affected by said mutation is the amino acid 955N.

A preferred mutein of the invention is one wherein the conserved amino acid affected by said mutation is the amino acid 957V.

A preferred mutein of the invention is one wherein the conserved amino acid affected by said mutation is the amino acid 963T.

A preferred mutein of the invention is one wherein the conserved amino acid affected by said mutation is the amino acid 965Q.

A preferred mutein of the invention is one wherein the conserved amino acid affected by said mutation is the amino acid 968Y.

A preferred mutein of the invention is one wherein the conserved amino acid affected by said mutation is the amino acid 971P.

A preferred mutein of the invention is one wherein the conserved amino acid affected by said mutation is the amino acid 975E.

A preferred mutein of the invention is one wherein the conserved amino acid affected by said mutation is the amino acid 977R.

A preferred mutein of the invention is one wherein the conserved amino acid affected by said mutation is the amino acid 987W.

A preferred mutein of the invention is one wherein the conserved amino acid affected by said mutation is the amino acid 1001R.

A preferred mutein of the invention is one wherein the conserved amino acid affected by said mutation is the amino acid 1003Q.

A preferred mutein of the invention is one wherein the conserved amino acid affected by said mutation is the amino acid 1017Y.

A preferred mutein of the invention is one wherein the conserved amino acid affected by said mutation is the amino acid 1019N.

A preferred mutein of the invention is one wherein the conserved amino acid affected by said mutation is the amino acid 1021L.

A preferred mutein of the invention is one wherein the conserved amino acid affected by said mutation is the amino acid 1025P.

A preferred mutein of the invention is one wherein the conserved amino acid affected by said mutation is the amino acid 1027G.

A preferred mutein of the invention is one wherein the conserved amino acid affected by said mutation is the amino acid 1031L.

A preferred mutein of the invention is one wherein the conserved amino acid affected by said mutation is the amino acid 1032E.

A preferred mutein of the invention is one wherein the conserved amino acid affected by said mutation is the amino acid 1035Y.

A preferred mutein of the invention is one wherein the conserved amino acid affected by said mutation is the amino acid 1048Y.

A preferred mutein of the invention is one wherein the conserved amino acid affected by said mutation is the amino acid 1052W.

A preferred mutein of the invention is one wherein the conserved amino acid affected by said mutation is the amino acid 1053L.

A preferred mutein of the invention is one wherein the conserved amino acid affected by said mutation is the amino acid 1055Y.

A preferred mutein of the invention is one wherein the conserved amino acid affected by said mutation is the amino acid 1056Q.

A preferred mutein of the invention is one wherein the conserved amino acid affected by said mutation is the amino acid 1058L.

A preferred mutein of the invention is one wherein the conserved amino acid affected by said mutation is the amino acid 1059W.

A preferred mutein of the invention is one wherein the conserved amino acid affected by said mutation is the amino acid 1062Q.

A preferred mutein of the invention is one wherein the conserved amino acid affected by said mutation is the amino acid 1063A.

A preferred mutein of the invention is one wherein the conserved amino acid affected by said mutation is the amino acid 1070L.

A preferred mutein of the invention is one wherein the conserved amino acid affected by said mutation is the amino acid 1071G.

A preferred mutein of the invention is one wherein the conserved amino acid affected by said mutation is the amino acid 1077W.

A preferred mutein of the invention is one wherein the conserved amino acid affected by said mutation is the amino acid 1081L.

A preferred mutein of the invention is one wherein the conserved amino acid affected by said mutation is the amino acid 1084I.

A preferred mutein of the invention is one wherein the conserved amino acid affected by said mutation is the amino acid 1088R.

A preferred mutein of the invention is one wherein the conserved amino acid affected by said mutation is the amino acid 1091F.

A preferred mutein of the invention is one wherein the conserved amino acid affected by said mutation is the amino acid 1092D.

A preferred mutein of the invention is one wherein the conserved amino acid affected by said mutation is the amino acid 1096T.

A preferred mutein of the invention is one wherein the conserved amino acid affected by said mutation is the amino acid 1102P.

A preferred mutein of the invention is one wherein the conserved amino acid affected by said mutation is the amino acid 1107Y.

A preferred mutein of the invention is one wherein the conserved amino acid affected by said mutation is the amino acid 1109K.

A preferred mutein of the invention is one wherein the conserved amino acid affected by said mutation is the amino acid 1111Q.

A preferred mutein of the invention is one wherein the conserved amino acid affected by said mutation is the amino acid 1113K.

A preferred mutein of the invention is one wherein the conserved amino acid affected by said mutation is the amino acid 1117K.

A preferred mutein of the invention is one wherein the conserved amino acid affected by said mutation is the amino acid 1118Y.

A preferred mutein of the invention is one wherein the conserved amino acid affected by said mutation is the amino acid 1119D.

A preferred mutein of the invention is one wherein the conserved amino acid affected by said mutation is the amino acid 1121W.

A preferred mutein of the invention is one wherein the conserved amino acid affected by said mutation is the amino acid 1122H.

A preferred mutein of the invention is one wherein the conserved amino acid affected by said mutation is the amino acid 1123K.

A preferred mutein of the invention is one wherein the conserved amino acid affected by said mutation is the amino acid 1124E.

A preferred mutein of the invention is one wherein the conserved amino acid affected by said mutation is the amino acid 1126L.

A preferred mutein of the invention is one wherein the conserved amino acid affected by said mutation is the amino acid 1128K.

A preferred mutein of the invention is one wherein the conserved amino acid affected by said mutation is the amino acid 1129F.

A preferred mutein of the invention is one wherein the conserved amino acid affected by said mutation is the amino acid 1130G.

A preferred mutein of the invention is one wherein the conserved amino acid affected by said mutation is the amino acid 1134G.

A preferred mutein of the invention is one wherein the conserved amino acid affected by said mutation is the amino acid 1137W.

A preferred mutein of the invention is one wherein the conserved amino acid affected by said mutation is the amino acid 1140F.

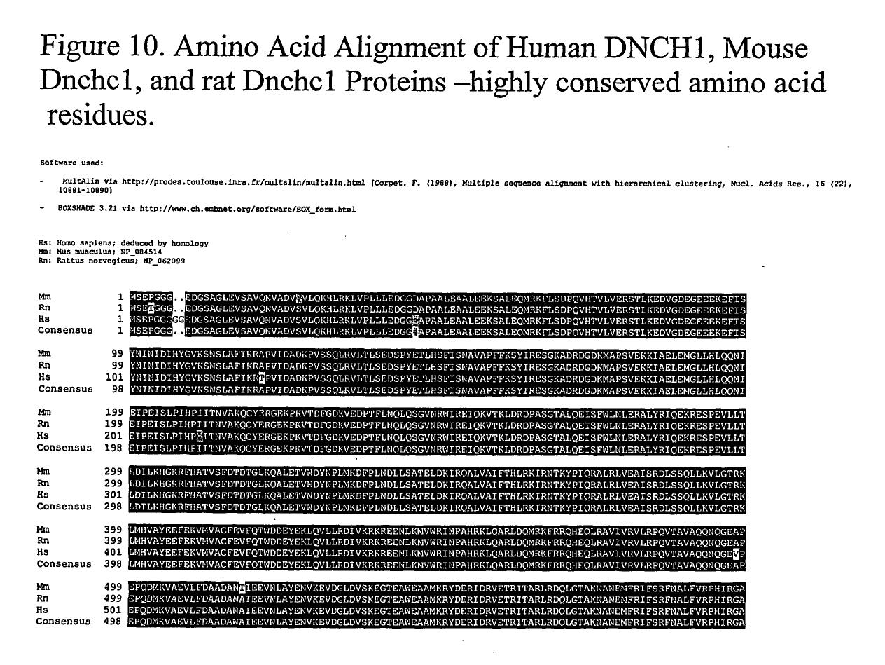

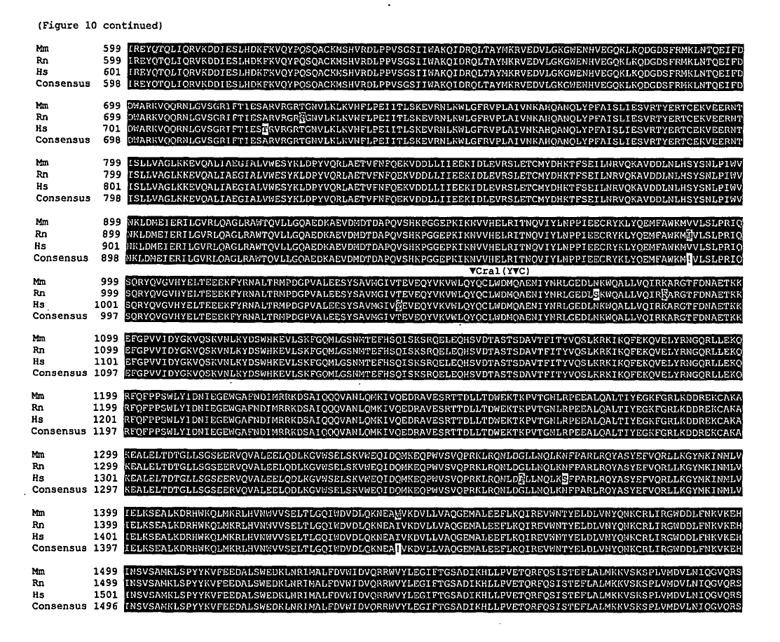

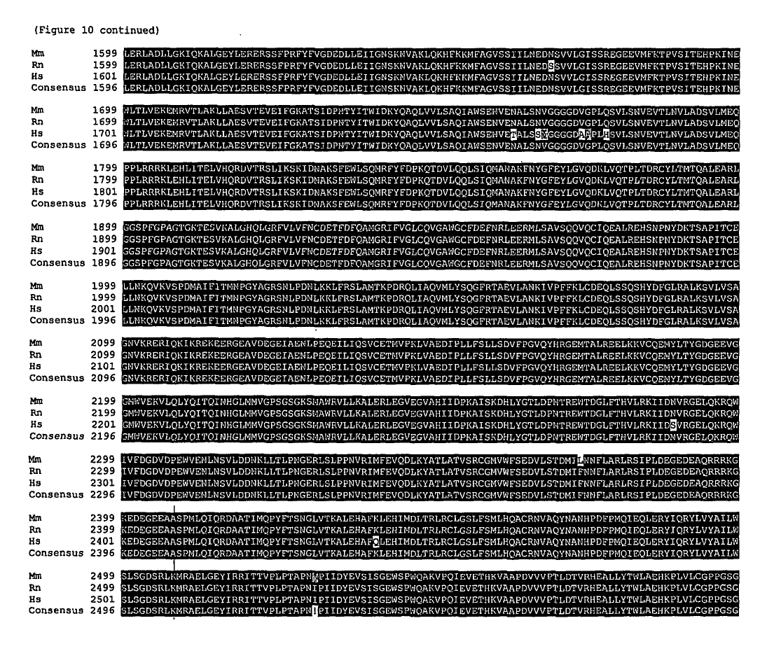

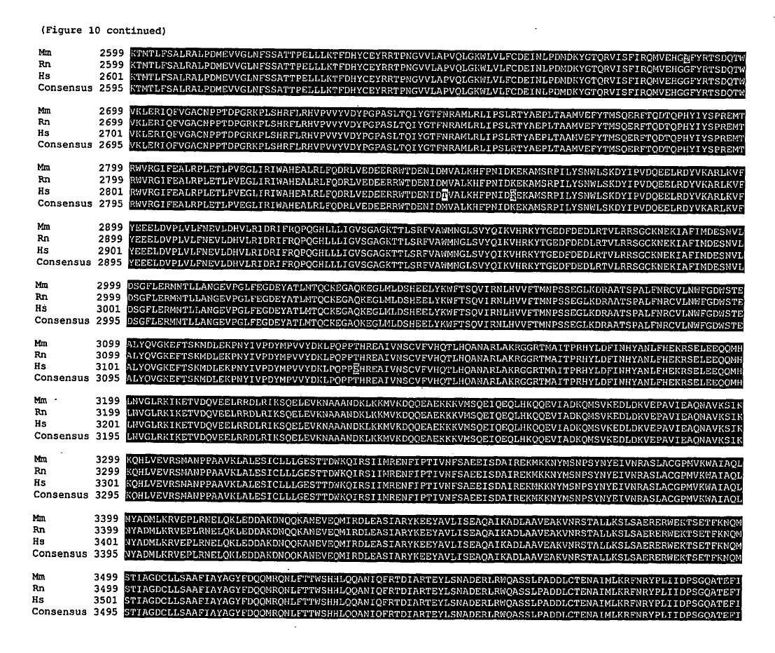

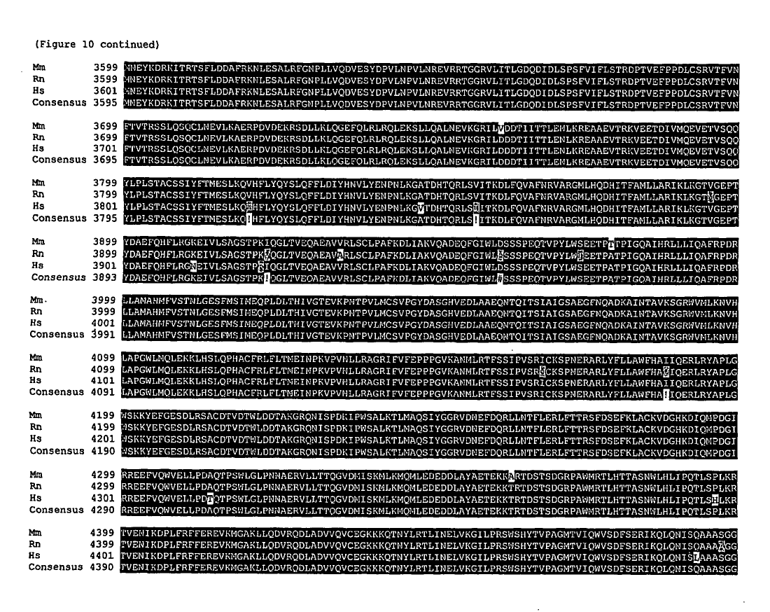

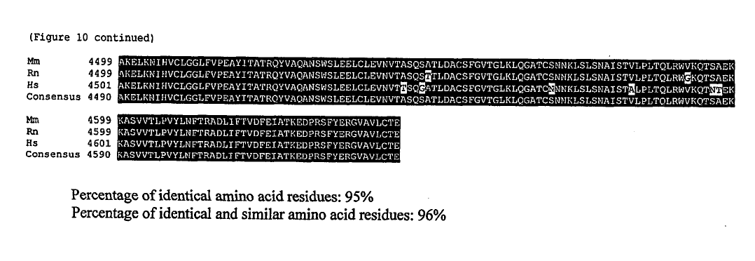

It will be understood that human muteins of the cytoplasmic dynein heavy chain1 having mutations as described herein, e.g., carrying a substitution, deletion, or insertion at a position within the human amino acid sequence corresponding to any of the above individualized conserved amino acids of the mouse amino acid sequence, are likewise within the scope of the invention. The corresponding human amino acids will, as they are conserved between, e.g. mouse and human, be identical while their relative position within the amino acid sequence may vary. The skilled person will be readily able to identify the corresponding human amino acids, e.g., within SEQ ID NO:18, or from FIG. 9 or 10.

In addition to the mutant proteins described by SEQ ID NO:4, and SEQ ID NO:6, the skilled artisan will further appreciate that changes can be introduced by mutation into the nucleotide sequence encoding a dynein heavy chain polypeptide (e.g., the sequences of SEQ ID NO:3 and SEQ ID NO:5), thereby leading to changes in the amino acid sequence of the encoded dynein heavy chain proteins without altering properties of the mutant dynein heavy chain proteins. For example, nucleotide substitutions leading to amino acid substitutions at “non-essential” amino acid residues can be made in the sequence of SEQ ID NO:3 and SEQ ID NO:5. A “non-essential” amino acid residue is a residue that can be altered from the wild-type sequence of a dynein heavy chain polypeptide without altering the biological activity, whereas an “essential” amino acid residue is required for biological activity. For example, amino acid residues that are conserved among the cytoplasmic dynein heavy chain1 polypeptide or polypeptide fragments of the present invention are predicted to be particularly unamenable to alteration.

Preferably, such alterations replace an amino acid with one of similar size and polarity (e.g., conservative substitution). In one embodiment, the polypeptide has at least 75%, preferably at least 80%, more preferably at least 90%, even more preferably 95% and most preferably at least 99% sequence identity with the wild type cytoplasmic dynein heavy chain1 sequence. In a particularly preferred embodiment, the polypeptide is identical with the wild type sequence except for a replacement of the Tyr residue at position 1055 of the amino acid sequence, as shown in SEQ ID NO: 4. In another particularly preferred embodiment, the polypeptide is identical to the wild type sequence except for a replacement of the Tyr residue at position 1057 of the amino acid sequence, as shown in SEQ ID NO: 6 or at the corresponding position in other cytoplasmic dynein heavy chain1 sequences. Preferred modifications of the amino acid sequence, in addition to the replacement at, e.g., position 1055 of the amino acid sequence shown in SEQ ID NO: 4, and at, e.g., position 1057 of the amino acid sequence shown in SEQ ID NO: 6, or at a corresponding position in other dynein heavy chains are at any of the positions which are conserved among the vertebrate dynein heavy chains as described in individualized manner in connection with the preferred muteins above.

Preferred modifications of the amino acid sequence, in addition to the replacement at position 1055 of the amino acid sequence shown in SEQ ID NO: 4, and at position 1057 of the amino acid sequence shown in SEQ ID NO: 6, or at a corresponding position in other dynein heavy chains are at positions which are not conserved among the vertebrate dynein heavy chains.

Amino acids in the cytoplasmic dynein heavy chain 1 proteins of the present invention that are essential for function can be identified by methods known in the art, such as site-directed mutagenesis or alanine-scanning mutagenesis (Cunningham and Wells, Science 244:1081-1085, 1989). The latter procedure introduces single alanine mutations at every residue in the molecule. The resulting mutant molecules are then tested for biological activity such as receptor binding or in vitro proliferative activity. Sites that are critical for ligand-receptor binding can also be determined by structural analysis such as crystallization, nuclear magnetic resonance or photoaffinity labeling (Smith et al., J. Mol. Biol. 224:899-904, 1992; de Vos et al., Science 255:306-312, 1992).

An “isolated” or “purified” polypeptide or protein or biologically-active portion thereof is substantially free of cellular material or other contaminating proteins from the cell or tissue source from which the polypeptide or protein is derived, or substantially free from chemical precursors or other chemicals when chemically synthesized. The language “substantially free of cellular material” includes preparations of dynein heavy chain protein in which the protein is separated from cellular components of the cells from which it the protein is isolated or in which it is recombinantly produced.

Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second comparison amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (e.g., as used herein amino acid or nucleic acid “homology” is equivalent to amino acid or nucleic acid “identity”). The nucleic acid sequence homology may be determined as the degree of identity between two sequences. The homology may be determined using computer programs known in the art, such as GAP software provided in the GCG program package. See, Needleman and Wunsch (1970) J Mol Biol 48, 443453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS:1, 3, 5, and 17.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (e.g. the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

The phrases “percent identity” or “% identity” refer to the percentage of sequence similarity found in a comparison of two or more amino acid or nucleic acid sequences. Percent identity can be determined electronically, e.g., by using the MEGALIGN program (DNASTAR, Inc., Madison Wis., USA). The MEGALIGN program can create alignments between two or more sequences according to different methods, e.g., the clustal method (see, for example, Higgins and Sharp (1988) Gene 73:237-244. The clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low homology or of no homology between the two amino acid sequences are not included in determining percentage similarity. Percent identity between nucleic acid sequences can also be counted or calculated by other methods known in the art, e.g., the method described in Hein (1990) Methods Enzymol 183, 626-645. Identity between sequences can also be determined by other methods known in the art, e.g. by varying hybridization conditions.

The comparison of two or more amino acid or nucleic acid sequences to determine sequence identity can be performed between ortholog sequences, preferably between mouse and human, more preferably between mouse, rat, and human sequences. When a position of an amino acid or nucleotide in one ortholog sequence is occupied by the same amino acid or nucleotide in at least a second ortholog sequence, this amino acid or nucleotide is “evolutionary conserved” for the purpose of this invention. The term “evolutionary conserved” also comprises amino acid substitutions, where an amino acid is replaced by another (i.e., different) amino acid that represents a conservative substitution as defined below.

When, e.g., residue 1055 of SEQ ID NO:2, or residue 1057 of SEQ ID NO:18 or any of the residues corresponding to the conserved residues described in individualized manner in connection with the preferred muteins of the invention above is replaced by an amino acid with different size and/or polarity (excluding the wild type residue at this position), this is termed a non-conservative amino acid substitution. Non-conservative substitutions are defined as exchanges of an amino acid by another amino acid listed in a different group of the five standard amino acid groups shown below:
1. small aliphatic, nonpolar or slightly polar residues: Ala, Ser, Thr, (Pro), (Gly);
2. negatively charged residues and their amides: Asn, Asp, Glu, Gln;
3. positively charged residues: His, Arg, Lys;
4. large aliphatic, nonpolar residues: Met, Leu, Ile, Val, (Cys);
5. large aromatic residues: Phe, Tyr, Trp.

Conservative substitutions are defined as exchanges of an amino acid by another amino acid listed within the same group of the five standard amino acid groups shown above. Three residues are parenthesized because of their special role in protein architecture. Gly is the only residue without a sidechain and therefore imparts flexibility to the chain. Pro has an unusual geometry which tightly constrains the chain. Cys can participate in disulfide bonds.

In a preferred embodiment, the cytoplasmic dynein heavy chain1 expressed in the animal model of the present invention carries a mutation, which affects the conserved amino acids of the muteins described in individualized manner in connection with the preferred muteins of the invention above.

In the most preferred embodiment, the cytoplasmic dynein heavy chain1 expressed in the animal model of the present invention has the amino acid sequence shown in SEQ ID NO:4.

In another most preferred embodiment, the cytoplasmic dynein heavy chain1 expressed in the animal model of the present invention has an amino acid sequence selected from the sequences shown in SEQ ID NO:6 and SEQ ID NO: 18.

The invention also provides novel chimeric or fusion proteins as used herein, a novel “chimeric protein” or “fusion protein” comprises a novel cytoplasmic dynein heavy chain1 polypeptide linked to a non-cytoplasmic dynein heavy chain1 polypeptide (e.g., a polypeptide that does not comprise cytoplasmic dynein heavy chain1 or a fragment therof).

In one embodiment, the fusion protein is a GST-cytoplasmic dynein heavy chain1 fusion protein in which the cytoplasmic dynein heavy chain1 sequences are fused to the C-terminus of the GST (glutathione S-transferase) sequences. Such fusion proteins can facilitate the purification of recombinant cytoplasmic dynein heavy chain1 polypeptides.

In yet another embodiment, the fusion protein is a cytoplasmic dynein heavy chain1-immunoglobulin fusion protein in which the cytoplasmic dynein heavy chain1 sequences are fused to sequences derived from a member of the immunoglobulin protein family, especially Fc region polypeptides. Also contemplated are fusions of cytoplasmic dynein heavy chain1 sequences (mutant or wild type or functional fragments) fused to amino acid sequences that are commonly used to facilitate purification or labeling, e.g. polyhistidine tails (especially hexahistidine segments), FLAG tags, streptavidin.

The amino acid sequences of the present invention may be made by using peptide synthesis techniques well known in the art, such as solid phase peptide synthesis (see, for example, Fields et al. “Principles and Practice of Solid Phase Synthesis” in Synthetic Peptides, A Users Guide, Grant G A (ed.), (W.H. Freeman Co. New York N.Y., USA, 1992) at Chapter 3, pp. 77-183; Barlos K and Gatos D “Convergent Peptide Synthesis” in FMOC Solid Phase Peptide Synthesis, Chan W C and White P D (eds.), (Oxford University Press, New York N.Y., USA, 2000) at Chapter 9, pp. 215-228) or by recombinant DNA manipulations and recombinant expression. Techniques for making substitution mutations at predetermined sites in DNA having known sequence are well known and include, for example, M13 mutagenesis. Manipulation of DNA sequences to produce variant proteins which manifest as substitution, insertion or deletion variants are conveniently described, for example, in Sambrook et al. (1989), supra.

Antibodies

Also included in the invention are antibodies to fragments of cytoplasmic dynein heavy chain1 polypeptides and muteins as described in individualized manner in connection with the preferred muteins of the invention above (including amino terminal fragments), as well as antibodies to fusion proteins containing cytoplasmic dynein heavy chain1 polypeptides or fragments of cytoplasmic dynein heavy chain1 polypeptides or muteins as described in individualized manner in connection with the preferred muteins of the invention above. The term “antibody” as used herein refers to immunoglobulin molecules and immunologically active portions of immunoglobulin (Ig) molecules, e.g., molecules that contain an antigen binding site that specifically binds (immunoreacts with) an antigen. Such antibodies include, e.g., polyclonal, monoclonal, chimeric, single chain, F_{ab}, F_{ab}, and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG2, and others. Furthermore, in humans, the light chain may be a kappa chain or a lambda chain. Reference herein to antibodies includes a reference to all such classes, subclasses and types of human antibody species.

A cytoplasmic dynein heavy chain1 polypeptide 1 of the invention may be intended to serve as an antigen, or a portion or fragment thereof, and additionally can be used as an immunogen to generate antibodies that immunospecifically bind the antigen, using standard techniques for polyclonal and monoclonal antibody preparation. Antigenic peptide fragments of the antigen for use as immunogens includes, e.g., at least 7 amino acid residues of the amino acid sequence of the mutated region such as an amino acid sequence shown in SEQ ID NO:4 or SEQ ID NO:6, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of cytoplasmic dynein heavy chain1 polypeptide that is located on the surface of the protein, e.g., a hydrophilic region. A hydrophobicity analysis of a cytoplasmic dynein heavy chain1 polypeptide will indicate which regions of a cytoplasmic dynein heavy chain1 protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. (See, for example, Hopp and Woods (1981) Proc Nat Acad Sci USA 78, 3824-3828; Kyte and Doolittle (1982) J Mol Biol 157, 105-142.) Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof. (See, for example, Harlow and Lane Antibodies: A Laboratory Manual, (Cold Spring Harbor Laboratory Press, Cold Spring Harbor, N.Y., USA, 1988).) Some of these antibodies are discussed below.

Polyclonal Antibodies

For the production of polyclonal antibodies, various suitable host animals (e.g., rabbit, goat, mouse or other mammal) may be immunized by one or more injections with the protein of the invention, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin and soybean trypsin inhibitor.

The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by Wilkinson (Wilkinson (2000) The Scientist, 14, 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen, the antigen binding site being characterized by its unique binding affinity for this epitope. Similarly, the epitope may be characterized by its binding affinity for antigen binding sites of one or more particular MAbs having binding sites directed towards that epitope.

Monoclonal antibodies can be prepared using hybridoma methods, such as those described by Kohler and Milstein (1975) Nature 256, 495. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice (Academic Press/Elsevier Science Sidcup, Kent, UK, 1986) at pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor (1984) J Immunol, 133, 3001; Brodeur et al., Monoclonal Antibody Production Techniques and Applications (Marcel Dekker, Inc., New York N.Y., USA, 1987) at pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard (1980) Anal. Biochem., 107:220. Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison (1994) Nature 368, 812-13) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin and contain minimal sequence derived from a non-human immunoglobulin. Humanization can be performed following the method of Winter and co-workers (Jones et al. (1986) Nature 321, 522-525; Riechmann et al. (1988) Nature 332, 323-327; Verhoeyen et al. (1988) Science 239, 1534-1536), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al. (1986) Nature 321, 522-525; Riechmann et al. (1988) Nature 332, 323-327; and Presta (1992) Curr Op Struct Biol 2, 593-596).

Human Antibodies

Fully human antibodies relate to antibody molecules in which essentially the entire sequences of both the light chain and the heavy chain, including the CDRs, arise from human genes. Such antibodies are termed “human antibodies”, or “fillly human antibodies” herein. Human monoclonal antibodies can be prepared by the trioma technique; the human B-cell hybridoma technique (see Kozbor et al. (1983) Immunol Today 4, 72) and the EBV hybridoma technique to produce human monoclonal antibodies (see Cole et al. in: Monoclonal Antibodies and Cancer Therapy (Alan R. Liss, Inc., New York N.Y., USA, 1985) at pp. 77-96). Human monoclonal antibodies may be utilized in the practice of the present invention and may be produced by using human hybridomas (see Cote et al. (1983) Proc Natl Acad Sci USA 80, 2026-2030) or by transforming human B-cells with Epstein Barr Virus in vitro (see Cole et al. in: Monoclonal Antibodies and Cancer Therapy (Alan R. Liss, Inc., New York N.Y., USA, 1985) at pp. 77-96).

In addition, human antibodies can also be produced using additional techniques, including phage display libraries (Hoogenboom and Winter (1992) J Mol Biol 227, 381-388; Marks et al. (1991) J Mol Biol 222, 581-597). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (1992) Bio/Technology 10, 779-783; Lonberg et al. (1994) Nature 368, 856-859; Morrison (1994) Nature 368, 812-13; Fishwild et al. (1996) Nature Biotechnology 14, 845-51; Neuberger (1996) Nature Biotechnology 14, 826; and Lonberg and Huszar (1995) Intern Rev Immunol 13, 65-93.

Human antibodies may additionally be produced using transgenic nonhuman animals which are modified so as to produce fully human antibodies rather than the animal's endogenous antibodies in response to challenge by an antigen (see PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO-A-96/33735 and WO-A-96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. Such a host can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. The method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO-A-99/53049.

F_{ab} Fragments and Single Chain Antibodies

According to the invention, techniques can be adapted for the production of single-chain antibodies specific to an antigenic protein of the invention (see for example U.S. Pat. No. 4,946,778). In addition, methods can be adapted for the construction of F_{ab} expression libraries (see for example Huse et al. (1989) Science 246, 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to:

(i) an F_{(ab′)2} fragment produced by pepsin digestion of an antibody molecule;

(ii) an F_{ab} fragment generated by reducing the disulfide bridges of an F_{(ab′)2} fragment;

(iii) an F_{ab} fragment generated by the treatment of the antibody molecule with papain and a reducing agent; and

(iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello (1983) Nature 305, 537-539). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO-A-93/08829 and in Traunecker et al. (1991) EMBO J 10, 3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy chain constant region (CH1) containing the site necessary for light-chain binding present in at least one of the fusions. DNAs encoding the immunoglobulin heavy-chain fusions and, if desired, the immunoglobulin light chain are inserted into separate expression vectors and are co-transfected into a suitable host organism. For further details of generating bispecific antibodies see, for example, Suresh et al. (1986) Methods in Enzymology 121, 210.

In accordance with another approach described in WO-A-96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al. ((1985) Science 229, 81) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be recovered directly from E. coli and chemically coupled to form bispecific antibodies. Shalaby et al. ((1992) J Exp Med 175, 217-225) describe the production of a fully humanized bispecific antibody F(ab′)₂ molecule. Each Fab′ fragment was secreted separately from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al. (1992) J Immunol 148, 1547-1553. The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al. ((1993) Proc Natl Acad Sci USA 90, 6444-6448) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported (see Gruber et al. (1994) J Immunol 152, 5368-74.

Antibodies with more than two valencies are contemplated. For example, trispecific antibodies can be prepared (Tutt et al. (1991) J Immunol 147, 60-69).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (Fc R), such as Fc RI (CD64), Fc RII (CD32) and Fc RIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO-A-91/00360; WO-A-92/20373; EP-A3-0308936). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC) (see Caron et al. (1992) J Exp Med 176; 1191-1195 and Shopes (1992) J Immunol 148, 2918-2922). Homodimeric antibodies with enhanced anti-tumor activity can also be prepared using heterobifunctional cross-linkers as described in Wolff et al. (1993) Cancer Res 53, 2560-2565. Alternatively, an antibody can be engineered that has dual Fc regions and can thereby have enhanced complement lysis and ADCC capabilities (see Stevenson et al. (1989) Anti-Cancer Drug Design 3, 219-230).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (e.g., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. (1987) Science 238, 1098-1104. Carbon-14-labeled 1-isothiocyanatobenzyl-3-methyldiethylene triaminepentaacetic acid (MX-DTPA) is an exemplary chelating agent for conjugation of radionucleotide to the antibody (see WO-A-94/11026).

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Vectors and Cells Expressing Cytoplasmic Dynein Heavy Chain1

Another aspect of the invention pertains to vectors, preferably expression vectors, containing a nucleic acid encoding a cytoplasmic dynein heavy chain1 protein or a mutein as described in individualized manner in connection with the preferred muteins of the invention above, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded circular DNA molecule into which additional DNA segments can be ligated. Another type of vector is a viral vector, wherein additional DNA segments can be ligated into the viral genome. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”.

A host cell of the invention, such as a prokaryotic or eukaryotic host cell in culture, can be used to produce (e.g., express) cytoplasmic dynein heavy chain1 protein or mutein as described in individualized manner in connection with the preferred muteins of the invention above. Accordingly, the invention further provides methods for producing cytoplasmic dynein heavy chain1 protein or mutein using the host cells of the invention. In one embodiment, the method comprises culturing the host cell of invention (into which a recombinant expression vector encoding cytoplasmic dynein heavy chain1 protein has been introduced) in a suitable medium such that cytoplasmic dynein heavy chain1 protein or mutein is produced. In another embodiment, the method further comprises isolating cytoplasmic dynein heavy chain1 protein or mutein from the medium or the host cell.

The host cells of the invention can also be used to produce non-human transgenic animals. For example, in one embodiment, a host cell of the invention is a fertilized oocyte or an embryonic stem cell into which cytoplasmic dynein heavy chain1 protein- or cytoplasmic dynein heavy chain1 mutein-coding sequences have been introduced. Such host cells can then be used to create non-human transgenic animals in which exogenous cytoplasmic dynein heavy chain1 sequences have been introduced into their genome or homologous recombinant animals in which endogenous cytoplasmic dynein heavy chain1 sequences have been altered. Such animals are useful for studying the function and/or activity of cytoplasmic dynein heavy chain1 protein and for identifying and/or evaluating modulators of cytoplasmic dynein heavy chain1 protein activity. As used herein, a “transgenic animal” is a non-human animal, preferably a mammal, more preferably a rodent such as a rat or mouse, which contains one or more cells bearing genetic information (a transgene), received, directly or indirectly, by deliberate genetic manipulation at the subcellular level, such as by microinjection or transfection with recombinant DNA, or infection with a recombinant virus. Other examples of transgenic animals include non-human primates; ungulates such as cows, horses, goats, sheep; dogs, cats, and also chickens, amphibians, etc. Standard methods are known in the art that may be used in conjunction with the polynucleotides and of the invention and methods described herein to produce a transgenic animal expressing a modified cytoplasmic dynein heavy chain1 of the invention.

As used herein, a “transgene” is a foreign gene that has been artificially introduced into the genome of of an non human organism. A transgene present in germ cells is inherited by offspring. If such offspring in fact possesses the transgene, they too are transgenic animal.s

A transgene present only in somatic cells is nor passed on to the gametes.

As used herein, the term “transgenic” describes an organism whose genome incorporates and expresses genes from another species.

Animal Models

The present invention provides a non-human animal model which expresses a cytoplasmic dynein heavy chain1 protein modified as described herein, e.g. for the muteins described in individualized manner in connection with the preferred muteins above, as compared to the amino acid sequence of the wild type protein. The cytoplasmic dynein heavy chain1 expressed may have similarity in sequence and secondary structure to a vertebrate dynein heavy chain, including, but not limited to mammalian dynein heavy chain proteins, preferably of bovine, rat, and preferably mouse origin. The animal is preferably from a genus selected from the group consisting of Mus (e.g., mice), Rattus (e.g., rats), Oryctologus (e.g., rabbits) and Mesocricetus (e.g., hamsters). In a particularly preferred embodiment the animal is a mouse.

Animals carrying a mutated cytoplasmic dynein heavy chain1 allele expressing the modified cytoplasmic dynein heavy chain1 proteins of the invention, e.g. a mutein as described in individualized manner in connection with the preferred muteins above, exhibit a variety of phenotypical features including: myoclonic cramps (which are especially pronounced in the hindlimbs), movement hyperactivity, reduced muscle endurance, excitatory neuronal damage (“dark neurons”) in the hippocampus (gyrus dentatus, CA4, CA3) as well as in the upper layer of the cortex and in the Purkinje cell layer of the cerebellum.

Compared with heteroygous animals, homozygous individuals exhibit an elevated incidence of perinatal lethality (100% in homozygous animals, versus approximately 20% amongst heterozygous animals), accelerated neurondegeneration in the anterior horns of the spinal cord, and accelerated neurodegeneration in the dorsal root ganglia. Neurodegeneration in heterozygous animals is apparent at a much later stage, i.e. late adulthood.

The term “phenotype” according to the invention refers to a collection of morphological, physiological, behavioral and biochemical traits possessed by a cell or organism that results from the interaction of the genotype and the environment. Thus, the animal model of the present invention displays readily observable abnormalities. In a preferred embodiment the animal of the invention shows at least 2, preferably at least 4, more preferably 6 and most preferably all of the above listed phenotypical features.

In the present invention, mice were generated carrying a point mutation in the thirteenth exon (exon 13) of the mouse cytoplasmic dynein heavy chain1 gene, which is illustrated in SEQ ID NO: 1, thereby replacing the tyrosine residue at position 1055 in the N-terminal domain of the protein. This modification results in the above mentioned phenotypical features. Amino acid position 1055 according to the present invention refers to the non-mature cytoplasmic dynein heavy chain1 protein, as numbered in SEQ ID NO:4. However, it will be appreciated by the person skilled in art that also the mature protein is encompassed by the present invention and may be expressed in the animal model of the present invention. A mature protein may result from a cleavage of several amino acids, and therefore may alter the numbering of the amino acids in the mature protein relative to the numbering of an unprocessed protein. The tyrosine residue corresponding to the Tyr located at position 1055 in the amino acid sequence of SEQ ID NO:2 is conserved among the cytoplasmic dynein heavy chain proteins of different species as can be seen in the alignment in Table 9.

TABLE 9
Clustal W comparison of wild-type Dynein Heavy Chains

| | |
|---|---|
| gi\|9717245\|MOUSE SEQ ID NO:7 | 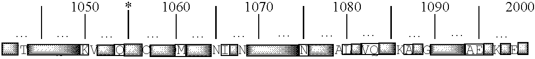 |
| gi\|729378\|RAT SEQ ID NO:8 | 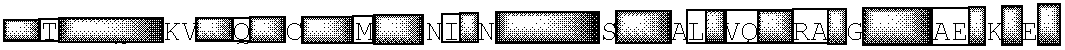 |
| gi\|7025519\|HUMAN SEQ ID NO:9 | 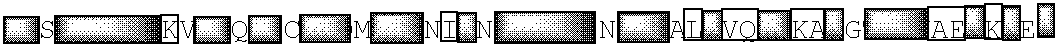 |
| gi\|17647333\|DROSOPHILA SEQ ID NO:10 | 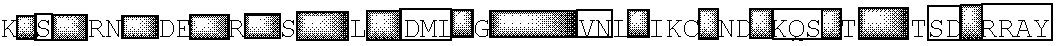 |
| gi\|987229\|PARAMECIUM SEQ ID NO:11 | 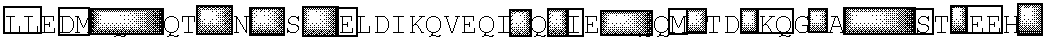 |
| gi\|2494203\|C. ELEGANSSEQ ID NO:12 | 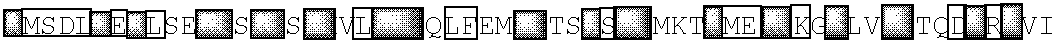 |

Thus, the non-human animal model of the present invention carries, e.g., a nucleic acid sequence encoding cytoplasmic dynein heavy chain1, whereby the codon for the amino acid at position 1055 of the amino acid sequence shown in SEQ ID NO:2 or the codon corresponding to said position in other dynein heavy chain proteins (especially cytoplasmic dynein heavy chain1 proteins), like position 1057 of the amino acid sequence shown in SEQ ID NO: 18, which encodes a Tyr in the wild type, is mutated to encode a different amino acid. In a preferred embodiment, the animal model of the present invention expresses the amino acid sequence shown in SEQ ID NO:4.

In a preferred embodiment the animal model of the invention carries a modified cytoplasmic dynein heavy chain1 nucleic acid sequence derived from a vertebrate, preferably from a mammal, in particular from mouse, for example, a cytoplasmic dynein heavy chain1 nucleic acid sequence encoding a cytoplasmic dynein heavy chain1 mutein as described in individualized manner in connection with the preferred muteins of the invention above. In a particularly preferred embodiment the nucleic acid sequence is derived from the nucleic acid sequence of SEQ ID NO:1, 3, 5, or 17.

The animal model of the invention preferably expresses a modified cytoplasmic dynein heavy chain1 protein of the invention (e.g. as shown in SEQ ID NO:4, or SEQ ID NO:6 or a mutein as described in individualized manner in connection with the preferred muteins above) in all of its cells, and particularly in the brain and spinal cord. However, animals which express the modified cytoplasmic dynein heavy chain1 protein in some, but not all cells, which are termed cellular mosaic animals, are also contemplated.

The present invention further provides for inbred successive lines of animals carrying the mutant cytoplasmic dynein heavy chain1 nucleic acid of the present invention that offer the advantage of providing a virtually homogenous genetic background. A genetically homogenous line of animals provides a functionally reproducible model system for disorders or symptoms associated with movement hyperactivity, hyperexcitability disorders (e.g. myoclonic cramping, epilepsy), excitotoxicity disorders and neurodegeneration; over-activity or undesirable activity of endogenous cytoplasmic dynein heavy chain1; over-expression, over-production or undesirable production of endogenous cytoplasmic dynein heavy chain1; excessive or undesirable condition shown to be modulated by endogenous cytoplasmic dynein heavy chain1.

The animal model of the present invention may use any of the cytoplasmic dynein heavy chain1 muteins described herein, e.g., those described in an individualized manner in connection with the preferred muteins of the invention above, and is not limited to the modification of the residue at position 1055 of the amino acid sequence shown in SEQ ID NO:4 or at a corresponding position in other dynein heavy chain orthologs (e.g. position 1057 of the amino acid sequence shown in SEQ ID NO:6) so long as the mutein used imparts the desired mutant dynein phenotype to the animal of the present invention. Suitable muteins include those with modifications in the amino acid sequence of the mouse or human cytoplasmic dynein heavy chain1 proteins so long as they impart a phenotype as described herein in connection with the animals of the invention. Such muteins may comprise mutations, such as single or multiple further amino acid substitutions, deletions and insertions, particularly mutations that affect the conserved amino acids of mouse or human cytoplasmic dynein heavy chain1, as described, e.g., in an individualized manner in connection with the preferred muteins of the invention above. Amino acid insertional derivatives of the present invention include amino and/or carboxyl terminal fusions as well as intra-sequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. Preferably, the cytoplasmic dynein heavy chain1 expressed in the animal model of the invention has a degree of identity preferably of at least 70%, 75%, 80%, 85%, 90%, 95%, 98%, or most preferably 99%, with the wild type cytoplasmic dynein heavy chain1 sequence from vertebrate, preferably from mammals, most preferably from bovine, and from rat and even most preferably from mouse (SEQ ID NO:2), excluding the wild type mouse cytoplasmic dynein heavy chain1 sequence itself. In another preferred embodiment, the cytoplasmic dynein heavy chain1 expressed in the animal model of the invention is human cytoplasmic dynein heavy chain1 (SEQ ID NO:18) or has a degree of identity preferably of at least 70%, 75%, 80%, 85%, 90%, 95%, 98%, or most preferably 99%, with the human cytoplasmic dynein heavy chain1 sequence in SEQ ID NO:18 (e.g. SEQ ID NO:6). The human wild type cytoplasmic dynein heavy chain1 sequence is a further embodiment of the invention.

In a particularly preferred embodiment the animal model expresses a polypeptide as shown in SEQ ID NO:4. Further preferred embodiments include the animal model of the invention, that expresses a polypeptide selected from those shown in SEQ ID NO:6 and SEQ ID NO:18. Preferred modifications of the cytoplasmic dynein heavy chain1 amino acid sequence in the animal models of the present invention, in addition to the modification at, e.g, position 1055 of the amino acid sequence shown in SEQ ID NO:4, (or in addition to the modification in the equivalent position 1057 of the amino acid sequence shown in SEQ ID NO:6) are at positions which are conserved among the vertebrate dynein heavy chain proteins (especially those conserved among vertebrate cytoplasmic dynein heavy chain1 proteins), as described in individualized manner in connection with the preferred muteins above.

Another preferred modification of the cytoplasmic dynein heavy chain1 amino acid sequence in the animal models of the present invention, in addition to the modification at position 1055 of the amino acid sequence shown in SEQ ID NO:4, (or in addition to the modification in the equivalent position 1057 of the amino acid sequence shown in SEQ ID NO:6) are at positions which are not conserved among the vertebrate dynein heavy chain proteins (especially those not conserved among vertebrate cytoplasmic dynein heavy chain1 proteins), as described above.

It will be appreciated that the animal model of the invention may carry a mutated cytoplasmic dynein heavy chain1 nucleic acid according to the present invention derived from the same species or from a different species. Preferably, the mutated cytoplasmic dynein heavy chain1 nucleic acid of the present invention is homozygous in the animals of the present invention. Preferably, transcription of the mutated cytoplasmic dynein heavy chain1 gene of the present invention is under the control of the promoter sequence controlling transcription of the endogenous wild type cytoplasmic dynein heavy chain1 sequence of the animal, although a different promoter may be used.

The animals of the invention can be produced by using any technique known to the person skilled in the art; including but not limited to micro-injection, electroporation, cell gun, cell fusion, micro-injection into embryos of teratocarcinoma stem cells or functionally equivalent embryonic stem cells. The animals of the present invention may be produced by the application of procedures which result in an animal with a genome that incorporates and/or integrates exogenous genetic material in such a manner as to modify or disrupt the function of the normal cytoplasmic dynein heavy chain1 gene or protein. The prefeffed procedure for generating animal models of this invention as described in Example 1.

Alternatively, the procedure may involve obtaining genetic material, or a portion thereof, which encodes a cytoplasmic dynein heavy chain1. The isolated native sequence is then genetically manipulated by the insertion of a mutation appropriate to replace the residue at position 1055 of the amino acid sequence shown in SEQ ID NO:2. The manipulated construct may then be inserted into embryonic stem cells, e.g. by electroporation. The cells subjected to said procedure are screened to find positive cells, e.g. cells which have integrated into their genome the desired construct encoding an altered cytoplasmic dynein heavy chain1. The positive cells may be isolated, cloned (or expanded) and injected into blastocysts obtained from a host animal of the same species or a different species. For example, positive cells are injected into blastocysts from mice, the blastocysts are then transferred into a female host animal and allowed to grow to term, following which the offspring of the female are tested to determine which animals are transgenic, e.g. which animals have an inserted exogenous mutated DNA sequence. One method involves the introduction of the recombinant gene at the fertilized oocyte stage ensuring that the gene sequence will be present in all of the germ cells and somatic cells of the “founder” animal. The term “founder animal” as used herein means the animal into which the recombinant gene was introduced at the one cell embryo stage.

The animals of the invention can also be used as a source of primary cells from a variety of tissues, for cell culture experiment, including but not restricted to, the production of immortalized cell lines by any methods known in the art, such as retroviral transformation. Cells from the animals may advantageously exhibit desirable properties of both normal and transformed cultured cells, e.g. they will be normal or nearly normal morphologically and physiologically, but can be cultured for long, and perhaps indefinite periods of time. The present invention provides such primary cells and cell lines derived therefrom, obtained from the animals of the present invention. These primary cells or cell lines derived thereof may be used for the construction of an animal model of the present invention.

In other embodiments cell lines may be prepared by the insertion of a nucleic acid construct comprising the nucleic acid sequence of the invention or a fragment thereof comprising the codon imparting the above-described phenotype to the animal model of the invention (vide infra). Suitable cells for the insertion include primary cells harvested from an animal as well as cells which are members of an immortalized cell line. Recombinant nucleic acid constructs of the invention, described below, may be introduced into the cells by any method known in the art, including but not limited to, transfection, retroviral infection, micro-injection, electroporation, transduction or DEAE-dextran. Cells which express the recombinant construct may be identified by, for example, using a second recombinant nucleic acid construct comprising a reporter gene which is used to produce selective expression. Cells that express the nucleic acid sequence of the invention or a fragment thereof may be identified indirectly by the detection of reporter gene expression.

Pharmaceutical Compositions

The invention also includes pharmaceutical compositions containing mutein nucleic acids or proteins of the invention, e.g. those muteins as described in individualized manner in connection with the preferred muteins of the invention above, as well as pharmaceutical compositions containing antibodies to them. The compositions are preferably suitable for internal use and include an effective amount of a pharmacologically active compound of the invention, alone or in combination, with one or more pharmaceutically acceptable carriers. The compounds are especially useful in that they have very low, if any toxicity.

The mutated nucleic acid sequences and muteins of this invention, and antibodies thereto, may be used in pharmaceutical compositions, when combined with a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences (e.g. currently the 18th edition), Alfonso R. Gennaro, ed. (Mack Publishing Co., Easton Pa., USA, 1990), a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g. intravenous, intradermal, subcutaneous, oral (e.g. inhalation), transdermal (e.g. topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J., U.S.A.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

For instance, for oral administration in the form of a tablet or capsule (e.g. a gelatin capsule), the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol and the like. Disintegrators include, without limitation, starch, methyl cellulose, agar, bentonite, xanthan gum starches, agar, alginic acid or its sodium salt, or effervescent mixtures, and the like. Examples of diluents include, without limitation, lactose, dextrose, sucrose, mannitol, sorbitol, cellulose and/or glycine.

Injectable compositions are preferably aqueous isotonic solutions or suspensions, and suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.1 to 75%, preferably about 1 to 50%, of the active ingredient.

The compounds of the invention can also be administered in such oral dosage forms as timed release and sustained release tablets or capsules, pills, powders, granules, elixers, tinctures, suspensions, syrups and emulsions.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in liquid prior to injection can be formulated. Injectable compositions are, preferably aqueous isotonic solutions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances.

The compounds of the present invention can be administered in intravenous (both bolus and infusion), intraperitoneal, subcutaneous or intramuscular form, all using forms well known to those of ordinary skill in the pharmaceutical arts. Injectables can be prepared in conventional forms, either as liquid solutions or suspensions.

Parental injectable administration is generally used for subcutaneous, intramuscular or intravenous injections and infusions. Additionally, one approach for parenteral administration employs the implantation of a slow-release or sustained-released systems, which assures that a constant level of dosage is maintained, according to U.S. Pat. No. 3,710,795, incorporated herein by reference.

Furthermore, preferred compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would range from 0.1% to 15%, w/w or w/v.

For solid compositions, excipients include pharmaceutical grades of mannitol, lactose, starch, magnesium stearate, sodium saccharin, talcum, cellulose, glucose, sucrose, magnesium carbonate, and the like may be used. The active compound defined above, may be also formulated as suppositories using for example, polyalkylene glycols, for example, propylene glycol, as the carrier. In some embodiments, suppositories are advantageously prepared from fatty emulsions or suspensions.

The compounds of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

If desired, the pharmaceutical composition to be administered may also contain minor amounts of non-toxic auxiliary substances such as wetting or emulsifying agents, pH buffering agents, and other substances such as for example, sodium acetate, triethanolamine oleate, etc.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

Oral dosages of the present invention, when used for the indicated effects, will range between about 0.05 to 1000 mg/day orally. The compositions are preferably provided in the form of scored tablets containing 0.5, 1.0, 2.5, 5.0, 10.0, 15.0, 25.0, 50.0, 100.0, 250.0, 500.0 and 1000.0 mg of active ingredient. Effective plasma levels of the compounds of the present invention range from 0.002 mg to 50 mg per kg of body weight per day.

Compounds of the present invention may be administered in a single daily dose, or the total daily dosage may be administered in divided doses of two, three or four times daily.

Any of the above pharmaceutical compositions may contain 0.1-99%, preferably 1-70% of the cytoplasmic dynein heavy chain1 polypeptide.

If desired, the pharmaceutical compositions can be provided with an adjuvant. Adjuvants are discussed above. In some embodiments, adjuvants can be used to increase the immunological response, depending on the host species, include Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Generally, animals are injected with antigen using several injections in a series, preferably including at least three booster injections.

Assays and Diagnostics

The animals of the present invention present a phenotype whose characteristics are representative of many symptoms associated with disorders associated with dynein heavy chain deficiency (especially cytoplasmic dynein heavy chain1 deficiency), therefore making the animal model of the present invention a particularly suitable model for the study of these diseases including movement hyperactivity, hyperexcitability disorders (e.g. myoclonic cramping, epilepsy), excitotoxicity disorders and neurodegeneration; over-activity or undesirable activity of endogenous cytoplasmic dynein heavy chain1; over-expression, over-production or undesirable production of endogenous cytoplasmic dynein heavy chain1; excessive or undesirable condition shown to be modulated by endogenous cytoplasmic dynein heavy chain1. In particular the animal model of the invention presents a phenotype characterized by cramping, movement hyperactivity; excitatory neuronal damage in the CA3 and CA4 sector of the hippocampus, as well as in the upper layer of the cortex and in the Purkinje cell layer of the cerebellum and neurondegeneration. These are all phenotypical characteristics of human diseases related to hyperexcitability and neurodegeneration, such as epilepsy, Alzheimer's disease, Huntington's disease, and Parkinson's disease. Therefore, the animals of the invention can be used to study diseases or symptoms associated with movement hyperactivity and impairment, hyperexcitability disorders (e.g. myoclonic cramping, epilepsy), excitotoxicity disorders, cognitive disorders and neurodegeneration in general.

The animals of the present invention can also be used to identify early diagnostic markers for diseases associated with cytoplasmic dynein heavy chain1 deficiency. Surrogate markers, including but not limited to ribonucleic acids or proteins, can be identified by performing procedures of proteomics or gene expression analysis known in the art. For example procedures of proteomics analysis include, but are not restricted to, ELISA, 2D-gel, protein microarrays or mass spectrophotometric analysis of any organ or tissue samples, such as blood samples, or derivatives thereof, preferably plasma, at different age or stage of cytoplasmic dynein heavy chain1 activity deficiency associated disease development, or symptom thereof. As a further example, gene expression analysis procedures include, but are not restricted to, differential display, cDNA microarrays, analysis of quality and quantity of ribonucleic acids species from any organ or tissue samples, such as blood samples, or derivatives thereof, at different age or stage of development of cytoplasmic dynein heavy chain1 activity deficiency associated disease, or symptom thereof.

The animal model of the present invention can be used to monitor the activity of agents useful in the prevention or treatment of the above-mentioned diseases and disorders. The agent to be tested can be administered to an animal of the present invention and various phenotypic parameters can be measured or monitored. In a further embodiment the animals of the invention may be used to test therapeutics against any disorders or symptoms that have been shown to be associated with cytoplasmic dynein heavy chain1 deficiency or overexpression.

The animals of the present invention can also be used as test model systems for materials, including but not restricted to chemicals and peptides, particularly medical drugs, suspected of promoting or aggravating the above-described diseases associated with cytoplasmic dynein heavy chain1 deficiency. For example, the material can be tested by exposing the animal of the present invention to different time, doses and/or combinations of such materials and by monitoring the effects on the phenotype of the animal of the present invention, including but not restricted to movement activity; latency to fall in a hanging wire assay, motor coordination test assays, such as rotarod, stationary beam and coat hanger motor performance, excitatory neuronal damage in the hippocampus (CA3, CA4 and gyrus dentatus) and in the pyramidal area of the cortex, histopathological and biochemical parameters of motor neuron degeneration and spinal muscular atrophy, as well as tracer experiments to determine the functionality of the retrograd axonal transport.

Furthermore, the animals of the present invention may be used for the dissection of the molecular mechanisms of the cytoplasmic dynein heavy chain1 pathway, that is for the identification of downstream genes or proteins thereof regulated by cytoplasmic dynein heavy chain1 activity and deregulated in cytoplasmic dynein heavy chain1 activity deficiency associated disorders. For example, this can be done by performing differential proteomics analysis, using techniques including but not restricted to 2D gel analysis, protein chip microarrays or mass spectrophotometry, on tissues of the animal of the present invention which express cytoplasmic dynein heavy chain1 and which respond to cytoplasmic dynein heavy chain1 stimuli.

The animal model of the present invention can be used to identify and clone so-called modifier genes which are able to modify, aggravate, reduce or inhibit the phenotype associated with a cytoplasmic dynein heavy chain1 activity deficiency. Particularly, for this purpose, the animal model of the present invention can be mated to mice of different strains carrying a different genetic background, which gives the possibility to map the genes modifying the phenotype. For example, the animal model of the present invention, when produced in an C3HeB/FeJ inbred strain background can be bred to C57B1/6Jico inbred mice. The hybrid animals of this progeny, are then further bred, either in back-cross strategy with C57B1/6Jico inbred mice again, or in an intercross strategy between each other. The modifier gene can be then mapped and cloned by using microsattelites or a single nucleotide polymorphism (SNP) strategy on the mice resulting from the backcross or intercross breeding that have been grouped with respect to their phenotype intensity.

An exemplary method for detecting the presence or absence of cytoplasmic dynein heavy chain1 in a biological sample, e.g. a mutein of the present invention, e.g., those described in individualized manner in connection with the preferred muteins of the invention above, involves obtaining a biological sample from a test subject and contacting the biological sample with a compound or an agent capable of detecting cytoplasmic dynein heavy chain1 protein, mutein, or nucleic acid (e.g. mRNA, genomic DNA) that encodes cytoplasmic dynein heavy chain1 protein or mutein such that the presence of cytoplasmic dynein heavy chain1 is detected in the biological sample. An agent for detecting cytoplasmic dynein heavy chain1 mRNA or genomic DNA is a labeled nucleic acid probe capable of hybridizing to cytoplasmic dynein heavy chain1 mRNA or genomic DNA.

The diagnostic methods described herein can furthermore be utilized to identify subjects having or at risk of developing a disease or disorder associated with aberrant cytoplasmic dynein heavy chain1 expression or activity. For example, the assays described herein, such as the preceding diagnostic assays or the following assays, can be utilized to identify a subject having or at risk of developing a disorder associated with cytoplasmic dynein heavy chain1 protein, nucleic acid expression or activity. Alternatively, the prognostic assays can be utilized to identify a subject having or at risk for developing a disease or disorder. Thus, the invention provides a method for identifying a disease or disorder associated with aberrant cytoplasmic dynein heavy chain1 expression or activity in which a test sample is obtained from a subject and cytoplasmic dynein heavy chain1 protein or nucleic acid (e.g. mRNA, genomic DNA) is detected, wherein the presence of cytoplasmic dynein heavy chain1 protein or nucleic acid is diagnostic for a subject having or at risk of developing a disease or disorder associated with aberrant cytoplasmic dynein heavy chain1 expression or activity. As used herein, a “test sample” refers to a biological sample obtained from a subject of interest. For example, a test sample can be a biological fluid (e.g., blood, plasma, serum), cell sample, or tissue.

Furthermore, the prognostic assays described herein can be used to determine whether a subject can be administered an agent (e.g. an agonist, antagonist, peptidomimetic, protein, peptide, nucleic acid, small molecule, or other drug candidate) to treat a disease or disorder associated with aberrant cytoplasmic dynein heavy chain1 expression or activity. For example, such methods can be used to determine whether a subject can be effectively treated with an agent for a disorder.

Agents, or modulators that have a stimulatory or inhibitory effect on cytoplasmic dynein heavy chain1 activity (e.g. cytoplasmic dynein heavy chain1 gene expression), as identified by a screening assay described herein can be administered to individuals to treat (prophylactically or therapeutically) cytoplasmic dynein heavy chain1-mediated disorders. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g. drugs) for prophylactic or therapeutic treatments based on a consideration of the individual's genotype. Such pharmacogenomics can further be used to determine appropriate dosages and therapeutic regimens. Accordingly, the activity of cytoplasmic dynein heavy chain1 protein, expression of cytoplasmic dynein heavy chain1 nucleic acid, or mutation content of cytoplasmic dynein heavy chain1 genes in an individual can be determined to thereby select one or more appropriate agents for therapeutic or prophylactic treatment of the individual.

The present invention also provides a diagnostic method for cytoplasmic dynein heavy chain1 activity deficiency. Patients' peptide material, particularly that in or from blood, serum or plasma, is subjected to analysis for one or more of the amino acid sequences of the present invention. The peptide material may be analyzed directly or after extraction, isolation and/or purification by standard methods.

In one embodiment of the invention, the diagnostic method comprises the identification of the modified cytoplasmic dynein heavy chain1, e.g., modified as described in individualized manner in connection with the preferred muteins of the invention above, whereby the modification is, e.g., associated with the replacement of an amino acid at a position corresponding to position 1055 in the amino acid sequence shown in SEQ ID NO:2. In another embodiment of the invention, the diagnostic method comprises the identification of the modified cytoplasmic dynein heavy chain1, e.g., the mutein as described in individualized manner in connection with the preferred muteins of the invention above, whereby the modification is, e.g., associated with the replacement of an amino acid at a position corresponding to position 1057 in the amino acid sequence shown in SEQ ID NO:18. In yet another embodiment of the invention, the diagnostic method comprises the identification of the modified cytoplasmic dynein heavy chain1, e.g., modified as described in individualized manner in connection with the preferred muteins of the invention above, wherein the modification is, e.g., associated with the replacement of an amino acid at a position that is conserved between the vertebrate cytoplasmic dynein heavy chain1 proteins. Such diagnostic methods include those employing detection of the modified cytoplasmic dynein heavy chain1 by its failure to activate a biological pathway. The diagnostic methods of the invention also include those employing detection of the modified cytoplasmic dynein heavy chain1 by its activity in competing with and blocking the action of native dynein heavy chain. Methods of identifying the modified cytoplasmic dynein heavy chain1 include any methods known in the art which are able to identify altered conformational properties of the amino acid sequence of the present invention compared to those of the wild type cytoplasmic dynein heavy chain1. These include, without limitation, the specific recognition of the modified protein by other proteins, particularly antibodies; individual or combined patterns of amino acid sequence digestion by known proteases or chemicals. In an additional, similar embodiment, the method exploits the failure of another protein to recognize the modified protein, examples being antibodies directed to an epitope of wild type cytoplasmic dynein heavy chain1 that incorporates residue 1055 of SEQ ID NO:2, or SEQ ID NO:18, and cytoplasmic dynein heavy chain1 receptors in which this portion of the molecular surface of wild type cytoplasmic dynein heavy chain1 is recognized or involved in cytoplasmic dynein heavy chain1 activation.

In a further embodiment of the present invention, the principle of the diagnostic method is the detection of a nucleic acid sequence encoding the modified cytoplasmic dynein heavy chain1 of the invention. This includes, but is not restricted to any methods known in the art using nucleic acid hybridizing properties, such as Northern blot, Southern blot, nucleic acid (genomic DNA, cDNA, MRNA, synthetic oligonucleotides) standard methods employing microarrays, and patterns of nucleic acid digestion by known restriction enzymes.

Subunits of the Dynactin/Dynein Complex

As explained above, the present invention is not only concerned with the identification of neurodegenerative disease relevant mutations in the dynein heavy chain 1 gene, but is also based on the recognition that other components (hereinafter also referred to as “subunits”) of the dynactin/dynein complex may likewise be subject to mutations that are involved in, or affect, the pathogenesis of such diseases. When reference is made in the present specification to subunits of the dynactin/dynein complex, these subunits include in particular the cytoplasmic dynein heavy chain 1, cytoplasmic dynein intermediate chain 1, cytoplasmic dynein intermediate chain 2, cytoplasmic dynein light intermediate chain 1, cytoplasmic dynein light intermediate chain 2; cytoplasmic dynein 10 kDa light chain, cytoplasmic dynein light chain Tctex 1, cytoplasmic dynein light chain 2B, DCTN 1, DCTN 2, DCTN 3, DCTN 4, DCTN 5, DCTN 6, ARP1, ARP11, HAP1, and CLIP-170, preferably encoded by the genomic nucleic acid sequences identified in Table 25, or defined by the amino acid sequences identified in Tables 26, 27, and 29-35.

TABLE 25
Subunits of the Dynein Complex and their genomic sequences
assigned as NCBI contig Accession Numbers (Gen Bank Acc. No.,
except where otherwise indicated) (NCBI contigs are derived
from genomic sequence data assembled by NCBI staff)

| | Gen Bank | |
|---|---|---|
| | Accession | SEQ |
| Name of the Subunit | Number | ID NO.: |
| Cytoplasmic dynein heavy chain 1 | Mm NW_000055 | 28 |
| Cytoplasmic dynein heavy chain 1 | Hs AL118558 | 29 |
| Cytplasmic dynein intermediate | Mm NW_000272 | 30 |
| chain 1 | | |
| Cytplasmic dynein intermediate | Hs NT_023947 | 31 |
| chain 1 | | |
| Cytoplasmic dynein | Mm NW_000176 | 32 |
| intermediate chain 2 | | |
| Cytoplasmic dynein | Hs NT_005332.11 | 33 |
| intermediate chain 2 | | |
| Cytoplasmic dynein light | Mm AC115796.2 | 34 |
| intermediate chain 1 | | |
| Cytoplasmic dynein light | Hs NT_034532.2 | 35 |
| intermediate chain 1 | | |
| Cytoplasmic dynein light | Mm AC118211.3 | 36 |
| intermediate chain 2 | | |
| Cytoplasmic dynein light | Hs NT_010478.11 | 37 |
| intermediate chain 2 | | |
| Cytoplasmic dynein 10 kDa light | Mm NW_000236 | 38 |
| chain | | |
| Cytoplasmic dynein 10 kDa light | Hs NT_009775 | 39 |
| chain | | |
| Cytoplasmic dynein light chain | Mm NW_000269 | 40 |
| Tctex 1 | | |
| Cytoplasmic dynein light chain | Hs NT_007422 | 41 |
| Tctex 1 | | |
| Cytoplasmic dynein light chain | Hs NT_024797 | 42 |
| 2B | | |
| DCTN 1 | Mm NT_003134 | 43 |
| DCTN 1 | Hs NT_022184 | 44 |
| DCTN 2 | Mm MGI107733* | 159 |
| DCTN 2 | Hs NT_009509 | 45 |
| DCTN 3 | Mm NW_000206 | 46 |
| DCTN 3 | Hs NT_023974 | 47 |
| DCTN 4 | Mm NW_000134 | 48 |
| DCTN 4 | Hs NT_034779 | 49 |
| DCTN 5 | Mm NT_036652 | 50 |
| DCTN 6 | Mm NW_000340 | 51 |
| DCTN 6 | Hs NT_007995 | 52 |
| ARP1 | Mm NW_000148 | 53 |
| ARP1 | Hs AL121928 | 54 |
| ARP11 | Mm NW_000053 | 55 |
| ARP11 | Hs AC005586 | 56 |
| HAP1 | Mm NT_033680 | 57 |
| HAP1 | Hs NT_010840 | 58 |
| CLIP-170 | Mm NW_000236 | 59 |
| CLIP-170 | Hs NT_009438 | 60 |

*Mouse Genome Informatics Tool of the Jackson Laboratory Accession No.

TABLE 26

Cytoplasmic dynein intermediate chain 1 amino acid
sequence (SEQ ID NO.:61)

GenBank Accession No: NP_0341931 (mouse)
MSDKSDLKAELERKKQRLAQIREEKKRKEEERKKKEADMQQKKEPVQDDS
DLDRKRRETEALLQSIGISPEPPLVPTPMSPSSKSVSTPSDAGSQDSGDL
GPLTRTLQWDTDPSVLQLQSDSELGRPLHKLGVSKVTQVDFLPREVVSYS
KETQTPLATHQSEEDEEDEEMVEPKIGHDSELENQEKKQETKEAPPRELT
EEEKQQILHSEEFLIFFDRTIRVIERALAEDSDIFFDYSGRELEEKDGDV
QAGANLSFNRQFYDEHWSKHRVVTCMDWSLQYPELMVASYSNNEDAPHEP
DGVALVWNMKFKKTTPEYVFHCQSSVMSVCFARFHPNLVVGGTYSGQIVL
WDNRSHRRTPVQRTPLSAAAHTHPVYCVNVVGTHNARNLITVSTDG4CSW
SLDMLSTPQESMELVYNKSKPVAVTGMAFPTGDV1FVVGSEEGTVYTACR
HGSKAGIGEVFEGHQGPVTGINCHMAVGPIDFSHLFVTSSFDWTVKLWTT
KHNKPVYSSEDNADYVYDVMWSPVHPALFACVDGMGRLDLWNLNSDTEVP
TASVAIEGASALNRVRWAQGGKEVAVGDSEGRIWIYDVGELAVPHNDEWT
RFARTLVEIRANRADSEEEGAVELAA

TABLE 27

Cytoplasmic dynein intermediate chain 1 amino acid
sequence (SEQ ID NO.:62)

GenBank Accession No.: NP_0044021 (human)
MSDKSDLKAELERKQRLAQIREEKKRKEEERKKKEADMQQKKEPVQDDSD
LDTEALLQSIGISFEPPLVQPLHFLTWDTCYFHYLVPTPMSPSSKSVSTP
SEAGSQDSGDLGPLTRTLQWDTDPSVLQLQSDSELGRRLHKLGVSKVTQV
DFLPREVVSYSKETQTPLATHQSEEDEEDEEMVESKVGQDSELENQDKKQ
EVKEAPPRELTEEEKQQILHSEEFLIFFDRTIRVIERALAEDSDIFFDYS
GRELEEKDGDVQAGANLSFNRQFYDEHWSKHRVVTCMDWSLQYPELMVAS
YNNNEDAPHEPDGVALVWNMKFKKTTPEYVFHCQSSVNSVCFARFHPNLV
VGGTYSGQIVLWDNRSHRRTPVQRTPLSAAAHTHPVYCVNVVGTQNAHNL
ITVSTDGKMCSWSLDMLSTPQESMELVYNKSKPVAVTGMAFPTGDVNNFW
GSEEGTVYTACRHGSGIGEVFEGHQGPVTGINCHMAVGPIDFSHLFVTSS
FDWTVKLWTTKHNKPLYSFEDNADYVYDVMWSPVHPALFACVDGMGRLDL
WNLNNDTEVPTASVAIEGASALNRVRWAQAGKEVAVGDSEGRIWVYDVGE
IAVPHNDEWTRFARTLVEIRANRADSEEEGTVELSA

TABLE 29

Cytoplasmic dynein intermediate chain 1 amino acid
sequence (SEQ ID NO.:63)

GenBank Accession No.: NP_062107.1 (rat)
MSDKSDLKAELEPKKQRLAQIREEKKRKEEERBCKKEADMQQKKEPVPDD
SDLDRKRRETEALLQSIGISPEPPLVQPLHFLTWDTCYFHYLVPTPMSPS
SKSVSTPSEAGSQDDLGPLTRTLQWDTDPSVLQLQSDSELGRRLNKLGVS
KVTQVDFLPREVVSYSKETQTPLATHQSEEDEEDEEMVEPKVGIIDSELE
NQDKKQETKEAPPRELTEEEKQQILHSEEFLIFFDRTIRVIERALAEDSD
IFFDYSGRELEEKDGDVQAGANLSFNRQFYDEHWSKHRVVTCMDWSLQYP
ELMVASYSNNEDAPHEPDGVALVWNMKFKKTTPEYVFHCQSSVMSVCFAR
FHPNLVVGGTYSGQIVLWDNRSHRRTPVQRTPLSAAAHTHPVYCVNVVGT
QNAHNLITVSTDGKMCSWSLDMLSTPQESMELVYNKSKPVAVTGMAFPTG
DVNNFVVGSEEGTVYTACRHGSKAGIGEVFEGHQGPVTGINCHMAVGPID
FSHLFVTSSFDWTVKLWTTKILNKPLYSFEDNADYVYDVMWSPVHPALFA
CVDGMGRLDLWNLNSDTEVPTASVAIEGAYALNRVRWAQGGKEVAVGDSE
GRIWIYDVGELAVPHNDEWTRFARTLVEIRANRADSEEEGAVELAA

TABLE 30

Cytoplasmic dynein intermediate chain 2 amino acid
sequence (SEQ ID NO.:64)

GenBank Accession No.: NP_034194.1 (mouse)
MSDKSDLKAELERKKQRLAQIREEKKRKEEERKKKETDQKKEAAVSVQEE
SDLEKKRREAEALLQSMGLTTDSPIVPPPMSPSSKSVSTPSEAGSQDSGD
GAVGSRRGPIKLGMAKITQVDFPPREIVTYTKETQTPVTAQPKEDEEEED
DVATPKPPVEPEEEKTLCKDEENDSKAPPHELTEEEKQQILHSEEFLSFF
DHSTRIVERALSEQINIFFDYSGRDLEDKEGEIQAGAXLSLNRQFFDERW
SKHRVVSCLDWSSQYPELLVASYNNNEEAPHEPDGVALVWNMKYKKTTPE
YVFHCQSAVNSATFAKFHPNLVVGGTYSGQIVLWDNRSNKRTPVQRTPLS
AAAHTHPVYCVNVVGTQNAHNLISISTDGKICSWSLDMLSHPQDSMELVH
KQSKAVAVTSMSFPVGDVNNFVVGSEEGSVYTACRHGSKAGISEMFEGHQ
GPITGIHCHAAVGAVDFSHLFVTSSFDWTVKLWTTKNNKPLYSFEDNSDY
VYDVMWSPTHPALFACVDGMGRLDLWNLNNDTEVPTASISVEGNPALNRV
RWTHSGREIAVGDSEGQIVIYDVGEQIAVPRNDEWARFGRTLAEINANRA
DAEEEAATRIPA

TABLE 31

Cytoplasmic dynein intermediate chain 2 amino acid
sequence (SEQ ID NO.:65)

GenBank Accession No.: NP_0013691 (human)
MSDKSELKAELERKKQRLAQIREEKKRKEEERKKKETDQKKEAVAPVQEE
SDLEKKRREAEALLQSMGLTPESPIVFSEYWVPPPMSPSSKSVSTPSEAG
SQDSGDGAVGSRTLHWDTDPSVLQLHSDSDLGRGPIKLGMAKITQVDFPP
REIVTYTKETQTFVMAQPKEDEEEDDDVVAPKPPIEPEEEKTLKKDEEND
SKAPPHELTEEEKQQILHSEEFLSFFDHSTRIVERALSEQUIIFFDYSGR
DLEDKEGEIQAGAKLSLNRQFFDERWSKHRVVSCLDWSSQYPELLVASYN
NNEDAPHEPDGVALVWNMKYKKTTPEYVFHCQSAV1SATFAKFHPNLVVG
GTYSGQIVLWDNSNKRTPVQRTPLSAAAHTHPVYCVNVVGTQNAHNLISI
STDGKICSWSLDMLSHPQDSMELVHKQSKAVAVTSMSFPVGDVNNFVVGS
EEGSVYTACRHGSKAGISEMFEGHQGPITGIHCHAAVGAVDFSHLFVTSS
FDWTVKLWTTKNNKPLYSFEDNADYVYDVMWSPTHPALFACVDGMGRLDL
WNLNNDTEVPTASISVEGNPALNRVRWTHSGREIAVGDSEGQIVIYDVGE
QIAVPRNDEWARFGRTLAEINANRADAEEEAATRIPA

TABLE 32

Cytoplasmic dynein intermediate chain 2 amino acid
sequence (SEQ ID NO.:66)

GenBank Accession No.: NP_4463321 (rat)
MSDKSELKAELERKKQRLAQIREEKKRKEEERKKKETDQKKEAAVSVQEE
SDLEKKRREAEALLQSMGLTTDSPIVFSEHWVPPPMSPSSKSVSTPSEAG
SQDSGDGAVGSRTLHWDTDPSALQLHSDSDLGRGPIKLGMAKITQVDFPP
REIVTYTKETQTPVTAQPKEDEEEEDDVAAPKPFVEPEEEKILKKDEEND
SKAPPHELTEEEKQQILHSEEFLSFFDHSTRIVERALSEQINIFFDYSGR
DLEDKEGEIQAGAKLSLNRQFFDERWSKHRVVSCLDWSSQYPELLVASYN
NNEEAPHEPDGVALVWNMKYKKTTPEYVFHCQSAVMSATFAKFHPNLVVG
GTYSGQIVLWDNRSNKRTPVQRTPLSAAAHTHEVYCVNVVGTQNAHNLIS
ISTDGKICSWSLDMLSHPQDSMELVHKQSKAVAVTSMSFPVGDVNNFVVG
SEEGSVYTACRHGSKAGISEMFEGHQGPITGIHCHAAVGAVDFSHLFVTS
SFDWTVKLWSTKNNKPLYSFEDNSDYVYDVIGSPTHPALFACVDGMGRLD
LWNLNNDTEVPTASISVEGNPALNRVRWTHSGREIAVGDSEGQIVIYDVG
EQIAVPRNDEWARFGRTLAEINASRADAEEFAATRIPA

TABLE 33

DCTN1; Dynactin subunit p150 amino acid sequence
(SEQ ID NO.:67)

GenBank Accession No.: NP_031861.1 p150 (mouse)
MAQSRRHMSSRTPSGSRMSTEASARPLRVGSRVEVIGKGHRGTVAYVGAT
LFATGVGVILDEGDGTVQGRKYFTCDEGHGIFVRQSQIQVFEDGADTTSP
ETPDSSASKVLKREGADAAAKTSKLRGLKPKKAPTARKTTTRRPKPTRFA
STGVAGFSSSLGPSGSASAGELSSSEFSTFAQTPAPIIPTPALTSPGAAP
PLPSPSKEEGLRAQVRDLEEKLETLRLKRSEDKAKLKELEKHKIQLEQVQ
EWKSKMQEQQADLQRRLKEARKEAKEALEKERYMEEMADTADAIEMATLD
KEMAEERAESLQQEVEKERVDELTTDLEILIEEKGSDGAASSYQLKQLEE
QNARLKDALVRMRDLSSSEKQEHVKLQKLMEKKNQELEVVRQQRERLQEE
LSQAESTIDELKEQVDAAGAEEMVEMLTDRNLNLEEKVRELRETVGDLEA
MNEMNDXLQENETELELREQLDMAGARVREAQKRVEAQETVADYQQTIKK
YRQLTAHLQDVNRELTNQQERQQQPPPETFDFKIKFAETKAIIAKAIEME
LRQMEVAQANRHMSLLTAFMPDSFLRPGGDHDCVLVLLIMPRLICELIRK
QAQEKFDLSENCSERPGLRGAAGEQLSFAAGLVYSLSLLQATLHRYEHAL
SQCSVDVYKKVGSLYFEMSAHERSLDFIIELLHKDQLDETVNVEPLNKGI
KYYQHLYRIHLAEQPEDSTMQLADHIKFTQSALDCMGVEVGRLRAFLQGG
QEATDIALLLRDLETSCSDTRQFCKKIRRRNPGTDAPGIPAALAFGSQVS
DTLLDCRKMLTWVVAVLQEVAAAAAQLIAPLAENEGLPVAALEELAFKAS
EQIYGSPSSSPYECLRQSCTILISTMNKLATAMQEGEYDAERPPSKPPPV
ELRAAALRAEITDAEGLGLKLEDRETVIKELSLKIKGEELSEANVBXSLL
EKKLDSAAKDADERIEKVQTRLDETQTLLRKKEKDFEETMDALQADIDQL
FAEKAELKQRLNSQSKRTIEGLRGPPPSGIATLVSGIAGEEPQRGGAPGQ
APGALPGPGLVKDSPLLLQQISAMRLHISQLQHENSILRGAQMKASLAAL
PPLHVAKLSLPPHEGPGGNLVAGMJYRKTSQLLEKLNQLSTHTHWDITRS
SPAAXSPSAQLMEQVAQLKSLSDTIEKLKDEVLKETVTQRFGATVPTDFA
TFPSSAFLRAKEEQQDDTVYMGKVTFSCAAGLGQRHRLVLTQEQLHQLHS
RLIS

TABLE 34

DCTN1; Dynactin subunit p150 amino acid sequence
(SEQ ID NO.:68)

GenBank Accession No.: AAD55811.1 (human)
MAQSKRHVYSRTPSGSRMSAEASARPLRVGSRVEVIGKGHRGTVAYVGAT
LFATGKWVGVILDEAKGKNDGTVQGRKYFTCDEGHGIFVRQSQIQVFEDG
ADTTSPETPDSSASKVLKREGTDTTAKTSKLRGLKPKKAPTARKTTTRRP
KPTRPASTGVAGASSSLGPSGSASAGELSSSEPSTPAQTPLAAPIIPTPV
LTSPGAVPPLPSPSKEEEGLRAQVRDLEEKLETLRLKRAEDKAKLKELEK
HKIQLEQVQEWKSKMQEQQADLQRRLKEARKEAKEALEAKERYMEEMADT
ADAIEMATLDKEMAEERAESLQQEVEALKERVDELTTDLEILKAEIEEKG
SDGAASSYQLKQLEEQNARLKDALVRMRDLSSSEKQEHVKLQKLMEKKNQ
ELEVVRQQRERLQEELSQAESTIDELKEQVDAALGAEEMVEMLTDRNLNL
EEKVRELRETVGDLEAMNEMNDELQENARETELELREQLDMAGARVREAQ
KRVEAAQETVADYQQTIKKYRQLTAHLQDVNRELTNQQEASVERQQQPPP
ETFDFKIKFAETKAMAKAIEMELRQMEVAQANRHMSLLTAFMPDSFLRPG
GDHDCVLVLLLMPRLICKAELIRKQAQEKFELSENCSERPGLRGAAGEQL
SFAAGLVYSLSLLQATLHRYEHALSQCSVDVYKKVGSLYPEMSAHERSLD
FLIELLHKDQLDETVNVEPLTKAIKYYQHLYSIHLAEQPEDCTMQLADHI
KFTQSALDCMSVEVGRLRAFLQGGQEATDIALLLRDLETSCSDIRQFCKK
IRRRMPGTDAPGIPAALAFGPQVSDTLLDCRKHLTWVVAVLQEVAAAAAQ
LIAPLAENEGLLVAALEELAFKASEQIYGTPSSSPYECLRQSCNILISTM
NKLATAMQEGEYDAERPPSKPPPVELRAAALRAEITDAEGLGLKLEDRET
VIKELKKSLKIKGEELSEANVRLSLLEKKLDSAAKDADERIEKVQTRLEE
TQALLRKKEKEFEETMDALQADIDQLEAEKAELKQRLNSQSKRTIEGLRG
PPPSGIATLVSGIAGEEQQRGAIPGQAPGSVPGFGLVKDSPLLLQQISAM
RLHISQLQHENSILKGAQMKASLASLPPLHVAKLSHEGPGSELPAGALYR
KTSQLLETLNQLSTHTHWDITRTSPAAKSPSAQLMEQVAQLKSLSDTVEK
LKDEVLKETVSQRPGATVPTDFATFPSSAFLRAKEEQQDDTVYMGKVTFS
CAAGFGQRHRLVLTQEQLHQLHSRLIS

TABLE 35

DCTN1; Dynactin subunit p150 amino acid sequence
(SEQ ID NO.:69)

GenBank Accession No.: NP_077044.1 (rat)
MAQSKRHMYNRTPSGSRMSTEASARPLRVGSRVEVIGKGHRGTVAYVGAT
LFATGKWVGVILDEAKGKNDGTVQGRKYFTCDEGHGIFVRQSQIQVFEDG
ADTTSPETPDSSASKILKREGADAAAKTSKLRGLKPKKAPTARKTTTRRP
KPTRPASTGVAGPSSSLGPSGSASAGELSSSEPSTPAQTPLAAPIIPTPA
LTSPGAAPPLPSPSKEEEGLRDQVRDLEEKLETLRLKRSEDKAKLKELEK
HKIQLEQVQEWKSKMQEQQADLQRRLKEAKEAKEALEAKERYMEEMADTA
DAIEMATLDKEMAEERAESLQQEVEALKERVDELTTDLEILKAEIEEKGS
DGAASSYQLKQLEEQNARLKDALVRMRDLSSSEKQEHVKLQKLMEKKNQE
LEVVRQQRERLQEELSQAESTIDELKEQVDAALGAEEMVEMLTDRNLNLE
EKVRELRETVGDLEAMNEMNDELQENARETELELREQLDMAGARVREAQK
RVEAAQETVADYQQTIKKYRQLTAHLQDVNRELTNQQEASVERQQQPPPE
TFDFKIKFAETKAHAKAIEMELRQMEVAQANRHMSLLTAFMPDSFLRPGG
DHDCVLVLLLMPRLICKAELIRKQAQEKFDLSENCSERPGLRGAAGEQLS
FAAGLVYSLSLLQATLHRYEHALSQCSVDVYKKVGSLYPEMSAHERSLDF
LIELLHKDQLDETVNVEPLTKAIKYYQHLYSIHLAEQPEESTMQLADHIK
FTQSALDCMSVEVGRLBAFLQGGQEATDIALLLRDLETSCSDIRQFCKKI
RRRMPGTDAPGIPAALAFGSQVSDTLLDCRKHLTWVVAVLQEVAAAAAQL
IAPLAENEGLPVAALEELAFKASEQIYGSPSSSPYECLRQSCSILISTMK
KLATAMQEGEYDAERPPSKPFPVEPWPAALRAEITDAEGLGLKLEDRETV
IKELKKSLKIKGEELSEANVRLSLLEKKLDSAAKDADERIEKVQTRLEET
QTLLRKKEKEFEETMDALQADIDQLEAEKTELKQRLNSQSKRTIEGLRGP
PPSGIATLVSGIAGEEQQRGGTPGQAPGALPGPGFVKDSPLLLQQISAMR
LHISQLQHENSILRGAQMKASLAALPPLHVAKFSLPPHEGPGGNLLSGAL
YRKTSQLLEKLNQLSTYTHVVDITRSSPACKSPSAQLMEQVAQLKSLSDT
IEKLKDEVLKETVTQRPGATVFTDFATFPSSAFLRAKEEQQDDTVYMGKV
TFSCAAGLGQRHRLVLTQEQLHQLHGRLIS

Methods of Screening for Disease-Relevant Mutations in Proteins, which are a Subunit of the Dynactin/Dynein Complex

The invention inter alia relates to the identification of a protein or a nucleic acid marker indicative of an increased risk of a mammalian subject, particularly a human subject, of developing a neurodegenerative disease or of an association of a neurodegenerative disease in a mammalian subject, particularly a human subject, with a mutation which leads to a neurodegenerative disease.

Preferably the above-mentioned neurodegenerative diseases are Alzheimer's Disease, Parkinson's Disease, or Huntington's Disease. In a preferred embodiment the neurodegenerative disease is a motorneuron degenerative disease. In an even more preferred embodiment, the motorneuron degenerative disease is Amyotrophic Lateral Sclerosis, Spinal Muscular Atrophy, Bulbo-Spinal Muscular Atrophy, Progressive Bulbar Palsy, Progressive Muscular Atrophy, and Primary Lateral Sclerosis.

In a preferred embodiment, the mutation selectively affects cell types associated with or suspected to be involved in neurodegenerative diseases. In a more preferred embodiment, these cell types are motorneurons. In an even more preferred embodiment, the motorneurons are α-motorneurons. In another preferred embodiment, the mutation affects cellular processes, e.g., neuronal axonal transport, cellular transport, proliferation, differentiation, or apoptosis. In another preferred embodiment, the mutation leads to a deficiency or malfunction, e.g. an alteration in the functional interaction or a disruption of the dynactin/dynein complex in neurons, preferably in motorneurons.

On a molecular basis, said mutation results in a deletion of an amino acid or an insertion of an additional amino acid not normally present in the amino acid sequence of the protein encoded by said allele or in a substitution by another amino acid of an amino acid encoded by said allele. In a preferred embodiment, said deletion, substitution, or insertion is encoded by both alleles of the gene coding for said protein and/or occurs in an evolutionary conserved region of said protein. In another preferred embodiment, the substitution of an amino acid which is identical between the corresponding mouse and human, preferably between the corresponding mouse, rat, and human protein encoded by said allele, by another amino acid, preferably by a non-conservative amino acid and/or the substitution of the amino acid occurs by a naturally occurring amino acid.

In another embodiment of the invention, the amino acid affected by the mutation is encoded by a codon within the open reading frame of a nucleic acid sequence, preferably by an exon, as set forth in the following tables.

TABLE 36

Mouse cytoplasmic dynein intermediate chain 1
(GenBank Acc. No.: NM_010063) (SEQ ID NO.:70)

Exon 1 (nucleotide)
ATGTCTGACAAGAGCGACCTAAAGGCCGAGCTGGAGCGCAAAAAGCAGCG
CTTAGCACAGATGAGAGGAGAAGAAACGGAAGGAAGAGGAGAGGAAGAAG
AAAGAG

TABLE 37

Mouse cytoplasmic dynein intermediate chain 1
(GenBank Acc. No.: NM_010063) (SEQ ID NO.:71)

Exon 2 (nucleotide)
GCAGATATGCAGCAAAAGAAAGAGCCCGTTCAAGATGACTCCGATCTGGA
TCGCAACGACGGGAGACAGAAGCTTTGCTTCAGAGCATTGGCATATCACC
GGAGCCCCCTCTAG

TABLE 38

Mouse cytoplasmic dynein intermediate chain 1
(GenBank Acc. No.: NM_010063) (SEQ ID NO.:72)

Exon 3 (nucleotide)
TCCCAACCCCTATGTCTCCCTCTTCGAAATCAGTGAGCACTCCCAGTGAT
GCTGGAAGCCAAGACTCGGGCGATCTGGGGCCATTAACAAG

TABLE 39

Mouse cytoplasmic dynein intermediate chain 1
(GenBank Acc. No.: NM_010063) (SEQ ID NO.:73)

Exon 4 (nucleotide)
GACCCTGCAGTGGGACACAGACCCCTCAGTGCTCCAGCTGCAGTCAGACT
CAGAACTTGG

TABLE 40

Mouse cytoplasmic dynein intermediate chain 1
(GenBank Acc. No.: NM_010063) (SEQ ID NO.:74)

Exon 1 to 4 (nucleotide)
ATTCTGACAAGAGCGACCTAAAGGCCGAGCTGGAGCGCAAAAAGCAGCGC
TTAGCACAGATWAGAGAGGAGAAGAAACGGAAGGAAGAGGAGAGGAAGAA
GAAAGAGGCAGATATGCAGCAPAAGAAAGAGCCCGTTCAAGATGACTCCG
ATCTGGATCGCAAACGACGGGAGACAGAAGCTTTGCTTCAGAGCATTGGC
ATATCACCGGAGCCCCCTCTAGTCCCAACCCCTATGTCTCCCTCTTCGAI
AATCAGTGAGCACTCCCAGTGATGCTGGAAGCCAAGACTCGGGCGATCTG
GGGCCATTAACAAGGACCCTGCAGTGGGACACAGACCCCTCAGTGCTCCA
GCTGCAGTCAGACTCAGAACTTGG

TABLE 41

Mouse cytoplasmic dynein intermediate chain 1
(GenBank Acc. No.: NM_010063) (SEQ ID NO.:75)

Exon 5 (nucleotide)
GAGACGACTGCACAAGCTGGGCGTGTCAAAGGTGACCCAGGTGGATTTCC
TGCCTAGGGGTAGTATCCTACTCCAAGGAGACACAGACTCCTCTTGCAAC
ACATCAGTCTGAAG

TABLE 42

Mouse cytoplasmic dynein intermediate chain 1
(GenBank Acc. No.: NM_010063) (SEQ ID NO.:76)

Exon 7 (nucleotide)
CCCCTCCAAGAGAGTTGACGGAGGAAGAAAAACAGCAGATCCTGCACTCA
GAAGAATTCCTCATCTTCTTTGACCGGACAATCCGAGTAATTGAAAGAGC
TCTTGCAGAGGACTCGGACATCTTTTTTGACTACAGCGGTCGAGAGCTGG
AGGAGAAAGATGG

TABLE 43

Mouse cytoplasmic dynein intermediate chain 1
(GenBank Acc. No.: NM_010063) (SEQ ID NO.:77)

Exon 8 (nucleotide)
GGATGTGCAGGCTGGAGCCAACCTGTCTTTCAACCGTCAGTTCTACGATG
AACATTGGTCTAAGCATCGGGTGGTCACATGTATGGACTGGTCTCTCCAG
G

TABLE 44

Mouse cytoplasmic dynein intermediate chain 1
(GenBank Acc. No.: NM_010063) (SEQ ID NO.:78)

Exon 9 (nucleotide)
TACCCTGAGCTGATGGTTGCTTCTTATAGCAACAACGAAGATGCTCCCCA
CGAGCCAGATGGAGTGGCCTTGGTTTGGAACATGAAGTTCAAGAAAACCA
CACCAGAATATGTCTTCCACTGTCAG

TABLE 45

Mouse cytoplasmic dynein intermediate chain 1
(GenBank Acc. No.: NM_010063) (SEQ ID NO.:79)

Exon 7 to 9 (nucleotide)
CCCCTCCAAGAGAGTTGACGGAGGAAGAAAAACAGCAGATCCTGCACTCA
GAAGAATTCCTCATCTTCTTTGACCGGACAATCCGAGTAATTGAAAGAGC
TCTTGCAGAGGACTCGGACATCTTTTTTGACTACAGCGGTCGAGAGCTGG
AGGAGAAAGATGGGGATGTGCAGGCTGGAGCCAACCTGTCTTTCAACCGT
CAGTTCTACGATGAACATTGGTCTAAGCATCGGGTGGTCACATGTATGGA
CTGGTCTCTCCAGGTACCCTGAGCTGATGGTTGCTTCTTATAGCAACAAC
GAAGATGCTCCCCACGAGCCAGATGGAGTGGCCTTGGTTTGGAACATGAA
GTTCAAGAAAACCACACCAGAATATGTCTTCCACTGTCAG

TABLE 46

Human cytoplasmic dynein intermediate chain 1
(GenBank NM_004411) (SEQ ID NO.:80)

Exon 1 (nucleotide)
TCCAAGGAAACCAACATGTCTGACAAAAGTGACTTAAAAGCTGAGCTAGA
GCGCAAAAAGCAGCGCTTAGCACAGATAAGAGAAGAGAAGAAACGGAAGG
AAGAGGAGAGGAAAAAGAAAGAG

TABLE 47

Human cytoplasmic dynein intermediate chain 1
(GenBank NM_004411) (SEQ ID NO.:81)

Exon 2 (nucleotide)
GCTGATATGCAGCAGAAGAAAGAACCCGTTCAGGACGACTCTGATCTGGA
TCGCAAACGACGAGAGACAGAGGCTTTGCTGCAAAGCATTGGTATCTCAC
CGGAGCCGCCTCTAG

TABLE 48

Human cytoplasmic dynein intermediate chain 1
(GenBank NM_004411) (SEQ ID NO.:82)

Exon 3 (nucleotide)
TGCAGCCGCTGCATTTTTTAACATGGGATACCTGTTATTTTCATTATTTA
GTCCCAACCCCTATGTCTCCCTCCTCGAAATCAGTGAGCACTCCCAGTGA
AGCTGGAAGCCAAGACTCAGGCGATCTGGGGCCATTAACAAG

TABLE 49

Human cytoplasmic dynein intermediate chain 1
(GenBank NM_004411) (SEQ ID NO.:83)

Exon 4 (nucleotide)
GACCCTGCAGTGGGACACAGACCCCTCAGTGCTCCAGCTGCAGTCAGACT
CAGAACTTGG

TABLE 50

Human cytoplasmic dynein intermediate chain 1
(GenBank NM_004411) (SEQ ID NO.:84)

Exon 1 to 4 (nucleotide)
TCCAAGGAAACCAACATGTCTGACAAAAGTGACTTAAAAGCTGAGCTAGA
GCGCAAAAAGCAGCGCTTAGCACAGATAAGAGAAGAGAAGAAACGGAAGG
AAGAGGAGAGGAAAAAGAAAGAGGCTGATATGCAGCAGAAGAAAGAACCC
GTTCAGGACGACTCTGATCTGGATCGCAAACGACGAGAGACAGAGGCTTT
GCTGCAAAGCATTGGTATCTCACCGGAGCCGCCTCTAGTGCAGCCGCTGC
ATTTTTTAACATGGGATACCTGTTATTTTCATTATTTAGTCCCAACCCCT
ATGTCTCCCTCCTCGAAATCAGTGAGCACTCCCAGTGAAGCTGGAAGCCA
AGACTCAGGCGATCTGGGGCCATTAACAAGGACCCTGCAGTGGGACACAG
ACCCTCAGTGCTCCAGCTGCAGTCAGACTCAGAACTTGG

TABLE 51

Human cytoplasmic dynein intermediate chain 1
(GenBank NM_004411) (SEQ ID NO.:85)

Exon 5 (nucleotide)
AAGAAGACTGCATAAACTGGGCGTGTCAAAGGTCACCCAAGTGGATTTCC
TGCCAAGGGAAGTAGTGTCCTACTCAAAGGAGACCCAGACTCCTCTTGCC
ACGCATCAGTCTGAAG

TABLE 52

Human cytoplasmic dynein intermediate chain 1
(GenBank NM_004411) (SEQ ID NO.:86)

Exon 7 (nucleotide)
CCCCTCCAAGAGAGTTGACAGAGGAAGAAAAACAGCAGATCCTTCATTCA
GAGGAATTTCTCATCTTTTTTGACCGGACAATACGGGTAATTGAAAGAGC
CCTGGCTGAAGATTCCGACATCTTTTTTGACTACAGCGGCCGAGAGTTAG
AGGAAAAAGATGG

TABLE 53

Human cytoplasmic dynein intermediate chain 1
(GenBank NM_004411) (SEQ ID NO.:87)

Exon 8 (nucleotide)
GGATGTTCAGGCTGGAGCCAATCTTTCTTTCAATCGTCAGTTCTATGATG
AACATTGGTCCAAGCATCGAGTGGTCACTTGTATGGACTGGTCCCTCCAG

TABLE 54

Human cytoplasmic dynein intermediate chain 1
(GenBank NM_004411) (SEQ ID NO.:88)

Exon 9 (nucleotide)
TACCCTGAGCTGATGGTGGCTTCTTACAACAACAATGAAGATGCTCCCCA
TGAACCAGATGGAGTGGCCTTGGTTTGGAACATGAAGTTTAAGAAAACCA
CACCAGAATACGTCTTCCACTGTCAG

TABLE 55

Human cytoplasmic dynein intermediate chain 1
(GenBank NM_004411) (SEQ ID NO.:89)

Exon 7 to 9 (nucleotide)
CCCCTCCAAGAGAGTTGACAGAGGAAGAAAAACAGCAGATCCTTCATTCA
GAGGAATTTCTCATCTTTTTTGACCGGACAATACGGGTAATTGAAAGAGC
CCTGGCTGAAGATTCCGACATCTTTTTTGACTACAGCGGCCGAGAGTTAG
AGGAAAAAGATGGGGATGTTCAGGCTGGAGCCAATCTTTCTTTCAATCGT
CAGTTCTATGATGAACATTGGTCCAAGCATCGAGTGGTCACTTGTATGGA
CTGGTCCCTCCAGTACCCTGAGCTTGGTGGCTTCTTACAACAACAATGAA
GATGCTCCCCATGAACCAGATGGAGTGGCCTTGGTTTGGMCATGAAGTTT
AAGAAAACCACACCAGAATACGTCTTCCACTGTCAG

TABLE 56

Mouse cytoplasmic dynein intermediate chain 2
(GenBank Acc. No. NM_010064) (SEQ ID NO.:90)

Exon 2 (nucleotide)
GTCACAACCATGTCGGACAAAAGTGATTTAAAAGCTGAGTTGGAACGTAA
GAAGCAACGGTTGGCCCAAATCAGAGAAGAAAAGAAGAGGAAAGAAGAGG
AAAGGAAAAAAAAGGAA

TABLE 57

Mouse cytoplasmic dynein intermediate chain 2
(GenBank Acc. No. NM_010064) (SEQ ID NO.:91)

Exon 3 (nucleotide)
ACTGATCAGAAGAAGGAAGCTGCTGTTTCTGTACAAGAAGAGTCTGATCT
TGAAAAAAAAAGAAGAGAAGCTGAGGCATTGCTTCAAAGCATGGGACTAA
CTACGGACTCCCCTATTG

TABLE 58

Mouse cytoplasmic dynein intermediate chain 2
(GenBank Acc. No. NM_010064) (SEQ ID NO.:92)

Exon 4 (nucleotide)
TCCCTCCTCCTATGTCTCCATCCTCCAAGTCGGTGAGCACGCCAAGTGAA
GCTGGAAGCCAGGATTCTGGAGATGGCGCCGTGGGATCTAG

TABLE 59

Mouse cytoplasmic dynein intermediate chain 2
(GenBank Acc. No. NM_010064) (SEQ ID NO.:93)

Exon 5 (nucleotide)
ACGAGGACCTATTAAACTTGGAATGGCCAAAATTACTCAAGTTGACTTTC
CCCCTCGAGAAATCGTCACATACACAAAGGAAACTCAGACCCCGGTTACA
GCTCAACCCAAAGAAG

TABLE 60

Mouse cytoplasmic dynein intermediate chain 2
(GenBank Acc. No. NM_010064) (SEQ ID NO.:94)

Exon 2 to 5 (nucleotide)
GTCACAACCATGTCGGACAAAAGTGATTTAAAAGCTGAGTTGGAACGTAA
GAAGCAACGGTTGGCCCAAATCAGAGAAGAAAAGAAGAGGAAAGAAGAGG
AAAGGAAAAAAAAGGAAACTGATCAGAAGAAGGAAGCTGCTGTTTCTGTA
CAAGAAGAGTCTGATCTTGAAAAAAAAAGAAGAGAAGCTGAGGCATTGCT
TCAAAGCATGGGACTAACTACGGACTCCCCTATTGTCCCTCCTCCTATGT
CTCCATCCTCCAAGTCGGTGAGCACGCCAAGTGAAGCTGGAAGCCAGGAT
TCTGGAGATGGCGCCGTGGGATCTAGACGAGGACCTATTAAACTTGGAAT
GGCCAAAATTACTCAAGTTGACTTTCCCCCTCGAGAAATCGTCACATACA
CAAAGGAAACTCAGACCCCGGTTACAGCTCAACCCAAAGAAG

TABLE 61

Mouse cytoplasmic dynein intermediate chain 2
(GenBank Acc. No. NM_010064) (SEQ ID NO.:95)

Exon 7 (nucleotide)
CTCCACCTCATGAACTAACTGAAGAAGAAAAGCAACAGATTTTACACTCA
GAAGAATTTTTAAGTTTCTTTGACCATTCTACGAGAATTGTAGAAAGAGC
CCTTTCTGAGCAGATTAACATCTTCTTTGACTACAGTGGGAGAGACTTGG
AAGACAAAGAAGG

TABLE 62

Mouse cytoplasmic dynein intermediate chain 2
(GenBank Acc. No. NM_010064) (SEQ ID NO.:96)

Exon 8 (nucleotide)
GGAGATTCAAGCAGGTGCTAAGCTGTCATTAAATCGACAGTTCTTTGATG
AGCGTTGGTCAAAGCATCGAGTTGTTAGTTGTTTGGATTGGTCATCCCAG

TABLE 63

Mouse cytoplasmic dynein intermediate chain 2
(GenBank Acc. No. NM_010064) (SEQ ID NO.:97)

Exon 9 (nucleotide)
TATCCAGAATTACTTGTGGCTTCCTATAATAACAATGAAGAGGCTCCTCA
TGAGCCTGATGGTGTGGCCCTCGTGTGGAATATGAAGTACAAAAAAACTA
CCCCAGAGTATGTGTTCCACTGCCAG

TABLE 64

Mouse cytoplasmic dynein intermediate chain 2
(GenBank Acc. No. NM_010064) (SEQ ID NO.:98)

Exon 7 to 9 (nucleotide)
CTCCACCTCATGAACTAACTGAAGAAGAAAAGCAACAGATTTTACACTCA
GAAGAATTTTTAAGTTTCTTTGACCATTCTACGAGAATTGTAGAAAGAGC
CCTTTCTGAGCAGATTAACATCTTCTTTGACTACAGTGGGAGAGACTTGG
AAGACAAAGAAGGGGAGATTCAAGCAGGTGCTAAGCTGTCATTAAATCGA
CAGTTCTTTGATGAGCGTTGGTCAAAGCATCGAGTTGTTAGTTGTTTGGA
TTGGTCATCCCAGTATCCAGAATTACTTGTGGCTTCCTATAATAACAATG
AAGAGGCTCCTCATGAGCCTGATGGTGTGGCCCTCGTGTGGAATATGAAG
TACAAAAAAACTACCCCAGAGTATGTGTTCCACTGCCAG

TABLE 65

Human cytoplasmic dynein intermediate chain 2,
transcript 2 (GenBank Acc. No. NM_001378
(SEQ ID NO.:99)

Exon 1 (nucleotide)
ATGTCAGACAAAGTGAATTAAAGGCTGAGTTGGAACGTAAGGCAGCGACT
GGCCCTCAGAGAGGAAAAGAAGAGAAAAGAAGAAGAAAGGAAAAAAAAAG
AAACAG

TABLE 66

Human cytoplasmic dynein intermediate chain 2,
transcript 2 (GenBank Acc. No. NM_001378)
(SEQ ID NO.:100)

Exon 2 (nucleotide)
ACCAGAAGAAGGAAGCTGTTGCTCCTGTGCAAGAAGAATCAGATCTTGAA
GGAGAGGCTGAAGCATTGCTTCAAAGCATGGGGCTAACTCCAGAATCCCC
CATTG

TABLE 67

Human cytoplasmic dynein intermediate chain 2
(GenBank Acc. No. NM_001378) (SEQ ID NO.:101)

Exon 3 (nucleotide)
TCCCTCCTCCTATGTCTCCATCCTCCAAATCTGTGAGCACTCCAAGTGAA
GCTGGAAGCCAAGACTCTGGAGATGGCGCCGTGGGATCTAG

TABLE 68

Human cytoplasmic dynein intermediate chain 2
(GenBank Acc. No. NM_001378 (SEQ ID NO.:102)

Exon 4 (nucleotide)
ACGAGGACCTATTAAACTTGGAATGGCTAAAATCACGCAAGTCGACTTTC
CTCCTCGAGAAATTGTCACGTATACAAAGGAAACTCAGACTCCAGTTATG
GCTCAACCCAAAGAAG

TABLE 69

Human cytoplasmic dynein intermediate chain 2
(GenBank Acc. No. NM_001378 (SEQ ID NO.:103)

Exon 1 to 4 (nucleotide)
ATGTCAGACAAAAGTGAATTAAAGGCTGAGTTGGAACGTAAGAAGCAGCG
ACTGGCCCAAATCAGAGAGGAAAAGAAGAGAAAAGAAGAAGAAAGGAAAA
AAAAAGAAACAGACCAGAAGAAGGAAGCTGTTGCTCCTGTGCAAGPAGAA
TCAGATCTTGAAAAAAAAAGGAGAGAAGCTGAAGCATTGCTTCAAAGCAT
GGGGCTAACTCCAGAATCCCCCATTGTCCCTCCTCCTATGTCTCCATCCT
CCAAATCTGTGAGCACTCCAAGTGAAGCTGGAAGCCAAGACTCTGGAGAT
GGCGCCGTGGGATCTAGACGAGGACCTATTAAACTTGGAATGGCTAAAAT
CACGCAAGTCGACTTTCCTCCTCGAGAAATTGTCACGTATACAAAGGAAA
CTCAGACTCCAGTTATGGCTCAACCCAAAGAAG

TABLE 70

Human cytoplasmic dynein intermediate chain 2
(GenBank Acc. No. NM_001378) (SEQ ID NO.:104)

Exon 6 (nucleotide)
CTCCCCCTCATGAGCTGACTGAAGAAGAAAAGCAACAAATCCTGCACTCT
GAGGAATTTTTAAGTTTCTTTGACCATTCTACAGAATTGTAGAAAGAGCT
CTTTCTGAGCAGATTAACATCTTCTTTGACTATAGTGGGAGAGATTTGGA
AGACAAAGAAGG

TABLE 71

Human cytoplasmic dynein intermediate chain 2
(GenBank Acc. No. NM_001378) (SEQ ID NO.:105)

Exon 7 (nucleotide)
AGAGATTCAAGCAGGTGCTAAACTGTCATTAAATCGACAATTTTTTGACG
AACGTTGGTCAAAGCATCGGGTGGTTAGTTGTTTGGATTGGTCATCTCAG

TABLE 72

Human cytopasmic dvnein intermediate chain 2
(GenBank Acc. No. NM_001378) (SEQ ID NO.:106)

Exon 8 (nucleotide)
TATCCGGAGTTACTCGTGGCTTCCTATAACAACAATGAAGATGCCCCTCA
TGAGCCTGATGGTGTGGCCCTTGTATGGAATATGAAATACAAAAAAACTA
CCCCAGAGTATGTGTTTCACTGCCAG

TABLE 73

Human cytoplasmic dynein intermediate chain 2
(GenBank Acc. No. NM_001378) (SEQ ID NO.:107)

Exon 6 to 8 (nucleotide)
CTCCCCCTCATGAGCTGACTGAAGAAGAAAAGCAACAAATCCTGCACTCT
GAGGAATTTTTAAGTTTCTTTGACCATTCTACAAGAATTGTAGAAAGAGC
TCTTTCTGAGCAGATTAACATCTTCTTTGACTATAGTGGGAGAGATTTGG
AAGACAAAGAAGGAGAGATTCAAGCAGGTGCTAAACTGTCATTAAATCGA
CAATTTTTTGACGAACGTTGGTCAAAGCATCGGGTGGTTAGTTGTTTGGA
TTGGTCATCTCAGTATCCGGAGTTACTCGTGGCTTCCTATAACAACAATG
AAGATGCCCCTCATGAGCCTGATGGTGTGGCCCTTGTATGGAATATGAAA
TACAAAAAAACTACCCCAGAGTATGTGTTTCACTGCCAG

TABLE 74
Mouse Dctn1 (P150) protein (SEQ ID NO.:108)

Exon 1 (nucleotide)
ATGGCCCAGAGCAGGAGGCACATGTCCAGTCGGACGCCGAGTGGCAGCAG
GATGAGTACGGAGGCAAGCGCCCGGCCCCTGCGGGTTGGCTCCCGCGTGG
AGGTGATTGGGAAGGGCCACCGAGGCACTGTGGCCTATGTTGGAGCCACA
CTCTTTGCCACTGGCAAATGGGTGGGCGTGATTCTGGATGAAGCAAAAGG
CAAAAATGATGGCACTGTCCAGGGAAGGAAGTATTTCACATGTGATGAAG
GCCACGGCATCTTTGTACGCCAGTCCCAG

TABLE 75
Mouse Dctn1 (P150) protein (SEQ ID NO.:109)

Exon 2 (nucleotide)
ATCCAAGTATTTGAAGATGGAGCAGATACTACTTCCCCAGAGACTCCTGA
TTCTTCTGCTTCAAAGGTCCTCAAGAGAG

TABLE 76
Mouse Dctn1 (P150) protein (SEQ ID NO.:110)

Exon 3 (nucleotide)
AGGGAGCCGATGCAGCTGCAAAGACCAGCAAACTG

TABLE 77
Mouse Dctn1 (P150) protein (SEQ ID NO.:111)

Exon 4 (nucleotide)
ACCACAACTCGACGGCCCAAG

TABLE 78
Mouse Dctn1 (P150) protein (SEQ ID NO.:112)

Exon 1 to 4 (nucleotide)
ATGGCCCAGAGCAGGAGGCACATGTCCAGTCGGACGCCGAGTGGCAGCAG
GATGAGTACGGAGGCAAGCGCCCGGCCCCTGCGGGTTGGCTCCCGCGTGG
AGGTGATTGGGAAGGGCCACCGAGGCACTGTGGCCTATGTTGGAGCCACA
CTCTTTGCCACTGGCAAATGGGTGGGCGTGATTCTGGATGAAGCAAAAGG
CAAAAATGATGGCACTGTCCAGGGAAGGAAGTATTTCACATGTGATGAAG
GCCACGGCATCTTTGTACGCCAGTCCCAGATCCAAGTATTTGAAGATGGA
GCAGATACTACTTCCCCAGAGACTCCTGATTCTTCTGCTTCAAAGGTCCT
CAAGAGAGAGGGAGCCGATGCAGCTGCAAAGACCAGCAAACTGCGGGGAC
TGAAGCCTAAGAAGGCACCGACAGCCCGAAAGACCACAACTCGACGGCCC
AAG

TABLE 80
Mouse Dctn1 (P150) protein (SEQ ID NO.:114)

Exon 5 (nucleotide)
CCTACTCGCCCAGCCAGCACTGGGGTGGCTGGGCCCAGTAGCTCCCTTGG
CCCCTCTGGCTCAGCGTCAGCCGGGGAACTAAGCAGCAGTGAGCCCAGCA
CCCCAGCTCAGACTCCGCTGGCAGCACCCATCATCCCCACACCGGCCCTC
ACCTCTCCTGGAGCAGCACCCCCACTTCCATCTCCCTCTAAG

TABLE 81
Mouse Dctn1 (P150) protein (SEQ ID NO.:115)

Exon 6 (nucleotide)
GAAGAGGAAGGGCTGAGGGCTCAGGTACGGGACCTGGAGGAGAAGCTGGA
GACCCTGCGCCTAAAACGCTCAGAAGACAAAGCAAAGCTGAAAGAGCTGG
AGAAGCACAAGATCCAGCTGGAGCAGGTGCAGGAATGGAAGAGCAAAATG
CAGGAGCAGCAGGCAGACCTGCAGCGGCGCCTCAAGGAGGCTCGGAAG

TABLE 82
Mouse Dctn1 (P150) protein (SEQ ID NO.:116)

Exon 7 (nucleotide)
GAAGCCAAGGAGGCGCTAGAGGCAAAGGAACGCTACATGGAGGAGATGGC
CGACACAGCCGACGCTATCGAGATGGCCACTCTGGACAAGGAGATGGCTG
AAGAGCGCGCTGAGTCTCTGCAGCAAGAGGTGGAGGCACTGAAGGAACGG
GTAGACGAGCTCACCACAGACCTGGAGATTCTCAAGGCTGAAATCGAAGA
GAAAG

TABLE 83
Mouse Dctn1 (P150) protein (SEQ ID NO.:117)

Exon 8 (nucleotide)
GCTCTGATGGGGCCGCATCAAGCTACCAGCTCAAGCAGCTGGAGGAGCAG
AATGCCCGCCTGAAGGATGCCCTGGTGAG

TABLE 84
Mouse Dctn1 (P150) protein (SEQ ID NO.:118)

Exon 9 (nucleotide)
GATGCGAGACCTCTCTTCCTCAGAGAAGCAGGAGCACGTGAAGCTGCAGA
AACTCATGGAAAAGAAAAACCAGGAGCTGGAGGTCGTGCGGCAGCAGCGC
GAGCGTCTTCAGGAGGAGCTGAGCCAGGCTGAGAGCACCATCGATGAGCT
CAAAGAGCAG

TABLE 85
Mouse Dctn1 (P150) protein (SEQ ID NO.:119)

Exon 10 (nucleotide)
GTGGACGCCGCTCTGGGAGCCGAGGAGATGGTGGAGATGCTGACCGACCG
GAACCTGAATCTAGAGGAGAAAGTGCGGGAGTTACGGGAGACTGTGGGGG
ACTTG

TABLE 86
Mouse Dctn1 (P150) protein (SEQ ID NO.:120)

Exon 11 (nucleotide)
GAAGCCATGAACGAGATGAACGATGAGCTGCAGGAGAACGCACGGGAGAC
GGAGCTGGAACTCCGAGAGCAGCTGGACATGGCGGGCGCCCGAGTGAGGG
AAGCGCAGAAGCGAGTGGAAGCCGCCCAGGAGACAGTCGCCGACTACCAG
CAGACCATCAAGAAGTACCGCCAGTTGACTGCCCACCTACAG

TABLE 87
Mouse Dctn1 (P150) protein (SEQ ID NO.:121)

Exon 12 (nucleotide)
GATGTCAATCGGGAGCTGACAAACGAGCAGGAAGCGTCTGTAGAGAGGCA
GCAGCAGCCGCCGCCAGAGACTTTTGATTTCAAAATCAAGTTTGCTGAGA
CCAAGGCTCATGCCAAG

TABLE 88
Mouse Dctn1 (P150) protein (SEQ ID NO.:122)

Exon 13 (nucleotide)
GCCATTGAGATGGAGTTGAGACAGATGGAAGTTGCCCAGGCCAACCGGCA
CATGTCCCTGCTGACAGCCTTTATGCCTGACAGCTTCCTTCGGCCAGGTG
GAGACCACGACTGTGTCCTGGTGCTGCTGCTCATGCCCCGACTCATTTGC
AAG

TABLE 89
Mouse Dctn1 (P150) protein (SEQ ID NO.:123)

Exon 14 (nucleotide)
GCAGAGCTCATCCGGAAGCAGGCCCAGGAGAAGTTTGACCTGAGCGAGAA
CTGTTCGGAGCGGCCCGGGCTGCGGGGAGCTGCCGGGGAGCAGCTGAGCT
TTGCTGCTGGACTGGTGTACTCGCTGAGTCTGCTGCAGGCCACGCTGCAC
CGCTATGAGCA

TABLE 90
Mouse Dctn1 (P150) protein (SEQ ID NO.:124)

Exon 15 (nucleotide)
TGCCCTCTCTCAGTGCAGTGTGGACGTGTATAAGAAGGTCGGCAGCCTGT
ACCCCGAGATGAGCGCCCACGAGCGCTCCTTAGATTTCCTCATTGAGCTG
CTGCACAAGGATCAGCTGGATGAGACTGTCAACGTGGAGCCCCTCACCAA
GGCCATCAAGTATTACCAG

TABLE 91
Mouse Dctn1 (P150) protein (SEQ ID NO.:125)

Exon 16 (nucleotide)
TGCCCTCTCTCAGTGCAGTGTGGACGTGTATAAGAAGGTCGGCAGCCTGT
ACCCCGAGATGAGCGCCCACGAGCGCTCCTTAGATTTCCTCATTGAGCTG
CTGCACAAGGATCAGCTGGATGAGACTGTCAACGTGGAGCCCCTCACCAA
GGCCATCAAGTATTACCAG

TABLE 92
Mouse Dctn1 (P150) protein (SEQ ID NO.:126)

Exon 17 (nucleotide)
TGCCCTCTCTCAGTGCAGTGTGGACGTGTATAAGAAGGTCGGCAGCCTGT
ACCCCGATGAGCGCCCAGAGCGCTCCTTAGATTTCCTCATTGCTGCTGCA
CAAGGATCAGCTGGATGAGACTGTCCGTGGGCCCCTCACCAAGGCCATCA
AGTATTACCAG

TABLE 93
Mouse Dctn1 (P150) protein (SEQ ID NO.:127)

Exon 18 (nucleotide)
GGTGGGCAGGAGGCAACAGATATTGCCCTTCTTCTCCGAGACCTGGAAAC
ATCATGTAGTGACACCCGTCAGTTCTGCAAGAAGATCCGAAGGCGGATGC
CGGGGACGGATGCTCCTGGGATCCCAGCAGCGCTGGCCTTTGGCTCACAG

TABLE 94
Mouse Dctn1 (P150) protein (SEQ ID NO.:128)

Exon 19 (nucleotide)
GTATCCGACACACTCCTGGACTGCAGGAAGCACTTGACGTGGGTGGTAGC
TGTTCTGCAGGAGGTGGCAGCTGCAGCCGCCCAGCTTATTGCCCCCTTGG
CAGAGAACGAGGGGCTGCCTGTGGCTGCACTGGAGGAGCTGGCCTTCAAA
GCAAGCGAGCAG

TABLE 95
Mouse Dctn1 (P150) protein (SEQ ID NO.:129)

Exon 20 (nucleotide)
ATCTACGGGAGCCCCTCCAGCAGCCCCTATGAGTGTCTACGCCAGTCATG
CACCATCCTCATCAGCACGATGAACAAGCTGGCCACAGCCATGCAAGAAG
GCGAGTATGACGCAGAGCGACCCCCGAGCG

TABLE 96
Mouse Dctn1 (P150) protein (SEQ ID NO.:130)

Exon 3 to 20 (nucleotide)
AGGGAGCCGATGCAGCTGCAAAGACCAGCAAACTGCGGGGACTGAAGCCT
AAGAAGGCACCGACAGCCCGAAAGACCACAACTCGACGGCCCAAGCCTAC
TCGCCCAGCCAGCACTGGGGTGGCTGGGCCCAGTAGCTCCCTTGGCCCCT
CTGGCTCAGCGTCAGCCGGGGAACTAAGCAGCAGTGAGCCCAGCACCCCA
GCTCAGACTCCGCTGGCAGCACCCATCATCCCCACACCGGCCCTCACCTC
TCCTGGAGCAGCACCCCCACTTCCATCTCCCTCTAAGGAAGAGGAAGGGC
TGAGGGCTCAGGTACGGGACCTGGAGGAGAAGCTGGAGACCCTGCGCCTA
AAACGCTCAGAAGACAAAGCAAAGCTGAAAGAGCTGGAGAAGCACAAGAT
CCAGCTGGAGCAGGTGCAGGAATGGAAGAGCAAAATGCAGGAGCAGCAGG
CAGACCTGCAGCGGCGCCTCAAGGAGGCTCGGAAGGAAGCCAAGGAGGCG
CTAGAGGCAAAGGAACGCTACATGGAGGAGATGGCCGACACAGCCGACGC
TATCGAGATGGCCACTCTGGACAAGGAGATGGCTGAAGAGCGCGCTGAGT
CTCTGCAGCAAGAGGTGGAGGCACTGAAGGAACGGGTAGACGAGCTCACC
ACAGACCTGGAGATTCTCAAGGCTGAAATCGAAGAGAAAGGCTCTGATGG
GGCCGCATCAAGCTACCAGCTCAAGCAGCTGGAGGAGCAGAATGCCCGCC
TGAAGGATGCCCTGGTGAGGATGCGAGACCTCTCTTCCTCAGAGAAGCAG
GAGCACGTGAAGCTGCAGAAACTCATGGAAAAGAAAAACCAGGAGCTGGA
GGTCGTGCGGCAGCAGCGCGAGCGTCTTCAGGAGGAGCTGAGCCAGGCTG
AGAGCACCATCGATGAGCTCAAAGAGCAGGTGGACGCCGCTCTGGGAGCC
GAGGAGATGGTGGATGCTGACCGACCGGAACCTGAATCTAGAGGAGAAAG
TGCGGGAGTTACGGGAGACTGTGGGGGACTTGGAAGCCATGAACGAGATG
AACGATGAGCTGCAGGAGAACGCACGGGAGACGGAGCTGGAACTCCGAGA
GCAGCTGGACATGGCGGGCGCCCGAGTGAGGGAAGCGCAGAAGCGAGTGG
AAGCCGCCCAGGAGACAGTCGCCGACTACCAGCAGACCATCAAGAAGTAC
CGCCAGTTGACTGCCCACCTACAGGATGTCAATCGGGAGCTGACAAACCA
GCAGGAAGCGTCTGTAGAGAGGCAGCAGCAGCCGCCGCCAGAGACTTTTG
ATTTCAAAATCAAGTTTGCTGAGACCAAGGCTCATGCCAAGGCCATTGAG
ATGGAGTTGAGACAGATGGAAGTTGCCCAGGCCAACCGGCACATGTCCCT
GCTGACAGCCTTTATGCCTGACAGCTTCCTTCGGCCAGGTGGAGACCACG
ACTGTGTCCTGGTGCTGCTGCTCATGCCCCGACTCATTTGCAAGGCAGAG
CTCATCCGGAAGCAGGCCCAGGAGAAGTTTGACCTGAGCGAGAACTGTTC
GGAGCGGCCCGGGCTGCGGGGAGCTGCCGGGGAGCAGCTGAGCTTTGCTG
CTGGACTGGTGTACTCGCTGAGTCTGCTGCAGGCCACGCTGCACCGCTAT
GAGCATGCCCTCTCTCAGTGCAGTGTGGACGTGTATAAGAAGGTCGGCAG
CCTGTACCCCGAGATGAGCGCCCACGAGCGCTCCTTAGATTTCCTCATTG
AGCTGCTGCACAAGGATCAGCTGGATGAGACTGTCAACGTGGAGCCCCTC
MACAAGGSCATCAAGTATTACCAGCATCTGTACAGSATCCACCTCGCTGA
ACAACCCGAGGACTCCACCATGCAGCTGGCTGACCACATCAAGTTCACCC
AGAGTGCCCTGGACTGCATGGGCGTGGAGGTGGGGCGGCTGCGTGCCTTC
TTGCAGGGTGGGCAGGAGGCAACAGATATTGCCCTTCTTCTCCGAGACCT
GGAAACATCATGTAGTGACACCCGTCAGTTCTGCAAGAAGATCCGAAGGC
GGATGCCGGGGACGGATGCTCCTGGGATCCCAGCAGCGCTGGCCTTTGGC
TCACAGGTATCCGACACACTCCTGGACTGCAGGAAGCACTTGACGTGGGT
GGTAGCTGTTCTGCAGGAGGTGGCAGCTGCAGCCGCCCAGCTTATTGCCC
CCTTGGCAGAGAACGAGGGGCTGCCTGTGGCTGCACTGGAGGAGCTGGCC
TTCAAAGCAAGCGAGCAGATCTACGGGAGCCCCTCCAGCAGCCCCTATGA
GTGTCTACGCCAGTCATGCACCATCCTCATCAGCACGATGAACAAGCTGG
CCACAGCCATGCAAGAAGGCGAGTATGACGCAGAGCGACCCCCGAGCAAG

TABLE 97
Mouse Dctn1 (P150) protein (SEQ ID NO.:131)

Exon 22 (nucleotide)
GGAGAGGAGCTGAGTGAGGCCAACGTGCGGCTCAGCCTCCTGGAGAAGAA
GTTGGACAGCGCTGCCAAGGATGCAGACGAGCGAATCGAGAAAGTTCAGA
CACGGCTGGACGAGACTCAGACCCTGCTGCGGAAGAAGGAGAA

TABLE 98
Mouse Dctn1 (P150) protein (SEQ ID NO.:132)

Exon 23 (nucleotide)
AGACTTTGAGGAGACAATGGACGCACTCCAGGCTGACATCGACCAGCTGG
AGGCAGAGAAGGCAGAGCTCAAGCAGCGCCTGAACAGCCAGTCCAAGCGC
ACAATCGAGGGGCTCCGGGGCCCCCCTCCGTCAGGCATCGCTACCCTGGT
CTCTGGCATCGCTGGTG

TABLE 99
Mouse Dctn1 (P150) protein (SEQ ID NO.:133)

Exon 22 to 23 (nucleotide)
GGAGAGGAGCTGAGTGAGGCCAACGTGCGGCTCAGCCTCCTGGAGAAGAA
GTTGGACAGCGCTGCCAAGGATGCAGACGAGCGAATCGAGAAAGTTCAGA
CACGGCTGGACGAGACTCAGACCCTGCTGCGGAAGAAGGAGAAAGACTTT
GAGGAGACAATGGACGCACTCCAGGCTGACATCGACCAGCTGGAGGCAGA
GAAGGCAGAGCTCAAGCAGCGCCTGAACAGCCAGTCCAAGCGCACAATCG
AGGGGCTCCGGGGCCCCCCTCCGTCAGGCATCGCTACCCTGGTCTCTGGC
ATCGCTGGTG

TABLE 100
Human DCTN (P150) protein (SEQ ID NO.:134)

Exon 2 (nucleotide)
ACGCCCAGCGGCAGCAGGATGAGTGCGGAGGCAAGCGCCCGGCCTCTGCG
GGTGGGCTCCCGTGTAGAGGTGATTGGAAAAGGCCACCGAGGCACTGTGG
CCTATGTTGGAGCCACACTGTTTGCCACTGGCAAATGGGTAGGCGTGATT
CTGGATGAAGCAAAGGGCAAAAATGATGGAACTGTTCAAGGCAGGAAGTA
CTTCACTTGTGATGAAGCGCATGGCATCTTTGTGCGCCAGTCCCAG

TABLE 101
Human DCTN 1 (P150) protein (SEQ ID NO.:135)

Exon 3 (nucleotide)
ATCCAGGTATTTGAAGATGGAGCAGATACTACTTCCCCAGAGACACCTGA
TTCTTCTGCTTCAAAAGTCCTCAAAAGAG

TABLE 102
Human DCTN 1 (P150) protein (SEQ ID NO.:136)

Exon 4 (nucleotide)
AGGGAACTGATACAACTGCAAAGACTAGCAAACTG

TABLE 103
Human DCTN 1 (P150) protein (SEQ ID NO.:137)

Exon 5 (nucleotide)
ACCACAACTCGGCGACCCAAG

TABLE 104
Human DCTN 1 (P150) protein (SEQ ID NO.:138)

Exon 2 to 5 (nucleotide)
ACGCCCAGCGGCAGCAGGATGAGTGCGGAGGCAAGCGCCCGGCCTCTGCG
GGTGGGCTCCCGTGTAGAGGTGATTGGAAAAGGCCACCGAGGCACTGTGG
CCTATGTTGGAGCCACACTGTTTGCCACTGGCAAATGGGTAGGCGTGATT
CTGGATGAAGCAAAGGGCAAAAATGATGGAAACTGTTCAAGGCAGGAAGT
ACTTCACTTGTGATGAAGGGCATGGCATCTTTGTGCGCCAGTCCCAGATC
CAGGTATTTGAAGATGGAGCAGATACTACTTCCCCAGAGACACCTGATTC
TTCTGCTTCAAAAGTCCTCAAAGAGAGGGAACTGATACAACTGCAAAGAC
TAGCAAACTGCGGGGACTGAAGCCTAAGAAGGCACCGACAGCCCGAAAGA
CCACAACTCGGCGACCCAAG

TABLE 105
Human DCTN 1 (P150) protein (SEQ ID NO.:139)

Exon 6 (nucleotide)
CCCACGCGCCCAGCCAGTACTGGGGTGGCTGGGGCCAGTAGCTCCCTGGG
CCCCTCTGGCTCAGCGTCAGCAGGTGAGCTGAGCAGCAGTGAGCCCAGCA
CCCCGGCTCAGACTCCGCTGGCAGCACCCATCATCCCCACGCCGGTCCTC
ACCTCTCCTGGAGCAGTCCCCCCGCTTCCTTCCCCATCCAAG

TABLE 106
Human DCTN 1 (P150) protein (SEQ ID NO.:140)

Exon 7 (nucleotide)
GAGGAGGAGGGACTAAGGGCTCAGGTGCGGGACCTGGAGGAGAAACTAGA
GACCCTGAGACTGAAACGGGCAGAAGACAAAGCAAAGCTAAAAGAGCTGG
AGAAACACAAAATCCAGCTGGAGCAGGTGCAGGAATGGAAGAGCAAAATG
CAGGAGCAGCAGGCCGACCTGCAGCGGCGCCTCAAGGAGGCGAGAAAG

TABLE 107
Human DCTN 1 (P150) protein (SEQ ID NO.:141)

Exon 8 (nucleotide)
GAAGCCAAGGAGGCGCTGGAGGCAAAGGAACGCTATATGGAGGAGATGGC
TGATACTGCTGATGCCATTGAGATGGCCACTTTGGACAAGGAGATGGCTG
AAGAGCGGGCTGAGTCCCTGCAGCAGGAGGTGGAGGCACTGAAGGAGCGG
GTGGACGAGCTCACTACTGACTTAGAGATCCTCAAGGCTGAGATTGAAGA
GAAGG

TABLE 108
Human DCTN 1 (P150) protein (SEQ ID NO.:142)

Exon 9 (nucleotide)
GCTCAGATGGCGCTGCATCCAGTTATCAGCTCAAGCAGCTTGAGGAGCAG
AATGCCCGCCTGAAGGATGCCCTGGTGAG

TABLE 109
Human DCTN 1 (P150) protein (SEQ ID NO.:143)

Exon 10 (nucleotide)
GATGCGGGATCTTTCTTCCTCAGAGAAGCAGGAGCATGTGAAGCTCCAGA
AGCTCATGGAAAAGAAGAACCAAGAGCTGGAAGTTGTGAGGCAACAGCGG
GAGCGTCTGCAGGAGGAGCTAAGCCAGGCAGAGAGCACCATTGATGAGCT
CAAGGAGCAG

TABLE 110
Human DCTN 1 (P150) protein (SEQ ID NO.:144)

Exon 11 (nucleotide)
GTGGATGCTGCTCTGGGTGCTGAGGAGATGGTGGAGATGCTGACAGATCG
GAACCTGTCTGGGAGAAAGTGCGCGAGTTGAGGGAGACTGTGGGAGACTT
G

TABLE 112
Human DCTN 1 (P150) protein (SEQ ID NO.:145)

Exon 12 (nucleotide)
GAAGCGATGAATGAGATGAACGATGAGCTGCAGGAGAATGCACGTGAGAC
AGAACTGGAGCTGCGGGAGCAGCTGGACATGGCAGGCGCGCGGGTTCGTG
AGGCCCAGAAGCGTGTGGAGGCAGCCCAGGAGACGGTTGCAGACTACCAG
CAGACCATCAAGAAGTACCGCCAGCTGACCGCCCATCTACAG

TABLE 113
Human DCTN 1 (P150) protein (SEQ ID NO.:146)

Exon 13 (nucleotide)
GATGTGAATCGGGAACTGACAAACCAGCAGGAAGCATCTGTGGAGAGGCA
ACAGCAGCCACCTCCAGAGACCTTTGACTTCAAAATCAAGTTTGCTGAGA
CTAAGGCCCATGCCAAG

TABLE 114
Human DCTN 1 (P150) protein (SEQ ID NO.:147)

Exon 14 (nucleotide)
GCAATTGAGATGGAATTGAGGCAGATGGAGGTGGCCCAGGCCAATCGACA
CATGTCCCTGCTGACAGCCTTCATGCCTGACAGCTTCCTTCGGCCAGGTG
GGGACCATGACTGCGTTCTGGTGCTGTTGCTCATGCCTCGTCTCATTTGC
AAG

TABLE 115
Human DCTN 1 (P150) protein (SEQ ID NO.:148)

Exon 15 (nucleotide)
GCAGAGCTGATCCGGAAGCAGGCCCAGGAGAAGTTTGAACTAAGTGAGAA
CTGTTCAGAGCGGCCTGGGCTGCGAGGAGCTGCTGGGGAGCAACTCAGCT
TTGCTGCTGGACTGGTGTACTCGCTGAGCCTGCTGCAGGCCACGCTACAC
CGCTATGAGCA

TABLE 116
Human DCTN 1 (P150) protein (SEQ ID NO.:149)

Exon 16 (nucleotide)
TGCCCTCTCTCAGTGCAGTGTGGATGTGTATAAGAAAGTGGGCAGCCTGT
ACCCTGAGATGAGTGCCCATGAGCGCTCCTTGGATTTCCTCATTGAACTG
CTGCACAAGGATCAGCTGGATGAGACTGTCAATGTGGAGCCTCTCACCAA
GGCCATCAAGTACTATCAG

TABLE 117
Human DCTN 1 (P150) protein (SEQ ID NO.:150)

Exon 17 (nucleotide)
CATCTGTACAGCATCCACCTTGCCGAACAGCCTGAGGACTGTACTATGCA
GCTGGCTGACCACATTAAG

TABLE 118
Human DCTN 1 (P150) protein (SEQ ID NO.:151)

Exon 18 (nucleotide)
TTCACGCAGAGTGCTCTGGACTGCATGAGTGTGGAGGTAGGACGGCTGCG
TGCCTTCTTGCAG

TABLE 119
Human DCTN 1 (P150) protein (SEQ ID NO.:152)

Exon 19 (nucleotide)
GGTGGGCAGGAGGCTACAGATATTGCCCTCCTGCTCCGGGATCTGGAAAC
TTCATGCAGTGACATCCGCCAGTTCTGCAAGAAGATCCGAAGGCGAATGC
CAGGGACAGATGCTCCTGGGATCCCAGCTGCACTGGCCTTTGGACCACAG

TABLE 120
Human DCTN 1 (P150) protein (SEQ ID NO.:153)

Exon 20 (nucleotide)
GTATCTGACACGCTCCTAGACTGCAGGAAACACTTGACGTGGGTCGTGGC
TGTGCTGCAGGAGGTGGCAGCTGCTGCTGCCCAGCTCATTGCCCCACTGG
CAGAGAATGAGGGGCTACTTGTGGCTGCTCTGGAGGAACTGGCTTTCAAA
GCAAGCGAGCAG

TABLE 121
Human DCTN 1 (P150) protein (SEQ ID NO.:154)

Exon 21 (nucleotide)
ATCTATGGGACCCCCTCCAGCAGCCCCTATGAGTGTCTGCGCCAGTCATG
CAACATCCTCATCAGTACCATGAACAAGCTGGCCACAGCCATGCAGGAGG
GGGAGTATGATGCAGAGCGGCCCCCCAGCAAG

TABLE 122
Human DCTN 1 (P150) protein (SEQ ID NO.:155)

Exon 4 to 21 (nucleotide)
AGGGAACTGATACAACTGCAAAGACTAGCAAACTGCGGGGACTGAAGCCT
AAGAAGGCACCGACAGCCCGAAAGACCACAACTCGGCGACCCAAGCCCAC
GCGCCCAGCCAGTACTGGGGTGGCTGGGGCCAGTAGCTCCCTGGGCCCCT
CTGGCTCAGCGTCAGCAGGTGAGCTGAGCAGCAGTGAGCCCAGCACCCCG
GCTCAGACTCCGCTGGCAGCACCCATCATCCCCACGCCGGTCCTCACCTC
TCCTGGAGCAGTCCCCCCGCTTCCTTCCCCATCCAAGGAGGAGGAGGGAC
TAAGGGCTCAGGTGCGGGACCTGGAGGAGAAACTAGAGACCCTGAGACTG
AAACGGGCAGAAGACAAAGCAAAGCTAAAAGAGCTGGAGAAACACAAAAT
CCAGCTGGAGCAGGTGCAGGAATGCAAGAGCAAAATGCAGGAGCAGCAGG
CCGACCTGCAGCGGCGCCTCAAGGAGGCGAGAAAGGAAGCCAAGGAGGCG
CTGGAGGCAAAGGAACGCTATATGGAGGAGATGGCTGATACTGCTGATGC
CATTGAGATGGCCACTTTGGACAAGGAGATGGCTGAAGAGCGGGCTGAGT
CCCTGCAGCAGGAGGTGGAGGCACTGAAGGAGCGGGTGGACGAGCTCACT
ACTGACTTAGAGATCCTCAAGGCTGAGATTGAAGAGGGGCTCAGATGGCG
CTGCATCCAGTTATCAGCTCAAGCAGCTTGAGGAGCAGAATGCCCGCCTG
AAGGATGCCCTGGTGAGGATGCGGGATCTTTCTTCCTCAGAGAAGCAGGA
GCATGTGAAGCTCCAGAAGCTCATGGAAAAGAAGAACCAAGAGCTGGAAG
TTGTGAGGCAACAGCGGGAGCGTCTGCAGGAGGAGCTAAGCCAGGCAGAG
AGCACCATTGATGAGCTCAAGGAGCAGGTGGATGCTGCTCTGGGTGCTGA
GGAGATGGTGGAGATGCTGACAGATCGGAACCTGAATCTGGAAGAGAAAG
TGCGCGAGTTGAGGGAGACTGTGGGAGACTTGGAAGCGATGAATGAGATG
AACGATGAGCTGCAGGAGAATGCACGTGAGACAGAACTGGAGCTGCGGGA
GCAGCTGGACATGGCAGGCGCGCGGGTTCGTGAGGCCCAGAAGCGTGTGG
AGGCAGCCCAGGAGACGGTTGCAGACTACCAGCAGACCATCAAGAAGTAC
CGCCAGCTGACCGCCCATCTACAGGATGTGAATCGGGAACTGACAAACCA
GCAGGAAGCATCTGTGGAGAGGCAACAGCAGCCACCTCCAGAGACCTTTG
ACTTCAAAATCAAGTTTGCTGAGACTAAGGCCCATGCCAAGGCAATTGAG
ATGGAATTGAGGCAGATGGAGGTGGCCCAGGCCAATCGACACATGTCCCT
GCTGACAGCCTTCATGCCTGACAGCTTCCTTCGGCCAGGTGGGGACCATG
ACTGCGTTCTGGTGCTGTTGCTCATGCCTCGTCTCATTTGCAAGGCAGAG
CTGATCCGGAAGCAGGCCCAGGAGAAGTTTGAACTAAGTGAGAACTGTTC
AGAGCGGCCTGGGCTGCGAGGAGCTGCTGGGGAGCAACTCAGCTTTGCTG
CTGGACTGGTGTACTCGCTGAGCCTGCTGCAGGCCACGCTACACCGCTAT
GAGCATGCCCTCTCTCAGTGCAGTGTGGATGTGTATAAGAAAGTGGGCAG
CCTGTACCCTGAGATGAGTGCCCATGAGCGCTCCTTGGATTTCCTCATTG
AACTGCTGCACAAGGATCAGCTGGATGAGACTGTCAATGTGGAGCCTCTC
ACCAAGGCCATCAAGTACTATCAGCATCTGTACAGCATCCACCTTGCCGA
ACAGCCTGAGGACTGTACTATGCAGCTGGCTGACCACATTAAGTTCACGC
AGAGTGCTCTGGACTGCATGAGTGTGGAGGTAGGACGGCTGCGTGCCTTC
TTGCAGGGTGGGCAGGAGGCTACAGATATTGCCCTCCTGCTCCGGGATCT
GGAAACTTCATGCAGTGACATCCGCCAGTTCTGCAAGAAGATCCGAAGGC
GAATGCCAGGGACAGATGCTCCTGGGATCCCAGCTGCACTGGCCTTTGGA
CCACAGGTATCTGACACGCTCCTAGACTGCAGGAAACACTTGACGTGGGT
CGTGGCTGTGCTGCAGGAGGTGGCAGCTGCTGCTGCCCAGCTCATTGCCC
CACTGGCAGAGAATGAGGGGCTACTTGTGGCTGCTCTGGAGGAACTGGCT
TTCAAAGCAAGCGAGCAGATCTATGGGACCCCCTCCAGCAGCCCCTATGA
GTGTCTGCGCCAGTCATGCAACATCCTCATCAGTACCATGAACAAGCTGG
CCACAGCCATGCAGGAGGGGGAGTATGATGCAGAGCGGCCCCCCAGCAAG

TABLE 123
Human DCTN 1 (P150) protein (SEQ ID NO.:156)

Exon 23 (nucleotide)
GGAGAGGAGCTAAGTGAGGCCAATGTGCGGCTGAGCCTCCTGGAGAAGAA
GTTGGACAGTGCTGCCAAGGATGCAGATGAGCGCATCGAGAAAGTCCAGA
CTCGGCTGGAGGAGACCCAGGCACTGCTGCGAAAGAAGGAGAA

TABLE 124
Human DCTN 1 (P150) protein (SEQ ID NO.:157)

Exon 24 (nucleotide)
AGAGTTTGAGGAGACAATGGATGCACTCCAGGCTGACATCGACCAGCTGG
AGGCAGAGAAGGCAGAACTAAAGCAGCGTCTGAACAGCCAGTCCAAACGC
ACGATTGAGGGACTCCGGGGCCCTCCTCCTTCAGGCATTGCTACTCTGGT
CTCTGGCATTGCTGGTG

TABLE 125
Human DCTN 1 (P150) protein (SEQ ID NO.:158)

Exon 23 to 24 (nucleotide)
GGAGAGGAGCTAAGTGAGGCCAATGTGCGGCTGAGCCTCCTGGAGAAGAA
GTTGGACAGTGCTGCCAAGGATGCAGATGAGCGCATCGAGAAAGTCCAGA
CTCGGCTGGAGGAGACCCAGGCACTGCTGCGAAAGAAGGAGAAAGAGTTT
GAGGAGACAATGGATGCACTCCAGGCTGACATCGACCAGCTGGAGGCAGA
GAAGGCAGAACTAAAGCAGCGTCTGAACAGCCAGTCCAAACGCACGATTG
AGGGACTCCGGGGCCCTCCTCCTTCAGGCATTGCTACTCTGGTCTCTGGC
ATTGCTGGTG

In another embodiment, the amino acid affected by the mutation is located in a domain of said protein, which is capable of binding to another subunit of the dynactin/dynein complex. Specifically, said domain comprises amino acids 147-157, amino acids 243-314, amino acids 140-157, or amino acids 1-123 of SEQ ID NO.: 61, in case the protein is mouse cytoplasmic dynein intermediate chain 1, or, in case the protein is human cytoplasmic dynein intermediate chain 1 amino acids 1-140, amino acids 157-174, amin acids 164-174, or amino acids 260-331 of SEQ ID NO.: 62. In case the protein is mouse cytoplasmic dynein intermediate chain 2, said domain comprises amino acids 1-123, amino acids 122-139, amino acids 129-130, or amino acids 226-297 of SEQ ID NO.: 64, or, in case the protein is human cytoplasmic dynein intermediate chain 2 amino acids 1-149, amino acids 148-165, amino acids 155-165, or amino acids 252-323 of SEQ ID NO.: 65. In case the protein is mouse DCTN 1 (SEQ ID NO.: 67), said domain, comprises amino acids 39-150, amino acids 1006-1021, or amino acids 133-899 of SEQ ID NO.: 67, or, in case the protein is human DCTN 1 amino acids 39-150, amino acids 133-899, or amino acids 1006-1021 of SEQ ID NO.: 68.

In another preferred embodiment, the amino acid affected by the mutation is any one as described in individualized manner in connection with the preferred muteins of the invention above, e.g., those specified in Tables 19, 20, 21, 22 or 24.

In case the protein is human DCTN 1, another preferred embodiment is one wherein the amino acid affected by said mutation is amino acid 39G.

In case the protein is human DCTN 1, another preferred embodiment is one wherein the amino acid affected by said mutation is amino acid 40H.

In case the protein is human DCTN 1, another preferred embodiment is one wherein the amino acid affected by said mutation is amino acid 41R.

In case the protein is human DCTN 1, another preferred embodiment is one wherein the amino acid affected by said mutation is amino acid 42G.

In case the protein is human DCTN 1, another preferred embodiment is one wherein the amino acid affected by said mutation is amino acid 43T.

In case the protein is human DCTN 1, another preferred embodiment is one wherein the amino acid affected by said mutation is amino acid 44V.

In case the protein is human DCTN 1, another preferred embodiment is one wherein the amino acid affected by said mutation is the amino acid 45A.

In case the protein is human DCTN 1, another preferred embodiment is one wherein the amino acid affected by said mutation is the amino acid 46Y.

In case the protein is human DCTN 1, another preferred embodiment is one wherein the amino acid affected by said mutation is the amino acid 47V.

In case the protein is human DCTN 1, another preferred embodiment is one wherein the amino acid affected by said mutation is the amino acid 48G.

In case the protein is human DCTN 1, another preferred embodiment is one wherein the amino acid affected by said mutation is the amino acid 49A.

In case the protein is human DCTN 1, another preferred embodiment is one wherein the amino acid affected by said mutation is the amino acid 50T.

In case the protein is human DCTN 1, another preferred embodiment is one wherein the amino acid affected by said mutation is the amino acid 51L.

In case the protein is human DCTN 1, another preferred embodiment is one wherein the amino acid affected by said mutation is the amino acid 52F.

In case the protein is human DCTN 1, another preferred embodiment is one wherein the amino acid affected by said mutation is the amino acid 53A.

In case the protein is human DCTN 1, another preferred embodiment is one wherein the amino acid affected by said mutation is the amino acid 54T.

In case the protein is human DCTN 1, another preferred embodiment is one wherein the amino acid affected by said mutation is the amino acid 55G.

In case the protein is human DCTN 1, another preferred embodiment is one wherein the amino acid affected by said mutation is the amino acid 56K.

In case the protein is human DCIN 1, another preferred embodiment is one wherein the amino acid affected by said mutation is the amino acid 57W.

In case the protein is human DCTN 1, another preferred embodiment is one wherein the amino acid affected by said mutation is the amino acid 58V.

In case the protein is human DCTN 1, another preferred embodiment is one wherein the amino acid affected by said mutation is the amino acid 59G.

In case the protein is human DCTN 1, another preferred embodiment is one wherein the amino acid affected by said mutation is the amino acid 60V

In case the protein is human DCTN 1, another preferred embodiment is one wherein the amino acid affected by said mutation is the amino acid 611.

In case the protein is human DCTN 1, another preferred embodiment is one wherein the amino acid affected by said mutation is the amino acid 62L.

In case the protein is human DCTN 1, another preferred embodiment is one wherein the amino acid affected by said mutation is the amino acid 63D.

In case the protein is human DCTN 1, another preferred embodiment is one wherein the amino acid affected by said mutation is the amino acid 64E.

In case the protein is human DCTN 1, another preferred embodiment is one wherein the amino acid affected by said mutation is the amino acid 65A.

In case the protein is human DCTN 1, another preferred embodiment is one wherein the amino acid affected by said mutation is the amino acid 66K.

In case the protein is human DCTN 1, another preferred embodiment is one wherein the amino acid affected by said mutation is the amino acid 67G.

In case the protein is human DCTN 1, another preferred embodiment is one wherein the amino acid affected by said mutation is the amino acid 68K.

In case the protein is human DCFN 1, another preferred embodiment is one wherein the amino acid affected by said mutation is the amino acid 69N.

In case the protein is human DCTN 1, another preferred embodiment is one wherein the amino acid affected by said mutation is the amino acid 70D.

In case the protein is human DCTN 1, another preferred embodiment is one wherein the amino acid affected by said mutation is the amino acid 71 G.

In case the protein is human DCTN 1, another preferred embodiment is one wherein the amino acid affected by said mutation is the amino acid 72T.

In case the protein is human DCTN 1, another preferred embodiment is one wherein the amino acid affected by said mutation is the amino acid 73V.

In case the protein is human DCTN 1, another preferred embodiment is one wherein the amino acid affected by said mutation is the amino acid 74Q.

In case the protein is human DCTN 1, another preferred embodiment is one wherein the amino acid affected by said mutation is the amino acid 75G.

In case the protein is human DCTN 1, another preferred embodiment is one wherein the amino acid affected by said mutation is the amino acid 76R.

In case the protein is human DCTN 1, another preferred embodiment is one wherein the amino acid affected by said mutation is the amino acid 77K.

In case the protein is human DCTN 1, another preferred embodiment is one wherein the amino acid affected by said mutation is the amino acid 78Y.

In case the protein is human DCTN 1, another preferred embodiment is one wherein the amino acid affected by said mutation is the amino acid 79F.

In case the protein is human DCTN 1, another preferred embodiment is one wherein the amino acid affected by said mutation is the amino acid 80T.

In case the protein is human DCTN 1, another preferred embodiment is one wherein the amino acid affected by said mutation is the amino acid 81C.

In case the protein is human DCTN 1, another preferred embodiment is one wherein the amino acid affected by said mutation is the amino acid 82D.

In case the protein is human DCTN 1, another preferred embodiment is one wherein the amino acid affected by said mutation is the amino acid 83E.

In case the protein is human DCTN 1, another preferred embodiment is one wherein the amino acid affected by said mutation is the amino acid 84G.

In case the protein is human DCTN 1, another preferred embodiment is one wherein the amino acid affected by said mutation is the amino acid 85H.

In case the protein is human DCTN 1, another preferred embodiment is one wherein the amino acid affected by said mutation is the amino acid 86G.

In case the protein is human DCTN 1, another preferred embodiment is one wherein the amino acid affected by said mutation is the amino acid 871.

In case the protein is human DCTN 1, another preferred embodiment is one wherein the amino acid affected by said mutation is the amino acid 88F.

In case the protein is human DCTN 1, another preferred embodiment is one wherein the amino acid affected by said mutation is the amino acid 89V.

In case the protein is human DCTN 1, another preferred embodiment is one wherein the amino acid affected by said mutation is the amino acid 90R.

In case the protein is human DCTN 1, another preferred embodiment is one wherein the amino acid affected by said mutation is the amino acid 91 Q.

In case the protein is human DCTN 1, another preferred embodiment is one wherein the amino acid affected by said mutation is the amino acid 92S.

In case the protein is human DCTN 1, another preferred embodiment is one wherein the amino acid affected by said mutation is the amino acid 93Q.

In case the protein is human DCTN 1, another preferred embodiment is one wherein the amino acid-affected by said mutation is the amino acid 94I.

In case the protein is human DCTN 1, another preferred embodiment is one wherein the amino acid affected by said mutation is the amino acid 95Q.

In case the protein is human DCTN 1, another preferred embodiment is one wherein the amino acid affected by said mutation is the amino acid 96V.

In case the protein is human DCTN 1, another preferred embodiment is one wherein the amino acid affected by said mutation is the amino acid 97F.

In case the protein is human DCTN 1, another preferred embodiment is one wherein the amino acid affected by said mutation is the amino acid 98E.

In case the protein is human DCTN 1, another preferred embodiment is one wherein the amino acid affected by said mutation is the amino acid 99D.

In case the protein is human DCTN 1, another preferred embodiment is one wherein the amino acid affected by said mutation is the amino acid 100G.

In case the protein is human DCTN 1, another preferred embodiment is one wherein the amino acid affected by said mutation is the amino acid 101A.

In case the protein is human DCTN 1, another preferred embodiment is one wherein the amino acid affected by said mutation is the amino acid 102D.

In case the protein is human DCTN 1, another preferred embodiment is one wherein the amino acid affected by said mutation is the amino acid 103T.

In case the protein is human DCTN 1, another preferred embodiment is one wherein the amino acid affected by said mutation is the amino acid 104T.

In case the protein is human DCTN 1, another preferred embodiment is one wherein the amino acid affected by said mutation is the amino acid 105S.

In case the protein is human DCTN 1, another preferred embodiment is one wherein the amino acid affected by said mutation is the amino acid 106P.

In case the protein is human DCTN 1, another preferred embodiment is one wherein the amino acid affected by said mutation is the amino acid 107E.

In case the protein is human DCTN 1, another preferred embodiment is one wherein the amino acid affected by said mutation is the amino acid 108T.

In case the protein is human DCTN 1, another preferred embodiment is one wherein the amino acid affected by said mutation is the amino acid 109P.

In case the protein is human DCTN 1, another preferred embodiment is one wherein the amino acid affected by said mutation is the amino acid 110D.

In case the protein is human DCTN 1, another preferred embodiment is one wherein the amino acid affected by said mutation is the amino acid 111S.

In case the protein is human DCTN 1, another preferred embodiment is one wherein the amino acid affected by said mutation is the amino acid 112S.

In case the protein is human DCTN 1, another preferred embodiment is one wherein the amino acid affected by said mutation is the amino acid 113A.

In case the protein is human DCTN 1, another preferred embodiment is one wherein the amino acid affected by said mutation is the amino acid 114S.

In case the protein is human DCTN 1, another preferred embodiment is one wherein the amino acid affected by said mutation is the amino acid 115K.

In case the protein is human DCTN 1, another preferred embodiment is one wherein the amino acid affected by said mutation is the amino acid 117L.

In case the protein is human DCTN 1, another preferred embodiment is one wherein the amino acid affected by said mutation is the amino acid 118K.

In case the protein is human DCTN 1, another preferred embodiment is one wherein the amino acid affected by said mutation is the amino acid 119R.

In case the protein is human DCTN 1, another preferred embodiment is one wherein the amino acid affected by said mutation is the amino acid 120E.

In case the protein is human DCTN 1, another preferred embodiment is one wherein the amino acid affected by said mutation is the amino acid 121G.

In case the protein is human DCTN 1, another preferred embodiment is one wherein the amino acid affected by said mutation is the amino acid 123D.

In case the protein is human DCTN 1, another preferred embodiment is one wherein the amino acid affected by said mutation is the amino acid 126A.

In case the protein is human DCTN 1, another preferred embodiment is one wherein the amino acid affected by said mutation is the amino acid 127K.

In case the protein is human DCTN 1, another preferred embodiment is one wherein the amino acid affected by said mutation is the amino acid 128T.

In case the protein is human DCTN 1, another preferred embodiment is one wherein the amino acid affected by said mutation is the amino acid 129S.

In case the protein is human DCTN 1, another preferred embodiment is one wherein the amino acid affected by said mutation is the amino acid 130K.

In case the protein is human DCTN 1, another preferred embodiment is one wherein the amino acid affected by said mutation is the amino acid 131L.

In case the protein is human DCTN 1, another preferred embodiment is one wherein the amino acid affected by said mutation is the amino acid 132R.

In case the protein is human DCTN 1, another preferred embodiment is one wherein the amino acid affected by said mutation is the amino acid 133G.

In case the protein is human DCTN 1, another preferred embodiment is one wherein the amino acid affected by said mutation is the amino acid 134L.

In case the protein is human DCTN 1, another preferred embodiment is one wherein the amino acid affected by said mutation is the amino acid 135K.

In case the protein is human DCTN 1, another preferred embodiment is one wherein the amino acid affected by said mutation is the amino acid 136P.

In case the protein is human DCTN I, another preferred embodiment is one wherein the amino acid affected by said mutation is the amino acid 137K.

In case the protein is human DCTN 1, another preferred embodiment is one wherein the amino acid affected by said mutation is the amino acid 138K.

In case the protein is human DCTN 1, another preferred embodiment is one wherein the amino acid affected by said mutation is the amino acid 139A.

In case the protein is human DCTN 1, another preferred embodiment is one wherein the amino acid affected by said mutation is the amino acid 140P.

In case the protein is human DCTN 1, another preferred embodiment is one wherein the amino acid affected by said mutation is the amino acid 141T.

In case the protein is human DCTN 1, another preferred embodiment is one wherein the amino acid affected by said mutation is the amino acid 142A.

In case the protein is human DCTN 1, another preferred embodiment is one wherein the amino acid affected by said mutation is the amino acid 143R.

In case the protein is human DCTN 1, another preferred embodiment is one wherein the amino acid affected by said mutation is the amino acid 144K.

In case the protein is human DCTN 1, another preferred embodiment is one wherein the amino acid affected by said mutation is the amino acid 145T.

In case the protein is human DCTN 1, another preferred embodiment is one wherein the amino acid affected by said mutation is the amino acid 146T.

In case the protein is human DCTN 1, another preferred embodiment is one wherein the amino acid affected by said mutation is the amino acid 147T.

In case the protein is human DCTN 1, another preferred embodiment is one wherein the amino acid affected by said mutation is the amino acid 148R.

In case the protein is human DCTN 1, another preferred embodiment is one wherein the amino acid affected by said mutation is the amino acid 149R.

In case the protein is human DCTN 1, another preferred embodiment is one wherein the amino acid affected by said mutation is the amino acid 150P.

The method of identifying a protein or nucleic acid marker indicative of an increased risk of a mammalian subject of developing a neurodegenerative disease, or the method of identifying a protein or nucleic acid marker indicative of an association of a neurodegenerative disease in a mammalian subject with a mutation in an allele of a gene coding for a protein, which is a subunit of the dynactin/dynein complex comprise the step of analyzing a test sample derived from said subject for the presence of a difference compared to a similar test sample if derived from a subject of the same species that is unaffected by, or known not to be at risk of, developing said disease, wherein said difference is indicative of the presence of a mutation in an allele of the gene coding for a protein, which is a subunit of the dynactin/dynein complex.

In the above methods, the test sample derived from a mammalian subject, particularly a human subject, may be directly obtained from said subject. It may, however, also be a sample that has been obtained previously. Also included test samples according to the invention are, for example, cDNA preparations that have been prepared from mRNA obtained from a tissue sample from a human subject at an earlier stage. It may also be cloned or PCR-amplified DNA that originates from DNA contained in such tissue sample obtained at an earlier stage.

According to the claimed method, the test sample will be analyzed for a difference to a similar test sample derived from a human subject unaffected by or known not to be at risk of developing a neurological disease. While the method may include actually deriving or directly obtaining a test sample from such a mammalian subject, particularly a human subject, for comparative purposes, the necessary information regarding the relevant structural features and properties of such similar test sample to be used for comparison will often already be available. Thus, it will often be sufficient for the purposes of the above methods of the invention to perform an analysis for a difference to a similar test sample as it would be observed if said similar test sample were in fact obtained from a subject of the same species, which subject is unaffected by or known not to be at risk of developing the above medical condition.

The test sample may be a nucleic acid sample, e.g., mRNA (or cDNA derived therefrom), or genomic DNA.

It may also be a protein sample. In a preferred embodiment the nucleic acid sample or the protein sample comprises one or more of the nucleic acids coding for proteins or the proteins itself, which are subunits of the dynactin/dynein complex, preferably the cytoplasmic dynein heavy chain 1, e.g. according to SEQ ID NOS.: 28 or 29, the cytoplasmic dynein intermediate chain 1, e.g. according to SEQ ID NOS.: 30 or 31, the cytoplasmic dynein intermediate chain 2, e.g. according to SEQ ID NOS.: 32 or 33, the cytoplasmic dynein light intermediate chain 1, e.g. according to SEQ ID NOS.: 34 or 35, the cytoplasmic dynein light intermediate chain 2, e.g. according to SEQ ID NOS.: 36 or 37; the cytoplasmic dynein 10 kDa light chain, e.g. according to SEQ ID NOS.: 38 or 39, the cytoplasmic dynein light chain Tctex 1, e.g. according to SEQ ID NOS.: 40 or 41, the cytoplasmic dynein light chain 2B, e.g. according to SEQ ID NO.: 42, DCTN 1, e.g. according to SEQ ID NOS.: 43 or 44, DCTN 2, e.g. according to SEQ ID NO.: 45, DCTN 3, e.g. according to SEQ ID NOS.: 46 or 47), DCTN 4, e.g. according to SEQ ID NOS.: 48 or 49, DCTN 5, e.g. according to SEQ ID NO.: 50, DCTN 6, e.g. according to SEQ ID NOS.: 51 or 52), ARP1, e.g. according to SEQ ID NOS. 53 or 54, ARP II, e.g. according to SEQ ID NOS. 55 or 56), HAP1, e.g. according to SEQ ID NO. 57 or 58, and CLIP-170, e.g. according to SEQ ID NOS.: 59 or 60), more preferably the cytoplasmic dynein heavy chain 1, cytoplasmic dynein intermediate chain 1, cytoplasmic dynein intermediate chain 2, and/or DCTN 1, e.g., according to the above-mentioned corresponding SEQ ID NOS.

The difference analyzed may furthermore be one relating to the expression level of said nucleic acid or protein. Alternatively, it may be analyzed whether there is a difference in terms of the nucleotide or the amino acid sequence level.

The step of analysis for differences between the test samples may comprises the partial or complete determination of the sequence of the nucleic acid, or a PCR-amplified portion of the nucleic acid, of the test sample, and optionally also of the nucleic acid or at PCR-amplified portion of the nucleic acid of the similar test sample (or the similar test samples).

Accordingly, another embodiment of the invention relates to oligonucleotides suitable for identifying a mutation in an allele coding for a protein, which is a subunit of the dynactin/dynein complex. In a preferred embodiment, the nucleic acid sequence of the oligonucleotide corresponds to one within said allele, which contains a mutation as defined above. In another preferred embodiment the oligonucleotide is suitable for hybridizing to the nucleic acid of said allele or a portion of its nucleic acid under stringent conditions. In another preferred embodiment, the oligonucleotide is suitable as primer for amplifying the nucleic acid of said allele or a portion of its nucleic acid.

Suitable methods for the determination of partial or complete nucleic acid sequences, and thus, detection of the above-mentioned differences, are well known to the skilled artisan. They include, for example, Southern blotting, TGGE (temperature gradient gel electrophoresis), DGGE (denaturing gradient gel electrophoresis), SCCP (single chain conformation polymorphism) detection, and the like. High throughput sequence analysis methods such as those described by Kristensen et al. (Kristensen et al., BioTechniques 30 (2001), 318-332), which is incorporated herein by reference in its entirety, are likewise suitable, and hence, contemplated in connection with the present invention.

One embodiment of the present invention relates to the determination whether the above-mentioned differences are present in one or both alleles of the gene coding for the protein, which is a subunit of the dynactin/dynein complex, i.e., whether the mammalian subject (or certain cell samples derived therefrom) is (are) homozygous or heterozygous with respect to said mutation. In a preferred embodiment, the portion of the allele to be tested is PCR-amplified and digested with a suitable restriction enzyme and the resulting restriction pattern obtained from the amplified portion is compared to the restriction pattern obtained from the corresponding amplified portion of the similar sample or the similar samples (see Example (8). In another preferred embodiment, the above-mentioned determination whether differences are present in one or both alleles of the gene coding for said protein is TGCE, as explained below.

Another preferred embodiment of the invention relates to the separation of PCR-amplified portions of the allele via electrophoresis through a gradient of increasing temperature via TGCE (see Example 20). Differences in the amplified portion of the allele compared to amplified portions of the alleles contained in the similar test sample(s) are indicated by a difference in migration during electrophoresis.

As mentioned above, the invention inter alia relates to high throughput screening methods. Preferably, the oligonucleotides according to the invention, used in these high throughput methods, are fixed on a solid support to form an array. The solid support can comprise microplates, standard blotting membranes, or glass chips. In a preferred embodiment, the solid support is a glass chip with a modified surface capable of covalently binding to said oligonucleotides. Nucleic acid samples, preferably genomic DNA, are tagged, e.g. with a fluorescent marker, and allowed to interact with said oligoncleotides bound to the solid support. Hybridization will occur at complementary sequences between nucleic acid sample and oligonucleotide, which is indicative for the presence of a mutation in said nucleic acid sample. Hybridization is then determined by scanning with a laser beam and analyzing the signal on a computer.

Suitable methods for the determination of partial or complete amino acid sequences are likewise well known, and include, for example, detection of particular epitopes within a protein sample via specific antibodies in dot blot, slot blot, or Western blot assays, or via ELISAs or RIAs, or partial amino acid sequence determination on a sequencer via Edman degradation. Also, high-throughput methods may again be employed.

A further aspect of the present invention is represented by a method for identifying a predisposition of a mammalian subject, particularly a human subject, for developing a neurodegenerative disease, said method comprising the step of determining whether a test sample derived from said subject indicates the presence of a mutation in an allele of the gene coding for a protein, which is a subunit of the dynactin/dynein complex, indicative of an increased risk of said human subject of developing said neurodegnereative disease.

Also contemplated in connection with the present invention is a method for determining whether a neurodegenerative disease in a mammalian subject, particularly a human subject, which is associated with a mutation in an allele of a gene coding for a protein, which is asubunit of the dynactinidynein complex, said method comprising the step of determining whether a test sample derived from said subject indicates the presence of a mutation in an allele of the gene coding for said protein.

As in the case of the methods described above, the test sample may also be a sample that has been obtained previously. Furthermore, suitable test samples according to the invention are, for example, cDNA preparations that have been prepared from mRNA obtained from a tissue sample from a mammalian subject, particularly a human subject, at an earlier stage. It may also again be cloned or PCR-amplified DNA that originates from DNA contained in such tissue sample obtained at an earlier stage.

Again, the previously mentioned methods of determining partial or complete nucleic acid or amino acid sequences may be employed for the step of determining whether the test sample (which may be a nucleic acid or protein test sample as previously defined) indicates the presence of said mutation.

According to the above methods of identifying a predisposition in a mammalian subject, particularly a human subject, of developing a neurodegenerative disease, or determining a potential association between such a neurodegenerative disease and certain mutations, the test sample is analyzed for the presence of a mutation in an allele of a gene of a protein, which is a subunit of the dynactin/dynein complex, which mutation is indicative of an increased risk of developing such a neurodegenerative disease.

On the molecular level, said methods would comprise analyzing said sample for the presence of amutation, which would indicate an altered expression or function of said protein. It will be appreciated that such mutations are inter alia those referred to herein in connection with the proteins and nucleic acids according to the invention, and that mutations of this kind may be readily identified, for example, by in vitro assays. Such in vitro assays to determine whether the mutation in an allele of a gene coding for said protein affects the function of the complex may comprise the quantitative and/or qualitative determination of the binding of the subunits to each other within the complex. Qualitative methods may comprise SDS-PAGE and subsequent Western blotting of homogenates of, e.g. the spinal cord, using antibodies specific for the dynactin/dynein subunits. Disruption of the dynactin/dynein complex by a mutation in said proteins can be determined by, e.g. a difference in migration of the subunits compared to proteins derived from homogenates of similar tissue comprising the corresponding proteins without mutation (LaMonte et al., see above.). In another embodiment of the invention, disruption, or at least functional alteration of the dynactin/dynein complex due to mutations in its subunits, may be analyzed by affinity chromatography as described in Karki et al. (J Biol Chem 270, 28806-28811, 1995) or co-precipitation experiments as described in Paschal et al. (J Biol Chem 268, 15318-23, 1993). Quantitative methods to determine quantitative binding of the subunits within the complex to each other are, e.g. those using surface plasmon resonance (SPR), such as, for example, the Biacore technology (Biacore AB, Uppsala, Sweden) (see also Example 18). Further suitable in vitro assays comprise the step of determining whether a mutation in one or more subunits of the dynactin/dynein complex affects dynein motility. Such tests comprise, e.g. immunohistology with appropiate antibodies. To determine an effect of the mutation on axonal transport, antibodies directed against tubulin or synaptotagmin may be suitable (Martin et al., 1999, Mol Biol Cell 10, 3717-3728).

In another embodiment of the invention, dynein motility may be determined by analysis of single-lipid-droplet motility as described by Welte et al. (Cell 92: 547-557, 1998) or a microtubule gliding assay (see Example 16). In another embodiment, the in vitro assays may comprise tests to determine whether the mutation in an allele of a gene coding for said protein affects the expression of the protein. In this regard, quantitative analysis of the mRNA expression levels may be applicable. These methods may comprise, for example, Northern blotting or quantitative Polymerase Chain Reaction, which are well known to those skilled in the art.

In a further embodiment of the present invention, the above-metioned mutations may be readily identified, for example, by an animal model (see Example 1).

The above-mentioned mutations may also be identified by any of the afore-mentioned methods of screening for disease-relevant alleles of said proteins.

The invention further comprises a kit for identifying a predisposition of a mammalian subject, particularly a human subject, for developing a neurodegenerative disease, or for identifying an association of a neurodegenerative disease of said subject with a mutation in an allele coding for a protein, which is a subunit of the dynactin/dynein complex. The kit according to the invention comprises one or more of the above-mentioned oligonucleotides suitable for identifying a mutation in an allele of a gene coding for a protein which is a subunit of the dynactin/dynein complex. In one preferred embodiment, the kit comprises oligonucleotide(s), the nucleotide sequence of which correspond(s) to a nucleotide sequence within said allele, which contains a mutation. In another preferred embodiment, the oligonucleotide(s) is(are) suitable for hybridizing to the nucleic acid of said allele or a portion of its nucleic acid under stringent conditions. In another preferred embodiment, the oligonucleotide(s) is(are) suitable as primer(s) for amplifying the nucleic acid of said allele or a portion of its nucleic acid. In another preferred embodiment, the kit further comprises instructions to use the oligonucleotide or the oligonucleotides for identifying said predisposition in said subject or said association of the neurodegenerative disease of said subject with said mutation.

The invention further comprises a solid support, wherein at least two oligonucleotides are individually fixed to separate areas of the solid support to form an array. Preferrably the oligonucleotides fixed on the solid support are suitable for for identifying a mutation in an allele of a gene coding for a protein, which is a subunit of the dynactin/dynein complex. Preferrably, the oligonucleotides fixed on the solid support have a nucleotide sequence, which corresponds to a nucleotide sequence within said allele, which contains a mutation and are suitable for hybridizing to the nucleic acid of said allele or a portion of its nucleic acid under stringent conditions. The solid support can be a microplate or standard blotting membranes, such as nylon or nitrocellulose membranes. In a preferred embodiment, the solid support is a glass chip, preferably a glass chip with a modified surface. The surface of the glass chip is modified as to bind nucleic acids, preferably oligonucleotides, more preferably oligonucleotides with a nucleotide sequence, which corresponds to a nucleotide sequence within said allele, which contains a mutation.

A further embodiment of the present invention comprises the use of the previously mentioned oligonucleotides, or the previously mentioned kit, or the above-mentioned solid support, in a method for identifying a predisposition of a mammalian subject, particularly a human subject, for developing a neurodegenerative disease or in a method for determining whether a neurodegenerative disease in a mammalian subject, particularly a human subject, is associated with a mutation in an allele of a gene coding for a protein, which is a subunit of the dynactin/dynein complex as mentioned previously.

The invention will be further described in the following examples, which do not limit the scope of the invention described in the claims. The following examples are offered for illustrative purposes only, and are not intended to limit the scope of the present invention in any way.

### EXAMPLE 1

### Production of Animals of the Invention and Breeding Strategies

To produce mouse mutants, a C3HeB/FeJ male mouse (The Jackson Laboratory, Bar Harbor Me., USA) was injected intraperitoneally three times in weekly intervals between 8-10 weeks of age with ethyl-nitroso-urea (ENU) (Serva Electrophoresis GmbH, Heidelberg, Germany) at 90 mg/kg body weight.

Generation of F1 Progeny:

50 days after the last injection the injected male mouse was mated to wild type C3HeB/FeJ female partners. The F1 progeny (up to 100 offspring) were then analyzed for phenotypes of dominant traits.

Generation of F2 Progeny-Genetic Confirmation of Dominant Phenotype:

Affected F1 individuals were mated to wild type C3HeB/FeJ mice and the resulting F2 progeny was screened for the identical pathological phenotype as identified in F1 generation.

Generation of F3 Progeny:

For maintenance purposes affected F2 individuals were mated to wild type C3HeB/FeJ mice. Affected F2 individuals were intercrossed to produce homozygous F3 individuals, which undergo perinatal lethality: therefore fetal tissues of these animals were analyzed. Heterozygous F3 individuals were used for the histochemical, neurological and physiological characterization of the Cra1 phenotype.

### EXAMPLE 2

### First Identification of Phenotype

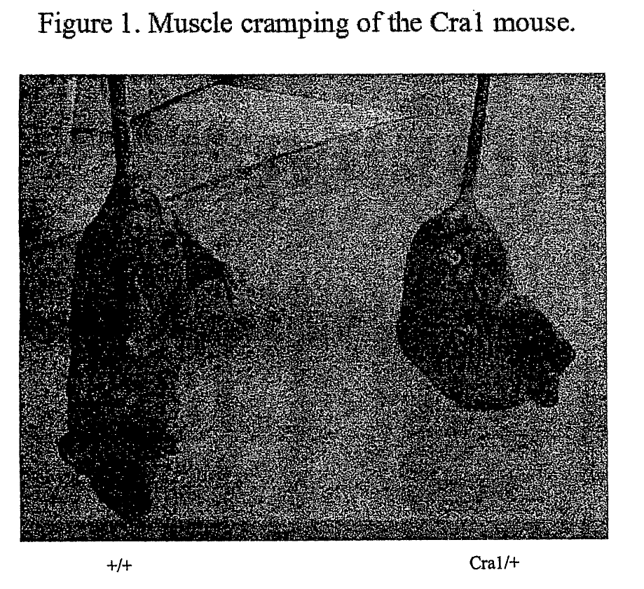

The Cra1 mouse mutant was identified by the observation of hind limb cramping during manual tail suspension in the heterozygous condition (FIG. 1). Adult mice were manually suspended by their tails for one minute. During this period it was observed whether the mice display cramping of their hind limbs. In all tests, the observation of hind limb cramping was invariably correlated to the heterozygous Cra1/+ genotype.

A broad range of standard physiological values were tested, but were within wild type (control) ranges.

### EXAMPLE 3

### Analysis of Muscle Endurance and Motor Coordination

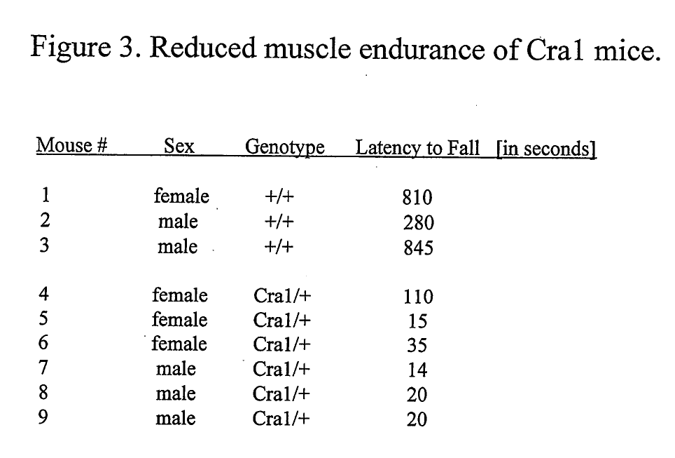

A semi-quantitative evaluation of the phenotype was accomplished by the application of the hanging wire assay (for results see FIG. 3), as a tool for measuring muscle endurance. Muscle endurance of adult heterozygous Cra1 individuals (Cra1/+) was measured in comparison to wildtype individuals by placing individual mice on a wire grid, mildly shaking the grid to cause the mouse to grip the wires with their paws, and then inverting the grid. The grid was held stationary in the inverted position and the time elapsed until the mouse fell was recorded as the latency to fall. Both sexes of Cra1 mice exhibit a significantly reduced latency to fall off the grid in comparison to wild type mice.

A reduction in motor coordination was subsequently suggested by a reduced performance in the coat hanger and stationary beam assays. In a coat hanger assay a single heterozygous Cra1 animal was placed in the middle of the horizontal part of a metal coat hanger. The movements of the animal were observed for 45 seconds and assessed in comparison to movements of a wildtype mice in a separate assay. The following classification was used to describe the performance of the animal: “normal”, the mouse climbs up the wire (horizontal and vertical movements); “abnormal”, the mouse displays only horizontal movements or it does not display any movements (freeze) or it falls from the metal coat hanger.

In the stationary beam assay, a heterozygous mouse was placed in the middle of a stationary beam, which was divided into fourteen 5 cm zones. The animal was observed for 45 seconds. The following classification was used to describe the performance of the animal: “normal”, the mouse moves between at least 3 zones; “abnormal”, the mouse moves within less than 3 zones, or the mouse is falls from the stationary beam.

### EXAMPLE 4

### Determination of Movement Activity

To determine movement activity during an 24 hour interval, individual heterozygous Cra1 or wild type mice were monitored with a photobeam activity system (San Diego Instruments, San Diego, Calif., USA), according to the manufacturer's instructions. A single animal was transferred into a type 3 mouse cage, with a photobeam frame assembled. Beam breaks caused by a moving mouse were counted automatically using a software package “SDI PAS Photobeam System V2” following the instructions of the manufacturer's handbook. Data were collected over a period of 24 hours after the animals had been acclimatized to the cages for 48 hours.

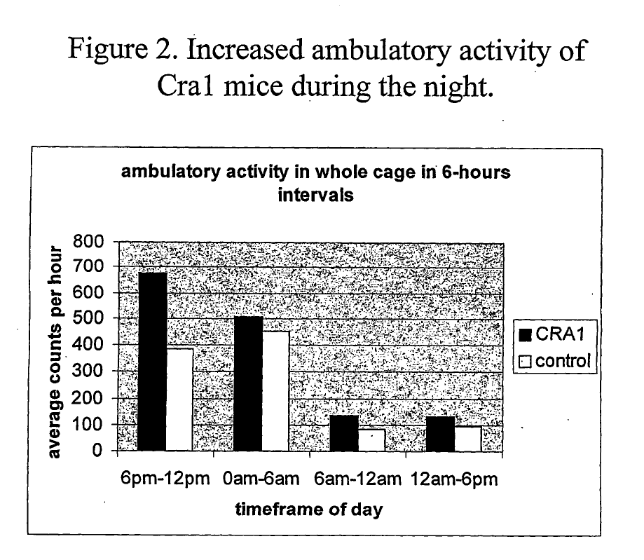

FIG. 2 indicates that heterozygous Cra1 mice exhibit increased ambulatory activity in the early dark phase of an alternating 12 hour light/dark cycle.

### EXAMPLE 5

### Necropsy and Organ Histology of Mutant Animals

For sampling of organs, adult animals were perfusion fixed with 4% paraformaldehyde. Muscle samples were collected from anaesthesised mice and cryopreserved in liquid nitrogen cooled isopentane at −50° C. Embryos were immersion fixed in paraformaldehyde. Tissues were sampled and processed to paraffin wax. Sections were cut at 5 microns and stained according to subsequent usage.

1. Heterozygotes

The following histopathological aberrations were observed in adult Cra1/+(as compared to wild type mice)

in I). Brain:

Slight enlargement of the lateral and 3^{rd} ventricles.

Neuronal eosinophilic inclusions in the arcuate and the preoptic nuclei of the hypothalamus.

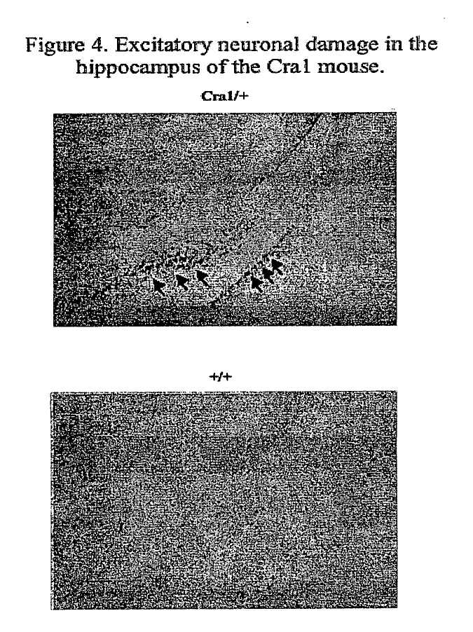

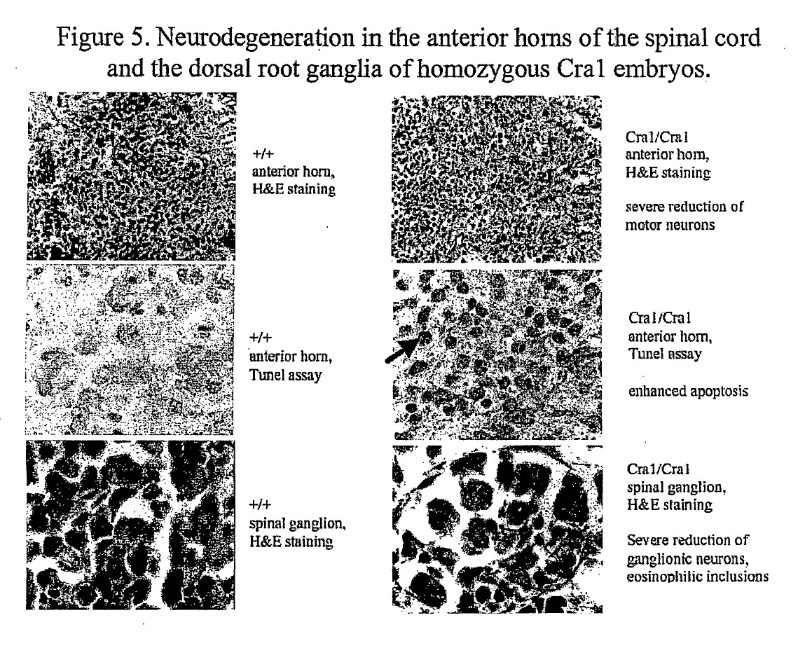

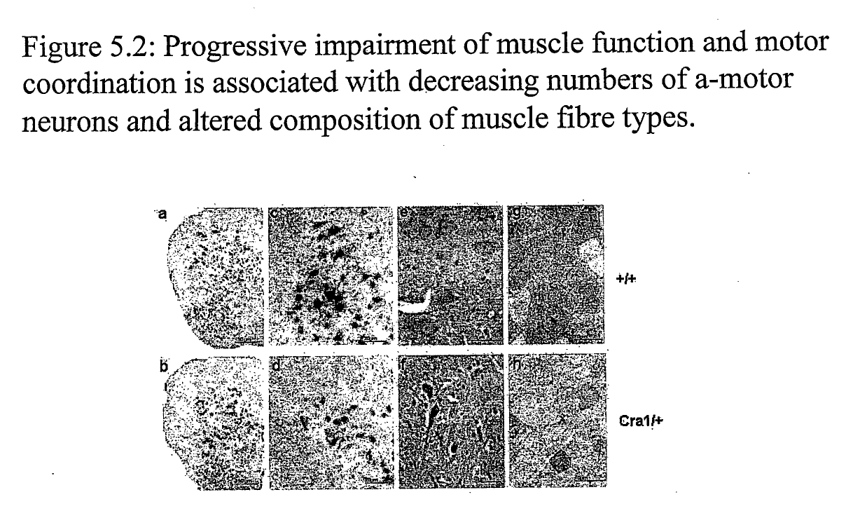

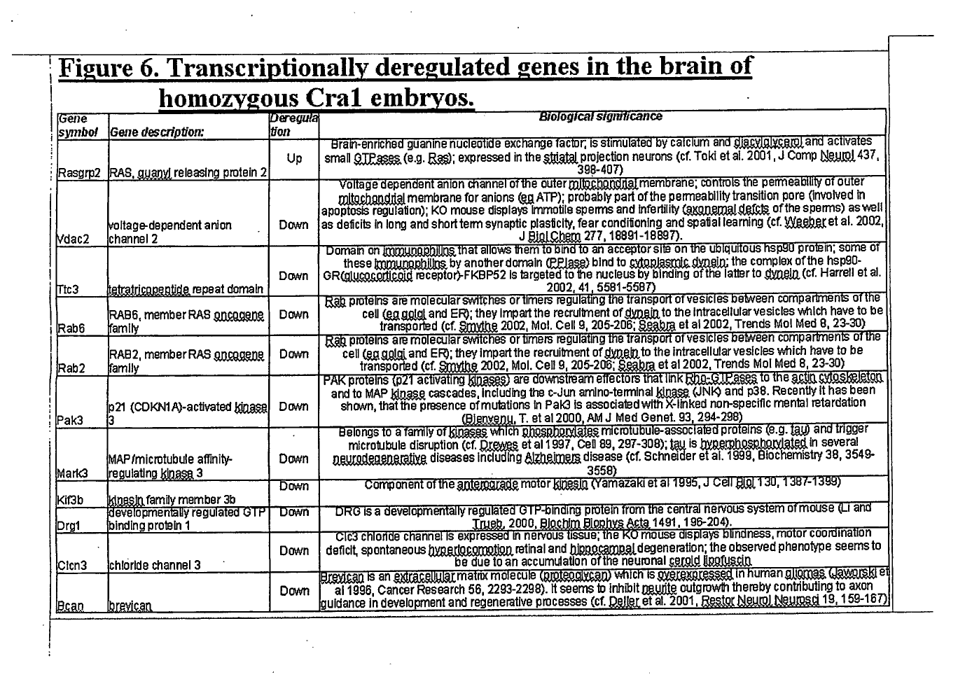

Excitatory neuronal damage (“dark neurons”) in hippocampus (gyrus dentatus, CA4, CA3), some areas of the upper cortex and the Purkinje cell layer of the cerebellum (see FIG. 4A: hippocampus):

This type of neuronal damage is reversible: it can be evoked by challenging the individuals by tail suspension for two minutes immediately before sacrifice by cervical dislocation, and can be demonstrated as follows. Brains were removed immediately, immersion fixed in paraformaldehyde and processed for paraffin embedding using standard histopathological techniques. Coronal sections of 5 μm were mounted on glass slides and stained with hematoxylin and eosin by standard histological protocols. The phenomenon of dark neurons after acute neuronal cell stress is well recognized since some years in the neuropathological literature and resembles a fixation effect by aldehyde fixation. This cellular pathomorphology is not accompanied by astro- or microglial activation, it occurs under normal neuronal cell count, and it is only detectable after aldehyde fixation, but not by cryosectioning.

in II). Muscle:

Decreasing numbers of α-motor neurons and altered composition of musculus vastus fibre types. Muscle samples were processed according to standard protocols and were cut at 5 μm. Staining of NeuN protein was performed by immunohistochemical analysis with an anti NeuN antibody Mab377 (Chemicon Int. Inc., Temecula, Calif., USA), detecting decreasing numbers of α-motor neurons. Periodic acid-Schiff (PAS) staining was performed according to standard protocols, detecting an altered composition of musculus vastus fiber tybes (see FIG. 4B).

2. Homozygotes

The following preliminary histopathological observations were made in organs of E17.5

Cra1/Cra1 fetus as compared to wild type individuals: Neurodegeneration in the anterior horns of the spinal cord:

Mature motor neurons were sparse in the anterior horn region of homozygotes when compared to heterozygote or wildtype littermates. From a histomorphological aspect approximately 50% of motor neuros were missing, the remaining neurons were smaller of size and did not have a normal mature aspect.

Detection of apoptotic cell death within the anterior horns of the spinal cord:

Apoptosis detection was performed as follows: deparaffinized and rehydrated paraffin sections were treated with proteinase K (20 μg/ml) for 15 minutes at room temperature. Apoptotic cells were detected in situ by the TUNEL (terminal deoxynucleotidyl transferase-mediated dUTP nick end labeling) assay using the Roche in situ cell death detection kit POD (Roche Diagnostics GmbH, Mannheim, Germany), according to the manufacturer's instructions, and using diaminobenzidine as a staining dye. The percentage of TUNEL positive cells was clearly increased in the anterior horn region of the homozygous embryos when compared to heterozygote and wildtype littermates, where only few positive neuronal cells were detected.

### EXAMPLE 6

### Mapping and Cloning of the Mutation in Mutant Animals of the Invention

1. Generation of F3- and F4-Hybrids

F3-hybrids were generated by mating affected F2-individuals to C57BL/6J wildtype mice. F3 hybrids were designated as DO1. DO1 individuals were screened for the Cra1 phenotype directly after weaning. For mapping and positional cloning of the mutated gene affected F3-hybrids were either intercrossed to generate F4 intercross individuals, designated as DIC individuals, or outcrossed to C57BL/6J wildtype mice to generate F4-individuals, designated as D02 individuals.

2. DNA Isolation from Rodent Tails and Macromapping

Mouse genomic DNA was purified from 1 cm long pieces of tails of DIC or DO2 mice by using the “DNeasy 96 Tissue Kit” (Qiagen, Hilden, Germany) according to the manufacturer's protocol.

For chromosomal mapping of the mutant locus in the Cra1 mouse line standard outcross/backcross and outcross/intercross strategies in strain C57B1/6Jico (Ifacredo, France) were used in combination with microsatellite markers specific for the C3HeB/FeJ and C57B1/6Jico strains equally distributed over all 19 mouse chromosomes.

Oligonucleotides were obtained from MWG-Biotech AG (Ebersberg, Germany). PCR reactions were performed with fluorescent-labeled primers, whose sequences were taken from Schalkwyk et al., 1999, http://www-genome.wi.mit.edu/ (site maintained by the Whitehead Institute for Genome Research, Cambridge Mass., USA). The PCR reactions were performed in a MJ tetrad thermocycler PTC 225 device (MJ Research, Inc., Waltham Mass., USA) in 25 μl reaction volumes, using BioTherm-DNA-polymerase (Genecraft, Germany). A four minute denaturation step was then followed by 28 amplification cycles, each comprising 30 seconds denaturation and 30 seconds annealing at the respective temperatures given in Schalkyk et al., see above, and 30 seconds extension at 72° C. Samples were amplified with different dyes and products were separated on an ABI 3700 DNA sequencing device (PE Applied Biosystems, Foster City, USA) using internal length standards in every lane. Analysis was performed with Genescan version 3.0 and Genotyper version 2.1 software from ABI. We macromapped the locus of the mutation by identifying a chromosomal region having an increased allele frequency of markers representing the C3H founder strain.

Our analysis of D02 and DIC mice indicated that the Cra1 mutation coseggregates with the marker D12Mit141 on chromosome 12.

3. Fine Mapping

A detailed haplotype analysis of affected and non-affected Cra1 mice using identified single nucleotide polymorphisms (SNPs) located in the critical region demonstrated, that the mutation was located between SNP markers in the Ppp2r5c gene (encoding the protein phosphatase 2, regulatory subunit B (B56), gamma isoform) and the Traf3 gene (encoding the Tnf receptor-associated factor 3).

4. PCR Amplification of Cytoplasmic Dynein Heavy Chain1 Gene

Oligonucleotides were obtained from MWG-Biotech AG (Ebersberg, Germany). The PCR reactions were performed in a MJ tetrad thermocycler PTC 225 device (MJ Research, Inc., Waltham Mass., USA) in 25 μl reaction volumes using BioTherm-DNA-polymerase (Genecraft, Germany). A four minute denaturation step was then followed by 28 amplification cycles comprising each 30 seconds denaturation and 30 seconds annealing at the respective temperatures given in Schalkwyk et al. (1999), and 30 seconds extension at 72° C. A total of 77 exon sequences of the murine Dnchc1 gene were identified by sequence alignment of the full-length mouse Dnchc1 cDNA sequence (GenBank Acc. No. AY004877) and mouse BAC clone RPCI-23-134019 (GenBank Acc. No. AL596265) using Sequencher™ Version 4.0.5 (Gene Codes Corp., Ann Arbor Mich., USA). Exon specific primers located in the flanking intronic sequences were designed using Primer3, maintained by the Whitehead Institute for Biomedical Research.

All 77 exons of the Dnchc1 gene were amplified using a DNA pool of 4 affected pups (Cra1/Cra1) as well as C3H and C57 control DNA as PCR templates.

5. DNA Sequence Analysis

PCR amplicons were purified by using the QIAquick PCR Purification Kit (Qiagen, Hilden, Germany) according to the manufacturers protocol. PCR products were sequenced using forward/reverse PCR primers and “Big Dye” thermal cycle sequencing Kit (ABI PRISM, Applied Biosysterns, Foster City Calif., USA). The reaction products were analyzed on an ABI 3700 DNA sequencing device.

The sequences were edited manually and contig assembly for mutation detection was performed using Sequencher version 4.0.5 (Gene Codes Corp., Ann Arbor Mich., USA). The sequence analysis detected an A to G (codon TAC to TGC) transition in exon 13 of the mouse gene, at a location corresponding to nucleotide position 3328 (position +3164 of the coding sequnce) in the Dnchc1 mRNA sequence (GenBank Acc. No. AY004877, SEQ ID NO:1) The PCR amplicons with 364 bp in size, and including exon 13, were generated by PCR amplification with oligonucleotide Dnchc 1-25 (5′-TCCAAGACTTTGAAGTGAGGTTC; located in intron 12, SEQ ID NO:15) and with oligonucleotide Dnchc1-26 (5′-CAGGGCCAAACACCAATACT; located in intron 13, SEQ ID NO 16). The PCR amplicons generated contain either the wild type sequence (SEQ ID NO:13) or the mutated sequence (SEQ ID NO: 14), as listed in Tables 10 and 11, respectively.

TABLE 10

Wild type Dnchc1 364 bp PCR Amplicon
(SEQ ID NO:13)

wild type:
TCCAAGACTTTGAAGTGAGGTTCTCTAATGGTGTGAAGACATGGACTGTT
GACATGGTGGGTTCTAGCATATTAATAGTCTACAGCACTGCAGACTAGAA
TTGAGCTGTGTTTCCTTTGCAGGTTTGGCTTCAGTACCAGTGTTTGTGGG
ATATGCAGGCAGAAAACATTTACAACAGGCTAGGGGAAGATCTCAACAAG
TGGCAAGCTCTCCTGGTCCAGATAAGGAAAGCCAGAGGAACCTTTGACAA
TGCGGAAACCAAGAAAGAGTTTGGTCCGGTGGTGATAGATTACGGCAAGG
TGAGCTTGGCTTGTTGTCAAAAGGCACCAGGCAGCCGTAAGATCAGTATT
GGTGTTTGGCCCTG

TABLE 11
Mutant Dnchc1 364 bp PCR Amplicon (SEQ ID NO:14)

mutant:
TCCAAGACTTTGAAGTGAGGTTCTCTAATGGTGTGAAGACATGGACTGTT
GACATGGTGGGTTCTAGCATATTAATAGTCTACAGCACTGCAGACTAGAA
TTGAGCTGTGTTTCCTTTGCAGGTTTGGCTTCAGTGCCAGTGTTTGTGGG
ATATGCAGGCAGAAAACATTTACAACAGGCTAGGGGAAGATCTCAACAAG
TGGCAAGCTCTCCTGGTCCAGATAAGGAAAGCCAGAGGAACCTTTGACAA
TGCGGAAACCAAGAAAGAGTTTGGTCCGGTGGTGATAGATTACGGCAAGG
TGAGCTTGGCTTGTTGTCAAAAGGCACCAGGCAGCCGTAAGATCAGTATT
GGTGTTTGGCCCTG

The A to G transition (see nucleotide exchange underlined in SEQ ID NO:14) results in an Tyr (Y) to Cys (C) amino acid exchange at aa position 1055 in SEQ ID NO:2. The mutation was confirmed by sequence analysis of 13 adult Cra1^{-/+} mice and 5 Cra1^{-/- pups, respectively. The complete nucleic acid sequence of the mutant transcript (SEQ ID NO:}3) and the complete amino acid sequence of the mutant protein (SEQ ID NO:4) are shown above.

### EXAMPLE 7

### Method for Production of Mutant Animals of the Invention by Gene Targeting Technology

The construction of a recombinant targeting vector to insert a point mutation in exon 13 of the mouse Dnchc1 gene may be performed according to well known techniques: for example by the Lambda-KO-Sfi system of Nehls and Wattler, WO-A2-0/75127.

1. Vector Construction

In a first step, a 1,5 kbp genomic DNA fragment was PCR amplified, representing the left arm of homology of the targeting vector to be constructed. After subsequent subcloning of the PCR fragment into a plasmid vector, for example pCR 2.1-TOPO (K4500-01, Invitrogen Corp., Carlsbad, Calif., USA), according to the manufacurer's instructions, plasmid DNA, bearing the correct Dnchc1 insert is subject to site-directed mutagenesis, using a QuickChange Site-Directed Mutagenesis Kit (200518, Stratagene, La Jolla Calif., USA), as outlined in the manufacurer's instructions. The plasmid vector (parental DNA template) and two oligonucleotide primers, each primer complementary to opposite strands of the vector insert and containing the desired point mutation (exon 13, position 3169 of Dnchc1cDNA), were denatured and subject to PCR amplification with a proof-reading DNA polymerase (Pfu Turbo), provided in the kit. Using the non-strand displacing action of Pfu Turbo DNA polymerase, mutagenic primers were incorporated and extended, resulting in nicked circular DNA strands. In a restriction digest with DpnI, only the methylated parental parental DNA template was susceptible to DpnI digestion. After transformation in XL1-Blue supercompetent cells, provided with the kit, nicks in the mutated (point mutation) plasmid DNA were repaired. Mutation positive colonies were selected and plasmid DNA was isolated, according to the manufacurer's instructions (Stratagene, La Jolla Calif., USA).

Plasmid DNA, bearing the point mutation in exon 13, as described in the present invention, was subject to PCR amplification with primers, bearing SfiC and SfiA sequence overhangs, respectively, as described in the published patent application WO-A2-01/75127. The PCR fragment, representing the left arm of homology was further processed as described in the aforementioned patent application. The vector described in WO-A2-01/75127, includes a linear lambda vector (lambda-KO-Sfi) that comprises a stuffer fragment, an E. coli origin of replication, an antibiotic resistance gene for bacteria selection, two negative selection markers suitable for use in mammalian cells, and LoxP sequences for cre-recombinase mediated conversion of linear lambda phages into high copy plasmids. In a final lambda targeting vector, the stuffer fragment was replaced by Sfi A,B,C,D ligation of the left arm of homology (bearing the Dnchc1 point mutation in exon 13), an ES cell selection cassette, and a right arm of homology, as described in the aforementioned patent application. In-vitro packaging of the ligation products, plating of a phage library, plasmid conversion, and DNA isolation of the homologous recombination plasmid vector is performed according to standard procedures known to persons skilled in the art.

2. ES Cell Transformation and Production of Mice

Targeting vectors containing the point mutation were used for mouse ES cell transformation and to producing chimeric mice by blastocyst injection and transfer using standard methodology, well known in the art. The chimeras were bred to wild type mice to determine germline transmission. Heterozygotes and subsequently homozygotes were generated according to well-known techniques.

### EXAMPLE 8

### Method for the Detection of Nucleic Acid of the Present Invention: Mutation Detection using a Polymorphic RsaI Restriction Site

The nucleotide transition A to G in the nucleic acid sequence of the Dnchc1 gene results in the disappearance of a restriction site for the restriction enzyme RsaI. As a result, the restriction pattern of the nucleic acid sequence of Dnchc1 will differ between the wild type and the mutant sequence. Diagnostic analysis was applied on amplified fragments from nucleic acids extracted from mutant and wild type mice as already described in the example. PCR and restriction steps were performed as follows.

The PCR reactions were carried out on long genomic DNAs using primers Dnchc1-25

(5′-TCCAAGACTTTGAAGTGAGGTTC, located in intron 12, SEQ ID NO:15) and Dnchc1-26 (5′-CAGGGCCAAACACCAATACT, located in intron 13, SEQ ID NO:16), generating PCR products of either SEQ ID NO:13 (including the wild type exon 13 sequence) or SEQ ID NO:14 (including the mutant exon 13 sequence). The products have a size of 364 bp.

PCR reactions were performed in 25 μl using Taq-DNA polymerase (SIGMA, Deisenhofen, Germany) according to the manufacturer's protocol. A further 4 minute denaturation step was followed by 28 cycles of denaturation at 94° C. for 30 seconds; annealing at 55° C. for 30 seconds; and 90 seconds extension at 72° C. or the enzymatic digestion, 7 μl of PCR reaction, 10 μl H₂O, 2 μl NEB buffer 1 and 1 unit RsaI (New England BioLabs, Inc., Beverly Mass., USA) were incubated at 37° C. for 2 hours. Restriction fragments were size separated by electrophoresis on 2% agarose gels (SeaKem ME agarose, Biozym Diagnostik Gmbh, Hess. Oldenburg, Germany) with 0.5 μg ethidium bromide ml⁻¹ agarose. Gels were photographed after 2 hours electrophoresis at 3 V m⁻¹. The presence of the point mutation deletes the RsaI site present in the PCR product of the wild type. This results in the appearance of a non-restricted fragment of 364 bp in the Cra1^{-/-} mutant samples in contrast to two restriction fragments of 136 bp and 228 bp in the wild type. This procedure even allows the discrimination of heterozygous and homozygous Cra1 mutants.

### EXAMPLE 9

### Identification of the Full-Length cDNA Sequence of Human DNCH1

No full-length cDNA sequences of the human DNCH1 gene have been published. Here, we disclose a full-length cDNA sequence of the human DNCH1 gene, which has the highest homology to the coding sequence of the orthologous mouse gene, Dnchc1 (mRNA sequence published as Genbank Accession No. AY004877).

Although incomplete human DNCH1 mRNA sequences, and several EST sequences containing partial sequences of human DNCH1, have been published, construction of the correct, full-length sequence for human DNCH1 cDNA has not previously been achieved, perhaps as a consequence of the large size of the transcript, approximately 14.5 kb. Sequencing of several full-length cDNA clones generated from RNA of various human tissues obtained from independent sources confirms the sequence disclosed herein as SEQ ID NO:17. Particular attention is required to prevent RNA degradation, especially in those tissues known to exhibit high RNase activity (e.g. brain tissues); the use of fresh tissue being especially important.

### EXAMPLE 10

### Comparison of the Full-Length cDNA Sequences of Human DNCH1 and Mouse Dnchc 1

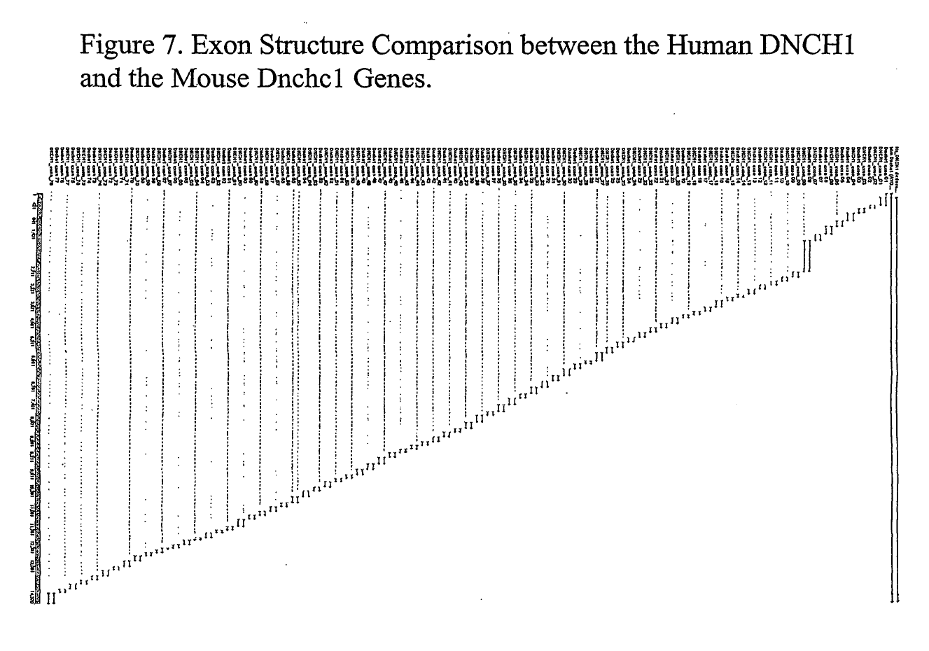

An exon structure comparison, as schematically depicted in FIG. 7, indicates an almost perfect identity in exon number und exon lengths between the mouse cytoplasmic dynein heavy chain1 (Dnchc 1) and the human cytoplasmic dynein heavy chain1 (DNCH 1) cDNAs. Exonic sequences were aligned with Sequencher software (version 4.0.5, GeneCodes Corp., Ann Arbor Mich., USA).

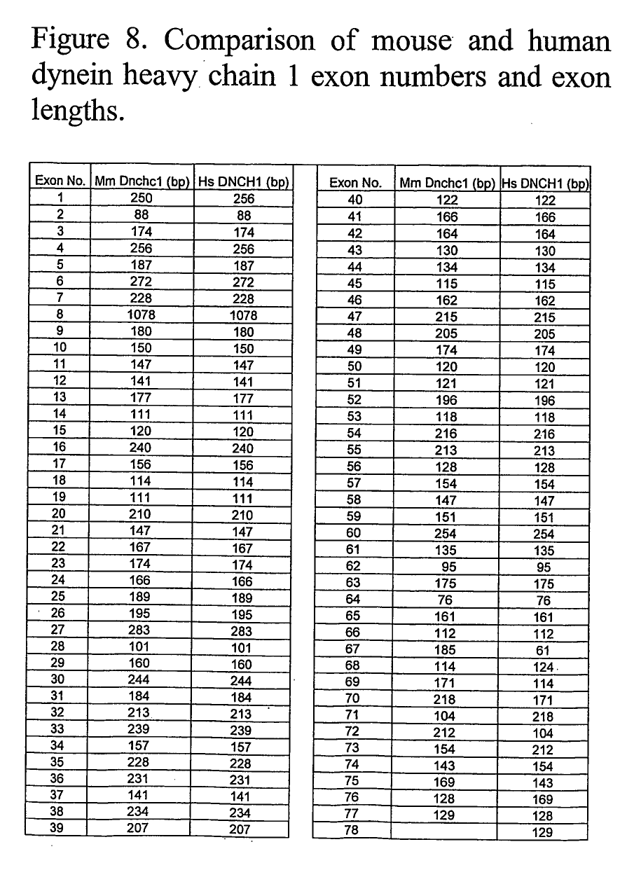

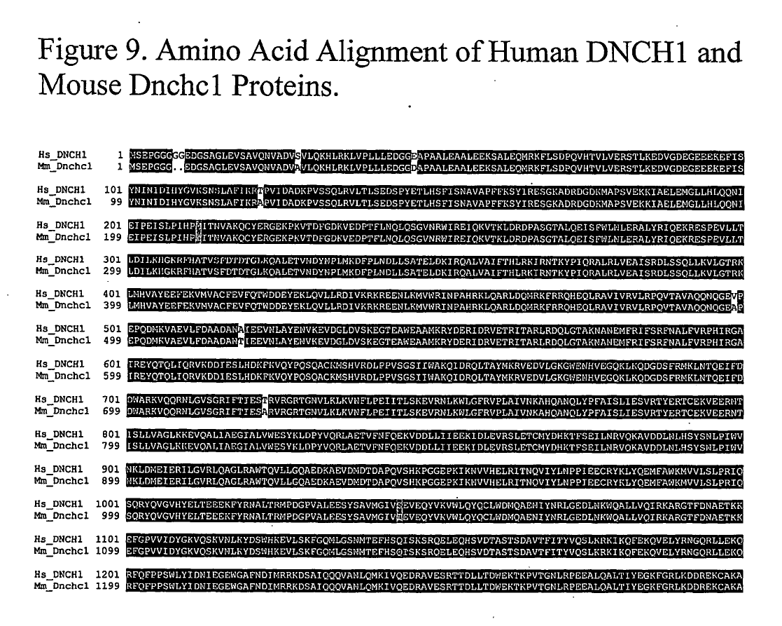

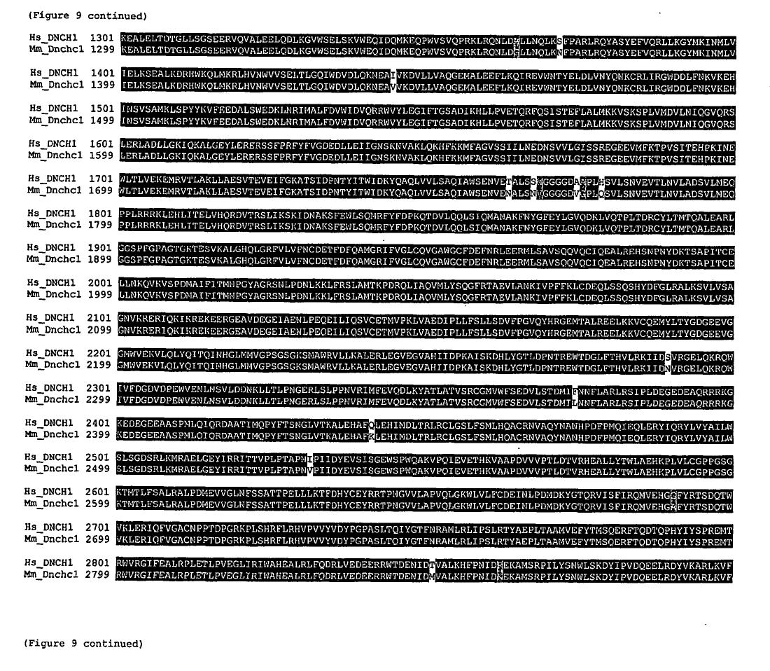

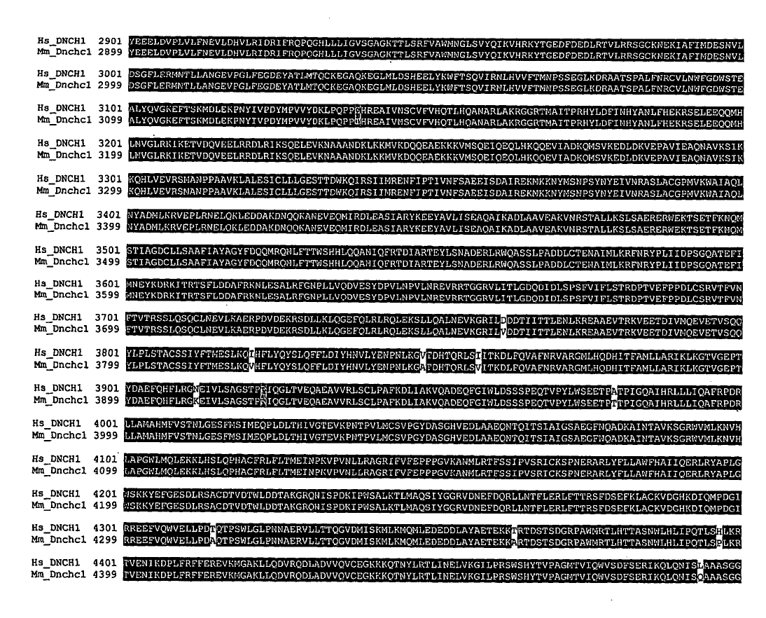

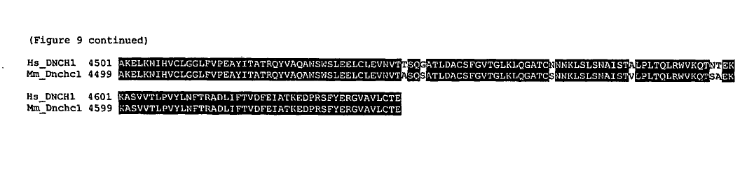

FIG. 8 exhibits a comparative listing of all mouse (MmDnchc1) and human (Hs DNCH1) cytoplasmic dynein heavy chain1 gene exons. The size of each exon is indicated in length by basepairs (bp). The human gene consists of 78 exons, and the mouse gene consists of 77 exons. The difference is caused by an additional RNA splice event in human, separating exon 67 (61 bp) and exon 68 (124 bp). The total lengths of the cDNAs are 13.941 bp in human DNCH 1, and 13.935 bp in mouse Dnchc 1.

### EXAMPLE 11

### Characteristics of Human and Mouse Cytoplasmic Dynein Heavy Chain1 Protein

The human ortholog of the mouse cytoplasmic dynein heavy chain1 protein, human cytoplasmic dynein heavy chain1 protein, has a length of 4646 amino acid residues (in comparison to 4644 for the corresponding mouse protein). FIG. 9 represents an amino acid alignment of human DNCH 1 and mouse Dnchc 1, with black boxes indicating amino acid identity, and grey boxes indicating amino acid similarity. The amino acid identity is 97%, indicating that these are true orthologs.

### EXAMPLE 12

### Detection of Transcriptionally Deregulated Genes Expressed in the Brain

A series of genes selected for their putative biological relevance to cytoplasmic dynein heavy chain1 function were analysed for altered RNA expression levels in the brain of homozygous Cra1 mice, in comparison to expression levels in brain of wild type mice.

Significantly altered expression levels were found for those genes listed in FIG. 6. Transcriptional deregulation was determined by quantitative PCR-Light Cycler technology (Roche Diagnostics GmbH, Mannheim, Germany), according to the manufacturer's instructions.

### EXAMPLE 13

### Exons 12 and 13

Exon 13 is critical for activity of the cytoplasmic dynein heavy chain1, as demonstrated by the altered activity of the mouse protein bearing the amino acid substitution at position 1055, encoded by exon 13, the causative point mutation being located close to the 5′ end of exon 13. The nucleotide sequence of exon 13 of the mouse wild type Dnchc 1 is provided as SEQ ID NO: 19.

TABLE 12

Nucleotide sequence of mouse exon 13
(SEQ ID NO:19)

GTTTGGCTTCAGTACCAGTGTTTGTGGGATATGCAGGCAGAAAACATTTA
CAACAGGCTAGGGGAAGATCTCAACAAGTGGCAAGCTCTCCTGGTCCAGA
TAAGGAAAGCCAGAGGAACCTTTGACAATGCGGAAACCAAGAAAGAGTTT
GGTCCGGTGGTGATAGATTACGGCAAG

As the causative point mutation is close to the 5′ end of exon 13, it is probable that the amino acid sequence encoded by exon 12 also has a significant influence on the activity of the protein.

SEQ ID NO:20 indicates the nucleotide sequence of exons 12 plus 13 of the mouse wild type Dnchc 1.

TABLE 13

Nucleotide sequence of mouse exon 12 and 13
(SEQ ID NO:20)

GTGGGAGTGCATTACGAGTTGACAGAGGAAGAGAAGTTCTATCGGAATGC
ACTGACAAGGATGCCCGAGGGCCCTGTCGCCCTGGAAGAGTCCTACTCTG
CGGTCATGGGCATAGTGACTGAAGTTGAGCAGTATGTTAAGGTTTGGCTT
CAGTACCAGTGTTTGTGGGATATGCAGGCAGAAAACATTTACAACAGGCT
AGGGGAAGATCTCAACAAGTGGCAAGCTCTCCTGGTCCAGATAAGGAAAG
CCAGAGGAACCTTTGACAATGCGGAAACCAAGAAAGAGTTTGGTCCGGTG
GTGATAGATTACGGCAAG

The amino acid sequences encoded by exons 12 plus 13 are located within a region known to be important for heavy chain dimerization (Tynan et al. (2000) J Biol Chem 275, 32769-32774). Therefore these amino acid sequences, and the peptide sequences encoded by the corresponding exons in other mammalian systems, may be used as targets for inactivation of the protein by small molecules, antibodies, antibody fragments, aptamers and other molecules designed to bind thereto.

TABLE 14

Amino acid sequence encoded in-frame by nucleotide
sequences of mouse exon 13 (SEQ ID NO:21)

VWLQYQCLWDMQAENIYNRLGEDLNKWQALLVQIRKARGTFDNAETKKEF
GPVVIDYGK

TABLE 15

Amino acid sequence encoded in-frame by nucleotide
sequences of mouse exon 12 and 13 (SEQ ID NO:22)

VGVHYELTEEEKFYRNALTRMPDGPVALEESYSAVMGIVTEVEQYVKVWL
QYQCLWDMQAENIYNRLGEDLNKWQALLVQIRKARGTFDNAETKKEFGPV
VIDYGK

The corresponding human peptide sequences, encoded by human exon 13 (SEQ ID NO:23), or human exon 12 and 13 (SEQ ID NO:24), are preferred embodiments of the invention.

TABLE 16

Amino acid sequence encoded in-frame by nucleotide
sequences of human exon 13 (SEQ ID NO:23)

VWLQYQCLWDMQAENIYNRLGEDLNKWQALLVQIRKARGTFDNAETKKEF
GPVVIDYGK

TABLE 17

Amino acid sequence encoded in-frame by nucleotide
sequences of human exon 12 and 13 (SEQ ID NO:24)

VGVHYELTEEEKFYRNALTRMPDGPVALEESYSAVMGIVSEVEQYVKVWL
QYQCLWDMQAENIYNRLGEDLNKWQALLVQIRKARGTFDNAETKKEFGPV
VIDYGK

TABLE 18

Amino acid sequence encoded in-frame by nucleotide
sequences of human exon 12 and 13 (SEQ ID NO:24)

VGVHYELTEEEKFYRNALTRMPDGPVALEESYSAVMGIVSEVEQYVKVWL
QYQCLWDMQAENIYNRLGEDLNKWQALLVQIRKARGTFDNAETKKEFGPV
VIDYGK

### EXAMPLE 14

### Gene Therapy

A number of viruses, including retroviruses, adenoviruses, herpes viruses, and pox viruses, have been developed as live viral vectors for gene therapy. A nucleic acid that codes for mutated cytoplasmic dynein heavy chain1 protein (e.g. SEQ ID NO:4, SEQ ID NO:6) is inserted into the genome of a parent virus to allow it to be expressed by that parent virus. This task is accomplished by first constructing a DNA donor vector for in vivo recombination with a parent virus.

The DNA donor vector contains:

(i) a prokaryotic origin of replication, so that the vector may be amplified in a prokaryotic host;

(ii) a gene encoding a marker which allows selection of prokaryotic host cells that contain the vector (e.g. a gene encoding antibiotic resistance);

(iii) at least one gene encoding a desired protein located adjacent to a transcriptional promoter capable of directing the expression of the gene; and

(iv) DNA sequences homologous to the region, of the parent virus genome where the foreign gene or genes will be inserted, flanking the construct of element (iii).

The donor vector further contain additional genes which encodes one or more marker which will allow identification of recombinant viruses containing inserted foreign DNA. The marker genes to be used include genes that encode antibiotic or chemical resistance (e.g. see Spyropoulos et al. (1988) J Virol 62, 1046-1054; Falkner and Moss (1988) J Virol 62, 1849-1854; Franke et al. (1985) Mol Cell Biol 5, 1918-1924), as well as genes such as the E. coli lacZ gene, that permit identification of recombinant viral plaques by calorimetric assay (Panicali et al. (1986) Gene 47, 193-199).

Homologous recombination between donor plasmid DNA and viral DNA in an infected cell are made using standard techniques. The recombination results in the formation of recombinant viruses that incorporate the nucleic acid encoding SEQ ID NO:4. Appropriate host cells for in vivo recombination are eukaryotic cells that can be infected by the virus and transfected by the plasmid vector such as, for example, chick embryo fibroblasts, HuTK143 cells (human cells), and CV-1 and BSC-40 cells (both monkey kidney cells). Infection of cells by the virus and transfection of these cells with plasmid vectors is accomplished by techniques standard in the art.

Following in vivo recombination, recombinant viral progeny are identified by co-integration of a gene encoding a marker or indicator gene with the foreign gene or genes of interest, which, in this case, is the β-galactosidase gene. The presence of the β-galactosidase gene is selected using the chromogenic substrate 5-bromo-4-chloro-3-indolyl-β-D-galactosidase (Panicali et al. (1986) Gene 47, 193). Recombinant virus appears as blue plaques in a lawn of the host cells. Expression of the polypeptide encoded by the inserted gene is further confirmed by in situ enzyme immunoassay performed on viral plaques and confirmed by Western blot analysis, radioimmunoprecipitation assay (RIPA), and/or enzyme immunoassay (EIA). Positive viruses are cultured and expanded and stored.

In gene therapy, B cells are collected from peripheral blood of a patient and infected at MOI of 1, 2, 4, 8, 10 and 20. Expression of the cytoplasmic dynein heavy chain1 is assayed 24 hours post-infection. Wild type virus is used as the negative control. The percent of cells transduced as a function of MOI and days after infection was determined by antibody and chromogenic substrate assays described above. B cells tranfected at high efficiency are reintroduced into the patient.

### EXAMPLE 15

### Transgenic Animal Model Bearing Transgenic Cytoplasmic Dynein Heavy Chain1

Transgenic mice carrying a mammalian dynein heavy chain 1 transgene are generated by either using the embryonic stem cell method, or the pronucleus method, both of them well-known methods in the art; preferably using the method of Nehls and Wattler, as described in W0-A2-01/75127. For transgenic methods see also U.S. Pat. No. 6,436,701, U.S. Pat. No. 6,018,097, U.S. Pat. No. 5,942,435, U.S. Pat. No. 5,824,837, U.S. Pat. No. 5,731,489, and U.S. Pat. No. 5,523,226.

### EXAMPLE 16

### In Vitro Dynein Motility Assay

Abnormalities in axonal transport are described in the human disease ALS (Sasaki and Iwata (1996) Neurology 47, 535-540). Such abnormalities are also observed in several mouse models with motor neuron degeneration, e.g. the wobbler mouse (Mitsumoto et al. (1986) Ann Neurol 19, 36-43) and transgenic mice overexpressing the heavy chain of neurofilament protein (Collard et al. (1995) Nature 375, 61-64), SOD-1 (Zhang et al. (1997) J Cell Biol 139, 1307-1315) and dynamitin (LaMonte et al. (2002) Neuron 34, 715-727). Studies in Drosophila have shown that mutations in the cytoplasmic dynein heavy chain (cDhc64c) lead to a disruption of axonal organelle transport in both directions and as a consequence to axonal swelling and posterior paralysis (Martin et al. (1999) Mol Biol Cell 10, 3717-3728).

A microtubule gliding assay is performed based on a commercial assay offered by Cytoskeleton, Inc. (Denver, Colo., USA). In brief, the assay involves coating a glass slide with the protein of interest and then examining, by time-lapse or by real time microscopy, the protein's ability to translocate microtubules in the presence of ATP. Cytplasmic dynein heavy chain 1 proteins, carrying non-conserevd amino acid substitutions at residue positions as highlighted in FIGS. 9 and 10, and listed in Example 19, are over-expressed either in a bacterial, or in an eucaryotic expresion system. A preferred method is the cDNA subcloning into expression vectors of the Gateway cloning and expression system (Invitrogen, California, USA), according to the manufacturer's instructions. Purification of recombinant dynein heavy chain 1 from host cells can be performed using standard methods well-known to those skilled in the art. For standard references, see in Sambrook et al. (eds.), MOLECULAR CLONING: A LABORATORY MANUAL (2^{nd} Ed.), Cold Spring Harbor Laboratory Press, Cold Spring Harbor, N.Y., 1989; and Ausubel et al. (eds.), CURRENT PROTOCOLS IN MOLECULAR BIOLOGY, John Wiley & Sons, New York, N.Y., 1993.

Differences in proteins ability to translocate microtubules in the presence of ATP, whereby the proteins carry particular non-conserved amino acid substitution, are indicative of functional domains of dynein heavy chain 1 in axonal transport.

### EXAMPLE 17

### Interaction of the Dynein Complex with the EAAT2 Glutamate Transporter

A direct link between the Cra1 mouse line and the excitotoxicity hypothesis for ALS, as described in the background of the Invention, is the excitatory neuronal damage detected in the brains of heterozygote Cra1 animals. Comparable defects have been found in the brains of mice lacking EAAT2, an glutamate transporter (Tanaka et al., Science vol. 276, 1699-1702, june 1997).

Co-immunoprecipitation studies using antibodies raised against the cytoplasmic dynein heavy chain 1, the 74 kDa intermediate chain, the dynactin p150 component and against the EAAT2 transporter in various combinations are performed using brain homogenates of wild type mice, Cra1 homozygotes, and Cra1 heterozygotes. Anti dynein heavy chain 1 antibodies are raised against epitopes, which carry non-conserved amino acid substitutions of those amino acid residues highlighted in FIGS. 9 and 10, and listed in Example 19. Protein expression vectors and antibodies are generated as described, in example, in Ausubel et al., Current protocols in Molecular Biology, Massachusetts general Hospital, Harvard Medical School, 1996 by John Wiley and Sons, Inc.). Antibodies raised against he 74 kDa intermediate chain, the dynactin p150 component and against the EAAT2 transporter are commmercially available (anti-EAAT2 antibody from Accurate Chemical and Scientific Corp; anti-74 kDa intermediate chain antibody from Chemicon Int. Corp.; anti-dynactin p150 antibody from BD Transduction Laboratories). Co-Immunoprecipitation studies are well known in the art and are performed as described, for example, in Ausubel et al., Current protocols in Molecular Biology, Massachusetts general Hospital, Harvard Medical School, 1996 by John Wiley and Sons, Inc.).

The results of the assay are indicative for dynein heavy chain 1 function in EAAT2 glutamate transporter/dynein motor complex interaction. Functional amino acid residues are identified.

### EXAMPLE 18

### Quantitative Binding Study

To investigate a possible influence of an cytoplasmic dynein heavy chain 1 mutation on the hole dynein complex, which might result in an impaired transport process, quantitative binding studies are performed. Surface plasmon resonance (SPR) spectroscopy, such as, in example, in the Biacore technology (Biacore AB, Uppsala, Sweden) is an appropriate tool. By this method the interaction of two molecules binding each other is described by two rate parameters, the association rate parameter k(a) and the dissociation rate parameter k(d). Under standardized conditions these kinetic parameters can be determined by analysis of their interaction in an affinity-based biosensor system. The association rate describes the cohesion frequency and the attraction between two molecules. The dissociation rate describes the stability of the complex.

Using the SPR technique, the affinity of mutated dynein heavy chain domains, carrying non-conserved amino acid substitutions of the residues highlighted in FIGS. 9 and 10, and listed in Example 19, to other components of the dynein complex are analysed.

In similar studies the influence of mutations on protein-protein affinities have been studied (Hobba et al., J Biol Chem 273(31):19691-19698) 1998; Starling et al., J Exp Med 185(8):1487-1492, 1997).

### EXAMPLE 19

### Characterization of Dynactin/Dynein Complex Proteins from Different Species—Amino Acid Conservation

Inter-species comparison of mouse and human dynein heavy chain 1, dynein intermediate chain 1, dynein intermediate chain 2, and DCTN lamino acids was perfomed. The degree of identity and the degree of similarity are summarized in the following table:

| Protein of the | | |
|---|---|---|
| dynactin/dynein complex | Degree of Identity | Degree of Similarity |
| Dynein heavy chain1 | 97% | 98% |
| Dynein intermediate chain 1 | 88% | 89% |
| Dynein intermediate chain 2 | 94% | 95% |
| DCTN 1 | 97% | 98% |

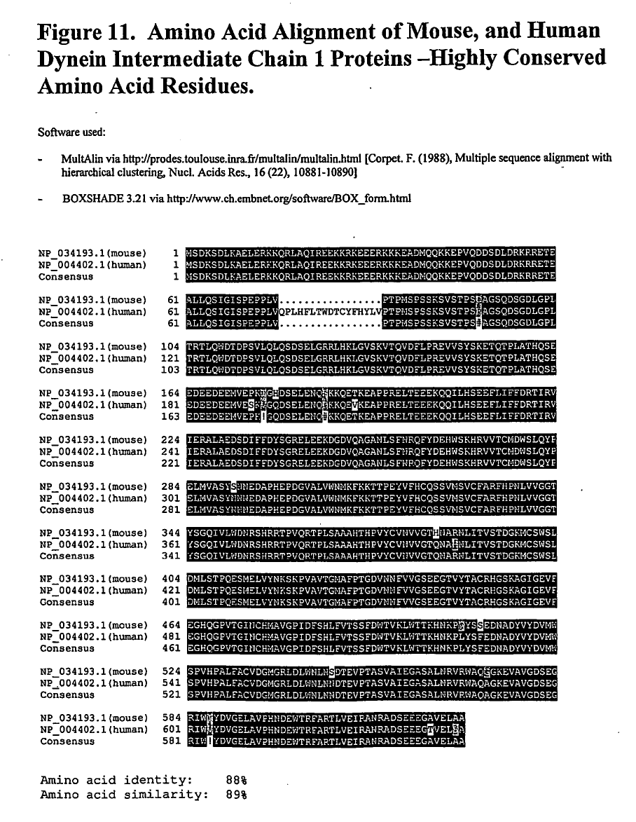

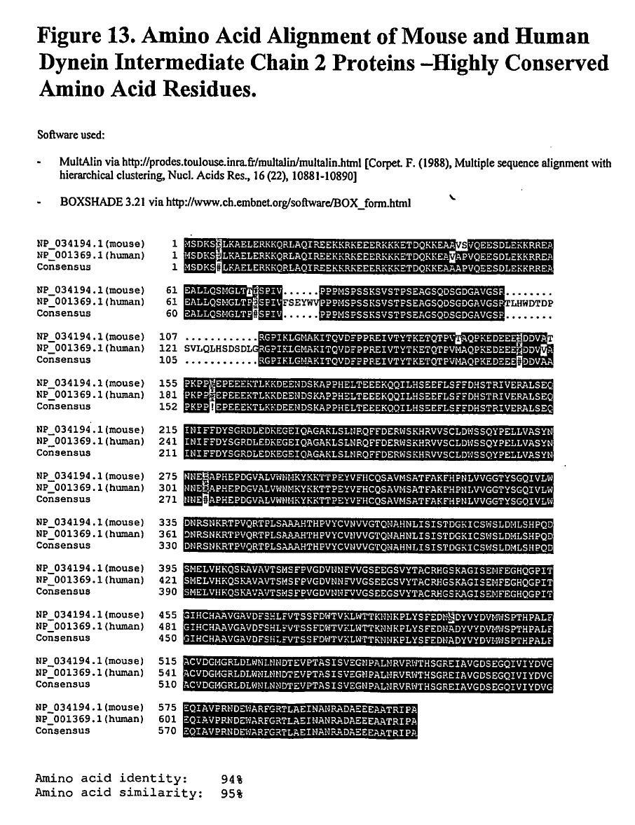

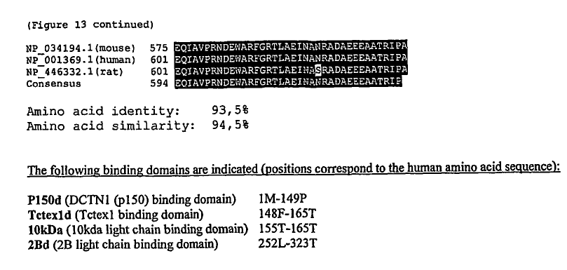

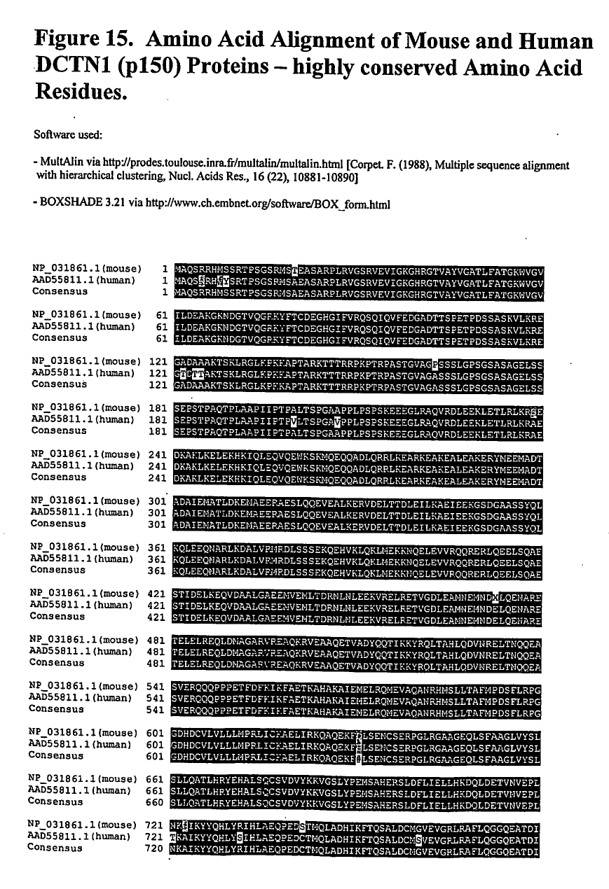

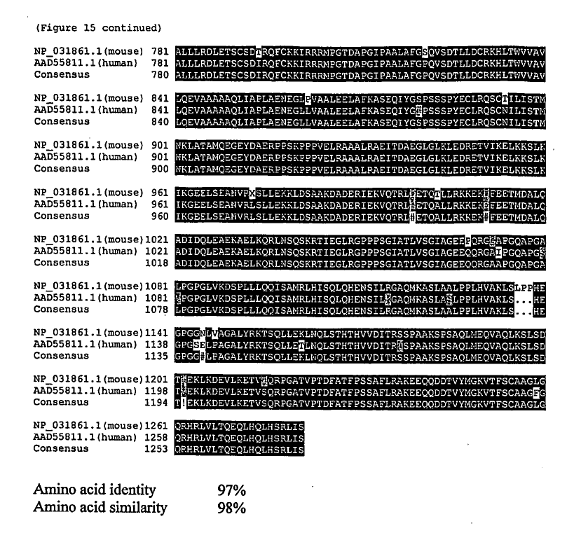

The high degree of amino acid identity and similarity is indicative for highly conserved residues between the species indicating functional significance of these conserved residues in the proteins compared in this Example, see FIGS. 9, 11, 13 and 15. The amino acid being exchanged in dynein heavy chain 1 (1055Y to C, as described in the invention herein) is conserved between the species compared, as indicated in FIG. 9.

In an inter-species comparison of mouse, human, and rat dynein heavy chain 1, dynein intermediate chain 1, dynein intermediate chain 2, and DCTN amino acids was perfomed, the degree of identity and the degree of similarity are summarized in the following table:

| Protein of the | | |
|---|---|---|
| dynactin/dynein complex | Degree of Identity | Degree of Similarity |
| Dynein heavy chain1 | 95% | 96% |
| Dynein intermediate chain 1 | 87% | 88% |
| Dynein intermediate chain 2 | 93.5% | 94.5% |
| DCTN 1 | 95% | 96% |

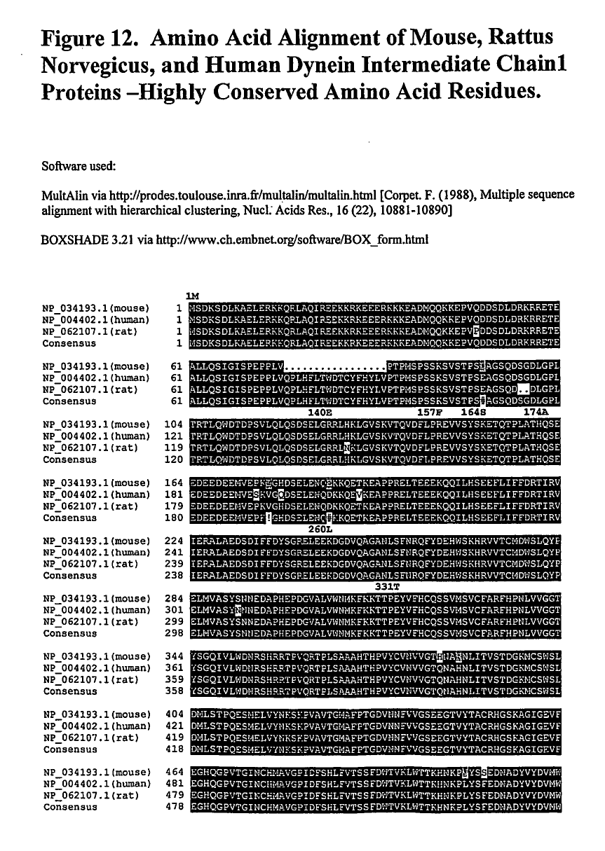

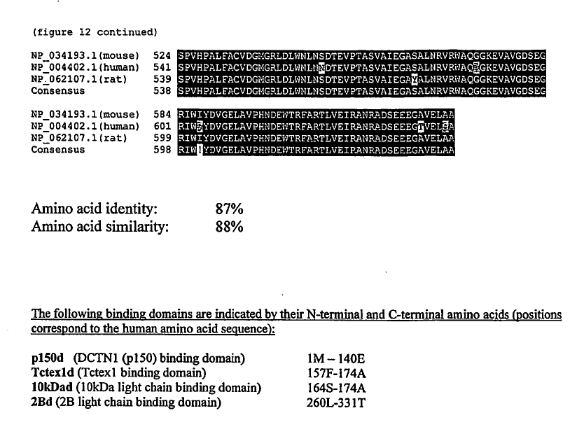

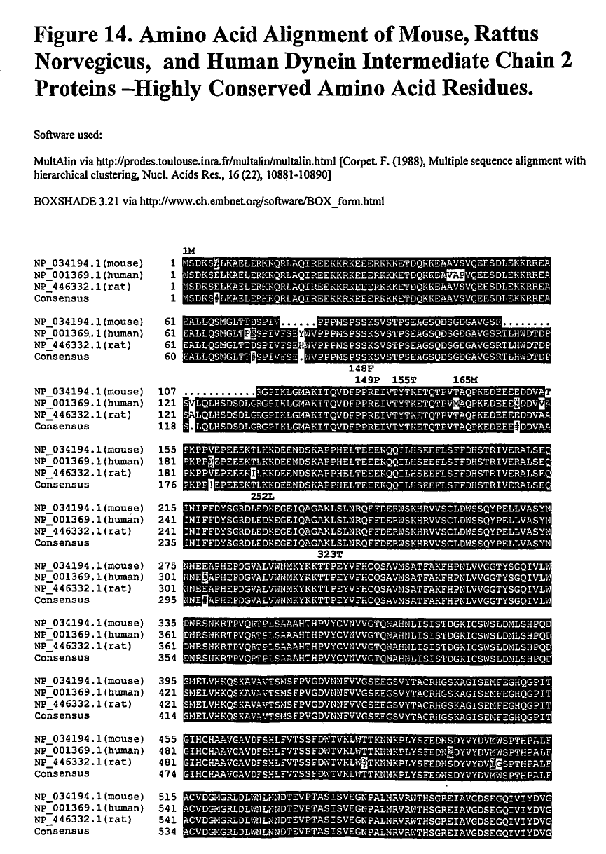

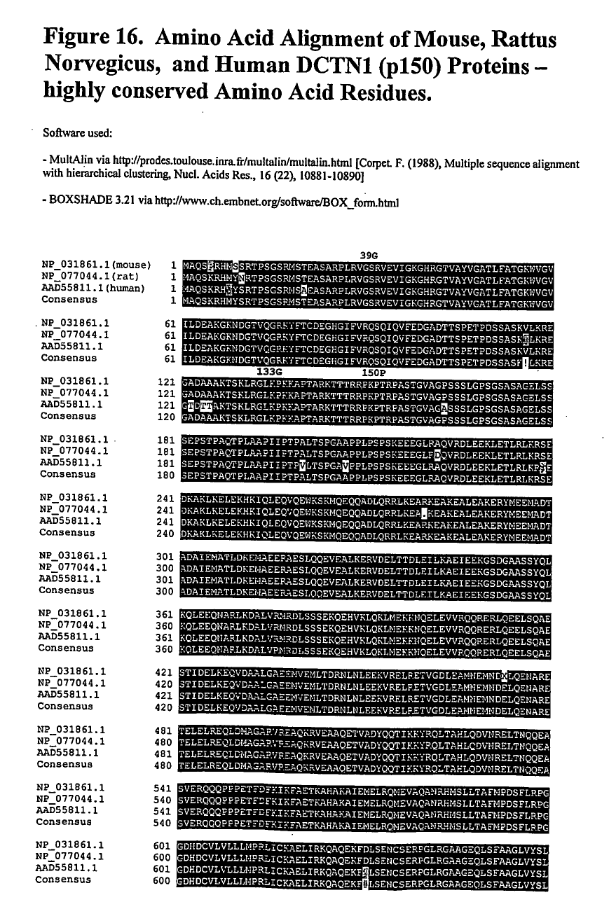

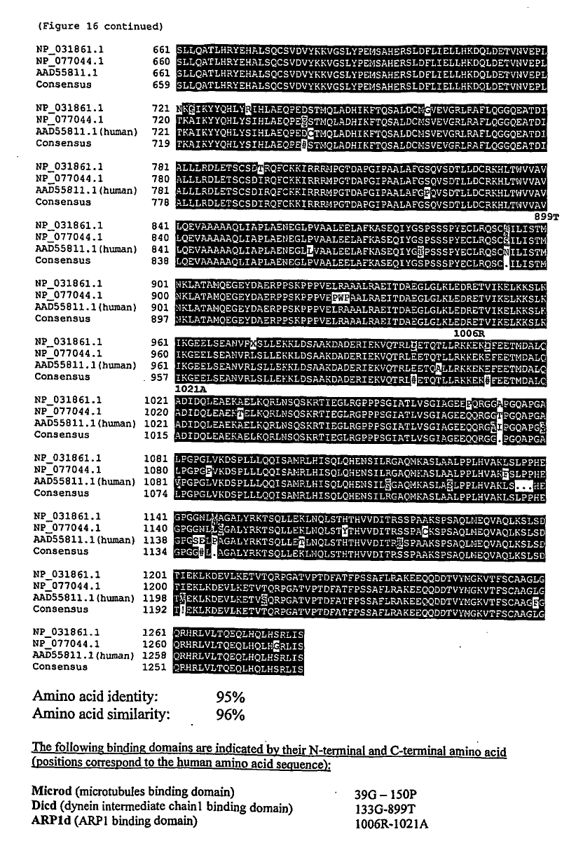

The high degree of amino acid identity and similarity is indicative for highly conserved residues between the species indicating functional significance of these conserved residues in the proteins compared in this Example, see FIGS. 10, 12, 14, and 16. The amino acid being exchanged in dynein heavy chain 1 (1055Y to C, as described in the invention herein) is conserved between the species compared, as indicated in FIG. 10.

For several subunits of the dynactin/dynein complex, several binding domains are predicted, with which the individual proteins of the dynactin/dynein complex binds to other subunits of the complex. The binding domains of several proteins, which are subunits of the dynactin/dynein complex are summarized in respect to the amino acids positions:

a) With respect to mouse Dnchc1 the following domains and binding sites are predicted, which are summarized in respect to the amino acid positions of the mouse Dnchc1 protein:

heavy chain dimerization domain (Hcd) between amino acids 300 and 1140; 74 kDa intermediate chains binding domain (led) between amino acids 446 and 701; 53-59 kDa light intermediate chains binding domains (Lcds) between amino acids 649 and 800 (Tynan et al. (2000) J Biol Chem. 275, 32769-32774).

b) With respect to cytoplasmic dynein intermediate chain 1 the following binding domains are predicted, which are summarized in respect to the amino acid positions of the mouse cytoplasmic dynein intermediate chain 1 protein:

10 kDa light chain binding domain (10 kDad) between amino acids 147 and 157 (Lo et al., J. Biol. Chem. 276(17):14059-14066, 2000); light chain 2B binding domain (2Bd) between amino acids 243 and 314 (Susalka et al., J. Biol. Chem. 277(36):32939-32946, 2002); Tctex1 binding domain (Tctex1d) between amino acid 140 and 157 (Mok et al. J. Biol. Chem. 276(17):14067-14074, 2001); p150 binding domain (p150d) between amino acid 1 and 123 (see in Susalka et al., J. Biol. Chem. 277(36):32939-32946, 2002).

c) For mouse cytoplasmic dynein intermediate chain 2 several binding domains are predicted, which are summarized in respect to the amino acid positions of the mouse cytoplasmic dynein intermediate chain 2 protein:

10 kDa light chain binding domain (10 kDad) between amino acids 129 and 139; light chain 2B binding domain (2Bd) between amino acids 226 and 297; Tctex1 binding domain (Tctex1d) between amino acid 122 and 139; p150 binding domain (p150d) between amino acid 1 and 123 (see in Susalka et al., J. Biol. Chem. 277(36):32939-32946, 2002).

d) For mouse p150 (DCTN 1) several binding domains are predicted, which are summarized in respect to the amino acid positions of the mouse p150 protein:

microtubule binding domain (Microtd) between amino acids 39 and 150 (Waterman-Storer et al., P.N.A.S. 92:1634-1638, 1995); ARP1 binding domain (ARP1d) between amino acids 1006 and 1021 (see in Waterman-Storer et al., P.N.A.S. 92:1634-1638, 1995); cytoplasmic dynein intermediate chain binding domain (Dicd) between amino acid 133 and 899 (Karki and Holzbaur, J. Biol. Chem. 270:28806-28811, 1995).

In an inter-species comparison of the amino acids of the corresponding mouse and human proteins, the degree of conserved (i.e. identical and similar) amino acids in these domains and binding sites is summarized in the following tables:

Cytoplasmic dynein heavy chain 1

| Binding site to | Degree of conservation |
|---|---|
| Hcd | 99.5% |
| Icd | 99% |
| Lcds | 99.5% |

Cytoplasmic dynein intermediate chain 1

| Binding site to | Degree of conservation |
|---|---|
| 10kDad | 100% |
| 2Bd | 98.6% |
| Tctex1d | 100% |
| P150d | 86.2% |

Cytoplasmic dynein intermediate chain 2

| Binding site to | Degree of conservation |
|---|---|
| 10kDad | 90% |
| 2Bd | 98.6% |
| Tctex1d | 94% |
| P150d | 74% |

P150 (DCTN 1)

| Binding site to | Degree of conservation |
|---|---|
| Microd | 98% |
| ARP1d | 100% |
| Dicd | 98.5% |

In an inter-species comparison of the amino acids of the corresponding mouse, rat, and human proteins, the degree of conserved (i.e. identical and similar) amino acids in these domains and binding sites is summarized in the following tables:

Cytoplasmic dynein heavy chain 1

| Binding site to | Degree of conservation |
|---|---|
| Hcd | 99% |
| Icd | 99% |
| Lcds | 99% |

Cytoplasmic dynein intermediate chain 1

| Binding site to | Degree of conservation |
|---|---|
| 10kDad | 100% |
| 2Bd | 98.6% |
| Tctex1d | 100% |
| P150d | 85.3% |

Cytoplasmic dynein intermediate chain 2*

| Binding site to | Degree of conservation |
|---|---|
| 10kDad | 90% |
| 2Bd | 98.6% |
| Tctex1d | 94% |
| P150d | 74% |

*(mouse and rat amino acid sequences are 100% identical for cytoplasmic dynein intermediate chain 2)

P150 (DCTN 1)

| Binding site to | Degree of conservation |
|---|---|
| Microd | 98% |
| ARP1d | 100% |
| Dicd | 98.7% |

In an inter-species comparison of cytoploasmic dynein heavy chain 1 amino acid sequences of the species mouse, human, rat, Drosophila, and C. elegans, the degree of conserved (i.e., identical and similar) amino acids in these domains and binding sites is the following:

Cytoplasmic dynein heavy chain 1

| Binding site to | Degree of conservation |
|---|---|
| Hcd | 64% |
| Icd | 70% |
| Lcds | 61% |

Conserved amino acid residues within the defined binding domains of the individual proteins are listed in Table 19, 20, 21, and 22.

Evolutionary pressure has conserved these residues at their particular locations in the molecule. It is predicted that any non-conservative amino acid substitution will modify the protein's normal biological function in a manner analogous to that observed in the present invention. Hence, identification of such an abnormal cytoplasmic dynein intermediate chain 1 protein sequence in a biological sample, or of the a DNA encoding such an abnormal cytoplasmic dynein intermediate chain 1 protein, will be indicative of an increased probability of developing the phenotype of the present invention.

### EXAMPLE 20

### Mutation Detection at Human Patient Samples by Heteroduplex Analysis Using Temperature Gradient Capillary Electrophoresis (TGCE)—Mutation Detection in Exon 13 of Human Cytoplasmic Dynein Heavy Chain 1 (DNCH1)

Assay Description: Human genomic DNA is isolated and is subject to PCR amplification according to standard methods in the art, in example, as described in Example 6. For detection of mutations with unknown positions in PCR amplified DNA fragments, double-stranded (ds) DNA is electrophoresed through a temporal gradient of increasing temperature (Temperature Gradient Capillary Electrophoresis (TGCE); RevealSystem, SCE9610, by SpectruMedix LLC, State College, Pa., USA). Because retardation of dsDNA during electrophoresis is greatest at the temperature of partial denaturation, DNA fragments of the same size can be separated according to their thermodynamic stabilities. Base mismatches within dsDNA molecules (heteroduplices) lead to a significant destabilization resulting in significant differences in melting temperatures (Tₘ) between heteoduplices and perfectly paired dsDNA (homoduplices). Such differences in Tₘ allow the separation of heteroduplices from homoduplices in a temperature gradient electrophoresis and serve as the basis for mutation detection by TGCE.

Detection of point mutations in exon 13: For detection of homo- or heterozygous mutations, an equimolar mixture of chromosomal DNA from 5 to 10 healthy individuals is prepared. An equimolar amount of this template DNA (healthy individuals) is added to each sample DNA (patient), and the target region is amplified using a standard PCR reaction with gene specific primers. For analysis of human DNCH1 exon 13, a 290 bp DNA fragment containing all exon 13 DNA sequence, was generated with the following intronic primers:

| | | location |
|---|---|---|
| | | in |
| | | intron |
| primer | sequence | no. |
| hDnchc1-21 | 5′-TGTGAACATAAGAGGTGCCAGT | 12 |
| (SEQ ID NO:26) | | |
| hDnchc1-22 | 5′-CTACCCGTGACACCT1TCAA | 13 |
| (SEQ ID NO:27) | | |

Following the manufacturers instructions, hybrids of wildtype and mutant sequences are formed in a denaturation/renaturation step, trasforming base pair exchanges into heteroduplices with lower thermal stability. A typical temperature profile for denaturation/renaturation is:
heat sample to 95° C. and hold for 3 mm
decrease from 95° C. to 80° C. at 3° C./min
decrease from 80° C. to 55° C. at 1° C./min
hold 20 min at 55° C.
decrease from 55° C. to 45° C. at 1GC/min
decrease from 45° C. to 25° C. at 2° C./min

Depending on the DNA concentration obtained in the PCR reaction the sample is diluted in a range of 1:10 to 1:50 and subjected to TGCE. Washing, dilution and running buffers are supplied by the manufacturer. Typical operation parameters for TGCE are:
Prerun: 25 min
Injection voltage: 3-8 kV
Injection time: 3-10 sec.
Running voltage: 10 kV
Electrophoresis time: 60 min

The applied temperature gradient during electrophoresis depends on the base composition (G+C content) of the analyzed fragment and ranges from 55° C. to 70° C. For the 290 bp exon 13 fragment of hDNCH1 a temperature gradient from 55° C. to 65° C. with a ramp time of 21 min was applied.

Upon completion the obtained electrophoresis pattern is analyzed for additional bands resulting from decreased mobility of heteroduplices during TGCE, using the manufacturers software program Revelation 2.10. Candidate fragments are further analyzed by DNA sequencing, using the method described in Example 6, Section DNA Sequence Analysis.

TABLE 19
Conserved amino acid residues in binding domains Hcd, Icd,
and Lcd of Dnchc1 of mouse (M. musculus), rat (R. norvegicus),
human (H. sapiens), Drosophila melanogaster, and Caenorhabditis elegans: -
conserved residues numbered as mouse Dnchc1 amino acid positions.

| | | | | | | | | | | | | | |
|---|---|---|---|---|---|---|---|---|---|---|---|---|---|
| 302L | 303K | 305G | 306K | 307R | 308F | 309H | 310A | 311T | 312V | 314F | 315D | 317D | 319G |
| 320L | 321K | 322Q | 324L | 327V | 329D | 330Y | 331N | 333L | 334M | 335K | 337F | 338P | 342L |
| 344S | 345A | 346T | 350K | 354A | 358I | 359F | 361H | 362L | 363R | 364K | 366R | 368T | 369K |
| 370Y | 371P | 373Q | 374R | 376L | 378L | 380E | 381A | 382I | 383S | 384R | 385D | 386L | 389Q |
| 390L | 391L | 392K | 393V | 394L | 399L | 400M | 406E | 407F | 411M | 414C | 418F | 421W | 422D |
| 423D | 424E | 425Y | 427K | 431L | 432L | 433R | 434D | 435I | 437K | 439K | 440R | 445K | 448W |
| 454H | 457L | 460R | 467F | 468R | 4700 | 471H | 472E | 473Q | 475R | 477V | 478I | 480R | 481V |
| 482L | 483R | 484P | 519E | 521V | 524A | 525Y | 526E | 529K | 531V | 532D | 534L | 535D | 543A |
| 544W | 545E | 547A | 549K | 550R | 551Y | 555I | 558V | 559E | 560T | 562I | 563T | 566L | 569Q |
| 570L | 575N | 577N | 578E | 579M | 580F | 582I | 583F | 584S | 585R | 587N | 588A | 589L | 590F |
| 592R | 593P | 595I | 596R | 597G | 598A | 599I | 601E | 602Y | 603Q | 604T | 606L | 607I | 609R |
| 610V | 611K | 613D | 614I | 617L | 621F | 639L | 640P | 641P | 646W | 655Q | 656L | 659Y | 661K |
| 662R | 663V | 664E | 665D | 666V | 667L | 668G | 669K | 671W | 672E | 674H | 677G | 680L | 681K |
| 683D | 684G | 685D | 687F | 690F | 691L | 697F | 700W | 704V | 705N | 715I | 717T | 731L | 733L |
| 736N | 744L | 764K | 747E | 748V | 752K | 755G | 756F | 757R | 758V | 759P | 760L | 762I | 763V |
| 764N | 766A | 767H | 768Q | 769A | 770N | 771Q | 774P | 776A | 778S | 779L | 7801 | 781E | 784R |
| 785T | 802L | 804A | 808K | 816E | 817G | 820L | 822W | 824S | 825Y | 826K | 828D | 830Y | 834L |
| 836E | 838V | 842Q | 843E | 848L | 849L | 862L | 865C | 867Y | 871T | 876L | 880Q | 883V | 884D |
| 886L | 888L | 891Y | 892S | 893N | 894L | 897W | 898V | 899N | 901L | 902D | 906E | 909L | 912R |
| 920W | 921T | 951P | 955N | 957V | 963T | 965Q | 968Y | 971P | 975E | 977R | 987W | 1001R | 1003Q |
| 1017Y | 1019N | 1021L | 1025P | 1027G | 1031L | 1032E | 1035Y | 1048Y | 1052W | 1053L | 1055Y | 1056Q | 1058L |
| 1059W | 1062Q | 1063A | 1070L | 1071G | 1077W | 1081L | 1084I | 1088R | 1091F | 1092D | 1096T | 1102P | 1107Y |
| 1109K | 1111Q | 1113K | 1117K | 1118Y | 1119D | 1121W | 1122H | 1123K | 1124E | 1126L | 1128K | 1129F | 1130G |
| 1134G | 1137W | 1140F | | | | | | | | | | | |

Explanation of amino acid single letter code:
A = Ala
C = Cys
D = Asp
E = Glu
F = Phe
G = Gly
H = His
I = Ile
N = Asn
P = Pro
Q = Gln
R = Arg
S = Ser
T = Thr
V = Val
K = Lys
L = Leu
M = Met
W = Trp
Y = Tyr
Binding Domains
P150d = binding domain for p150 protein
Tctex1d = binding domain for Tctex1 protein
10kDad = binding domain for 10kDa light chain
2Bd = binding domain for 2B light chain protein

TABLE 20
Conserved amino acid residues in binding domains p105d, Tctex1d,
10kDad, and 2Bd of cytoplasmic dynein intermediate chain1 of
mouse (Mus musculus), rat (Rattus norvegicus), and human (Homo sapiens)
conserved residues numbered as human cytoplasmic dynein heavy
chain 1 amino acid positions
A. Conserved amino acid residues in binding domain p150d

| | | | | | | | | | | | | | |
|---|---|---|---|---|---|---|---|---|---|---|---|---|---|
| 1M | 2S | 3D | 4K | 5S | 6D | 7L | 8K | 9A | 10E | 11L | 12E | 13R | 14K |
| 15K | 16Q | 17R | 18L | 19A | 20Q | 21I | 22R | 23E | 24E | 25K | 26K | 27R | 28K |
| 29E | 30E | 31E | 32R | 33K | 34K | 35K | 36E | 37A | 38D | 39M | 40Q | 41Q | 42K |
| 43K | 44E | 45P | 46V | 48D | 49D | 50S | 51D | 52L | 53D | 54R | 55K | 56R | 57R |
| 58E | 59T | 60E | 61A | 62L | 63L | 64Q | 65S | 66I | 67G | 68I | 69S | 70P | 71E |
| 72P | 73P | 74L | 75V | 93P | 94T | 95P | 96M | 97S | 98P | 99S | 100S | 101K | 102S |
| 103V | 104S | 105T | 106P | 107S | 109A | 110G | 111S | 112Q | 113D | 116D | 117L | 118G | 119P |
| 120L | 121T | 122R | 123T | 124L | 125Q | 126W | 127D | 128T | 129D | 130P | 131S | 132V | 133L |
| 134Q | 135L | 136Q | 137S | 138D | 139S | 140E | | | | | | | |

B. Conserved amino acid residues in binding domain Tctex1d

| | | | | | | | | | | | | | |
|---|---|---|---|---|---|---|---|---|---|---|---|---|---|
| 157F | 158L | 159P | 160R | 161E | 162V | 163V | 164S | 165Y | 166S | 167K | 168E | 169T | 170Q |
| 171T | 172P | 173L | 174A | | | | | | | | | | |

C. Conserved amino acid residues in binding domain 10kDad

| | | | | | | | | | | |
|---|---|---|---|---|---|---|---|---|---|---|
| 164S | 165Y | 166S | 167K | 168E | 169T | 170Q | 171T | 172P | 173L | 174A |

D. Conserved amino acid residues in binding domain 2Bd

| | | | | | | | | | | | | | |
|---|---|---|---|---|---|---|---|---|---|---|---|---|---|
| 260L | 261E | 262E | 263K | 264D | 265G | 266D | 267V | 268Q | 269A | 270G | 271A | 272N | 273L |
| 274S | 275F | 276N | 277R | 278Q | 279F | 280Y | 281D | 282E | 283H | 284W | 285S | 286K | 287H |
| 288R | 289V | 290V | 291T | 292C | 293M | 294D | 295W | 296S | 297L | 298Q | 299Y | 300P | 301E |
| 302L | 303M | 304V | 305A | 306S | 307Y | 309N | 310N | 311E | 312D | 313A | 314P | 315H | 316E |
| 317P | 318D | 319G | 320V | 321A | 322L | 323V | 324W | 325N | 326M | 327K | 328F | 329k | 330K |
| 331T | | | | | | | | | | | | | |

Explanation of amino acid single letter code:
A = Ala
C = Cys
D = Asp
E = Glu
F = Phe
G = Gly
H = His
I = Ile
N = Asn
P = Pro
Q = Gln
R = Arg
S = Ser
T = Thr
V = Val
K = Lys
L = Leu
M = Met
W = Trp
Y = Tyr
Binding Domains
P150d = binding domain for p150 protein
Tctex1d = binding domain for Tctex1 protein
10kDad = binding domain for 10kDa light chain
2Bd = binding domain for 2B light chain protein

TABLE 21
Conserved amino acid residues in binding domains p150d, Tctex1d,
10kDad, and 2Bd of cytoplasmic dynein intermediate chain 2 of
mouse (Mus musculus), rat (Rattus norvegicus), and human (Homo sapiens
conserved residues numbered as human cytoplasmic dynein heavy
chain 2 amino acid positions
A. Conserved amino acid residues in binding domain p150d

| | | | | | | | | | | | | | |
|---|---|---|---|---|---|---|---|---|---|---|---|---|---|
| 1M | 2S | 3D | 4K | 5S | 7L | 8K | 9A | 10E | 11L | 12E | 13R | 14K | 15K |
| 16Q | 17R | 18L | 19A | 20Q | 21I | 22R | 23E | 24E | 25K | 26K | 27R | 28K | 29E |
| 30E | 31E | 32R | 33K | 34K | 35K | 36E | 37T | 38D | 39Q | 40K | 41K | 42E | 43A |
| 47V | 48Q | 49E | 50E | 51S | 52D | 53L | 54E | 55K | 56K | 57R | 58R | 59E | 60A |
| 61E | 62A | 63L | 64L | 65Q | 66S | 67M | 68G | 69L | 70T | 73S | 74P | 75I | 76V |
| 77P | 84P | 85P | 86M | 87S | 88P | 89S | 90S | 91K | 92S | 93V | 94S | 95T | 96P |
| 97S | 98E | 99A | 100G | 101S | 102Q | 103D | 104S | 105G | 106D | 107G | 108A | 109V | 110G |
| 111S | 112R | 133R | 134G | 135P | 136I | 137K | 138L | 139G | 140M | 141A | 142K | 143I | 144T |
| 145Q | 146V | 147D | 148F | 149P | | | | | | | | | |

B. Conserved amino acid residues in binding domain Tctex1

| | | | | | | | | | | | | | |
|---|---|---|---|---|---|---|---|---|---|---|---|---|---|
| 148F | 149P | 150P | 151R | 152E | 153I | 154V | 155T | 156Y | 157T | 158K | 159E | 160T | 161Q |
| 162T | 163P | 164V | 165M | | | | | | | | | | |

C. Conserved amino acid residues in binding domain 10kDad

| | | | | | | | | | | |
|---|---|---|---|---|---|---|---|---|---|---|
| 155T | 156Y | 157T | 158K | 159E | 160T | 161Q | 162T | 163P | 164V | 165M |

D. Conserved amino acid residues in binding domain 2Bd

| | | | | | | | | | | | | | |
|---|---|---|---|---|---|---|---|---|---|---|---|---|---|
| 252L | 253E | 254D | 255K | 256E | 257G | 258E | 259I | 260Q | 261A | 262G | 263A | 264K | 265L |
| 266S | 267L | 268N | 269R | 270Q | 271F | 272F | 273D | 274E | 275R | 276W | 277S | 278K | 279H |
| 280R | 281V | 282V | 283S | 284C | 285I | 286D | 287W | 288S | 289S | 290Q | 291Y | 291P | 293E |
| 294L | 295L | 296V | 297A | 298S | 299Y | 300N | 301N | 302N | 303E | 305A | 306P | 307h | 308E |
| 309P | 310D | 311G | 312V | 313A | 314L | 315V | 316W | 317N | 318M | 319K | 320Y | 321K | 322K |
| 323T | | | | | | | | | | | | | |

Explanation of amino acid single letter code:
A = Ala
C = Cys
D = Asp
E = Glu
F = Phe
G = Gly
H = His
I = Ile
N = Asn
P = Pro
Q = Gln
R = Arg
S = Ser
T = Thr
V = Val
K = Lys
L = Leu
M = Met
W = Trp
Y = Tyr
Binding Domains
P150d = binding domain for p150 protein
Tctex1d = binding domain for Tctex1 protein
10kDad = binding domain for 10kDa light chain
2Bd = binding domain for 2B light chain protein

TABLE 22
Conserved amino acid residues in binding domains Microd, Arp1d,
and Dicd of p150 of mouse (M. musculus), rat (rattus norvegicus) and human (Homo sapiens).
conserved residues numbered as human p150 amino acid positions
A. Conserved amino acid residues in binding domain Microd

| | | | | | | | | | | | | | |
|---|---|---|---|---|---|---|---|---|---|---|---|---|---|
| 39G | 40H | 41R | 42G | 43T | 44V | 45A | 46Y | 47V | 48G | 49A | 50T | 51L | 52F |
| 53A | 54T | 55G | 56K | 57W | 58V | 59G | 60V | 61I | 62L | 63D | 64E | 65A | 66K |
| 67G | 68K | 69N | 70D | 71G | 72T | 73V | 74Q | 75G | 76R | 77K | 78Y | 79F | 80T |
| 81C | 82D | 83E | 84G | 85H | 86G | 87I | 88F | 89V | 90R | 91Q | 92S | 93Q | 94I |
| 95Q | 95Q | 96V | 97F | 98E | 99D | 100G | 101A | 102D | 103T | 104T | 105S | 106P | 107E |
| 107E | 108T | 109P | HOD | 111S | 112S | 113A | 114S | 115K | 117L | 118K | 119R | 120E | 121G |
| 123D | 126A | 127K | 128T | 129S | 130K | 131L | 132R | 133G | 134L | 135K | 136P | 137K | 138K |
| 139A | 140P | 141T | 142A | 143R | 144K | 145T | 146T | 147T | 148R | 149R | 150P | | |

B. Conserved amino acid residues in binding domain ARP1

| | | | | | | | | | | | | |
|---|---|---|---|---|---|---|---|---|---|---|---|---|
| 1006R | 1007K | 1008K | 1009E | 1010K | 1012F | 1013E | 1014E | 1015T | 1016M | 1017D | 1018A | 1019L |
| 10200 | 1021A | | | | | | | | | | | |

C. Conserved amino acid residues in binding domain Dicd.

| | | | | | | | | | | | | | |
|---|---|---|---|---|---|---|---|---|---|---|---|---|---|
| 133G | 134L | 135K | 136P | 137K | 138K | 139A | 140P | 141T | 142A | 143R | 144K | 145T | 146T |
| 147T | 148R | 149R | 150P | 151K | 152P | 153T | 154R | 155P | 156A | 157S | 158T | 159G | 160V |
| 161A | 162G | 164S | 165S | 166S | 167L | 168G | 169P | 170S | 171G | 172S | 173a | 174S | 175A |
| 176G | 177E | 178L | 179S | 180S | 181S | 182E | 183P | 184S | 185T | 186P | 187A | 188Q | 189T |
| 190P | 191L | 192A | 193A | 194P | 195I | 1961 | 197P | 198T | 199P | 201L | 202T | 203S | 204P |
| 205G | 206A | 208P | 209P | 210L | 211P | 212S | 213P | 214S | 215K | 216E | 217E | 218E | 219G |
| 220L | 221R | 223Q | 224V | 225R | 226D | 227L | 228E | 229E | 230K | 231L | 232E | 233T | 234L |
| 235R | 236L | 237K | 238R | 240E | 241DE | 242K | 243A | 244K | 245L | 246K | 247E | 248L | 249E |
| 250K | 251H | 252K | 2531 | 254Q | 255L | 256E | 2570 | 258V | 259Q | 260E | 261W | 262K | 263S |
| 264K | 265M | 266Q | 267E | 268Q | 269Q | 270A | 271D | 272L | 273Q | 274R | 275R | 276L | 277K |
| 278E | 279A | 281K | 282E | 283A | 284K | 285E | 286A | 287L | 288E | 289A | 290K | 291E | 292R |
| 293Y | 294M | 295E | 296E | 297M | 298A | 299D | 300T | 301A | 302D | 303A | 304I | 305E | 306M |
| 307A | 308T | 309L | 310D | 311K | 312E | 313M | 314A | 315E | 316E | 317R | 318A | 319E | 320S |
| 321L | 322Q | 323Q | 324E | 325V | 326E | 327A | 328L | 329K | 330E | 331R | 332V | 333D | 334E |
| 335L | 336T | 337T | 338D | 339L | 340E | 341I | 342L | 343K | 344A | 345E | 346I | 347E | 348E |
| 349K | 350G | 351S | 352D | 353G | 354A | 355A | 356S | 357S | 358Y | 359Q | 360L | 361K | 362Q |
| 363L | 364E | 365E | 366Q | 367N | 368A | 369R | 370L | 371K | 372D | 373A | 374L | 375V | 376R |
| 377M | 378R | 379D | 380L | 381S | 382S | 383S | 384E | 385K | 386Q | 387E | 388H | 389V | 390K |
| 391L | 392Q | 393K | 394L | 395M | 396E | 397K | 398K | 399N | 4000 | 401E | 402L | 403E | 404V |
| 405V | 406R | 407Q | 408Q | 409R | 410E | 411R | 412L | 413q | 414E | 415E | 416L | 417S | 418Q |
| 419A | 420E | 421S | 422T | 423I | 424D | 425E | 426L | 427K | 428E | 429Q | 430V | 431D | 432A |
| 433A | 434L | 435G | 436A | 437E | 438E | 439M | 440V | 441E | 442M | 443L | 444T | 445D | 446R |
| 447N | 448L | 449N | 450L | 451E | 452E | 453K | 454V | 455R | 456E | 457L | 458R | 459E | 460T |
| 461V | 462G | 463D | 464L | 465E | 466A | 467M | 468N | 469E | 470M | 471N | 472D | 474L | 475Q |
| 476E | 477N | 478A | 479R | 480E | 481T | 482E | 483L | 484E | 485L | 486R | 487E | 488Q | 489L |
| 490D | 491M | 492A | 493G | 494A | 495R | 496V | 497R | 498E | 499A | 500Q | 501K | 502R | 503V |
| 504E | 505A | 506A | 507Q | 508E | 509T | 510V | 511A | 512D | 513Y | 514Q | 515Q | 516T | 517I |
| 518K | 519K | 520Y | 521R | 522Q | 523L | 524T | 525A | 526H | 527L | 5280 | 529D | 530V | 531NR |
| 532R | 533E | 534L | 535T | 536N | 537Q | 538Q | 539E | 540A | 541S | 542V | 543E | 544R | 5450 |
| 546Q | 547Q | 548P | 549P | 550P | 551E | 552T | 553F | | 554D | 555F | 556K | 557I | 558K |
| 559F | 560A | 561E | 562T | 563K | 564A | 565H | 566A | 567K | 568A | 569I | 570E | 571M | 572E |
| 573L | 574R | 575Q | 576M | 577E | 578V | 579A | 580Q | 581A | 582N | 583R | 584H | 585M | 586S |
| 587L | 588L | 589T | 590A | 591F | 592M | 593P | 594D | 595S | 596F | 597L | 598R | 599P | 600G |
| 601G | 602D | 603H | 604D | 605C | 606V | 607L | 608V | 609L | 610L | 611L | 612M | 613P | 614R |
| 615L | 616I | 617C | 618K | 619A | 620E | 621L | 622I | 623R | 624K | 625Q | 626A | 627Q | 628E |
| 629K | 630F | 632L | 633S | 634E | 635N | 636C | 637S | 638E | 639R | 640P | 641G | 642L | 643R |
| 644G | 645A | 646A | 647G | 648E | 649Q | 650L | 651S | 652F | 653A | 654A | 655G | 656L | 657V |
| 658Y | 659S | 660L | 661S | 662L | 663L | 664Q | 665A | 666T | 667L | 668H | 669R | 670Y | 671E |
| 672H | 673A | 674L | 675S | 676Q | 677C | 678S | 679V | 680D | 681V | 682Y | 683K | 684K | 685V |
| 686G | 687S | 688L | 689Y | 690P | 691E | 692M | 693S | 694A | 695H | 696E | 697R | 698S | 699L |
| 700D | 701F | 702L | 703I | 704E | 705L | 706L | 707H | 708K | 709D | 7100 | 711L | 712D | 713E |
| 714T | 715V | 716N | 717V | 718E | 719P | 720L | 722K | 724I | 725K | 726Y | 727Y | 728Q | 729H |
| 730L | 731Y | 733I | 734H | 735L | 736A | 737E | 738Q | 739P | 740E | 743T | 744M | 745Q | 746L |
| 747A | 748D | 749H | 750I | 751K | 752F | 753T | 754Q | 755S | 756A | 757L | 758D | 759C | 760M |
| 762V | 763E | 764V | 765G | 766R | 767L | 768R | 769A | 770F | 771L | 772Q | 773G | 774G | 7759 |
| 776E | 777A | 778T | 779D | 780I | 781A | 782L | 783L | 784L | 785R | 786D | 787L | 788E | 789T |
| 790S | 791C | 792S | 793D | 795R | 796Q | 797F | 798C | 799K | 800K | 801I | 802R | 803R | 804R |
| 805M | 806P | 807G | 808T | 809D | 810A | 811P | 812G | 813I | 814P | 815A | 816A | 817L | 818A |
| 819F | 820G | 822Q | 823V | 824S | 825D | 826T | 8271 | 828L | 829D | 830C | 831R | 832K | 833H |
| 834L | 835T | 836W | 837V | 838V | 839A | 840V | 841L | 842Q | 843E | 844V | 845A | 846A | 847A |
| 848A | 849A | 850Q | 851L | 852I | 853A | 854P | 855L | 856A | 857E | 858N | 859E | 860G | 861L |
| 863V | 864A | 865A | 866L | 867E | 868E | 869L | 870A | 871F | 872K | 873A | 874S | 875E | 876Q |
| 877I | 878Y | 879G | 881P | 882S | 883S | 884S | 885P | 886Y | 887E | 888C | 889L | 890R | 891Q |
| 892S | 893C | 895I | 896L | 897I | 898S | 899T | | | | | | | |

Explanation of amino acid single letter code:
A = Ala
C = Cys
D = Asp
E = Glu
F = Phe
G = Gly
H = His
I = Ile
N = Asn
P = Pro
Q = Gln
R = Arg
S = Ser
T = Thr
V = Val
K = Lys
L = Leu
M = Met
W = Trp
Y = Tyr
Binding Domains
P150d = binding domain for p150 protein
Tctex1d = binding domain for Tctex1 protein
10kDad = binding domain for 10kDa light chain
2Bd = binding domain for 2B light chain protein

This invention has been described in detail including the preferred embodiments thereof. However, it will be appreciated that those skilled in the art, upon consideration of this disclosure, may make modifications and improvements thereon without departing from the spirit and scope of the invention as set forth in the claims. All references, patents, patent applications and Genbank references recited in this patent application is hereby incorporated by reference in their entirety.

TABLE 23
List of certain sequences provided in this application

| SEQ ID | |
|---|---|
| NO: | Description |
| 1 | Coding sequence (cDNA) of wild type mouse cytoplasmic |
| | dynein heavy chain1. |
| 2 | Amino acid sequence of wild type mouse cytoplasmic dynein |
| | heavy chain1. |
| 3 | Coding sequence (cDNA) of mutant mouse cytoplasmic dynein |
| | heavy chain1. |
| 4 | Amino acid sequence of mutant mouse cytoplasmic dynein |
| | heavy chain1. |
| 5 | Coding sequence (cDNA) of mutant human cytoplasmic dynein |
| | heavy chain1. |
| 6 | Amino acid sequence of mutant human cytoplasmic dynein |
| | heavy chain1. |
| 7 | ClustalW mouse sequence cytoplasmic dynein heavy chain1. |
| 8 | ClustalW rat sequence dynein heavy chain. |
| 9 | ClustalW human sequence cytoplasmic dynein heavy chain1. |
| 10 | ClustalW Drosophila sequence dynein heavy chain. |
| 11 | ClustalW Paramecium sequence dynein heavy chain. |
| 12 | ClustalW C. elegans sequence dynein heavy chain. |
| 13 | Wildtype PCR amplicon of Dnchc1, exon 13. |
| 14 | Mutant PCR amplicon of Dnchc1, exon 13. |
| 15 | Primer Dnchc1-25. |
| 16 | Primer Dnchc1-26. |
| 17 | Coding sequence (cDNA) of wild type human cytoplasmic |
| | dynein heavy chain1. |
| 18 | Amino acid sequence of wild type human cytoplasmic dynein |
| | heavy chain1. |
| 19 | Nucleotide sequence of wild type mouse cytoplasmic dynein |
| | heavy chain1, exon 13. |
| 20 | Nucleotide sequence of wild type mouse cytoplasmic dynein |
| | heavy chain1, exons 12 and 13. |
| 21 | In-frame amino acid sequence derived from SEQ ID NO: 19. |
| 22 | In-frame amino acid sequence derived from SEQ ID NO: 20. |
| 23 | In-frame amino acid sequence derived from human Dchc1 |
| | exon13. |
| 24 | In-frame amino acid sequence derived from human Dchc1 |
| | exons 12 + 13. |
| 25 | Amino acid sequence of residues positioned within ten |
| | residues of Tyr 1055 in wild type cytoplasmic dynein |
| | heavy chain1 of mouse, rat and human. |
| 26 | Primer hDnchc1-21 |
| 27 | Primer hDnchc1-22 |

1. A modified cytoplasmic dynein heavy chain1 polypeptide, wherein:
(a) the amino acid sequence of said modified cytoplasmic dynein heavy chain1 differs from the corresponding wild type sequence by substitution, deletion, or insertion of at least one amino acid in the wild type cytoplasmic dynein heavy chain1 sequence;
(b) a biological activity of said modified cytoplasmic dynein heavy chain1 polypeptide is altered by at least 10% in comparison to the activity of the wild type cytoplasmic dynein heavy chain1 polypeptide.

2. The modified cytoplasmic dynein heavy chain1 polypeptide of claim 1, wherein said biological activity is altered by at least 50% or by at least 75% in comparison to the activity of the wild type cytoplasmic dynein heavy chain1 polypeptide.

3. (canceled)

4. The modified cytoplasmic dynein heavy chain1 polypeptide of claim 1, wherein the alteration of said biological activity is any one of a reduction or an increase of activity.

5. (canceled)

6. The modified cytoplasmic dynein heavy chain1 polypeptide of claim 1, wherein the alteration of said biological activity is demonstrated by a method selected from the group consisting of:
(a) determining the relative proportions of cytoplasmic dynein heavy chain1 dimer and cytoplasmic dynein heavy chain1 monomer in a test sample;
(b) quantifying the proportion of cytoplasmic dynein complex that is fully assembled from its subunits in an in vitro assembly assay;
(c) quantifying the proportion of cytoplasmic dynein complex subunits that remain unassembled in an in vitro assembly assay;
(d) quantifying the proportion of cytoplasmic dynein heavy chain1 that binds to microtubules in vitro;
(e) assaying the rate of dimerization of the cytoplasmic dynein heavy chain1 polypeptide in vitro;
(f) assaying the rate of assembly of the cytoplasmic dynein complex from constituent subunits in vitro;
(g) assaying the rate of binding of cytoplasmic dynein heavy chains to microtubules in vitro;
(h) assaying motor activity of the cytoplasmic dynein complex in vitro; and
(i) assaying motor activity of the cytoplasmic dynein heavy chain1 in vitro; and
wherein the method compares the results provided by said modified cytoplasmic dynein heavy chains polypeptide to those provided by the corresponding wild type cytoplasmic dynein heavy chain1 polypeptide.

7. (canceled)

8. The modified cytoplasmic dynein heavy chain1 polypeptide of claim 1, wherein the alteration of said biological activity is demonstrated by assaying motor activity of the cytoplasmic dynein heavy chain1 in vitro, wherein motor activity of the cytoplasmic dynein heavy chain1 is determined in vitro by at least one parameter selected from the group consisting of:
(a) rate of total protein transport through cellular Golgi apparatus;
(b) axonal transport;
(c) retrograde axonal transport;
(d) microtubule gliding rate;
(e) phagosome movement along microtubules;
(f) rate of intracellular trafficking of membranous organelles;
(g) nuclear migration rate; and
(h) prometaphase chromosome movement.

9. The modified cytoplasmic dynein heavy chain1 polypeptide according to claim 1, wherein expression of the protein within a non-human animal model heterozygous for the modified and wild type cytoplasmic dynein heavy chain1 gene results in the animal developing at least one of the following phenotypical features:
(a) epilepsy;
(b) myoclonic cramping;
(c) neuronal excitotoxicity;
(d) cell damage in hippocampus;
(e) cell damage in cerebellum;
(f) neurodegenerative disease;
(g) under-activity or undesirable activity of endogenous cytoplasmic dynein heavy chain1;
(h) under-expression, under-production or undesirable production of endogenous cytoplasmic dynein heavy chain 1; and
(i) undesirable medical condition shown to be modulated by endogenous cytoplasmic dynein heavy chain1.

10. The modified cytoplasmic dynein heavy chain1 polypeptide according to claim 9 wherein the phenotypical features developed by said non-human animal model comprise at least one feature selected from the following group:
(a) myoclonic cramping of the fore limbs;
(b) myoclonic cramping of the hind limbs;
(c) cell damage in CA3 and CA4 sectors of hippocampus;
(d) cell damage in gyrus dentatus;
(e) neuronal damage in the upper layer of the cortex and in the Purkinje cell layer of the cerebellum;
(f) motor neuron impairment;
(g) Alzheimer's disease;
(h) Parkinson's disease;
(i) amyotropic lateral sclerosis;
(j) spinal muscular atrophy; and
(k) fiber type grouping in Musculus tibialis anterior.

11. A modified cytoplasmic dynein heavy chain1 polypeptide, wherein:
(a) the amino acid sequence of said modified cytoplasmic dynein heavy chain1 differs from the corresponding wild type sequence by substitution, deletion, or insertion of at least one amino acid residue in the wild type cytoplasmic dynein heavy chain1 sequence;
(b) the position of said amino acid residue in the cytoplasmic dynein heavy chain1 sequence is selected from:
those positions at which, in each of the wild type cytoplasmic dynein heavy chain1 reference sequences, the identity of the wild type amino acid residue is conserved; and
(c) said wild type cytoplasmic dynein heavy chain1 reference sequences consist of:
(i) SEQ ID NO: 18 (Homo sapiens);
(ii) Genbank Accession No. NP_{—}062099 (Rattus norvegicus); and
(iii) Genbank Accession No. NP_{—}084514 (Mus musculus).

12-13. (canceled)

14. The modified cytoplasmic dynein heavy chain1 polypeptide of claim 11, wherein said amino acid residue corresponds to a residue selected from the group of conserved amino acid residues between Leu302 (302L) and Phe1140 (1140F) of SEQ ID NO:2 (Mus musculus) specified in Table 19.

15. The modified cytoplasmic dynein heavy chain1 polypeptide of claim 11, wherein said amino acid residue is positioned in a cytoplasmic dynein heavy chain1 domain capable of binding a subunit of the cytoplasmic dynein complex selected from the group consisting of:
(a) cytoplasmic dynein heavy chain1;
(b) cytoplasmic dynein intermediate chain; and
(c) cytoplasmic dynein light intermediate chain.

16. The modified cytoplasmic dynein heavy chain1 polypeptide of claim 11, wherein said amino acid residue is positioned in the cytoplasmic dynein heavy chain1 dimerization binding domain.

17. The modified cytoplasmic dynein heavy chain1 polypeptide of claim 11, wherein said amino acid residue is located in a sequence selected from the group consisting of:
(a) sequences encoded by cytoplasmic dynein heavy chain1 exon 13;
(b) SEQ ID NO:21;
(c) SEQ ID NO:23;
(d) sequences encoded by cytoplasmic dynein heavy chain1 exons 12 and 13;
(e) SEQ ID NO:22; and
(f) SEQ ID NO:24.

18. The modified cytoplasmic dynein heavy chain1 polypeptide of claim 11, wherein said amino acid residue is located in a position corresponding to a residue selected from the group consisting of:
(a) in SEQ ID NO:2 (Mus musculus),
(i) residue 300 to residue 1140;
(ii) residue 416 to residue 701;
(iii) residue 649 to residue 800;
(b) in SEQ ID NO: 18 (Homo sapiens); and
(i) residue 302 to residue 1142;
(ii) residue 418 to residue 703;
(iii) residue 651 to residue 802.

19. The modified cytoplasmic dynein heavy chain1 polypeptide of claim 18 wherein said amino acid residue is located in a position corresponding to a residue selected from the group consisting of:
(a) in SEQ ID NO:2 (Mus musculus), residue 649 to residue 701; and
(b) in SEQ ID NO: 18 (Homo sapiens), residue 651 to residue 703.

20. (canceled)

21. The modified cytoplasmic dynein heavy chain1 polypeptide of claim 11, wherein the amino acid sequence of said modified cytoplasmic dynein heavy chain1 differs from the corresponding wild type sequence by substitution, deletion, or insertion of at least ten of said amino acid residues.

22. (canceled)

23. The modified cytoplasmic dynein heavy chain1 polypeptide of claim 11, wherein said amino acid residue is selected from the following group:
(a) a residue at a position within ten residues of position 1055 of the amino acid sequence as shown in SEQ ID NO:2;
(b) a residue at a position within ten residues of position 1057 of the amino acid sequence as shown in SEQ ID NO:18; and
(c) a residue corresponding to one of the residues of the amino acid sequence VEQYVKVWLQYQCLWDMQAEN (SEQ ID NO:113).

24-26. (canceled)

27. A modified cytoplasmic dynein heavy chain1 polypeptide, wherein the modification is an amino acid substitution in the wild type cytoplasmic dynein heavy chain1 sequence at a position selected from the group consisting of:
(a) a position corresponding to position 1055 of the amino acid sequence as shown in SEQ ID NO:2; and
(b) a position corresponding to position 1057 of the amino acid sequence as shown in SEQ ID NO: 19.

28. The modified cytoplasmic dynein heavy chain1 polypeptide according to claim 1, wherein the modified cytoplasmic dynein heavy chain1 polypeptide is a mammalian cytoplasmic dynein heavy chain1 polypeptide selected from the group consisting of:
(a) human cytoplasmic dynein heavy chain1 polypeptide;
(b) murine cytoplasmic dynein heavy chain1 polypeptide; and
(c) rat cytoplasmic dynein heavy chain1 polypeptide.

29-30. (canceled)

31. The modified cytoplasmic dynein heavy chain1 polypeptide of claim 1, wherein said substitution, deletion, or insertion of at least one amino acid residue in the wild type cytoplasmic dynein heavy chain1 sequence is located within a region of the polypeptide selected from the group consisting of:
(a) the region encoded by exon 12;
(b) the region encoded by exon 13;
(c) the cytoplasmic dynein heavy chain1 dimerization domain; and
(d) a cytoplasmic dynein heavy chain1 binding domain for a subunit of the cytoplasmic dynein complex distinct from cytoplasmic dynein heavy chain1.

32. The modified cytoplasmic dynein heavy chain1 polypeptide of claim 1, wherein said deletion is of more than one amino acid residue in the wild type cytoplasmic dynein heavy chain1 sequence and extends into a region of the polypeptide selected from the group consisting of:
(a) exon 12;
(b) exon 13;
(c) the cytoplasmic dynein heavy chain1 dimerization domain; and
(d) a cytoplasmic dynein heavy chain1 binding domain for a subunit of the cytoplasmic dynein complex distinct from cytoplasmic dynein heavy chain1.

33. The modified cytoplasmic dynein heavy chain1 polypeptide according to claim, wherein the amino acid substitution replaces a Tyr residue.

34. The modified cytoplasmic dynein heavy chain1 polypeptide according to claim 1, wherein said amino acid substitution is with an amino acid residue selected from the group consisting of Met, Leu, Ile, Val and Cys.

35. The modified cytoplasmic dynein heavy chain1 polypeptide according to claim 34, wherein the amino acid substitution is with a Cys residue.

36. The modified cytoplasmic dynein heavy chain1 polypeptide according to claim 35, wherein the modified cytoplasmic dynein heavy chain1 polypeptide has an amino acid sequence selected from the group consisting of SEQ ID NO:4 and SEQ ID NO:6.

37. A cytoplasmic dynein heavy chain1 polypeptide comprising the polypeptide according to claim 1.

38. A murine cytoplasmic dynein heavy chain1 polypeptide having an amino acid sequence of SEQ ID NO:4.

39. A human cytoplasmic dynein heavy chain1 polypeptide having an amino acid sequence selected from the group consisting of:
(a) the amino acid sequence as shown in SEQ ID NO:6; and
(b) the amino acid sequence as shown in SEQ ID NO: 18.

40-43. (canceled)

44. A composition comprising:
(a) a polypeptide selected from the group consisting of:
(i) the cytoplasmic dynein heavy chain1 polypeptide of claim 1;
(ii) the in-frame amino acid sequence derived from exon 12 of Dchc;
(iii) the in-frame amino acid sequence derived from exon 13 of Dchc; and
(iv) the in-frame amino acid sequence derived from exons 12 and 13 of Dchc; and
(b) a pharmaceutically acceptable carrier.

45. (canceled)

46. A polynucleotide, preferably isolated, comprising a nucleic acid sequence encoding a cytoplasmic dynein heavy chain1 polypeptide of claim 1.

47-48. (canceled)

49. A polynucleotide, preferably isolated, comprising a nucleic acid sequence encoding a polypeptide comprising an amino acid sequence selected from the following group:
(a) SEQ ID NO: 18;
(b) SEQ ID NO:21;
(c) SEQ ID NO: 22;
(d) SEQ ID NO: 23; and
(e) SEQ ID NO:24.

50-62. (canceled)

63. A medicament for the prevention, treatment or amelioration in a mammal of at least one medical condition selected from the group consisting of:
(a) epilepsy;
(b) myoclonic cramping;
(c) neuronal excitotoxicity;
(d) cell damage in hippocampus;
(e) cell damage in cerebellum;
(f) neurodegenerative disease;
(g) under-activity or over-activity or undesirable activity of endogenous cytoplasmic dynein heavy chain1;
(h) under-expression or over-expression, under-production or over-production or undesirable production of endogenous cytoplasmic dynein heavy chain1; and
(i) undesirable medical condition shown to be modulated by endogenous cytoplasmic dynein heavy chain1;
wherein said medicament comprises a polypeptide selected from the group consisting of:
(i) a cytoplasmic dynein heavy chain1 polypeptide;
(ii) the in-frame amino acid sequence derived from exon 12 of mouse or human Dchc;
(iii) the in-frame amino acid sequence derived from exon 13 of mouse or human Dchc; and
(iv) the in-frame amino acid sequence derived from exons 12 and 13 of mouse or human Dchc.

64. The medicament according to claim 63 wherein said medical condition comprises at least one feature selected from the following group:
(a) myoclonic cramping of the fore limbs;
(b) myoclonic cramping of the hind limbs;
(c) cell damage in CA3 and CA4 sectors of hippocampus;
(d) cell damage in gyrus dentatus;
(e) neuronal damage in the upper layer of the cortex and in the Purkinje cell layer of the cerebellum;
(f) motor neuron impairment;
(g) Alzheimer's disease;
(h) Parkinson's disease;
(i) amyotropic lateral sclerosis;
(j) spinal muscular atrophy; and
(k) fiber type grouping in Musculus tibialis anterior.

65. The medicament according to claim 63, wherein said polypeptide is a wild type mammalian cytoplasmic dynein heavy chain1 polypeptide.

66. The medicament according to claim 65, wherein said polypeptide is human cytoplasmic dynein heavy chain1 polypeptide.

67. The medicament according to claim 63, wherein said polypeptide has an amino acid sequence selected from the following group:
(i) SEQ ID NO:2;
(ii) SEQ ID NO: 18;
(iii) SEQ ID NO:21;
(iv) SEQ ID NO:22;
(v) SEQ ID NO:23; and
(vi) SEQ ID NO:24.

68-74. (canceled)

75. A medicament for the prevention, treatment or amelioration in a mammal of at least one medical condition selected from the group consisting of:
(a) over-activity or undesirable activity of endogenous cytoplasmic dynein heavy chain1;
(b) over-expression, over-production or undesirable production of endogenous cytoplasmic dynein heavy chain1; and
(c) undesirable medical condition shown to be modulated by endogenous cytoplasmic dynein heavy chain1;
wherein the medicament comprises a polynucleotide selected from the group consisting of:
(i) a polynucleotide according to claim 46;
(ii) a polynucleotide comprising a nucleic acid sequence encoding an in-frame amino acid sequence selected from exon 12 of mouse or human Dchc;
(iii) a polynucleotide comprising a nucleic acid sequence encoding an in-frame amino acid sequence selected from exon 13 of mouse or human Dchc;
(iv) a polynucleotide comprising a nucleic acid sequence encoding an in-frame amino acid sequence selected from exons 12 and 13 of mouse or human Dchc; and
(v) a polynucleotide comprising a nucleic acid sequence encoding a polypeptide comprising an amino acid sequence selected from the following group:
(a) SEQ ID NO:4;
(b) SEQ ID NO:6;
(c) SEQ ID NO:21;
(d) SEQ ID NO:22;
(e) SEQ ID NO:23; and
(D) SEQ ID NO:24.

76-78. (canceled)

79. A method for the prevention, treatment or amelioration of a cytoplasmic dynein heavy chain I-mediated medical condition in a mammalian subject, particularly a human subject, comprising the step of administering to the subject a cytoplasmic dynein heavy chain1 polypeptide of claim 1.

80. (canceled)

81. A method of preventing, treating or ameliorating a cytoplasmic dynein heavy chain1-mediated medical condition in a mammalian subject, particularly a human subject, comprising the step of administering to the subject the composition of claim 44.

82. A method of diagnosing in a mammalian subject, particularly a human subject, at least one of:
(a) a cytoplasmic dynein heavy chain1-mediated medical condition;
(b) a susceptibility to develop a cytoplasmic dynein heavy chain 1-mediated medical condition;
wherein the method comprises the steps of:
(i) isolating a biological sample from the subject;
(ii) comparing expression levels of cytoplasmic dynein heavy chain1 in the subject sample against a reference value, wherein said reference value is obtained by performing the method with a wild type sample equivalent to the biological sample from the subject, wherein a positive diagnosis is indicated by determining a cytoplasmic dynein heavy chain1 expression level in the subject sample that differs from that in the wild type sample by a factor selected from the group consisting of:
(a) expression in subject sample being less than 50% of expression in wild type sample;
(b) expression in subject sample being less than 20% of expression in wild type sample;
(c) expression in subject sample being less than 5% of expression in wild type sample;
and wherein the expression level of cytoplasmic dynein heavy chain1 in the subject sample is at least 0.5% of that in the wild type sample.

83-84. (canceled)

85. The method of claim 82, wherein said expression level of cytoplasmic dynein heavy chain1 is determined by detecting the existence of a mutation in a nucleic acid sequence controlling the level of expression of the cytoplasmic dynein heavy chain1 polypeptide.

86. A method of diagnosing in a mammalian subject, particularly a human subject, at least one of:
(a) a cytoplasmic dynein heavy chain1-mediated medical condition;
(b) a susceptibility to develop a cytoplasmic dynein heavy chain 1-mediated medical condition;
wherein the method comprises the steps of:
(i) isolating a sample of nucleic acid from the subject; and
(ii) determining that said sample comprises a mutation in the nucleic acid sequence of a polynucleotide encoding the cytoplasmic dynein heavy chain1 polypeptide, wherein said mutation is responsible for a substitution, deletion or insertion of at least one amino acid residue in the encoded polypeptide.

87. The method of claim 86, wherein said method comprises the additional step of identifying said mutation.

88. (canceled)

89. The method of claim 86, wherein the method comprises the additional step of determining that the cytoplasmic dynein heavy chain1 encoded by said polynucleotide is a modified cytoplasmic dynein heavy chain1 polypeptide of claim 1.

90-91. (canceled)

92. The method of claim 86, wherein said method comprises the additional step of confirming that said mutation is associated with at least one of:
(a) a cytoplasmic dynein heavy chain1-mediated medical condition;
(b) a susceptibility to develop a cytoplasmic dynein heavy chain 1-mediated medical condition;
wherein said confirmation is obtained by an analysis selected from the group consisting of:
(i) a structural analysis of cytoplasmic dynein heavy chain1 comprising said mutation;
(ii) assaying a biological function of cytoplasmic dynein heavy chain1 comprising said mutation,
wherein said analysis comprises at least one method selected from the group consisting of:
(a) assaying whether the cytoplasmic dynein heavy chain1 comprising said mutation is capable of dimerization;
(b) assaying whether the cytoplasmic dynein heavy chain1 comprising said mutation binds a recombinantly produced peptide corresponding to the dimerization domain of cytoplasmic dynein heavy chain1, preferably corresponding to the dimerization domain of human cytoplasmic dynein heavy chain1;
(c) assaying whether the cytoplasmic dynein heavy chain1 comprising said mutation binds other components of the dynein complex selected from the group comprising:
(i) dynein intermediate chain;
(ii) dynein light intermediate chain;
(iii) dynein light chain;
(iv) dynactin;
(v) dynamitin;
(d) assaying whether the cytoplasmic dynein heavy chain1 comprising said mutation is capable of integration into an intact dynein complex;
(e) assaying whether formation of intact dynein complex is inhibited in the presence of the cytoplasmic dynein heavy chain1 comprising said mutation;
(f) assaying whether the cytoplasmic dynein heavy chain1 comprising said mutation is capable of actin movement; and
(g) assaying whether the cytoplasmic dynein heavy chain1 comprising said mutation is capable of axonal transport.

93-95. (canceled)

96. The method of claim 86, wherein the mutation is located in the nucleic acid sequence encoding cytoplasmic dynein heavy chain1, wherein cytoplasmic dynein heavy chain1 is selected from SEQ ID NO:17.

97. The method of claim 86, wherein the mutation is located in a selected region of the nucleic acid sequence encoding cytoplasmic dynein heavy chain1, wherein said selected region is selected from the group consisting of:
(a) exon 12;
(b) exon 12 and exon 13; and
(c) the dimerization binding site of cytoplasmic dynein heavy chain1.

98. The method of claim 97, wherein the mutation is located in cytoplasmic dynein heavy chain1, wherein said selected region comprises the amino acid sequences, selected from the group consisting of:
(a) SEQ ID NO:23;
(b) SEQ ID NO:24; and
(c) residues 302 to residues 1142 in SEQ ID NO: 18.

99. The method of claim 86, wherein said medical condition is a neurodegenerative disease.

100. The method of claim 99, wherein said neurodegenerative disease is a progressive neurodegenerative disease and is selected from the group of Alzheimer's disease, Parkinson's disease, amyotropic lateral sclerosis and spinal muscular atrophy.

101-121. (canceled)

122. A method of identifying a protein or nucleic acid marker indicative of an increased risk of a mammalian subject, particularly a human subject, of developing a neurodegenerative disease, said method comprising the step of analyzing a test sample derived from said subject for the presence of a difference compared to a similar test sample if derived from a subject of the same species unaffected by or known not to be at risk of developing said disease, wherein said difference is indicative of the presence of a mutation in an allele of a gene coding for a protein, which is a subunit of the dynactin/dynein complex.

123. A method of identifying a protein or nucleic acid marker indicative of an association of a neurodegenerative disease in a mammalian subject, particularly a human subject, with a mutation in an allele of a gene coding for a protein, which is a subunit of the dynactin/dynein complex, said method comprising the step of analyzing a test sample derived from said subject for the presence of a difference compared to a similar test sample if derived from a subject of the same species, unaffected by or known not to be at risk of developing said disease, wherein said difference is indicative of the presence of a mutation in an allele of a gene coding for a protein, which is a subunit of the dynactin/dynein complex.

124. The method of claim 122, wherein said test sample is analyzed for a difference compared to similar test samples if derived from a group of mammalian subjects of the same species as said subject, which subjects are unaffected by, or known not to be at risk of developing, said neurodegenerative disease.

125. The method according to claim 122, wherein said mammalian subject whose test sample is analyzed has a neurodegenerative disease or is known or suspected to be at risk of developing a neurodegenerative disease.

126-151. (canceled)

152. A method for identifying a predisposition of a mammalian subject, particularly a human subject, for developing a neurodegenerative disease, said method comprising the step of determining whether a test sample derived from said subject indicates the presence of a mutation in an allele of a gene coding for a protein, which is a subunit of the dynactin/dynein complex, indicative of an increased risk of said subject of developing said neurodegenerative disease.

153. (canceled)

154. A method for determining whether a neurodegenerative disease in a mammalian subject, particularly a human subject, is associated with a mutation in an allele of a gene coding for a protein, which is a subunit of the dynactin/dynein complex, said method comprising the step of determining whether a test sample derived from said subject indicates the presence of a mutation in an allele of a gene coding for said protein.

155. The method according to claim 152, wherein said neurodegenerative disease is selected from the group consisting of Alzheimer's Disease, Parkinson's Disease, and Huntington's Disease.

156. The method according to claim 152, wherein said neurodegenerative disease is a motorneuron degenerative disease.

157. The method according to claim 156, wherein said motorneuron degenerative disease is selected from the group consisting of: Amyotrophic Lateral Sclerosis, Spinal Muscular Atrophy, Bulbo-Spinal Muscular Atrophy, Progressive Bulbar Palsy, Progressive Muscular Atrophy, and Primary Lateral Sclerosis.

158. The method according to claim 152, wherein said test sample is a nucleic acid sample, selected from the group consisting of mRNA, cDNA, and genomic DNA.

159. (canceled)

160. (canceled)

161. The method according to claim 152, wherein said test sample is a protein sample.

162. (canceled)

163. The method according to claim 152, wherein said mutation selectively affects cell types associated with or suspected to be involved in developing a neurodegenerative disease.

164. The method according to claim 152, wherein said mutation selectively affects motorneurons, preferably α-motorneurons.

165. The method according to claim 152, wherein said mutation affects a cellular process selected from the group of processes consisting of neuronal axonal transport, cellular transport, proliferation, differentiation and apoptosis.

166. The method according to claim 152, wherein said mutation leads to a disruption of, or alteration in the functional interaction within the dynactin/dynein complex in neurons, preferably in motorneurons.

167. The method according to claim 152, wherein said mutation results in a deletion or substitution by another amino acid of an amino acid encoded by said allele, or an insertion of additional amino acids not normally present in the amino acid sequence of said protein, encoded by said allele.

168. The method according to claim 167, wherein said deletion, substitution, or insertion is encoded by both alleles of the gene coding for said protein.

169. The method according to claim 167, wherein said deletion, substitution, or insertion occurs in an evolutionary conserved region of said protein.

170. The method according to claim 152, wherein said mutation results in the substitution of an amino acid which is identical between the corresponding mouse and human protein, preferably between the corresponding mouse, rat, and human protein encoded by said allele, by another amino acid.

171. The method according to claim 167, wherein said amino acid is substituted by a naturally occurring amino acid.

172. The method according to claim 167, wherein said amino acid is encoded by a codon within the open reading frame of a nucleic acid sequence selected from the group consisting of SEQ ID NO.:28-59 or SEQ ID NO.: 70-112 or SEQ ID NO.:114-126.

173. The method according to claim 167, wherein said amino acid is located in a domain of said protein, which is capable of binding to another subunit of the dynactin/dynein complex.

174. The method according to claim 173, wherein said domain is defined by an amino acid sequence selected from the group of sequences consisting of
in case the protein is mouse cytoplasmic dynein intermediate chain 1 (SEQ ID NO.: 61):
i) amino acids 147-157;
ii) amino acid 243-314;
iii) amino acids 140-157; and
iv) amino acids 1-123;
in case the protein is human cytoplasmic dynein intermediate chain 1 (SEQ ID NO.: 62):
v) amino acids 164-174;
vi) amino acids 260-331;
vii) amino acids 157-174; and
viii) amino acids 1-140;
in case the protein is mouse cytoplasmic dynein intermediate chain 2 (SEQ ID NO.: 64):
i) amino acids 1-123;
ii) amino acids 122-139;
iii) amino acids 129-130; and
iv) amino acids 226-297;
in case the protein is human cytoplasmic dynein intermediate chain 2 (SEQ ID NO.: 65):
v) amino acids 155-165;
vi) amino acids 252-323;
vii) amino acids 148-165; and
viii) amino acids 1-149;
in case the protein is mouse DCTN 1 (SEQ ID NO.: 67):
i) amino acids 39-150;
ii) amino acids 1006-1021; and
iii) amino acids 133-899; or
in case the protein is human DCTN1 (SEQ ID NO.: 68):
iv) amino acids 39-150;
v) amino acids 1006-1021; and
vi) amino acids 133-899.

175. The method according to claim 174, wherein a) if the protein is cytoplasmic dynein intermediate chain 1 (SEQ ID NOS.: 61 or 62), said amino acid is any one of the amino acids specified in Table 20;
b) if the protein is cytoplasmic dynein intermediate chain 2 (SEQ ID NOS.: 64 or 65), said amino acid is any one of the amino acids specified in Table 21;
c) if the protein is DCTN 1 (SEQ ID NOS.: 67 or 68), said amino acid is any one of the amino acids specified in Table 22.

176. An oligonucleotide suitable for identifying a mutation in an allele of a gene coding for a protein, which is a subunit of the dynactin/dynein complex.

177. An oligonucleotide according to claim 176, the nucleotide sequence of which corresponds to a nucleotide sequence within said allele, which contains a mutation that is indicative of an increased risk of a mammalian subject, particularly a human subject, of developing a neurodegenerative disease.

178. An oligonucleotide according to claim 176, which is suitable for hybridizing to the nucleic acid of said allele or a portion of its nucleic acid under stringent conditions.

179. An oligonucleotide according to claim 176, which is suitable as primer for amplifying the nucleic acid of said allele or a portion of its nucleic acid.

180. A kit for identifying a predisposition of a mammalian subject, particularly a human subject, for developing a neurodegenerative disease, or for identifying an association of a neurodegenerative disease of said subject with a mutation in an allele coding for a protein, which is a subunit of the dynactin/dynein complex, said kit comprising one or more oligonucleotides according to claim 176.

181. A kit for identifying a predisposition of a mammalian subject, particularly a human subject, for developing a neurodegenerative disease, or for identifying an association of a neurodegenerative disease of said subject with a mutation in an allele coding for a protein, which is a subunit of the dynactin/dynein complex, said kit comprising at least two oligonucleotides according to claim 179.

182. The kit according to claim 180, further comprising instructions to use the oligonucleotide or the oligonucleotides for identifying said predisposition in said subject or said association of the neurodegenerative disease of said subject with said mutation.

183. A solid support, wherein at least two oligonucleotides according to claim 175 are individually fixed to separate areas of the solid support to form an array, wherein said solid support is a glass chip, preferably a glass chip with a modified surface.

184-185. (canceled)

186. A composition comprising:
(a) the cytoplasmic dynein heavy chain1 polypeptide of claim 11; and
(b) a pharmaceutically acceptable carrier.

187. A composition comprising:
(a) the cytoplasmic dynein heavy chain1 polypeptide of claim 27; and
(b) a pharmaceutically acceptable carrier.

188. A composition comprising:
(a) the cytoplasmic dynein heavy chain1 polypeptide of claim 37; and
(b) a pharmaceutically acceptable carrier.

189. A composition comprising:
(a) the murine cytoplasmic dynein heavy chain1 polypeptide of claim 38; and
(b) a pharmaceutically acceptable carrier.

190. A composition comprising:
(a) the human cytoplasmic dynein heavy chain1 polypeptide of claim 39; and
(b) a pharmaceutically acceptable carrier.

191. A polynucleotide, preferably isolated, comprising a nucleic acid sequence encoding a cytoplasmic dynein heavy chain1 polypeptide of claim 11.

192. A polynucleotide, preferably isolated, comprising a nucleic acid sequence encoding a cytoplasmic dynein heavy chain1 polypeptide of claim 27.

193. A polynucleotide, preferably isolated, comprising a nucleic acid sequence encoding a cytoplasmic dynein heavy chain1 polypeptide of claim 37.

194. A polynucleotide, preferably isolated, comprising a nucleic acid sequence encoding a cytoplasmic dynein heavy chain1 polypeptide of claim 38.

195. A polynucleotide, preferably isolated, comprising a nucleic acid sequence encoding a cytoplasmic dynein heavy chain1 polypeptide of claim 39.

196. A medicament for the prevention, treatment or amelioration in a mammal of at least one medical condition selected from the group consisting of:
(a) epilepsy;
(b) myoclonic cramping;
(c) neuronal excitotoxicity;
(d) cell damage in hippocampus;
(e) cell damage in cerebellum;
(f) neurodegenerative disease;
(g) under-activity or over-activity or undesirable activity of endogenous cytoplasmic dynein heavy chain1;
(h) under-expression or over-expression, under-production or over-production or undesirable production of endogenous cytoplasmic dynein heavy chain1; and
(i) undesirable medical condition shown to be modulated by endogenous cytoplasmic dynein heavy chain1;
wherein said medicament comprises the polynucleotide of claim 4.

197. A medicament for the prevention, treatment or amelioration in a mammal of at least one medical condition selected from the group consisting of:
(a) epilepsy;
(b) myoclonic cramping;
(c) neuronal excitotoxicity;
(d) cell damage in hippocampus;
(e) cell damage in cerebellum;
(f) neurodegenerative disease;
(g) under-activity or over-activity or undesirable activity of endogenous cytoplasmic dynein heavy chain1;
(h) under-expression or over-expression, under-production or over-production or undesirable production of endogenous cytoplasmic dynein heavy chain1; and
(i) undesirable medical condition shown to be modulated by endogenous cytoplasmic dynein heavy chain1;
wherein said medicament comprises the polynucleotide of claim 191.

198. A medicament for the prevention, treatment or amelioration in a mammal of at least one medical condition selected from the group consisting of:
(a) epilepsy;
(b) myoclonic cramping;
(c) neuronal excitotoxicity;
(d) cell damage in hippocampus;
(e) cell damage in cerebellum;
(f) neurodegenerative disease;
(g) under-activity or over-activity or undesirable activity of endogenous cytoplasmic dynein heavy chain1;
(h) under-expression or over-expression, under-production or over-production or undesirable production of endogenous cytoplasmic dynein heavy chain1; and
(i) undesirable medical condition shown to be modulated by endogenous cytoplasmic dynein heavy chain1;
wherein said medicament comprises the polynucleotide of claim 192.

199. A medicament for the prevention, treatment or amelioration in a mammal of at least one medical condition selected from the group consisting of:
(a) epilepsy;
(b) myoclonic cramping;
(c) neuronal excitotoxicity;
(d) cell damage in hippocampus;
(e) cell damage in cerebellum;
(f) neurodegenerative disease;
(g) under-activity or over-activity or undesirable activity of endogenous cytoplasmic dynein heavy chain1;
(h) under-expression or over-expression, under-production or over-production or undesirable production of endogenous cytoplasmic dynein heavy chain1; and
(i) undesirable medical condition shown to be modulated by endogenous cytoplasmic dynein heavy chain1;
wherein said medicament comprises the polynucleotide of claim 193.

200. A medicament for the prevention, treatment or amelioration in a mammal of at least one medical condition selected from the group consisting of:
(a) epilepsy;
(b) myoclonic cramping;
(c) neuronal excitotoxicity;
(d) cell damage in hippocampus;
(e) cell damage in cerebellum;
(f) neurodegenerative disease;
(g) under-activity or over-activity or undesirable activity of endogenous cytoplasmic dynein heavy chain1;
(h) under-expression or over-expression, under-production or over-production or undesirable production of endogenous cytoplasmic dynein heavy chain1; and
(i) undesirable medical condition shown to be modulated by endogenous cytoplasmic dynein heavy chain1;
wherein said medicament comprises the polynucleotide of claim 194.

201. A medicament for the prevention, treatment or amelioration in a mammal of at least one medical condition selected from the group consisting of:
(a) epilepsy;
(b) myoclonic cramping;
(c) neuronal excitotoxicity;
(d) cell damage in hippocampus;
(e) cell damage in cerebellum;
(f) neurodegenerative disease;
(g) under-activity or over-activity or undesirable activity of endogenous cytoplasmic dynein heavy chain1;
(h) under-expression or over-expression, under-production or over-production or undesirable production of endogenous cytoplasmic dynein heavy chain1; and
(i) undesirable medical condition shown to be modulated by endogenous cytoplasmic dynein heavy chain1;
wherein said medicament comprises the polynucleotide of claim 195.

202. A medicament for the prevention, treatment or amelioration in a mammal of at least one medical condition selected from the group consisting of:
(a) epilepsy;
(b) myoclonic cramping;
(c) neuronal excitotoxicity;
(d) cell damage in hippocampus;
(e) cell damage in cerebellum;
(f) neurodegenerative disease;
(g) under-activity or over-activity or undesirable activity of endogenous cytoplasmic dynein heavy chain1;
(h) under-expression or over-expression, under-production or over-production or undesirable production of endogenous cytoplasmic dynein heavy chain1; and
(i) undesirable medical condition shown to be modulated by endogenous cytoplasmic dynein heavy chain1;
wherein said medicament comprises the polynucleotide of claim 49.

203. A medicament for the prevention, treatment or amelioration in a mammal of at least one medical condition selected from the group consisting of:
(a) over-activity or undesirable activity of endogenous cytoplasmic dynein heavy chain1;
(b) over-expression, over-production or undesirable production of endogenous cytoplasmic dynein heavy chain1; and
(c) undesirable medical condition shown to be modulated by endogenous cytoplasmic dynein heavy chain1;
wherein the medicament comprises a polynucleotide according to claim 191.

204. A medicament for the prevention, treatment or amelioration in a mammal of at least one medical condition selected from the group consisting of:
(a) over-activity or undesirable activity of endogenous cytoplasmic dynein heavy chain1;
(b) over-expression, over-production or undesirable production of endogenous cytoplasmic dynein heavy chain1; and
(c) undesirable medical condition shown to be modulated by endogenous cytoplasmic dynein heavy chain1;
wherein the medicament comprises a polynucleotide according to claim 192.

205. A medicament for the prevention, treatment or amelioration in a mammal of at least one medical condition selected from the group consisting of:
(a) over-activity or undesirable activity of endogenous cytoplasmic dynein heavy chain1;
(b) over-expression, over-production or undesirable production of endogenous cytoplasmic dynein heavy chain1; and
(c) undesirable medical condition shown to be modulated by endogenous cytoplasmic dynein heavy chain1;
wherein the medicament comprises a polynucleotide according to claim 193.

206. A medicament for the prevention, treatment or amelioration in a mammal of at least one medical condition selected from the group consisting of:
(a) over-activity or undesirable activity of endogenous cytoplasmic dynein heavy chain1;
(b) over-expression, over-production or undesirable production of endogenous cytoplasmic dynein heavy chain1; and
(c) undesirable medical condition shown to be modulated by endogenous cytoplasmic dynein heavy chain1;
wherein the medicament comprises a polynucleotide according to claim 194.

207. A medicament for the prevention, treatment or amelioration in a mammal of at least one medical condition selected from the group consisting of:
(a) over-activity or undesirable activity of endogenous cytoplasmic dynein heavy chain1;
(b) over-expression, over-production or undesirable production of endogenous cytoplasmic dynein heavy chain1; and
(c) undesirable medical condition shown to be modulated by endogenous cytoplasmic dynein heavy chain1;
wherein the medicament comprises a polynucleotide according to claim 195.

208. A medicament for the prevention, treatment or amelioration in a mammal of at least one medical condition selected from the group consisting of:
(a) over-activity or undesirable activity of endogenous cytoplasmic dynein heavy chain1;
(b) over-expression, over-production or undesirable production of endogenous cytoplasmic dynein heavy chain1; and
(c) undesirable medical condition shown to be modulated by endogenous cytoplasmic dynein heavy chain1;
wherein the medicament comprises a polynucleotide according to claim 49.

209. A method for the prevention, treatment or amelioration of a cytoplasmic dynein heavy chain1-mediated medical condition in a mammalian subject, particularly a human subject, comprising the step of administering to the subject a cytoplasmic dynein heavy chain1 polypeptide of claim 11.

210. A method for the prevention, treatment or amelioration of a cytoplasmic dynein heavy chain1-mediated medical condition in a mammalian subject, particularly a human subject, comprising the step of administering to the subject a cytoplasmic dynein heavy chain1 polypeptide of claim 27.

211. A method for the prevention, treatment or amelioration of a cytoplasmic dynein heavy chain1-mediated medical condition in a mammalian subject, particularly a human subject, comprising the step of administering to the subject a cytoplasmic dynein heavy chain1 polypeptide of claim 37.

212. A method for the prevention, treatment or amelioration of a cytoplasmic dynein heavy chain1-mediated medical condition in a mammalian subject, particularly a human subject, comprising the step of administering to the subject a cytoplasmic dynein heavy chain1 polypeptide of claim 38.

213. A method for the prevention, treatment or amelioration of a cytoplasmic dynein heavy chain 1-mediated medical condition in a mammalian subject, particularly a human subject, comprising the step of administering to the subject a cytoplasmic dynein heavy chain1 polypeptide of claim 39.

214. The method of claim 86, wherein the method comprises the additional step of determining that the cytoplasmic dynein heavy chain1 encoded by said polynucleotide is a modified cytoplasmic dynein heavy chain1 polypeptide, wherein:
(a) the amino acid sequence of said modified cytoplasmic dynein heavy chain1 differs from the corresponding wild type sequence by substitution, deletion, or insertion of at least one amino acid residue in the wild type cytoplasmic dynein heavy chain1 sequence;
(b) the position of said amino acid residue in the cytoplasmic dynein heavy chain1 sequence is selected from those positions at which, in each of the wild type cytoplasmic dynein heavy chain1 reference sequences, the identity of the wild type amino acid residue is conserved; and
(c) said wild type cytoplasmic dynein heavy chain1 reference sequences consist of:
(i) SEQ ID NO: 18 (Homo sapiens);
(ii) Genbank Accession No. NP_{—}062099 (Rattus norvegicus); and
(iii) Genbank Accession No. NP_{—}084514 (Mus musculus).

215. The method of claim 86, wherein the method comprises the additional step of determining that the cytoplasmic dynein heavy chain1 encoded by said polynucleotide is a modified cytoplasmic dynein heavy chain1 polypeptide, wherein the modification is an amino acid substitution in the wild type cytoplasmic dynein heavy chain1 sequence at a position selected from the group consisting of:
(a) a position corresponding to position 1055 of the amino acid sequence as shown in SEQ ID NO:2; and
(b) a position corresponding to position 1057 of the amino acid sequence as shown in SEQ ID NO:19.

216. The method of claim 86, wherein the method comprises the additional step of determining that the cytoplasmic dynein heavy chain1 encoded by said polynucleotide is a modified cytoplasmic dynein heavy chain1 polypeptide, wherein:
(a) the amino acid sequence of said modified cytoplasmic dynein heavy chain1 differs from the corresponding wild type sequence by substitution, deletion, or insertion of at least one amino acid in the wild type cytoplasmic dynein heavy chain1 sequence;
(b) a biological activity of said modified cytoplasmic dynein heavy chain1 polypeptide is altered by at least 10% in comparison to the activity of the wild type cytoplasmic dynein heavy chain1 polypeptide.

217. The kit according to claim 181, further comprising instructions to use the oligonucleotide or the oligonucleotides for identifying said predisposition in said subject or said association of the neurodegenerative disease of said subject with said mutation.
